SEQUENCE LISTING (139 sequences)

SEQ ID NO: 1 - RNA, 1229 nt, synthetic construct
  misc_feature 1..1229: note = GFP ledRNA
  source 1..1229: mol_type = other RNA
gggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcactatggatcaactagggatccccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgccaagctttaggtgatccaagcttgatccgggctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcacca

SEQ ID NO: 2 - RNA, 1326 nt, synthetic construct
  misc_feature 1..1326: note = GUS ledRNA
  source 1..1326: mol_type = other RNA
gggaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgtgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttcccgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgatgtggttaatcaggaactgttggcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtc

SEQ ID NO: 3 - RNA, 1485 nt, synthetic construct
  misc_feature 1..1485: note = FAD2.1 ledRNA
  source 1..1485: mol_type = other RNA
gaagatctgtagcctctcgcggtcattgtagattgggccgtaagggtcatagtgacatgcaaagcgatcataatgtcggccagaaacattgaaagccaagtacaaaggccagccaagagtaagggtgatcgtaagtgaaataacccggcctggtggattgttcaagtacttggaataccatccgagttgtgatttcggcttaggcacaaaaacctcatcgcgctcgagtgagccagtgttggagtggtggcgacgatgactatatttccaagagaagtagggcaccatcagagcagagtggaggataagcccgacagtgtcatcaacccactggtagtcactaaaggcatggtggccacattcgtgcgcaataacccaaataccagtgcaaacacaaccctgacaaatccagtaaataggccatgcaaggtagcaatcctaggcactctgctctgatggtgccctacttctcttggaaatatagtcatcgtcgccaccactccaacactggctcactcgagcgcgatgaggtttttgtgcctaagccgaaatcacaactcggatggtattccaagtacttgaacaatccaccaggccgggttatttcacttacgatcacccttactcttggctggcctttgtacttggctttcaatgtttctggccgacattatgatcgctttgcatgtcactatgacccttacggcccaatctacaatgaccgcgagaggctacagatcttcctttctgatgctggagttattggagctggttatctactatatcgtattgccttggtaaaagggctagcttggctcgtgtgtatgtatggcgtaccactcctaatcgtgaacggcttccttgtcttgatcacttatttgcagcacactcacccgtcattgcctcactacgattcatccgaatgggattggctaaggggagctttggcaaccgtcgacagagactatggcattctaaacaaggtcttccacaacatcaccgatactcacgtagtccaccatctgttctcgaccatgccacactctagagtgatgcttcatctttctccacatagatacactcttttgcttccctccacattgccttgaaaaccggggttccgtcaaattggtagtagtctccgagtaatggcttgactgcttttgttgcctccattgcattgtagtgtggcatggtcgagaacagatggtggactacgtgagtatcggtgatgttgtggaagaccttgtttagaatgccatagtctctgtcgacggttgccaaagctccccttagccaatcccattcggatgaatcgtagtgaggcaatgacgggtgagtgtgctgcaaataagtgatcaagacaaggaagccgttcacgattaggagtggtacgccatacatacacacgagccaagctagcccttttaccaaggcaatacgatatagtagataaccagctccaataactccagcatcagaaag

SEQ ID NO: 4 - DNA, 1258 bp, synthetic construct
  misc_feature 1..1258: note = DNA construct encoding GFP ledRNA
  source 1..1258: mol_type = other DNA
taatacgactcactatagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcactatggatcaactagggatccccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgccaagctttaggtgatccaagcttgatccgggctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccattgtcgggatac

SEQ ID NO: 5 - DNA, 1346 bp, synthetic construct
  misc_feature 1..1346: note = DNA construct encoding Gus ledRNA
  source 1..1346: mol_type = other DNA
taatacgactcactatagggaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgtgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttcccgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgatgtggttaatcaggaactgttggcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaac

SEQ ID NO: 6 - DNA, 1512 bp, synthetic construct
  misc_feature 1..1512: note = DNA construct encoding FAD2.1 ledRNA
  source 1..1512: mol_type = other DNA
atttaggtgacactatagaagatctgtagcctctcgcggtcattgtagattgggccgtaagggtcatagtgacatgcaaagcgatcataatgtcggccagaaacattgaaagccaagtacaaaggccagccaagagtaagggtgatcgtaagtgaaataacccggcctggtggattgttcaagtacttggaataccatccgagttgtgatttcggcttaggcacaaaaacctcatcgcgctcgagtgagccagtgttggagtggtggcgacgatgactatatttccaagagaagtagggcaccatcagagcagagtggaggataagcccgacagtgtcatcaacccactggtagtcactaaaggcatggtggccacattcgtgcgcaataacccaaataccagtgcaaacacaaccctgacaaatccagtaaataggccatgcaaggtagcaatcctaggcactctgctctgatggtgccctacttctcttggaaatatagtcatcgtcgccaccactccaacactggctcactcgagcgcgatgaggtttttgtgcctaagccgaaatcacaactcggatggtattccaagtacttgaacaatccaccaggccgggttatttcacttacgatcacccttactcttggctggcctttgtacttggctttcaatgtttctggccgacattatgatcgctttgcatgtcactatgacccttacggcccaatctacaatgaccgcgagaggctacagatcttcctttctgatgctggagttattggagctggttatctactatatcgtattgccttggtaaaagggctagcttggctcgtgtgtatgtatggcgtaccactcctaatcgtgaacggcttccttgtcttgatcacttatttgcagcacactcacccgtcattgcctcactacgattcatccgaatgggattggctaaggggagctttggcaaccgtcgacagagactatggcattctaaacaaggtcttccacaacatcaccgatactcacgtagtccaccatctgttctcgaccatgccacactctagagtgatgcttcatctttctccacatagatacactcttttgcttccctccacattgccttgaaaaccggggttccgtcaaattggtagtagtctccgagtaatggcttgactgcttttgttgcctccattgcattgtagtgtggcatggtcgagaacagatggtggactacgtgagtatcggtgatgttgtggaagaccttgtttagaatgccatagtctctgtcgacggttgccaaagctccccttagccaatcccattcggatgaatcgtagtgaggcaatgacgggtgagtgtgctgcaaataagtgatcaagacaaggaagccgttcacgattaggagtggtacgccatacatacacacgagccaagctagcccttttaccaaggcaatacgatatagtagataaccagctccaataactccagcatcagaaagcccgggactc

SEQ ID NO: 7 - DNA, 732 bp, Aequorea victoria
  source 1..732: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcccggatctc

SEQ ID NO: 8 - DNA, 1855 bp, Escherichia coli
  source 1..1855: mol_type = other DNA
atggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttcccgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgg

SEQ ID NO: 9 - DNA, 1152 bp, Nicotiana benthamiana
  source 1..1152: mol_type = other DNA
atgggagctggtggtaatatgtctcttgtaaccagcaagactggcgaaaagaagaatcctcttgaaaaggtaccaacctcaaagcctcctttcacagttggtgatatcaagaaggccatcccacctcactgctttcagcggtctctcgttcgttcgttctcctatgttgtgtatgaccttttactggtgtccgtcttctactacattgccaccacttacttccacctcctcccgtccccatattgctaccttgcatggcctatttactggatttgtcagggttgtgtttgcactggtatttgggttattgcgcacgaatgtggccaccatgcctttagtgactaccagtgggttgatgacactgtcgggcttatcctccactctgctctgatggtgccctacttctcttggaaatatagtcatcgtcgccaccactccaacactggctcactcgagcgcgatgaggtttttgtgcctaagccgaaatcacaactcggatggtattccaagtacttgaacaatccaccaggccgggttatttcacttacgatcacccttactcttggctggcctttgtacttggctttcaatgtttctggccgacattatgatcgctttgcatgtcactatgacccttacggcccaatctacaatgaccgcgagaggctacagatcttcctttctgatgctggagttattggagctggttatctactatatcgtattgccttggtaaaagggctagcttggctcgtgtgtatgtatggcgtaccactcctaatcgtgaacggcttccttgtcttgatcacttatttgcagcacactcacccgtcattgcctcactacgattcatccgaatgggattggctaaggggagctttggcaaccgtcgacagagactatggcattctaaacaaggtcttccacaacatcaccgatactcacgtagtccaccatctgttctcgaccatgccacactacaatgcaatggaggcaacaaaagcagtcaagccattactcggagactactaccaatttgacggaaccccggttttcaaggcaatgtggagggaagcaaaagagtgtatctatgtggagaaagatgaagcatcacaaggcaaaggtgttttctggtacaaaaacaaattctga

SEQ ID NO: 10 - DNA, 225 bp, synthetic construct
  misc_feature 1..225: note = GUS sense region for constructs encoding hairpin RNA moleculestargeting the GUS mRNA
  source 1..225: mol_type = other DNA
cctcgaggatcctcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgaagcttggtaccc

SEQ ID NO: 11 - DNA, 216 bp, synthetic construct
  misc_feature 1..216: note = GUS sense region for the construct encoding the hairpin RNAmolecule hpGUS[G:U]
  source 1..216: mol_type = other DNA
ccctcgagttgtgttggtatttggttagtggtagtgaagggtgaatagtttttgattaattataaattgttttattttattggttttggttgttatgaagatgtggatttgtgtggtaaaggatttgataatgtgttgatggtgtatgattatgtattaatggattggattggggttaatttttattgtattttgtattatttttatgggtacccc

SEQ ID NO: 12 - DNA, 216 bp, synthetic construct
  misc_feature 1..216: note = GUS sense region for constructs encoding the hairpin RNA moleculehpGUS[1:4]
  source 1..216: mol_type = other DNA
ccctcgagtcgggtccgcaaccgctcactgggagtcaagcgcgtacacttcgtgaataagcactaacggttgtacattagtgggtttcgtcctcaagaacatggggagttgggtgccaatggaatcgttaaggtggtgaaggtccaccacctcgctttattggtctgcattcggggcaagtccaaccctacgtcggatttcccataccggtacccc

SEQ ID NO: 13 - DNA, 216 bp, synthetic construct
  misc_feature 1..216: note = GUS sense region for constructs encoding the hairpin RNA moleculehpGUS[2:10]
  source 1..216: mol_type = other DNA
ccctcgagtcgcgtcgcgatccggtctctggcagtgttgggcgaactcttcctgatataccacaaagggttctactaaactggcttacgtcgtcatctagatgcggtgttgcgtgggtaaggattccttaacgtgcacatggtgcagcaccacgcaaaaatggactccattggggcgtactcctacgctacctcgctatacccttagcggtacccc

SEQ ID NO: 14 - DNA, 240 bp, synthetic construct
  misc_feature 1..240: note = DNA sequence of nucleotides 781-1020 of the protein coding regionof the GUS gene
  source 1..240: mol_type = other DNA
gagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgg

SEQ ID NO: 15 - RNA, 463 nt, synthetic construct
  misc_feature 1..463: note = hairpin structure (including its loop) of the hpGUS[wt] RNA
  source 1..463: mol_type = other RNA
ggatcctcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgaagcttggtaccccagcttgttgggaagctgggttcgaaatcgataagcttcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaggatcc

SEQ ID NO: 16 - RNA, 457 nt, synthetic construct
  misc_feature 1..457: note = hairpin structure (including its loop) of the hpGUS[G:U] RNA
  source 1..457: mol_type = other RNA
ctcgagttgtgttggtatttggttagtggtagtgaagggtgaatagtttttgattaattataaattgttttattttattggttttggttgttatgaagatgtggatttgtgtggtaaaggatttgataatgtgttgatggtgtatgattatgtattaatggattggattggggttaatttttattgtattttgtattatttttatgggtaccccagcttgttgggaagctgggttcgaaatcgataagcttcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaggatcc

SEQ ID NO: 17 - RNA, 457 nt, synthetic construct
  misc_feature 1..457: note = hairpin structure (including its loop) of the hpGUS[1:4] RNA
  source 1..457: mol_type = other RNA
ctcgagtcgggtccgcaaccgctcactgggagtcaagcgcgtacacttcgtgaataagcactaacggttgtacattagtgggtttcgtcctcaagaacatggggagttgggtgccaatggaatcgttaaggtggtgaaggtccaccacctcgctttattggtctgcattcggggcaagtccaaccctacgtcggatttcccataccggtaccccagcttgttgggaagctgggttcgaaatcgataagcttcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaggatcc

SEQ ID NO: 18 - RNA, 457 nt, synthetic construct
  misc_feature 1..457: note = hairpin structure (including its loop) of the hpGUS[2:10] RNA
  source 1..457: mol_type = other RNA
ctcgagtcgcgtcgcgatccggtctctggcagtgttgggcgaactcttcctgatataccacaaagggttctactaaactggcttacgtcgtcatctagatgcggtgttgcgtgggtaaggattccttaacgtgcacatggtgcagcaccacgcaaaaatggactccattggggcgtactcctacgctacctcgctatacccttagcggtaccccagcttgttgggaagctgggttcgaaatcgataagcttcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaggatcc

SEQ ID NO: 19 - DNA, 4851 bp, Arabidopsis thaliana
  source 1..4851: mol_type = other DNA
atctctctctttcgatggaactgagctctttctctctttcctcttcttttctctctctatctctatctctcgtagcttgataagagtttctctcttttgaagatccgtttctctctctctcactgagactattgttgttaggtcaacttgcgatcatggcgatttcgaaggtctgaagctgatttcgaatggtttggagatatccgtagtggttaagcatatggaagtctatgttctgctcttggttgctctgttagggcttcctccatttggaccaacttagctgaatgttgtatgatctctctccttgaagcagcaaataagaagaaggtctggtccttaacttaacatctggttactagaggaaacttcagctattattaggtaaagaaagactgtacagagttgtataacaagtaagcgttagagtggctttgtttgcctcggtgatagaagaaccgactgattcgttgttgtgtgttagctttggagggaatcagatttcgcgagggaaggtgttttagatcaaatctgtgaattttactcaactgaggcttttagtgaaccacgactgtagagttgaccttgaatcctactctgagtaattatattatcagatagatttaggatggaagctgaaattgtgaatgtgagacctcagctagggtttatccagagaatggttcctgctctacttcctgtccttttggtttctgtcggatatattgatcccgggaaatgggttgcaaatatcgaaggaggtgctcgtttcgggtatgacttggtggcaattactctgcttttcaattttgccgccatcttatgccaatatgttgcagctcgcataagcgttgtgactggtaaacacttggctcagatctgcaatgaagaatatgacaagtggacgtgcatgttcttgggcattcaggcggagttctcagcaattctgctcgaccttaccatggttgtgggagttgcgcatgcacttaaccttttgtttggggtggagttatccactggagtgtttttggccgccatggatgcgtttttatttcctgttttcgcctctttccttgaaaatggtatggcaaatacagtatccatttactctgcaggcctggtattacttctctatgtatctggcgtcttgctgagtcagtctgagatcccactctctatgaatggagtgttaactcggttaaatggagagagcgcattcgcactgatgggtcttcttggcgcaagcatcgtccctcacaatttttatatccattcttattttgctggggaaagtacatcttcgtctgatgtcgacaagagcagcttgtgtcaagaccatttgttcgccatctttggtgtcttcagcggactgtcacttgtaaattatgtattgatgaatgcagcagctaatgtgtttcacagtactggccttgtggtactgacttttcacgatgccttgtcactaatggagcaggtatttatgagtccgctcattccagtggtctttttgatgctcttgttcttctctagtcaaattaccgcactagcttgggctttcggtggagaggtcgtcctgcatgacttcctgaagatagaaatacccgcttggcttcatcgtgctacaatcagaattcttgcagttgctcctgcgctttattgtgtatggacatctggtgcagacggaatataccagttacttatattcacccaggtcttggtggcaatgatgcttccttgctcggtaataccgcttttccgcattgcttcgtcgagacaaatcatgggtgtccataaaatccctcaggttggcgagttcctcgcacttacaacgtttttgggatttctggggttgaatgttgtttttgttgttgagatggtatttgggagcagtgactgggctggtggtttgagatggaataccgtgatgggcacctcgattcagtacaccactctgcttgtatcgtcatgtgcatccttatgcctgatactctggctggcagccacgccgctgaaatctgcgagtaacagagcggaagctcaaatatggaacatggatgctcaaaatgctttatcttatccatctgttcaagaagaggaaattgaaagaacagaaacaaggaggaacgaagacgaatcaatagtgcggttggaaagcagggtaaaggatcagttggatactacgtctgttactagctcggtctatgatttgccagagaacattctaatgacggatcaagaaatccgttcgagccctccagaggaaagagagttggatgtaaagtactctacctctcaagttagtagtcttaaggaagactctgatgtaaaggaacagtctgtattgcagtcaacagtggttaatgaggtcagtgataaggatctgattgttgaaacaaagatggcgaaaattgaaccaatgagtcctgtggagaagattgttagcatggagaataacagcaagtttattgaaaaggatgttgaaggggtttcatgggaaacagaagaagctaccaaagctgctcctacaagcaactttactgtcggatctgatggtcctccttcattccgcagcttaagtggggaagggggaagtgggactggaagcctttcacggttgcaaggtttgggacgtgctgcccggagacacttatctgcgatccttgatgaattttggggacatttatatgattttcatgggcaattggttgctgaagccagggcaaagaaactagatcagctgtttggcactgatcaaaagtcagcctcttctatgaaagcagattcgtttggaaaagacattagcagtggatattgcatgtcaccaactgcgaagggaatggattcacagatgacttcaagtttatatgattcactgaagcagcagaggacaccgggaagtatcgattcgttgtatggattacaaagaggttcgtcaccgtcaccgttggtcaaccgtatgcagatgttgggtgcatatggtaacaccactaataataataatgcttacgaattgagtgagagaagatactctagcctgcgtgctccatcatcttcagagggttgggaacaccaacaaccagctacagttcacggataccagatgaagtcatatgtagacaatttggcaaaagaaaggcttgaagccttacaatcccgtggagagatcccgacatcgagatctatggcgcttggtacattgagctatacacagcaacttgctttagccttgaaacagaagtcccagaatggtctaacccctggaccagctcctgggtttgagaattttgctgggtctagaagcatatcgcgacaatctgaaagatcttattacggtgttccatcttctggcaatactgatactgttggcgcagcagtagccaatgagaaaaaatatagtagcatgccagatatctcaggattgtctatgtccgcaaggaacatgcatttaccaaacaacaagagtggatactgggatccgtcaagtggaggaggagggtatggtgcgtcttatggtcggttaagcaatgaatcatcgttatattctaatttggggtcacgggtgggagtaccctcgacttatgatgacatttctcaatcaagaggaggctacagagatgcctacagtttgccacagagtgcaacaacagggaccggatcgctttggtccagacagccctttgagcagtttggtgtagcggagaggaatggtgctgttggtgaggagctcaggaatagatcgaatccgatcaatatagacaacaacgcttcttctaatgttgatgcagaggctaagcttcttcagtcgttcaggcactgtattctaaagcttattaaacttgaaggatccgagtggttgtttggacaaagcgatggagttgatgaagaactgattgaccgggtagctgcacgagagaagtttatctatgaagctgaagctcgagaaataaaccaggtgggtcacatgggggagccactaatttcatcggttcctaactgtggagatggttgcgtttggagagctgatttgattgtgagctttggagtttggtgcattcaccgtgtccttgacttgtctctcatggagagtcggcctgagctttggggaaagtacacttacgttctcaaccgcctacagggagtgattgatccggcgttctcaaagctgcggacaccaatgacaccgtgcttttgccttcagattccagcgagccaccagagagcgagtccgacttcagctaacggaatgttacctccggctgcaaaaccggctaaaggcaaatgcacaaccgcagtcacacttcttgatctaatcaaagacgttgaaatggcaatctcttgtagaaaaggccgaaccggtacagctgcaggtgatgtggctttcccaaaggggaaagagaatttggcttcggttttgaagcggtataaacgtcggttatcgaataaaccagtaggtatgaatcaggatggacccggttcaagaaaaaacgtgactgcgtacggatcattgggttgaagaagaagaacattgtgagaaatctcatgatcaaagtgacgtcgagagggaagccgaagaatcaaaactctcgcttttgattgctcctctgcttcgttaattgtgtattaagaaaagaagaaaaaaaatggatttttgttgcttcagaatttttcgctctttttttcttaatttggttgtaatgttatgtttatatacatatatcatcatcataggaccatagctacaaaccgaatccggtttgtgtaattctatgcggaatcataaagaaatcgtcggtttgaaatgttaaatctcctaaaccggatctctgcacgtagctgacacatcgacgctag

SEQ ID NO: 20 - DNA, 1703 bp, Arabidopsis thaliana
  source 1..1703: mol_type = other DNA
gttgcaaatatataaatcaatcaaaagatttaaaacccaccattcaatcttggtaagtaacgaaaaaaaagggaagcaagaagaaccacagaaaagggggctaacaactagacacgtagatcttcatctgcccgtccatctaacctaccacactctcatcttctttttcccgtgtcagtttgttatataagctctcactctccggtatatttccaaatacacctaacttgtttagtacacaacagcaacatcaaactctaataaacccaagttggtgtatactataatggtgatggctggtgcttcttctttggatgagatcagacaggctcagagagctgatggacctgcaggcatcttggctattggcactgctaaccctgagaaccatgtgcttcaggcggagtatcctgactactacttccgcatcaccaacagtgaacacatgaccgacctcaaggagaagttcaagcgcatgtgcgacaagtcgacaattcggaaacgtcacatgcatctgacggaggaattcctcaaggaaaacccacacatgtgtgcttacatggctccttctctggacaccagacaggacatcgtggtggtcgaagtccctaagctaggcaaagaagcggcagtgaaggccatcaaggagtggggccagcccaagtcaaagatcactcatgtcgtcttctgcactacctccggcgtcgacatgcctggtgctgactaccagctcaccaagcttcttggtctccgtccttccgtcaagcgtctcatgatgtaccagcaaggttgcttcgccggcggtactgtcctccgtatcgctaaggatctcgccgagaacaatcgtggagcacgtgtcctcgttgtctgctctgagatcacagccgttaccttccgtggtccctctgacacccaccttgactccctcgtcggtcaggctcttttcagtgatggcgccgccgcactcattgtggggtcggaccctgacacatctgtcggagagaaacccatctttgagatggtgtctgccgctcagaccatccttccagactctgatggtgccatagacggacatttgagggaagttggtctcaccttccatctcctcaaggatgttcccggcctcatctccaagaacattgtgaagagtctagacgaagcgtttaaacctttggggataagtgactggaactccctcttctggatagcccaccctggaggtccagcgatcctagaccaggtggagataaagctaggactaaaggaagagaagatgagggcgacacgtcacgtgttgagcgagtatggaaacatgtcgagcgcgtgcgttctcttcatactagacgagatgaggaggaagtcagctaaggatggtgtggccacgacaggagaagggttggagtggggtgtcttgtttggtttcggaccaggtctcactgttgagacagtcgtcttgcacagcgttcctctctaaacagaacgcttgccttctatctgcctacctacctacgcaaaactttaatcctgtcttatgttttatataatataatcattatatgtttacgcaataattaaggaagaattcatttgatgtgatatgtgatatgtgctggacaggtctattcgactgtttttgtactctcttttttgtgtctttttacaatattaaatctatgggtcttgaatgacatcaaatctttgtt

SEQ ID NO: 21 - DNA, 229 bp, Arabidopsis thaliana
  source 1..229: mol_type = other DNA
cctcgaggatcctctagacctcagctagggtttatccagagaatggttcctgctctacttcctgtccttttggtttctgtcggatatattgatcccgggaaatgggttgcaaatatcgaaggaggtgctcgtttcgggtatgacttggtggcaattactctgcttttcaattttgccgccatcttatgccaatatgttgcagctcgcataagcgttaagcttggtaccc

SEQ ID NO: 22 - DNA, 218 bp, synthetic construct
  misc_feature 1..218: note = fragment comprising the 200nt sense sequence of EIN2
  source 1..218: mol_type = other DNA
cctcgagtctagattttagttagggtttatttagagaatggtttttgttttattttttgtttttttggtttttgttggatatattgattttgggaaatgggttgtaaatattgaaggaggtgtttgttttgggtatgatttggtggtaattattttgttttttaattttgttgttattttatgttaatatgttgtagtttgtataagtgttggtaccc

SEQ ID NO: 23 - DNA, 230 bp, Arabidopsis thaliana
  source 1..230: mol_type = other DNA
cctcgaggatccgttgtctgctctgagatcacagccgttaccttccgtggtccctctgacacccaccttgactccctcgtcggtcaggctcttttcagtgatggcgccgccgcactcattgtggggtcggaccctgacacatctgtcggagagaaacccatctttgagatggtgtctgccgctcagaccatccttccagactctgatggtgctctagaagcttggtaccc

SEQ ID NO: 24 - DNA, 219 bp, synthetic construct
  misc_feature 1..219: note = fragment comprising the 200nt sense sequence of CHS
  source 1..219: mol_type = other DNA
cctcgaggttgtttgttttgagattatagttgttattttttgtggtttttttgatatttattttgatttttttgttggttaggttttttttagtgatggtgttgttgtatttattgtggggttggattttgatatatttgttggagagaaatttatttttgagatggtgtttgttgtttagattatttttttagattttgatggtgtctagaggtaccc

SEQ ID NO: 25 - DNA, 219 bp, synthetic construct
  misc_feature 1..219: note = fragment comprising the 200nt antisense sequence of EIN2
  source 1..219: mol_type = other DNA
caagcttaatgtttatgtgagttgtaatatattggtataagatggtggtaaaattgaaaagtagagtaattgttattaagttatatttgaaatgagtattttttttgatatttgtaatttattttttgggattaatatatttgatagaaattaaaaggataggaagtagagtaggaattattttttggataaattttagttgaggttctagaggatccc

SEQ ID NO: 26 - DNA, 219 bp, synthetic construct
  misc_feature 1..219: note = fragment comprising the 200nt antisense sequence of CHS
  source 1..219: mol_type = other DNA
caagcttctagagtattattagagtttggaaggatggtttgagtggtagatattattttaaagatgggtttttttttgatagatgtgttagggtttgattttataatgagtgtggtggtgttattattgaaaagagtttgattgatgagggagttaaggtgggtgttagagggattatggaaggtaatggttgtgattttagagtagataatggatccc

SEQ ID NO: 27 - DNA, 300 bp, Arabidopsis thaliana
  source 1..300: mol_type = other DNA
agtaattatattatcagatagatttaggatggaagctgaaattgtgaatgtgagacctcagctagggtttatccagagaatggttcctgctctacttcctgtccttttggtttctgtcggatatattgatcccgggaaatgggttgcaaatatcgaaggaggtgctcgtttcgggtatgacttggtggcaattactctgcttttcaattttgccgccatcttatgccaatatgttgcagctcgcataagcgttgtgactggtaaacacttggctcagatctgcaatgaagaatatgacaa

SEQ ID NO: 28 - DNA, 300 bp, Arabidopsis thaliana
  source 1..300: mol_type = other DNA
tccgtatcgctaaggatctcgccgagaacaatcgtggagcacgtgtcctcgttgtctgctctgagatcacagccgttaccttccgtggtccctctgacacccaccttgactccctcgtcggtcaggctcttttcagtgatggcgccgccgcactcattgtggggtcggaccctgacacatctgtcggagagaaacccatctttgagatggtgtctgccgctcagaccatccttccagactctgatggtgccatagacggacatttgagggaagttggtctcaccttccatctcctcaagg

SEQ ID NO: 29 - DNA, 240 bp, Arabidopsis thaliana
  source 1..240: mol_type = other DNA
tcttcattgcagatctgagccaagtgtttaccagtcacaacgcttatgcgagctgcaacatattggcataagatggcggcaaaattgaaaagcagagtaattgccaccaagtcatacccgaaacgagcacctccttcgatatttgcaacccatttcccgggatcaatatatccgacagaaaccaaaaggacaggaagtagagcaggaaccattctctggataaaccctagctgaggtctc

SEQ ID NO: 30 - DNA, 300 bp, Arabidopsis thaliana
  source 1..300: mol_type = other DNA
gatggaaggtgagaccaacttccctcaaatgtccgtctatggcaccatcagagtctggaaggatggtctgagcggcagacaccatctcaaagatgggtttctctccgacagatgtgtcagggtccgaccccacaatgagtgcggcggcgccatcactgaaaagagcctgaccgacgagggagtcaaggtgggtgtcagagggaccacggaaggtaacggctgtgatctcagagcagacaacgaggacacgtgctccacgattgttctcggcgagatccttagcgatacggaggacagtac

SEQ ID NO: 31 - DNA, 4035 bp, Arabidopsis thaliana
  source 1..4035: mol_type = other DNA
atgggatctagggttccaatagaaaccatcgaagaagacggcgaattcgattgggaagcagcagtcaaagaaatcgacttggcttgtcttaaaaccacaaacgcttcttcttcttcgtcatcccatttcactcctttggctaatccaccaattacggcaaatctcactaagccacctgcgaagagacaatctactctcgataaattcatcggcagaaccgaacataaaccggagaatcatcaagttgtttccgagtgtggtgttaacgataacgataatagtcctttagttgggattgatcctgaggcagctaaaacttggatttatccagtgaatgggagtgttcctttaagagattatcagtttgctataacgaagactgctttgttttcgaatacattggtggctttgcctacgggacttggtaaaacgcttatagctgcggttgttatgtataattacttcagatggtttccacaaggtaaaatagtatttgcggcgccttctaggcctcttgtgatgcagcagattgaggcgtgtcataatattgttggaataccacaagaatggacgattgacttgacgggtcagacatgtccttcgaaaagagcttttttgtggaaaagcaaacgggttttctttgtcactccacaagtgttagagaaggatatacagtcaggaacatgtcttactaactacttggtttgcttggtgatcgacgaggcacatcgagctttagggaattattcttattgtgttgtagttcgtgagttgatggcggtaccgatacagctgagaatactggctcttactgcaactcctggatcaaagacacaggccatccagggtatcattgataatttgcagatatccacacttgaatatcgaaatgagagtgaccatgatgtttgcccttatgtccacgacagaaaattagaagtcatcgaggttcccttgggtcaagatgcagatgatgtatcgaaacgcctgtttcatgttatacgtccatatgcagtcaggcttaaaaactttggggttaatctaaatagagatatacaaactttaagtccacacgaagtacttatggcaagggataagtttcgtcaagcacctctaccaggccttccccatgtaaatcacggagatgtagaatcttgctttgcagctcttatcactctttatcatattcgtaagctcctttctagtcatggaataagaccagcgtatgagatgctagaagagaaattgaaagaagggccatttgctaggttgatgagtaagaatgaagatattaggatgacgaagcttttgatgcagcaaaggttgtcacatggagcaccaagcccaaaattgtcgaagatgttagaaatactggttgatcatttcaaagtgaaagatccgaagacatcacgggtcattattttctcaaatttcagaggaagcgtaagagacataatgaacgcattaagtaatattggagatatggtcaaagcaactgagtttattggtcaaagttcaggtaagacattgaaaggccagtcgcaaaaaattcagcaggctgttttggagaaatttagagctggggggttcaatgttattgtcgcaacatctattggtgaagaaggcttggatatcatggaagttgacctagttatatgttttgatgctaatgtatctcctctgaggatgattcaacggatgggaagaactggaaggaaaaataatggtcgagttgtagttcttgcttgtgaaggatcagaaaagaacagctatatgcgaaagcaagcaagtggacgggctattaaaaaacacatgcggaatggaggaacaaatagttttaattttcatcctagtccaaggatgattccccatgtttataagccagaagttcagcatgttgagttttcaatcaagcaattcgttccacgtggaaagaaactacaagaggagtatgccactgagactccagctttccagaaaaagcttacacctgcagagacgcatatgctcgctaagtattacaacaaccccgatgaggaaaagttgagagtgtccttaattgcgttccctcacttccagacattgccatccaaggtgcacaaagtaatgcattcacgtcaaacaggcatgttaattgacgctatgcagcacttgcaagagccaactttttcagaacagagtaaaagcttcttcactgagtttcgagctcctttgggtgaaagagaagagcttgatacaggtctgagggttactaatgatccaaaagatctacactctgtccgtgatttggaagtcaacacatcacagagaaaggcaaaacaagttgaatctcccacaagcaccttagagacaacagagaaggattacgaagaatcttcacccacacaccgttatcttttcagttcagaatgtgcatccgttgatactctggggaacgtcttcgtaatgccagttcctcttttattctttcctaatgttctggagtcagacaatacgcctctgcctaaaacagaaaaacaacattcttgccggaatacatctcacattgacttagttccagtagatacttcggaaaaacatcggcaagataatatctcatgcaagttaaaggaaagattctcgccagacggtgccagcgagacactagagactcatagccttgtgaaaaggaactccaccagagtaggtgaagatgatgtagcgaattctgttggagaaattgtgttatcatcggatgaagatgactgtgagggattggagcttagtccacggctcactaacttcatcaagagcggcattgttccagagtcacctgtctatgaccaaggggaagcgaacagagaagaagatcttgaatttcctcagctttcttcacccatgaggttcagtaacgaattggcaggagagtcttctttccctgagagaaaggttcagcataagtgcaacgattataacattgtgtctacaaccactgaattgagaactcctcagaaggaggtaggtttggccaacggaacagaatgcttggctgtttctcctattcctgaggattggagaactcccttggcgaatctgacaaacacaaacagcagcgctcgcaaagattggcgggtgagttctggagaaaagttagaaactcttcgacagcctcgcaagttgaagagactacgtagacttggagattgctcgagtgctgtaaaggagaattatcctggtattacagaggcagaccatatcagatctcgttctcgcggtaaaaagcacattagaggtaagaagaagatgatcatggatgatgatgtccaagtcttcattgacgaggaagctgaggtctcttcgggagcagagatgtcggctgatgagaacgaagatgtgactggcgattcatttgaagatagtttcatagatgacggaacaatgcctacagcaaatactcaagccgagtctggtaaagttgacatgatggctgtttacaggcgttctcttctcagccagtcaccattaccggcgagatttcgtgatttagccgcatcaagtctgagtccttattctgctggacccttgacgagaataaatgagagcagaagcgactcagataaatcattgtcttctcttcgaacaccaaaaacaacaaactctgagtcaaaccaagatgcaatgatgataggaaacctttcggtagtacaaatctcgtcagatagccggaaaaggaaatttagcttatgcaactcggcgaatgcccccgtgattaacttagaaagcaagtttgcagctcatgcacaagccacggagaaggaaagccatgaaggcgtgagaagcaatgcaggtgcgttagagtacaatgatgatgatgatgatgcattctttgcgacactagactttgatgcaatggaagcacaagccacattgttattgtcgaaacagagatccgaagcaaaagagaaagaagacgcaacggttatacctaatccaggcatgcagagaagtgatggtatggagaaagatgcaccatcttttgatcttggtctgtggtga

SEQ ID NO: 32 - DNA, 2310 bp, Brassica napus
  source 1..2310: mol_type = other DNA
atgtcaaatgaaaataaaaatataaaaactaaatttcatcctagttcaaggatgattccccatgtttataagccagaagttcagcatgttaagttttcgatcgagcaattcattccacgtggaaagaagctacaagatgagcctgccactgagactccagctttcaagaaaaagcttacaccggaagagatggatatgctcgccaagtatttcaaacccaacgaggaaaagtggagagtttccttgattgctttccctcacttccaaacattgccatccaaagtgcacaaagtaatgcattcacgccaaacaagcatattaattgatgctatgcagcatctgcaagagacaactttgacagagcaaagtaaaagtttcttcattaagtatggagctcctttggctgaaagagatgagcttgacgcaggtctgagggttggtgatgatccgaaagatttaccctcttccgatgatttggatgtcaacacatcacagagaaaggcaaaacaaattttagaatctcccacaagcacattagagactacagagaaggatttcgaagcatcttcacccacacactgttatcttttcagttcagaatgtgcgtccgttgatactctggggaaggtctttgtattgccggttcctctctcattctcttctaatgtaccagggtcagactgcgtgggaagagaaaaagaactttcttccccgaataagtcccacactgacgttgttccgatagatagttcctcaaaacatcggcaagataatatttcatgcaagttaaagcaaggattcttgccagattgtgccaacgagactttggagtcccaaagccttttgaaaaggcactccaccgatgtaggtaaaggagatatagagaattgtgctggagaaattatgatatcatcggatgaagaagacgactgtgaggatttggagcttagtccaaggctcactaacttcatcaagagtggcgttgttccagattcacctgtctatgaccaagttgcatacgaagcaaacagagaagaagaccttgatcttccacacacgagtttaactaatgaattggcagaagagccatcgacacctgagaaaaaggttcacattgcttctacggccaatgaattcagaactcctcagaaggaagaagatttagccaacgaaacagaaagcttcgctgtttctccaatgcctgaggagtggagaactcccttggcgaatatcaccaacgcaagcagcagcgctagcaaagattggcgcgtgagttcgggagaaaagtcagaaactcttcgacagcctcgcaagttgaagagacttcgtagacttggagattgctcgagtgctgtgaaggagaataatcctggtattgcaaagacagaccatatcagatctcgttctcgcagtgtaaagaacataagaggtaaaatgattctgtatttccttttgctctgtgttcaaggcaagaagaagatacgcgcggataataatgctagaatcttcattgaagcggaagctgaggtgtcttcggaatcagaaatgtcggttgatgagaacgtagatttgaccagcgattcatttgaagatagcttcatagatgacggtacaatgcctacagcaaatactcaagccgagtgtgctaaagttgacatgatggccgtttacaggtatatatcgaatcaaaacaagtctttcttctactatgatttactaagaatcataagctatggtttccacagacgttctctactcagccaatcaccattaccggcaagatttcgtgatgtagctgcatcaagtccgagtccttattcttctggtctcttgaagacaataaatgagagcagaagcgactcagataaatcattgtcttctcttagaaccccacaaacaacgaacaacgagtcaaacaaggatgcaatggccacaggagacctttcggtagcacaaatctcaacagacagccggaaaaggaaattcagcttatgcaactcagcgaatgtcccagtgattaacttggaaaacaagtttgaagctcatgcacaagccacggagaaggaaagccatgaaggtccgagaagcaatgcaggtgcatcacagtacaaggatgaggatgaagatgatgatgcattctacgcgacactggactttgatgccatggaagcgcatgcgacattgctattgtcgaaacaaaggtcagaaacgaaaacaaaagaagatgcatcggtgaaacctcatttgggcaatcagaggaatgatggtttgccgaaggatgggccatcttttgatcttggtttgtggtga

SEQ ID NO: 33 - DNA, 1822 bp, synthetic construct
  misc_feature 1..1822: note = hpFANCM-At[wt]
  source 1..1822: mol_type = other DNA
ggctcgagaaccgaattctaatacgactcactatagggtcaggaacatgtcttactaactacttggtttgcttggtgatcgacgaggcacatcgagctttagggaattattcttattgtgttgtagttcgtgagttgatggcggtaccgatacagctgagaatactggctcttactgcaactcctggatcaaagacacaggccatccagggtatcattgataatttgcagatatccacacttgaatatcgaaatgagagtgaccatgatgtttgcccttatgtccccgacagaaaattagaagtcatcgaggttcccttgggtcaagatgcagatgatgtatcgaaacgcctgtttcatgttatacgtccatatgcagtcaggcttaaaaactttggggttaatctaaatagagatatacaaactttaagtccacacgaagtacttatggcaagggataagtttcgtcaagcacctctaccaggccttccccatgtaaatcacggagatgtagaatcttgctttgcagctcttatcaggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagttgataagagctgcaaagcaagattctacatctccgtgatttacatggggaaggcctggtagaggtgcttgacgaaacttatcccttgccataagtacttcgtgtggacttaaagtttgtatatctctatttagattaaccccaaagtttttaagcctgactgcatatggacgtataacatgaaacaggcgtttcgatacatcatctgcatcttgacccaagggaacctcgatgacttctaattttctgtcggggacataagggcaaacatcatggtcactctcatttcgatattcaagtgtggatatctgcaaattatcaatgataccctggatggcctgtgtctttgatccaggagttgcagtaagagccagtattctcagctgtatcggtaccgccatcaactcacgaactacaacacaataagaataattccctaaagctcgatgtgcctcgtcgatcaccaagcaaaccaagtagttagtaagacatgttcctgaccccgggatccaagctt

SEQ ID NO: 34 - DNA, 1822 bp, synthetic construct
  misc_feature 1..1822: note = hpFANCM-At[G:U]
  source 1..1822: mol_type = other DNA
ggctcgagaaccgaattctaatacgactcactatagggttaggaatatgttttattaattatttggtttgtttggtgattgatgaggtatattgagttttagggaattatttttattgtgttgtagtttgtgagttgatggtggtattgatatagttgagaatattggtttttattgtaatttttggattaaagatataggttatttagggtattattgataatttgtagatatttatatttgaatattgaaatgagagtgattatgatgtttgtttttatgtttttgatagaaaattagaagttattgaggtttttttgggttaagatgtagatgatgtattgaaatgtttgttttatgttatatgtttatatgtagttaggtttaaaaattttggggttaatttaaatagagatatataaattttaagtttatatgaagtatttatggtaagggataagttttgttaagtatttttattaggttttttttatgtaaattatggagatgtagaattttgttttgtagtttttattaggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagttgataagagctgcaaagcaagattctacatctccgtgatttacatggggaaggcctggtagaggtgcttgacgaaacttatcccttgccataagtacttcgtgtggacttaaagtttgtatatctctatttagattaaccccaaagtttttaagcctgactgcatatggacgtataacatgaaacaggcgtttcgatacatcatctgcatcttgacccaagggaacctcgatgacttctaattttctgtcggggacataagggcaaacatcatggtcactctcatttcgatattcaagtgtggatatctgcaaattatcaatgataccctggatggcctgtgtctttgatccaggagttgcagtaagagccagtattctcagctgtatcggtaccgccatcaactcacgaactacaacacaataagaataattccctaaagctcgatgtgcctcgtcgatcaccaagcaaaccaagtagttagtaagacatgttcctgaccccgggatccaagctt

SEQ ID NO: 35 - DNA, 1818 bp, synthetic construct
  misc_feature 1..1818: note = hpFANCM-Bn[wt]
  source 1..1818: mol_type = other DNA
ggatccttggtacctaatacgactcactatagggagaaattatgatatcatcggatgaagaagacgactgtgaggatttggagcttagtccaaggctcactaacttcatcaagagtggcgttgttccagattcacctgtctatgaccaagttgcatacgaagcaaacagagaagaagaccttgatcttccacacacgagtttaactaatgaattggcagaagagccatcgacacctgagaaaaaggttcacattgcttctacggccaatgaattcagaaccccaacgaaggaagaagatttagccaacgaaacagaaagcttcgctgtttctccaatgcctgaggagtggagaactcccttggcgaatatcaccaacgcaagcagcagcgctagcaaagattggcgcgtgagttcgggagaaaagtcagaaactcttcgacagcctcgcaagttgaagagacttcgtagacttggagattgctcgagtgctgtgaaggagaataatcctggtattgcaaagacagaccatatcgtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacaggatatggtctgtctttgcaataccaggattattctccttcacagcactcgagcaatctccaagtctacgaagtctcttcaacttgcgaggctgtcgaagagtttctgacttttctcccgaactcacgcgccaatctttgctagcgctgctgcttgcgttggtgatattcgccaagggagttctccactcctcaggcattggagaaacagcgaagctttctgtttcgttggctaaatcttcttccttcgttggggttctgaattcattggccgtagaagcaatgtgaacctttttctcaggtgtcgatggctcttctgccaattcattagttaaactcgtgtgtggaagatcaaggtcttcttctctgtttgcttcgtatgcaacttggtcatagacaggtgaatctggaacaacgccactcttgatgaagttagtgagccttggactaagctccaaatcctcacagtcgtcttcttcatccgatgatatcataatttctctctagaaaggatcccggg

SEQ ID NO: 36 - DNA, 1818 bp, synthetic construct
  misc_feature 1..1818: note = hpFANCM-Bn[G:U]
  source 1..1818: mol_type = other DNA
ggatccttggtacctaatacgactcactatagggagaaattatgatattattggatgaagaagatgattgtgtggatttggagtttagtttaaggtttattaattttattaagagtggtgttgttttagatttatttgtttatgattaagttgtatatgaagtaaatagtgaagaagattttgattttttatatatgagtttaattaatgaattggtagaagagttattgatatttgagaaaaaggtttatattgtttttatggttaatgaatttagaattttaatgaaggaagaagatttagttaatgaaatagaaagttttgttgtttttttaatgtttgaggagtggagaatttttttggtgaatattattaatgtaagtagtagtgttagtaaagattggtgtgtgagtttgggagaaaagttagaaattttttgatagttttgtaagttgaagagattttgtagatttggagattgtttgagtgttgtgaaggagaataattttggtattgtaaagatagattatattgtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacaggatatggtctgtctttgcaataccaggattattctccttcacagcactcgagcaatctccaagtctacgaagtctcttcaacttgcgaggctgtcgaagagtttctgacttttctcccgaactcacgcgccaatctttgctagcgctgctgcttgcgttggtgatattcgccaagggagttctccactcctcaggcattggagaaacagcgaagctttctgtttcgttggctaaatcttcttccttcgttggggttctgaattcattggccgtagaagcaatgtgaacctttttctcaggtgtcgatggctcttctgccaattcattagttaaactcgtgtgtggaagatcaaggtcttcttctctgtttgcttcgtatgcaacttggtcatagacaggtgaatctggaacaacgccactcttgatgaagttagtgagccttggactaagctccaaatcctcacagtcgtcttcttcatccgatgatatcataatttctctctagaaaggatcccggg

SEQ ID NO: 37 - DNA, 2274 bp, Brassica napus
  source 1..2274: mol_type = other DNA
atggttagtctgcgctccacagaaaacactccggcttcggaaatggccagcgacggcaaaacggagaaagatggctccggcgactcacccacttctgttctcagcgatgaggaaaactgtgaagagaaaactgctactgttgctgtagaggaagagatacttctagccaagaatggagattcgtctcttatctctgaggccatggctcaggaagaagagcagcttctcaaaatccgggaagatgaagagattgctaaacgtgctgctggctctggtgaagctcctgatctgaatgatactcagtttactaaacttgatgagctcttgacccaaacccagctctactctgagtttctccttgagaaaatggaggatatcaccaaaaatgggatagaaggtgagacccaaaaggccgagcctgagcctgagcctgagcccgagaagaaaggccgtggacgtaaaagaaaggctgctcctcagggcgacagtatgaaggctaagaaagctgttgctgctatgatttcaagatccaaagaaggccgtgaatctgccgactcagatctgacagaggaagaaagagtcatgaaagagcagggtgaacttgttcctcttctgactggcggaaagttaaagtcttatcagctcaaaggtgtcaaatggctgatatcattgtggcaaaatggtttgaatggaattttagctgatcaaatgggtcttggaaagacaattcaaaccattggtttcctatcacacctcaaaggaaatgggttggatggtccatatctagtcattgccccactctctactctttcaaactggatgaatgagatcgctaggttcacgccttccattaatgcaatcatttaccatggagataagaaagaaagggatgagctcaggaagaggcacatgcccagaactgttggtccgaagttccctatagtcataacttcttatgaggttgctatgaatgatgctaaaaagaatctgcggcactatccatggaaatatgttgtgattgatgagggtcacaggttgaaaaaccacaagtgtaaactgctgagggagctaagatacttgaatatggagaacaaacttctgctgacaggaacacctctgcaaaataatttgtctgagctttggtcactgttgaattttattctgcctgacatctttgcatcacatgacgaatttgaatcatggtttgatttttctggaaagaataataatgaagcaactaaggaagaaggagaagagaaaagaagagctcaagtggttgcgaaacttcataatatactacgacctttcatcctccggagaatgaaatgtgatgttgagctctcacttccccggaaaaaagagattatcatctatgctacaatgacggaccatcagaagaagttccaggaacatcttgtgaaccacaccttggaagcacacattagagatgatactgtccgaggtcatggcttgaagggaaagcttaacaatcttgctattcaacttcgaaagaactgcaaccatcctgaccttcttgtggggcaactagatggctcatatctctacccacctttggaagacattgtgggacagtgcggtaaattccgcttattggagagattgcttgttcggttatttgccaaaaatcacagagtccttatcttctcccagtggacaaaaatactggacattatggattactacttcagtgagaaggggtttgaggtttgccgaatcgacggtagtgtgaaactagaagaaaggagaagacagatccaagaattcaatgatgagaagagcaactgcaggatatttcttctcagtaccagagccggaggactcggaattaatcttactgctgcagatacatgcatcctctacgatagcgattggaaccctcaaatggacttgcaagccatggacagatgccacagaattggtcagacaaaacctgttcatgtttacaggcttgcgacggctcagtcaatagagggccgagttctgaaacgagcatacagtaagcttaagctggaacatgtggttattggcaaggggcagtttcatcaagaacgtgccaagtcttcaacaccgttagaggaagatgacatactggcgttgcttaaggacgacgaaaatgctgaagataaactgatacaaaccgacataagcgaggaggatcttgacagggtgcttgaccgtagtgatctgatgattaccttaccgggcgagactcaagcacatgaagcttttccagtgaagggtccgggttgggaa

SEQ ID NO: 38 - DNA, 1824 bp, synthetic construct
  misc_feature 1..1824: note = hpDDM1-Bn[wt]
  source 1..1824: mol_type = other DNA
ggatccttggtacctaatacgactcactatagggagctgttgctgctatgatttcaagatccaaagaaggccgtgaatctgccgactcagatctgacagaggaagaaagagtcatgaatgagcagggtgaacttgttcctcttctgactggcggaaagttaaagtcttatcagctcaaaggtgtcaaatggctgatatcattgtggcaaaatggtttgaatggaattttagctgatcaaatgggtcttggaaagacaattcaaaccattggtttcctatcaccccaacaaggaaatgggttggatggtccatatctagtcattgccccactctctactctttcaaaagcgattggaaccctcaaatggacttgcaagccatggacagatgccacagaattggtcagacaaaacctgttcatgtttacaggcttgcgacggctcagtcaatagagggccgagttctgaaacgagcatacagtaagcttaagctggaacatgtggttattggcaaggggcagtttcatcaagaacgtggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagtcacgttcttgatgaaactgccccttgccaataaccacatgttccagcttaagcttactgtatgctcgtttcagaactcggccctctattgactgagccgtcgcaagcctgtaaacatgaacaggttttgtctgaccaattctgtggcatctgtccatggcttgcaagtccatttgagggttccaatcgcttttgaaagagtagagagtggggcaatgactagatatggaccatccaacccatttccttgttggggtgataggaaaccaatggtttgaattgtctttccaagacccatttgatcagctaaaattccattcaaaccattttgccacaatgatatcagccatttgacacctttgagctgataagactttaactttccgccagtcagaagaggaacaagttcaccctgctctttcatgactctttcttcctctgtcagatctgagtcggcagattcacggccttctttggatcttgaaatcatagcagcaacagcttctagaaaggatcccggg

SEQ ID NO: 39 - DNA, 1823 bp, synthetic construct
  misc_feature 1..1823: note = hpDDM1-Bn[G:U]
  source 1..1823: mol_type = other DNA
ggatccttggtacctaatacgactcactatagggttgttgttgttatgattttaagatttaaagaaggttgtgaatttgttgatttagatttgatagaggaagaaagagttatgaatgagtagggtgaatttgttttttttttgattggtggaaagttaaagttttattagtttaaaggtgttaaatggttgatattattgtggtaaaatggtttgaatggaattttagttgattaaatgggttttggaaagataatttaaattattggttttttattatattttaaaggaaatgggttggatggtttatatttagttattgttttattttttatttttttaaaagtgattggaatttttaaatggatttgtaagttatggatagatgttatagaattggttagataaaatttgtttatgtttataggtttgtgatggtttagttaatagagggttgagttttgaaatgagtatatagtaagtttaagttggaatatgtggttattggtaaggggtagttttattaagaatgtgtggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagtcacgttcttgatgaaactgccccttgccaataaccacatgttccagcttaagcttactgtatgctcgtttcagaactcggccctctattgactgagccgtcgcaagcctgtaaacatgaacaggttttgtctgaccaattctgtggcatctgtccatggcttgcaagtccatttgagggttccaatcgcttttgaaagagtagagagtggggcaatgactagatatggaccatccaacccatttccttgttggggtgataggaaaccaatggtttgaattgtctttccaagacccatttgatcagctaaaattccattcaaaccattttgccacaatgatatcagccatttgacacctttgagctgataagactttaactttccgccagtcagaagaggaacaagttcaccctgctctttcatgactctttcttcctctgtcagatctgagtcggcagattcacggccttctttggatcttgaaatcatagcagcaacagttctagaaaggatcccggg

SEQ ID NO: 40 - DNA, 720 bp, synthetic construct
  misc_feature 1..720: note = EGFP
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 41 - DNA, 1262 bp, synthetic construct
  misc_feature 1..1262: note = hpEGFP[wt]
  source 1..1262: mol_type = other DNA
gctagctaatacgactcactatagggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatgtatacgttgtggctgttgaagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaagcgaaggtggtcactagtgtgggccagggcacgggcagcttgccggtggtgcagatgaacttcagggtctagaccgcgtcggcatccggtcagtggcagtgaagggcgaacagttcctgattagggggatgaagctacctggtccgaaccacaaaccgttctactttactggctttggtcgtcatgaagatgcggacttgcgtggcaaaggattcgataacgtgctgatggtgcacgaccacgcattaatggactttaccttttaatggggaatgaagctacctggtccgaactcctaccgtacctcgcattacccttacgctgaagagatgctcgactgggcagatgaacatggcatcgtatttaggtgacactatagccctgaagttcatctgcaccaccggcaagctgcccgtgccctggcccacactagtgaccaccttcgcttacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacttcaacagccacaacgtatacatcatggccgacaagcagaagaacggcatcaaggtgaacttcaagatccgccacaacatcgaggacggcagcgtgcagctcgccgaccactaccagcagaacacccccatcggcgacggccccgtgctgctgccgtcgac

SEQ ID NO: 42 - DNA, 1262 bp, synthetic construct
  misc_feature 1..1262: note = hpEGFP[G:U]
  source 1..1262: mol_type = other DNA
gctagctaatacgactcactatagggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatgtatacgttgtggctgttgaagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaagcgaaggtggtcactagtgtgggccagggcacgggcagcttgccggtggtgcagatgaacttcagggtctagaccgcgtcggcatccggtcagtggcagtgaagggcgaacagttcctgattagggggatgaagctacctggtccgaaccacaaaccgttctactttactggctttggtcgtcatgaagatgcggacttgcgtggcaaaggattcgataacgtgctgatggtgcacgaccacgcattaatggactttaccttttaatggggaatgaagctacctggtccgaactcctaccgtacctcgcattacccttacgctgaagagatgctcgactgggcagatgaacatggcatcgtatttaggtgacactatagccttgaagtttatttgtattattggtaagttgtttgtgttttggtttatattagtgattatttttgtttatggtgtgtagtgttttagttgttattttgattatatgaagtagtatgatttttttaagtttgttatgtttgaaggttatgtttaggagtgtattattttttttaaggatgatggtaattataagatttgtgttgaggtgaagtttgagggtgatattttggtgaattgtattgagttgaagggtattgattttaaggaggatggtaatattttggggtataagttggagtataattttaatagttataatgtatatattatggttgataagtagaagaatggtattaaggtgaattttaagatttgttataatattgaggatggtagtgtgtagtttgttgattattattagtagaatatttttattggtgatggttttgtgttgttgttgtcgac

SEQ ID NO: 43 - DNA, 1259 bp, synthetic construct
  misc_feature 1..1259: note = ledEGFP[wt]
  source 1..1259: mol_type = other DNA
gctagctaatacgactcactatagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtgaaggtggtcacgagggtgggccagggcacgggcagcttgccggtggtgcagatgaacttcagggtctagaccgcgtcggcatccggtcagtggcagtgaagggcgaacagttcctgattagggggatgaagctacctggtccgaaccacaaaccgttctactttactggctttggtcgtcatgaagatgcggacttgcgtggcaaaggattcgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcctaacgtgctgatggtgcacgaccacgcattaatggactttaccttttaatggggaatgaagctacctggtccgaactcctaccgtacctcgcattacccttacgctgaagagatgctcgactgggcagatgaacatggcatcgtggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccattgtcgggatacactcgtcgac

SEQ ID NO: 44 - DNA, 1259 bp, synthetic construct
  misc_feature 1..1259: note = ledEGFP[G:U]
  source 1..1259: mol_type = other DNA
gctagctaatacgactcactatagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtgaaggtggtcacgagggtgggccagggcacgggcagcttgccggtggtgcagatgaacttcagggtctagaccgcgtcggcatccggtcagtggcagtgaagggcgaacagttcctgattagggggatgaagctacctggtccgaaccacaaaccgttctactttactggctttggtcgtcatgaagatgcggacttgcgtggcaaaggattcgattttgaagtttatttgtattattggtaagttgtttgtgttttggtttatttttgtgattatttttatttatggtgtgtagtgttttagttgttattttgattatatgaagtagtatgatttttttaagtttgttatgtttgaaggttatgtttaggagtgtattattttttttaaggatgatggtaattataagatttgtgttgaggtgaagtttgagggtgatattttggtgaattgtattgagttgaagggtattgattttaaggaggatggtaatattttggggtataagttggagtataattataatagttataatgtttatattatggttgataagtagaagaatggtattaaggtgaattttaagatttgttataatattgaggatggtagtgtgtagtttgttgattattattagtagaatatttttattggtgatggttttgtgttgttgtttaacgtgctgatggtgcacgaccacgcattaatggactttaccttttaatggggaatgaagctacctggtccgaactcctaccgtacctcgcattacccttacgctgaagagatgctcgactgggcagatgaacatggcatcgtggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccattgtcgggatacactcgtcgac

SEQ ID NO: 45 - DNA, 200 bp, synthetic construct
  misc_feature 1..200: note = hpGUS[G:U]
  source 1..200: mol_type = other DNA
ttgtgttggtatttggttagtggtagtgaagggtgaatagtttttgattaattataaattgttttattttattggttttggttgttatgaagatgtggatttgtgtggtaaaggatttgataatgtgttgatggtgtatgattatgtattaatggattggattggggttaatttttattgtattttgtattatttttatg

SEQ ID NO: 46 - DNA, 200 bp, synthetic construct
  misc_feature 1..200: note = hpGUS[1:4]
  source 1..200: mol_type = other DNA
tcgggtccgcaaccgctcactgggagtcaagcgcgtacacttcgtgaataagcactaacggttgtacattagtgggtttcgtcctcaagaacatggggagttgggtgccaatggaatcgttaaggtggtgaaggtccaccacctcgctttattggtctgcattcggggcaagtccaaccctacgtcggatttcccatacc

SEQ ID NO: 47 - DNA, 200 bp, synthetic construct
  misc_feature 1..200: note = hpGUS[2:10]
  source 1..200: mol_type = other DNA
tcgcgtcgcgatccggtctctggcagtgttgggcgaactcttcctgatataccacaaagggttctactaaactggcttacgtcgtcatctagatgcggtgttgcgtgggtaaggattccttaacgtgcacatggtgcagcaccacgcaaaaatggactccattggggcgtactcctacgctacctcgctatacccttagc

SEQ ID NO: 48 - DNA, 200 bp, synthetic construct
  misc_feature 1..200: note = hpEIN2[G:U]
  source 1..200: mol_type = other DNA
gattttagttagggtttatttagagaatggtttttgttttattttttgtttttttggtttttgttggatatattgattttgggaaatgggttgtaaatattgaaggaggtgtttgttttgggtatgatttggtggtaattattttgttttttaattttgttgttattttatgttaatatgttgtagtttgtataagtgtt

SEQ ID NO: 49 - DNA, 200 bp, synthetic construct
  misc_feature 1..200: note = hpCHS[G:U]
  source 1..200: mol_type = other DNA
gttgtttgttttgagattatagttgttattttttgtggtttttttgatatttattttgatttttttgttggttaggttttttttagtgatggtgttgttgtatttattgtggggttggattttgatatatttgttggagagaaatttatttttgagatggtgtttgttgtttagattatttttttagattttgatggtgt

SEQ ID NO: 50 - DNA, 200 bp, synthetic construct
  misc_feature 1..200: note = hpEIN2[G:U/U:G]
  source 1..200: mol_type = other DNA
aatgtttatgtgagttgtaatatattggtataagatggtggtaaaattgaaaagtagagtaattgttattaagttatatttgaaatgagtattttttttgatatttgtaatttattttttgggattaatatatttgatagaaattaaaaggataggaagtagagtaggaattattttttggataaattttagttgaggtt

SEQ ID NO: 51 - DNA, 199 bp, synthetic construct
  misc_feature 1..199: note = hpCHS[G:U/U:G]
  source 1..199: mol_type = other DNA
gtattattagagtttggaaggatggtttgagtggtagatattattttaaagatgggtttttttttgatagatgtgttagggtttgattttataatgagtgtggtggtgttattattgaaaagagtttgattgatgagggagttaaggtgggtgttagagggattatggaaggtaatggttgtgattttagagtagataa

SEQ ID NO: 52 - DNA, 30 bp, synthetic construct
  misc_feature 1..30: note = GUS-WT-F
  source 1..30: mol_type = other DNA
cctcgaggatcctcgcgtcggcatccggtc

SEQ ID NO: 53 - DNA, 33 bp, synthetic construct
  misc_feature 1..33: note = GUS-WT-R
  source 1..33: mol_type = other DNA
gggtaccaagcttcgtaagggtaatgcgaggta

SEQ ID NO: 54 - DNA, 118 bp, synthetic construct
  misc_feature 1..118: note = GUS-GU-F
  source 1..118: mol_type = other DNA
ccctcgagttgtgttggtatttggttagtggtagtgaagggtgaatagtttttgattaattataaattgttttattttattggttttggttgttatgaagatgtggatttgtgtggta

SEQ ID NO: 55 - DNA, 118 bp, synthetic construct
  misc_feature 1..118: note = GUS-GU-R
  source 1..118: mol_type = other DNA
ggggtacccataaaaataatacaaaatacaataaaaattaaccccaatccaatccattaatacataatcatacaccatcaacacattatcaaatcctttaccacacaaatccacatct

SEQ ID NO: 56 - DNA, 118 bp, synthetic construct
  misc_feature 1..118: note = GUS-4M-F
  source 1..118: mol_type = other DNA
ccctcgagtcgggtccgcaaccgctcactgggagtcaagcgcgtacacttcgtgaataagcactaacggttgtacattagtgggtttcgtcctcaagaacatggggagttgggtgcca

SEQ ID NO: 57 - DNA, 118 bp, synthetic construct
  misc_feature 1..118: note = GUS-4M-R
  source 1..118: mol_type = other DNA
ggggtaccggtatgggaaatccgacgtagggttggacttgccccgaatgcagaccaataaagcgaggtggtggaccttcaccaccttaacgattccattggcacccaactccccatgt

SEQ ID NO: 58 - DNA, 118 bp, synthetic construct
  misc_feature 1..118: note = GUS-10M-F
  source 1..118: mol_type = other DNA
ccctcgagtcgcgtcgcgatccggtctctggcagtgttgggcgaactcttcctgatataccacaaagggttctactaaactggcttacgtcgtcatctagatgcggtgttgcgtgggt

SEQ ID NO: 59 - DNA, 118 bp, synthetic construct
  misc_feature 1..118: note = GUS-10M-R
  source 1..118: mol_type = other DNA
ggggtaccgctaagggtatagcgaggtagcgtaggagtacgccccaatggagtccatttttgcgtggtgctgcaccatgtgcacgttaaggaatccttacccacgcaacaccgcatct

SEQ ID NO: 60 - DNA, 20 bp, synthetic construct
  misc_feature 1..20: note = Forward primer (35S-F3)
  source 1..20: mol_type = other DNA
tggctcctacaaatgccatc

SEQ ID NO: 61 - DNA, 21 bp, synthetic construct
  misc_feature 1..21: note = Reverse primer (GUSwt-R2)
  misc_feature 3..4: note = R - A or G
  misc_feature 9: note = R - A or G
  misc_feature 13: note = R - A or G
  source 1..21: mol_type = other DNA
carraactrttcrcccttcac

SEQ ID NO: 62 - DNA, 21 bp, synthetic construct
  misc_feature 1..21: note = Forward primer (GUSgu-R2)
  source 1..21: mol_type = other DNA
caaaaactattcacccttcac

SEQ ID NO: 63 - DNA, 21 bp, synthetic construct
  misc_feature 1..21: note = reverse primer (GUS4m-R2)
  misc_feature 4: note = R - A or G
  misc_feature 7: note = R - A or G
  misc_feature 9: note = R - A or G
  misc_feature 13: note = R - A or G
  misc_feature 15: note = R - A or G
  misc_feature 19: note = R - A or G
  source 1..21: mol_type = other DNA
cacraartrtacrcrcttrac

SEQ ID NO: 64 - DNA, 20 bp, synthetic construct
  misc_feature 1..20: note = Forward primer (35S-F2)
  source 1..20: mol_type = other DNA
gaggatctaacagaactcgc

SEQ ID NO: 65 - DNA, 23 bp, synthetic construct
  misc_feature 1..23: note = reverse primer (35S-R1)
  source 1..23: mol_type = other DNA
ctctccaaatgaaatgaacttcc

SEQ ID NO: 66 - DNA, 36 bp, synthetic construct
  misc_feature 1..36: note = EIN2wt-F
  source 1..36: mol_type = other DNA
cctcgaggatcctctagacctcagctagggtttatc

SEQ ID NO: 67 - DNA, 33 bp, synthetic construct
  misc_feature 1..33: note = EIN2wt-R
  source 1..33: mol_type = other DNA
gggtaccaagcttaacgcttatgcgagctgcaa

SEQ ID NO: 68 - DNA, 32 bp, synthetic construct
  misc_feature 1..32: note = CHSwt-F
  source 1..32: mol_type = other DNA
cctcgaggatccgttgtctgctctgagatcac

SEQ ID NO: 69 - DNA, 38 bp, synthetic construct
  misc_feature 1..38: note = CHSwt-R
  source 1..38: mol_type = other DNA
gggtaccaagcttctagagcaccatcagagtctggaag

SEQ ID NO: 70 - DNA, 119 bp, synthetic construct
  misc_feature 1..119: note = EIN2gu-F
  source 1..119: mol_type = other DNA
cctcgagtctagattttagttagggtttatttagagaatggtttttgttttattttttgtttttttggtttttgttggatatattgattttgggaaatgggttgtaaatattgaaggag

SEQ ID NO: 71 - DNA, 119 bp, synthetic construct
  misc_feature 1..119: note = EIN2gu-R
  source 1..119: mol_type = other DNA
gggtaccaacacttatacaaactacaacatattaacataaaataacaacaaaattaaaaaacaaaataattaccaccaaatcatacccaaaacaaacacctccttcaatatttacaacc

SEQ ID NO: 72 - DNA, 119 bp, synthetic construct
  misc_feature 1..119: note = CHSgu-F
  source 1..119: mol_type = other DNA
cctcgaggttgtttgttttgagattatagttgttattttttgtggtttttttgatatttattttgatttttttgttggttaggttttttttagtgatggtgttgttgtatttattgtgg

SEQ ID NO: 73 - DNA, 119 bp, synthetic construct
  misc_feature 1..119: note = CHSgu-R
  source 1..119: mol_type = other DNA
gggtacctctagacaccatcaaaatctaaaaaaataatctaaacaacaaacaccatctcaaaaataaatttctctccaacaaatatatcaaaatccaaccccacaataaatacaacaac

SEQ ID NO: 74 - DNA, 120 bp, synthetic construct
  misc_feature 1..120: note = asEIN2gu-F
  source 1..120: mol_type = other DNA
caagcttaatgtttatgtgagttgtaatatattggtataagatggtggtaaaattgaaaagtagagtaattgttattaagttatatttgaaatgagtattttttttgatatttgtaattt

SEQ ID NO: 75 - DNA, 120 bp, synthetic construct
  misc_feature 1..120: note = asEIN2gu-R
  source 1..120: mol_type = other DNA
gggatcctctagaacctcaactaaaatttatccaaaaaataattcctactctacttcctatccttttaatttctatcaaatatattaatcccaaaaaataaattacaaatatcaaaaaaa

SEQ ID NO: 76 - DNA, 120 bp, synthetic construct
  misc_feature 1..120: note = asCHSgu-F
  source 1..120: mol_type = other DNA
caagcttctagagtattattagagtttggaaggatggtttgagtggtagatattattttaaagatgggtttttttttgatagatgtgttagggtttgattttataatgagtgtggtggtg

SEQ ID NO: 77 - DNA, 120 bp, synthetic construct
  misc_feature 1..120: note = asCHSgu-R
  source 1..120: mol_type = other DNA
gggatccattatctactctaaaatcacaaccattaccttccataatccctctaacacccaccttaactccctcatcaatcaaactcttttcaataataacaccaccacactcattataaa

SEQ ID NO: 78 - DNA, 20 bp, synthetic construct
  misc_feature 1..20: note = CHS-200-F2
  source 1..20: mol_type = other DNA
gacatgcctggtgctgacta

SEQ ID NO: 79 - DNA, 20 bp, synthetic construct
  misc_feature 1..20: note = CHS-200-R2
  source 1..20: mol_type = other DNA
ccttagcgatacggaggaca

SEQ ID NO: 80 - DNA, 20 bp, synthetic construct
  misc_feature 1..20: note = Actin2-For
  source 1..20: mol_type = other DNA
tccctcagcacattccagca

SEQ ID NO: 81 - DNA, 22 bp, synthetic construct
  misc_feature 1..22: note = Actin2-Rev
  source 1..22: mol_type = other DNA
gatcccattcataaaaccccag

SEQ ID NO: 82 - DNA, 24 bp, synthetic construct
  misc_feature 1..24: note = Top-35S-F2
  misc_feature 8: note = Y - C or T
  misc_feature 11: note = Y - C or T
  misc_feature 14: note = Y - C or T
  misc_feature 17: note = Y - C or T
  source 1..24: mol_type = other DNA
agaaaatyttygtyaayatggtgg

SEQ ID NO: 83 - DNA, 24 bp, synthetic construct
  misc_feature 1..24: note = Top-35S-R2
  misc_feature 4: note = R - A or G
  misc_feature 6..7: note = R - A or G
  misc_feature 9: note = R - A or G
  misc_feature 12: note = R - A or G
  source 1..24: mol_type = other DNA
tcartrraratrtcacatcaatcc

SEQ ID NO: 84 - DNA, 23 bp, synthetic construct
  misc_feature 1..23: note = Link-35S-F2
  misc_feature 1..2: note = Y - C or T
  misc_feature 5: note = Y - C or T
  misc_feature 10: note = Y - C or T
  source 1..23: mol_type = other DNA
yyatyattgygataaaggaaagg

SEQ ID NO: 85 - DNA, 22 bp, synthetic construct
  misc_feature 1..22: note = Link-EIN2-R2
  misc_feature 6: note = R - A or G
  misc_feature 14: note = R - A or G
  source 1..22: mol_type = other DNA
taattrccaccaartcataccc

SEQ ID NO: 86 - RNA, 22 nt, synthetic construct
  misc_feature 1..22: note = sense si22
  source 1..22: mol_type = other RNA
gcaagctgaccctgaagttcat

SEQ ID NO: 87 - RNA, 22 nt, synthetic construct
  misc_feature 1..22: note = antisense si22
  source 1..22: mol_type = other RNA
gaacttcagggtcagcttgccg

SEQ ID NO: 88 - DNA, 20 bp, synthetic construct
  misc_feature 1..20: note = Forward primer
  source 1..20: mol_type = other DNA
ttttagtatatgtgctgccg

SEQ ID NO: 89 - DNA, 83 bp, synthetic construct
  misc_feature 1..83: note = reverse primer
  source 1..83: mol_type = other DNA
ctcgagttccaaaaaagctgaccctgaagttcatctctcttgaagatgaacttcagggtcagccaaacaaggcttttctccaa

SEQ ID NO: 90 - DNA, 20 bp, synthetic construct
  misc_feature 1..20: note = forward primer
  source 1..20: mol_type = other DNA
ttttagtatatgtgctgccg

SEQ ID NO: 91 - DNA, 83 bp, synthetic construct
  misc_feature 1..83: note = reverse primer
  source 1..83: mol_type = other DNA
ctcgagttccaaaaaaataagtcgcagcagtacaatctcttgaattgtactgctgcgacttatgaataccgcttcctcctgag

SEQ ID NO: 92 - RNA, 88 nt, synthetic construct
  misc_feature 1..88: note = dsRNA molecules
  source 1..88: mol_type = other RNA
aagtgatttgtgaatggattcggttaagttagtgataggtatcacgctggccattactgacatgaccggattcgttcgcgagtcactt

SEQ ID NO: 93 - DNA, 2653 bp, Brassica napus
  source 1..2653: mol_type = other DNA
agaaaaatcgaaaaatgtgacagtgcgtcttttcacttaataccctcgttttgaatttgctctcggaaagcgtctgagagagtgttcggtgatttctcccgccgcttggggttttttccgttaccggaatatccttctcctccgatggttagtctgcgctccacagaaaacactccggcttcggaaatggccagcgacggcaaaacggagaaagatggctccggcgactcacccacttctgttctcagcgatgaggaaaactgtgaagagaaaactgctactgttgctgtagaggaagagatacttctagccaagaatggagattcgtctcttatctctgaggccatggctcaggaagaagagcagcttctcaaaatccgggaagatgaagagattgctaaacgtgctgctggctctggtgaagctcctgatctgaatgatactcagtttactaaacttgatgagctcttgacccaaacccagctctactctgagtttctccttgagaaaatggaggatatcaccaaaaatgggatagaaggtgagacccaaaaggccgagcctgagcctgagcctgagcccgagaagaaaggccgtggacgtaaaagaaaggctgctcctcagggcgacagtatgaaggctaagaaagctgttgctgctatgatttcaagatccaaagaaggccgtgaatctgccgactcagatctgacagaggaagaaagagtcatgaaagagcagggtgaacttgttcctcttctgactggcggaaagttaaagtcttatcagctcaaaggtgtcaaatggctgatatcattgtggcaaaatggtttgaatggaattttagctgatcaaatgggtcttggaaagacaattcaaaccattggtttcctatcacacctcaaaggaaatgggttggatggtccatatctagtcattgccccactctctactctttcaaactggatgaatgagatcgctaggttcacgccttccattaatgcaatcatttaccatggagataagaaagaaagggatgagctcaggaagaggcacatgcccagaactgttggtccgaagttccctatagtcataacttcttatgaggttgctatgaatgatgctaaaaagaatctgcggcactatccatggaaatatgttgtgattgatgagggtcacaggttgaaaaaccacaagtgtaaactgctgagggagctaagatacttgaatatggagaacaaacttctgctgccaggaacacctctgcaaaataatttgtctgagcttcggtcactgttgaattttattctgcctgacatctttgcatcacatgacgaatttgaatcatggtttgatttttcttgaaagaataataatgaagcaactaaggaagaaggagaagagaaaagaagagctcaagtggttgcgaaacttcataatatactacgacctttcatcctccggagaatgaaatgtgatgttgagctctcacttccccggaaaaaagagattatcatctatgctacaatgacggaccatcagaagaagttccaggaacatcttgtgaaccacaccttggaagcacacattagagatgatactgtccgaggtcatggcttgaagggaaagcttaacaatcttgctattcaacttcgaaagaactgcaaccatcctgaccttcttgtggggcaactagatggctcatatctctacccacctttggaagacattgtgggacagtgcggtaaattccgcttattggagagattgcttgttcggttatttgccaaaaatcacagagtccttatcttctcccagtggacaaaaatactggacattatggattactacttcagtgagaaggggtttgaggtttgccgaatcgacggtagtgtgaaactagaagaaaggagaagacagatccaagaattcaatgatgagaagagcaactgcaggatatttcttctcagtaccagagccggaggactcggaattaatcttactgctgcagatacatgcatcctctacgatagcgattggaaccctcaaatggacttgcaagccatggacagatgccacagaattggtcagacaaaacctgttcatgtttacaggcttgcgacggctcagtcaatagagggccgagttctgaaacgagcatacagtaagcttaagctggaacatgtggttattggcaaggggcagtttcatcaagaacgtgccaagtcttcaacaccgttagaggaagatgacatactggcgttgcttaaggacgacgaaaatgctgaagataaactgatacaaaccgacataagcgaggaggatcttgacagggtgcttgaccgtagtgatctgatgattaccttaccgggcgagactcaagcacatgaagcttttccagtgaagggtccgggttgggaagtggtctcgtctagctcagctggagggatgctgtcttccctcaacagttagaaccactctttgcaaaaccacttcggtgtgtttttttttccggaacataaccggttacttttgcctgctactcggaagttttaacttgaaaccttggaaacatctgatgaaaacaattgcggatattatgttattagactatttatttatgccttttgaaatttggcagtaattttttagttaa

SEQ ID NO: 94 - DNA, 1773 bp, synthetic construct
  misc_feature 1..1773: note = Chimeric DNA encoding a hairpin RNAi (hpRNA) construct targetinga DDM1 gene of B. napus
  source 1..1773: mol_type = other DNA
gggagctgttgctgctatgatttcaagatccaaagaaggccgtgaatctgccgactcagatctgacagaggaagaaagagtcatgaatgagcagggtgaacttgttcctcttctgactggcggaaagttaaagtcttatcagctcaaaggtgtcaaatggctgatatcattgtggcaaaatggtttgaatggaattttagctgatcaaatgggtcttggaaagacaattcaaaccattggtttcctatcaccccaacaaggaaatgggttggatggtccatatctagtcattgccccactctctactctttcaaaagcgattggaaccctcaaatggacttgcaagccatggacagatgccacagaattggtcagacaaaacctgttcatgtttacaggcttgcgacggctcagtcaatagagggccgagttctgaaacgagcatacagtaagcttaagctggaacatgtggttattggcaaggggcagtttcatcaagaacgtggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagtcacgttcttgatgaaactgccccttgccaataaccacatgttccagcttaagcttactgtatgctcgtttcagaactcggccctctattgactgagccgtcgcaagcctgtaaacatgaacaggttttgtctgaccaattctgtggcatctgtccatggcttgcaagtccatttgagggttccaatcgcttttgaaagagtagagagtggggcaatgactagatatggaccatccaacccatttccttgttggggtgataggaaaccaatggtttgaattgtctttccaagacccatttgatcagctaaaattccattcaaaccattttgccacaatgatatcagccatttgacacctttgagctgataagactttaactttccgccagtcagaagaggaacaagttcaccctgctctttcatgactctttcttcctctgtcagatctgagtcggcagattcacggccttctttggatcttgaaatcatagcagcaacag

SEQ ID NO: 95 - DNA, 1773 bp, synthetic construct
  misc_feature 1..1773: note = Chimeric DNA encoding a hairpin RNAi (hpRNA) construct with G:Ubasepairs, targeting a DDM1 gene of B. napus
  source 1..1773: mol_type = other DNA
gggttgttgttgttatgattttaagatttaaagaaggttgtgaatttgttgatttagatttgatagaggaagaaagagttatgaatgagtagggtgaatttgttttttttttgattggtggaaagttaaagttttattagtttaaaggtgttaaatggttgatattattgtggtaaaatggtttgaatggaattttagttgattaaatgggttttggaaagataatttaaattattggttttttattatattttaaaggaaatgggttggatggtttatatttagttattgttttattttttatttttttaaaagtgattggaatttttaaatggatttgtaagttatggatagatgttatagaattggttagataaaatttgtttatgtttataggtttgtgatggtttagttaatagagggttgagttttgaaatgagtatatagtaagtttaagttggaatatgtggttattggtaaggggtagttttattaagaatgtgtggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagtcacgttcttgatgaaactgccccttgccaataaccacatgttccagcttaagcttactgtatgctcgtttcagaactcggccctctattgactgagccgtcgcaagcctgtaaacatgaacaggttttgtctgaccaattctgtggcatctgtccatggcttgcaagtccatttgagggttccaatcgcttttgaaagagtagagagtggggcaatgactagatatggaccatccaacccatttccttgttggggtgataggaaaccaatggtttgaattgtctttccaagacccatttgatcagctaaaattccattcaaaccattttgccacaatgatatcagccatttgacacctttgagctgataagactttaactttccgccagtcagaagaggaacaagttcaccctgctctttcatgactctttcttcctctgtcagatctgagtcggcagattcacggccttctttggatcttgaaatcatagcagcaacag

SEQ ID NO: 96 - DNA, 1260 bp, synthetic construct
  misc_feature 1..1260: note = Chimeric DNA encoding a ledRNA construct, targeting a DDM1 geneof B. napus
  source 1..1260: mol_type = other DNA
ggggtgataggaaaccaatggtttgaattgtctttccaagacccatttgatcagctaaaattccattcaaaccattttgccacaatgatatcagccatttgacacctttgagctgataagactttaactttccgccagtcagaagaggaacaagttcaccctgctctttcatgactctttcttcctctgtcagatctgagtcggcagattcacggccttctttggatcttgaaatcatagcagcaacagctttcttagccttcatactgtcgccctgaggagcagcctttcttttacgtccacggcctttcttctcgggctcaggctcaggctcaggctcggccttttgggtctcaccttctatcccatttttggtgatagctgttgctgctatgatttcaagatccaaagaaggccgtgaatctgccgactcagatctgacagaggaagaaagagtcatgaatgagcagggtgaacttgttcctcttctgactggcggaaagttaaagtcttatcagctcaaaggtgtcaaatggctgatatcattgtggcaaaatggtttgaatggaattttagctgatcaaatgggtcttggaaagacaattcaaaccattggtttcctatcaccccaacaaggaaatgggttggatggtccatatctagtcattgccccactctctactctttcaaaagcgattggaaccctcaaatggacttgcaagccatggacagatgccacagaattggtcagacaaaacctgttcatgtttacaggcttgcgacggctcagtcaatagagggccgagttctgaaacgagcatacagtaagcttaagctggaacatgtggttattggcaaggggcagtttcatcaagaacgtcactacggtcaagcaccctgtcaagatcctcctcgcttatgtcggtttgtatcagtttatcttcagcattttcgtcgtccttaagcaacgccagtatgtcatcttcctctaacggtgttgaagacttggcacgttcttgatgaaactgccccttgccaataaccacatgttccagcttaagcttactgtatgctcgtttcagaactcggccctctattgactgagccgtcgcaagcctgtaaacatgaacaggttttgtctgaccaattctgtggcatctgtccatggcttgcaagtccatttgagggttccaatcgcttttgaaagagtagagagtggggcaatgactagatatggaccatccaacccatttccttgtt

SEQ ID NO: 97 - DNA, 4421 bp, Arabidopsis thaliana
  source 1..4421: mol_type = other DNA
tcttccaaaatttgcccgccattctctgtgtctcttcgtctaagggtttcttccaaagaacgacgacaaaaccactgaacctaaaatccgaaatccaaaagattcatgcgaaaaaatcgttaaagagtaccaatttcagaaagttacattcttacagagaacaagtaattccccagaaatgggatctagggttccaatagaaaccatcgaagaagacggcgaattcgattgggaagcagcagtcaaagaaatcgacttggcttgtcttaaaaccacaaacgcttcttcttcttcgtcatcccatttcactcctttggctaatccaccaattacggcaaatctcactaagccacctgcgaagagacaatctactctcgataaattcatcggcagaaccgaacataaaccggagaatcatcaagttgtttccgagtgtggtgttaacgataacgataatagtcctttagttgggattgatcctgaggcagctaaaacttggatttatccagtgaatgggagtgttcctttaagagattatcagtttgctataacgaagactgctttgttttcgaatacattggtggctttgcctacgggacttggtaaaacgcttatagctgcggttgttatgtataattacttcagatggtttccacaaggtaaaatagtatttgcggcgccttctaggcctcttgtgatgcagcagattgaggcgtgtcataatattgttggaataccacaagaatggacgattgacttgacgggtcagacatgtccttcgaaaagagcttttttgtggaaaagcaaacgggttttctttgtcactccacaagtgttagagaaggatatacagtcaggaacatgtcttactaactacttggtttgcttggtgatcgacgaggcacatcgagctttagggaattattcttattgtgttgtagttcgtgagttgatggcggtaccgatacagctgagaatactggctcttactgcaactcctggatcaaagacacaggccatccagggtatcattgataatttgcagatatccacacttgaatatcgaaatgagagtgaccatgatgtttgcccttatgtccacgacagaaaattagaagtcatcgaggttcccttgggtcaagatgcagatgatgtatcgaaacgcctgtttcatgttatacgtccatatgcagtcaggcttaaaaactttggggttaatctaaatagagatatacaaactttaagtccacacgaagtacttatggcaagggataagtttcgtcaagcacctctaccaggccttccccatgtaaatcacggagatgtagaatcttgctttgcagctcttatcactctttatcatattcgtaagctcctttctagtcatggaataagaccagcgtatgagatgctagaagagaaattgaaagaagggccatttgctaggttgatgagtaagaatgaagatattaggatgacgaagcttttgatgcagcaaaggttgtcacatggagcaccaagcccaaaattgtcgaagatgttagaaatactggttgatcatttcaaagtgaaagatccgaagacatcacgggtcattattttctcaaatttcagaggaagcgtaagagacataatgaacgcattaagtaatattggagatatggtcaaagcaactgagtttattggtcaaagttcaggtaagacattgaaaggccagtcgcaaaaaattcagcaggctgttttggagaaatttagagctggggggttcaatgttattgtcgcaacatctattggtgaagaaggcttggatatcatggaagttgacctagttatatgttttgatgctaatgtatctcctctgaggatgattcaacggatgggaagaactggaaggaaaaataatggtcgagttgtagttcttgcttgtgaaggatcagaaaagaacagctatatgcgaaagcaagcaagtggacgggctattaaaaaacacatgcggaatggaggaacaaatagttttaattttcatcctagtccaaggatgattccccatgtttataagccagaagttcagcatgttgagttttcaatcaagcaattcgttccacgtggaaagaaactacaagaggagtatgccactgagactccagctttccagaaaaagcttacacctgcagagacgcatatgctcgctaagtattacaacaaccccgatgaggaaaagttgagagtgtccttaattgcgttccctcacttccagacattgccatccaaggtgcacaaagtaatgcattcacgtcaaacaggcatgttaattgacgctatgcagcacttgcaagagccaactttttcagaacagagtaaaagcttcttcactgagtttcgagctcctttgggtgaaagagaagagcttgatacaggtctgagggttactaatgatccaaaagatctacactctgtccgtgatttggaagtcaacacatcacagagaaaggcaaaacaagttgaatctcccacaagcaccttagagacaacagagaaggattacgaagaatcttcacccacacaccgttatcttttcagttcagaatgtgcatccgttgatactctggggaacgtcttcgtaatgccagttcctcttttattctttcctaatgttctggagtcagacaatacgcctctgcctaaaacagaaaaacaacattcttgccggaatacatctcacattgacttagttccagtagatacttcggaaaaacatcggcaagataatatctcatgcaagttaaaggaaagattctcgccagacggtgccagcgagacactagagactcatagccttgtgaaaaggaactccaccagagtaggtgaagatgatgtagcgaattctgttggagaaattgtgttatcatcggatgaagatgactgtgagggattggagcttagtccacggctcactaacttcatcaagagcggcattgttccagagtcacctgtctatgaccaaggggaagcgaacagagaagaagatcttgaatttcctcagctttcttcacccatgaggttcagtaacgaattggcaggagagtcttctttccctgagagaaaggttcagcataagtgcaacgattataacattgtgtctacaaccactgaattgagaactcctcagaaggaggtaggtttggccaacggaacagaatgcttggctgtttctcctattcctgaggattggagaactcccttggcgaatctgacaaacacaaacagcagcgctcgcaaagattggcgggtgagttctggagaaaagttagaaactcttcgacagcctcgcaagttgaagagactacgtagacttggagattgctcgagtgctgtaaaggagaattatcctggtattacagaggcagaccatatcagatctcgttctcgcggtaaaaagcacattagaggtaagaagaagatgatcatggatgatgatgtccaagtcttcattgacgaggaagctgaggtctcttcgggagcagagatgtcggctgatgagaacgaagatgtgactggcgattcatttgaagatagtttcatagatgacggaacaatgcctacagcaaatactcaagccgagtctggtaaagttgacatgatggctgtttacaggcgttctcttctcagccagtcaccattaccggcgagatttcgtgatttagccgcatcaagtctgagtccttattctgctggacccttgacgagaataaatgagagcagaagcgactcagataaatcattgtcttctcttcgaacaccaaaaacaacaaactctgagtcaaaccaagatgcaatgatgataggaaacctttcggtagtacaaatctcgtcagatagccggaaaaggaaatttagcttatgcaactcggcgaatgcccccgtgattaacttagaaagcaagtttgcagctcatgcacaagccacggagaaggaaagccatgaaggcgtgagaagcaatgcaggtgcgttagagtacaatgatgatgatgatgatgcattctttgcgacactagactttgatgcaatggaagcacaagccacattgttattgtcgaaacagagatccgaagcaaaagagaaagaagacgcaacggttatacctaatccaggcatgcagagaagtgatggtatggagaaagatgcaccatcttttgatcttggtctgtggtgattcttctttcatacgaagatactaagttatgtatatagattgacaaaggagacagtagagcataggcatttggatgtatgttttgtgtattaagtttaggtatatcctattgaagtacagtgcttaaggcagtgcacatggttaaatcaaggttaatgcctcaattcgttgaaccctttaagtaatgacacaaatatgactacatcgg

SEQ ID NO: 98 - DNA, 1771 bp, synthetic construct
  misc_feature 1..1771: note = Chimeric DNA encoding a hairpin RNAi (hpRNA) construct targetinga FANCM gene of A. thaliana
  source 1..1771: mol_type = other DNA
gggtcaggaacatgtcttactaactacttggtttgcttggtgatcgacgaggcacatcgagctttagggaattattcttattgtgttgtagttcgtgagttgatggcggtaccgatacagctgagaatactggctcttactgcaactcctggatcaaagacacaggccatccagggtatcattgataatttgcagatatccacacttgaatatcgaaatgagagtgaccatgatgtttgcccttatgtccccgacagaaaattagaagtcatcgaggttcccttgggtcaagatgcagatgatgtatcgaaacgcctgtttcatgttatacgtccatatgcagtcaggcttaaaaactttggggttaatctaaatagagatatacaaactttaagtccacacgaagtacttatggcaagggataagtttcgtcaagcacctctaccaggccttccccatgtaaatcacggagatgtagaatcttgctttgcagctcttatcaggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagttgataagagctgcaaagcaagattctacatctccgtgatttacatggggaaggcctggtagaggtgcttgacgaaacttatcccttgccataagtacttcgtgtggacttaaagtttgtatatctctatttagattaaccccaaagtttttaagcctgactgcatatggacgtataacatgaaacaggcgtttcgatacatcatctgcatcttgacccaagggaacctcgatgacttctaattttctgtcggggacataagggcaaacatcatggtcactctcatttcgatattcaagtgtggatatctgcaaattatcaatgataccctggatggcctgtgtctttgatccaggagttgcagtaagagccagtattctcagctgtatcggtaccgccatcaactcacgaactacaacacaataagaataattccctaaagctcgatgtgcctcgtcgatcaccaagcaaaccaagtagttagtaagacatgttcctgac

SEQ ID NO: 99 - DNA, 1771 bp, synthetic construct
  misc_feature 1..1771: note = Chimeric DNA encoding a hairpin RNAi (hpRNA) construct with G:Ubasepairs, targeting a FANCM gene of A. thaliana
  source 1..1771: mol_type = other DNA
gggttaggaatatgttttattaattatttggtttgtttggtgattgatgaggtatattgagttttagggaattatttttattgtgttgtagtttgtgagttgatggtggtattgatatagttgagaatattggtttttattgtaatttttggattaaagatataggttatttagggtattattgataatttgtagatatttatatttgaatattgaaatgagagtgattatgatgtttgtttttatgtttttgatagaaaattagaagttattgaggtttttttgggttaagatgtagatgatgtattgaaatgtttgttttatgttatatgtttatatgtagttaggtttaaaaattttggggttaatttaaatagagatatataaattttaagtttatatgaagtatttatggtaagggataagttttgttaagtatttttattaggttttttttatgtaaattatggagatgtagaattttgttttgtagtttttattaggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagttgataagagctgcaaagcaagattctacatctccgtgatttacatggggaaggcctggtagaggtgcttgacgaaacttatcccttgccataagtacttcgtgtggacttaaagtttgtatatctctatttagattaaccccaaagtttttaagcctgactgcatatggacgtataacatgaaacaggcgtttcgatacatcatctgcatcttgacccaagggaacctcgatgacttctaattttctgtcggggacataagggcaaacatcatggtcactctcatttcgatattcaagtgtggatatctgcaaattatcaatgataccctggatggcctgtgtctttgatccaggagttgcagtaagagccagtattctcagctgtatcggtaccgccatcaactcacgaactacaacacaataagaataattccctaaagctcgatgtgcctcgtcgatcaccaagcaaaccaagtagttagtaagacatgttcctgac

SEQ ID NO: 100 - DNA, 1259 bp, synthetic construct
  misc_feature 1..1259: note = Chimeric DNA encoding a ledRNA construct, targeting a FANCM geneof A. thaliana
  source 1..1259: mol_type = other DNA
gggacataagggcaaacatcatggtcactctcatttcgatattcaagtgtggatatctgcaaattatcaatgataccctggatggcctgtgtctttgatccaggagttgcagtaagagccagtattctcagctgtatcggtaccgccatcaactcacgaactacaacacaataagaataattccctaaagctcgatgtgcctcgtcgatcaccaagcaaaccaagtagttagtaagacatgttcctgactgtatatccttctctaacacttgtggagtgacaaagaaaacccgtttgcttttccacaaaaaagctcttttcgaaggacatgtctgacccgtcaagtcaatcgtccattcttgtggtattccaacaatatgtcaggaacatgtcttactaactacttggtttgcttggtgatcgacgaggcacatcgagctttagggaattattcttattgtgttgtagttcgtgagttgatggcggtaccgatacagctgagaatactggctcttactgcaactcctggatcaaagacacaggccatccagggtatcattgataatttgcagatatccacacttgaatatcgaaatgagagtgaccatgatgtttgcccttatgtccccgacagaaaattagaagtcatcgaggttcccttgggtcaagatgcagatgatgtatcgaaacgcctgtttcatgttatacgtccatatgcagtcaggcttaaaaactttggggttaatctaaatagagatatacaaactttaagtccacacgaagtacttatggcaagggataagtttcgtcaagcacctctaccaggccttccccatgtaaatcacggagatgtagaatcttgctttgcagctcttatcattcgtcatcctaatatcttcattcttactcatcaacctagcaaatggcccttctttcaatttctcttctagcatctcatacgctggtcttattccatgactagaaaggagcttacgaatatgataaagagtgataagagctgcaaagcaagattctacatctccgtgatttacatggggaaggcctggtagaggtgcttgacgaaacttatcccttgccataagtacttcgtgtggacttaaagtttgtatatctctatttagattaaccccaaagtttttaagcctgactgcatatggacgtataacatgaaacaggcgtttcgatacatcatctgcatcttgacccaagggaacctcgatgacttctaattttctgtc

SEQ ID NO: 101 - DNA, 4228 bp, Brassica napus
  source 1..4228: mol_type = other DNA
tccaaaattggttttgcccgccaatgtggcttcggcgagggtttcttccacaaaaccccactcaacctaaaatctgattcggcgagaaacgctgtctacttatctcacgcgaaaagaaaggcgtagatccaccctaaactaaaacagagcatcaagtgaaatgggacccgagtttccgatcgaactcgttgaagaagaagatggattcgattgggaagcagcagtcagagaaatcgacttggcttgcctcaaatccttaaacccttcttcttcttcttcgacccatttcaccaacggcaatggcactaaacctgctaaaagacaatctactcttgatcgattcatcgcaagagccgaccacaagcctcctcctccgtatcctcctgttgtttccgacccgagtttcgagtgtggtactaacgacaacactcccagcgtcgggattgatcctgagacagctaaaacttggatttatccaatgaacgttcctctaagagattatcagtttgctataacgaagactgctttgttttcaaacacattagttgctttaccaacaggccttggtaaaacgctcatagctgcagttgtaatgtataattacttcagatggtttccacaaggtaaaattgtctttgccgcaccttctaggcctcttgtgatgcagcagattgaggcctgccataatatcgtggggataccacaagaatggacgattgacttgacgggtcagacttgcccttccaaaagagcttccttgtggaaaaccaaaagggttttcttcgtcactccacaagttcttgagaaggatatacagtcaggaacgtgtgttaccaactgcttggtttgcttggtgatcgacgaggcacatcgagctttagggaattattcttattgtgttgtagttcgtgagttgatggcagtaccagtgcagttgagaatattggctcttactgcaactcctggatcaaagacacaggccatacagggtatccttgataatttgcagatatcaacacttgaatatcgaaacgagagtgaccatgatgtctgcccttatgtccacgacagaaaagtagaactaatcgaggttcccttgggtaaagatgcagatgaggtatctaaacgcctattagatgttatacgtccatatgctgtcaggcttaaaaatttcggggtcattctaagcagggattatcaaactttgagtccacacgaattacttatggcaagggataagtttcgtgaagcacctgtaccaggcattccccatataagtcacggagatgtagaatcttgctttgcagctcttatcacgctttatcacattcgcaagcttctttctagtcatggaataaggccagcgtatgagatgcttgaagaaaaacttcaggaagggccatttgctaggttgatgagtaagaatgaagatattaggatgacgaagcttttgatgcagcaaaggttgtcgaacggagcaccaagcccgaaattgtccaagatgttggagattctagttgatcactacaaaataaaagatccgaggacatcacgggtcattattttctcgaatttcagaggaagcgtaagagacataatggacgcattaagtaatattgaagatgttgtcaaagcaactgagtttattggtcaaagttcaggtaagacactgaagggacagtcgcaaaaagttcagcaagctgttctggagaaatttagatctggtgggtttaatgttattgttgcaacatctatcggcgaagaaggcttggatatcatggaagtcgacttagttatatgttttgatgctaatgtatcccctctgaggatgatccaacgcatgggaagaactggaaggaaaaataatggccgagttgtagttcttgcttgtgaaggatctgaaaagaatagctatatgcgaaagaaagcaaatggccaagccattaaaaaacacatgcggaatggaggaatgaatagttttaattttcatcctagtccaaggatgattccccatgtttataagccagaagttcagcatgttaagttttcgatcgagcaattcattccacgtggaaagaagctacaagatgagcctgccactgagactccagctttcaagaaaaagcttacaccggaagagatggatatgctcgccaagtatttcaaacccaacgaggaaaagtggagagtttccttgattgctttccctcacttccaaacattgccatccaaagtgcacaaagtaatgcattcacgccaaacaagcatattaattgatgctatgcagcatctgcaagagacaactttgacagagcaaagtaaaagtttcttcattaagtatggagctcctttggctgaaagagatgagcttgacgcaggtctgagggttggtgatgatccgaaaggtaaatttagtctcaatgatttggatggcaacacatcacagagaaaggcaaaacaaattttagaatctcccacaagcacattagagactacagagaaggatttcgaagcatcttcacccacacactgttatcttttcagttcagaatgtgcgtccgttgatactctggggaaggtctttgtattgccggttcctctctcattctcttctaatgtaccagggtcagactgcgtgggaagagaaaaagaactttcttccccgaataagtcccacactgacgttgttccgatagatagttcctcaaaacatcggcaagataatatttcatgcaagttaaagcaaggattcttgccagattgtgccaacgagactttggagtcccaaagccttttgaaaaggcactccaccgatgtaggtaaaggagatatagagaattgtgctggagaaattatgatatcatcggatgaagaagacgactgtgaggatttggagcttagtccaaggctcactaacttcatcaagagtggcgttgttccagattcacctgtctatgaccaaggagttgcatacgaagcaaacagagaagaagaccttgatcttccacccacgagtttaactaatgaattggcagaagagccatcgacacctgagaaaaaggttcacattgcttctacggccaatgaattcagaactcctcagaaggaagaagatttagccaacgaaacagaaagcttcgctgtttctccaatgcctgaggagtggagaactcccttggcgaatatcaccaacgcaagcagcagcgctagcaaagattggcgcgtgagttcgggagaaaagtcagaaactcttcgacagcctcgcaagttgaagagacttcgtagacttggagattgctcgagtgctgtgaaggagaataatcctggtattgcaaagacagaccatatcagatctcgttctcgcagtgtaaagaacataagaggcaagaagaagatacgcgcggataataatgctagaatcttcattgaagcggaagctgaggtgtcttcggaatcagaaatgtcggttgatgagaacgtagatttgaccagcgattcatttgaagatagcttcatagatgacggtacaatgcctacagcaaatactcaagccgagtgtgctaaagttgacatgatggccgtttacagacgttctctactcagccaatcaccattaccggcaagatttcgtgatgtagctgcatcaagtccgagtccttattcttctggtctcttgaagacaataaatgagagcagaagcgactcagataaatcattgtcttctcttagaaccccacaaacaacgaacaacgagtcaaacaaggatgcagtggccacaggagactttttggtagcacaaatctcaacagacagccggaaaaggaaattcagcttatgcaactcagcgaatgtcccagtgattaacttggaaaacaagtttgaagctcatgcacaagccacggagaaggaaagccatgaaggtccgagaagcaatgcaggtgcatcacagtacaaggatgaggatgaagatgatgatgcattctacgcgacactggactttgatgccatggaagcgcatgcgacattgctattgtcgaaacaaaggtcagaaacgaaaacaaaagaagatgcatcggtgaaacctcatttgggcaatcagaggaatgatggtttgccgaaggatgggccatcttttgatcttggtttgtggtgattattctcctattaagttaaagtgtataaaggttgacatttggatgtatgttttgtgtatttagtttgtgtcata

SEQ ID NO: 102 - DNA, 1769 bp, synthetic construct
  misc_feature 1..1769: note = Chimeric DNA encoding a hairpin RNAi (hpRNA) construct targetinga FANCM gene of B. napus
  source 1..1769: mol_type = other DNA
gggagaaattatgatatcatcggatgaagaagacgactgtgtggatttggagcttagtccaaggctcactaacttcatcaagagtggcgttgttccagattcacctgtctatgaccaagttgcatacgaagcaaacagtgaagaagaccttgatcttccacacacgagtttaactaatgaattggcagaagagccatcgacacctgagaaaaaggttcacattgcttctacggccaatgaattcagaaccccaacgaaggaagaagatttagccaacgaaacagaaagcttcgctgtttctccaatgcctgaggagtggagaactcccttggcgaatatcaccaacgcaagcagcagcgctagcaaagattggcgcgtgagttcgggagaaaagtcagaaactcttcgacagcctcgcaagttgaagagacttcgtagacttggagattgctcgagtgctgtgaaggagaataatcctggtattgcaaagacagaccatatcgtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacaggatatggtctgtctttgcaataccaggattattctccttcacagcactcgagcaatctccaagtctacgaagtctcttcaacttgcgaggctgtcgaagagtttctgacttttctcccgaactcacgcgccaatctttgctagcgctgctgcttgcgttggtgatattcgccaagggagttctccactcctcaggcattggagaaacagcgaagctttctgtttcgttggctaaatcttcttccttcgttggggttctgaattcattggccgtagaagcaatgtgaacctttttctcaggtgtcgatggctcttctgccaattcattagttaaactcgtgtgtggaagatcaaggtcttcttctctgtttgcttcgtatgcaacttggtcatagacaggtgaatctggaacaacgccactcttgatgaagttagtgagccttggactaagctccaaatcctcacagtcgtcttcttcatccgatgatatcataatttctc

SEQ ID NO: 103 - DNA, 1769 bp, synthetic construct
  misc_feature 1..1769: note = Chimeric DNA encoding a hairpin RNAi (hpRNA) construct with G:Ubasepairs, targeting a FANCM gene of B. napus
  source 1..1769: mol_type = other DNA
gggagaaattatgatattattggatgaagaagatgattgtgtggatttggagtttagtttaaggtttattaattttattaagagtggtgttgttttagatttatttgtttatgattaagttgtatatgaagtaaatagtgaagaagattttgattttttatatatgagtttaattaatgaattggtagaagagttattgatatttgagaaaaaggtttatattgtttttatggttaatgaatttagaattttaatgaaggaagaagatttagttaatgaaatagaaagttttgttgtttttttaatgtttgaggagtggagaatttttttggtgaatattattaatgtaagtagtagtgttagtaaagattggtgtgtgagtttgggagaaaagttagaaattttttgatagttttgtaagttgaagagattttgtagatttggagattgtttgagtgttgtgaaggagaataattttggtattgtaaagatagattatattgtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacaggatatggtctgtctttgcaataccaggattattctccttcacagcactcgagcaatctccaagtctacgaagtctcttcaacttgcgaggctgtcgaagagtttctgacttttctcccgaactcacgcgccaatctttgctagcgctgctgcttgcgttggtgatattcgccaagggagttctccactcctcaggcattggagaaacagcgaagctttctgtttcgttggctaaatcttcttccttcgttggggttctgaattcattggccgtagaagcaatgtgaacctttttctcaggtgtcgatggctcttctgccaattcattagttaaactcgtgtgtggaagatcaaggtcttcttctctgtttgcttcgtatgcaacttggtcatagacaggtgaatctggaacaacgccactcttgatgaagttagtgagccttggactaagctccaaatcctcacagtcgtcttcttcatccgatgatatcataatttctc

SEQ ID NO: 104 - DNA, 1259 bp, synthetic construct
  misc_feature 1..1259: note = Chimeric DNA encoding a ledRNA construct, targeting a FANCM geneof B. napus
  source 1..1259: mol_type = other DNA
gggttctgaattcattggccgtagaagcaatgtgaacctttttctcaggtgtcgatggctcttctgccaattcattagttaaactcgtgtgtggaagatcaaggtcttcttctctgtttgcttcgtatgcaacttggtcatagacaggtgaatctggaacaacgccactcttgatgaagttagtgagccttggactaagctccaaatcctcacagtcgtcttcttcatccgatgatatcataatttctccagcacaattctctatatctcctttacctacatcggtggagtgccttttcaaaaggctttgggactccaaagtctcgttggcacaatctggcaagaatccttgctttaacttgcatgaaatattatcttggagaaattatgatatcatcggatgaagaagacgactgtgtggatttggagcttagtccaaggctcactaacttcatcaagagtggcgttgttccagattcacctgtctatgaccaagttgcatacgaagcaaacagtgaagaagaccttgatcttccacacacgagtttaactaatgaattggcagaagagccatcgacacctgagaaaaaggttcacattgcttctacggccaatgaattcagaaccccaacgaaggaagaagatttagccaacgaaacagaaagcttcgctgtttctccaatgcctgaggagtggagaactcccttggcgaatatcaccaacgcaagcagcagcgctagcaaagattggcgcgtgagttcgggagaaaagtcagaaactcttcgacagcctcgcaagttgaagagacttcgtagacttggagattgctcgagtgctgtgaaggagaataatcctggtattgcaaagacagaccatatccagcttccgcttcaatgaagattctagcattattatccgcgcgtatcttcttcttgccttgaacacagagcaaaaggaaatacagaatcattttacctcttatgttctttacactgcgagaacgagatctgatatggtctgtctttgcaataccaggattattctccttcacagcactcgagcaatctccaagtctacgaagtctcttcaacttgcgaggctgtcgaagagtttctgacttttctcccgaactcacgcgccaatctttgctagcgctgctgcttgcgttggtgatattcgccaagggagttctccactcctcaggcattggagaaacagcgaagctttctgtttcgttggctaaatcttcttccttcgtt

SEQ ID NO: 105 - DNA, 7074 bp, Nicotiana benthamiana
  source 1..7074: mol_type = other DNA
atgcgagctgtttttgaaacaaagcgtgatcgtgaaatcttggtccttgctagtaaagttctgggtcacctagctagatctggcggtgcaatgactgcagatgaagtggaacgtcagataaaagttgcactaggatggcttcgtggtgaaagaattgagtatcgtttctttgctgctgtcttaatattgaaggaaatggcggaaaatgcttcaactgttttcaatgttcatgtgccggactttgtggaggttgtttgggttgctctgaaggatccaacattggctgttcgagagaaggctgttgaggcattgcgtgcctgccttcgcgttattgaaaagcgcgagacacgatggcgtgttcagtggtattataagatgtttgaggctacccaagatggattgaccagaactgcgcctgttcatagtatacatggctcccttctcgcagtgggagagctgctaaggaatacaggagagttcatgatgtcaagatacagggaggttgcagaaattgttataagatacttggagcaccgagatcgcctagttcggctcagcataacttctctacttcctcgaattgctcatttcctgcgtgatcgatttgtgactaactacttaacgatatgcatgaatcatatacttcatgtccttaaaatacctgcagaacgtgccagtgggttcattgctcttggggagatggctggtgctctggatggtgaactcattaactatttgccgacaataacctctcacttgcgtgatgcgattgctccccgtagaggcaggccctcatttgaggctctggcatgtgttggaaatattgctaaagcaatgggacctgcaatggagcctcatgttcgtggtctcttggatgctatgttttctgctgggctttccctgacactagtggaagccttggagcaaataactgaaagcattccatctttgttgccgaccattcaagatcggcttcttgaatgtatttcagcaattctctccagatctaatcatgcactctcaagacaatcaactgctatgagtcgaggacatattgcaacagttaccccccaagtaccagaactgagtggtcctgcactagttcaacttgctttgcagactcttgctcgttttaatttcaagggccatgatcttcttgagtttgcaagggagtctgttgttgtgtatttagaagatgaggatggagctacacgaaaagatgctgcgctatgttgctgcaaactagtagcaaattctttcttggcgatatcttctacccagtttagtcctagtagaatcaatcgtgccagtggaaagcgacgtcgacttgttgaagagattgtgcaaaaacttctcatcgctgctgttgccgacgctgatgttactgttcggcattcaattttttcttctctgtatgctgatggaggattcgatgagtttctagctcaggctgatagtttgacagctatatttgccactttaaatgatgaggattttgaagttcgtgactatgcaatttcactagctggtagactatctgaaaagaatccagcatatgttcttccagcacttcgtcgccatcttattcagctgttaacttacctagagcaaagtgcagataataaatgtaaagaagagagtgcaaagttattgggttgcttgattcgcaattgtgaacgattagttcttccatacattgctcccatacacaaggctcttgttgcgaaactctgtgaaggcacaggagtcaatgcgaatagtggcattattagtggagttctagtgactgttggagatcttgccagagtgggtggctttgccatgcggcagtatatttcagaacttatgccattaatcgttgaagctctactggatggggcagctgccaccaaacgtgaagtggccgtttcaacacttggtcaagttgtacagagtacaggatatgtcataactccatacaatgagtatcctcagttgcttggtttactcttgaaactgctcaatggtgaactggcttggtcaaccagaagagaggttttgaaggttctcggcatcatgggtgcattagatccccatgtgcacaagcgcaatcagcaaagcttacccggatcccatggtgaagttacccgggtgactggtgatcctggtcaacatatcagatcaatggatgaattgcctatggatctttggccctcctttgcaacatctgaagattattattccactgttgctatcaactcactcatgcggatactcagggatccatctctgtcaagttaccaccagaaagtggttggatctcttatgtttattttcaagtccatgggccttggctgtgtcccttatttgcctaaggttttgcctgatctctttcacattgtacgaacatgtgaggatggtctaaaagaatttataacatggaagcttggaaccttggtatctattgtccgccagcacatccgtaagtatctgccagagttactctctctgatatcagaaatatggtcatctttcagcttgcctgttgctaacagacctgttcacattgctcctattttgcatctcgtggagcaactttgcttggctctcaacgatgaatttagaaagtaccttgctgatatacttccctgctgtattcaagttcttactgatgcagagaggtttagtgactacacatacgttattcctattctccacacacttgaagtttttggtgggacattagatgagcatatgcatctgcttcttcctgcacttattcggttgtttaaattggatgcttcagtagaagtaagacgcggtgcaatcaaaactctcacaagattgatacctcgtgtgcaggtcactggacacatatcttctcttgtgcatcacttgaagcttgtcttggacgggaacaaagaagagctcaggaaggatgctgttgatgcactttgttgtctagctcatgctcttggagaggacttcaccatttttattcattctattcacaagcttatggttaaacataggctgcagcacaaggaatttgaagaaatccgaggacgactggaaaaacgtgagccactgattttggggagcaccgcagctcagagattaaatcggcggttcccagttgaggtcatcagtgatcctttgagtgatggagagaatgagcactacgaggttgggacggacatgcataagcagcttaaaagccatcaggttaatgatggtagattgcgtaccgctggtgaggcttctcaacgaagcactaaagaggattgggcagagtggatgaggcatttcagcattgaacttctgaaagaatcacctagtccagcattgcgaacttgtgcaagactcgctcaactgcagccttttgttgggcgagagttgtttgctgcaggttttgttagctgctggtcacaacttaatgaggctagtcaaaggcagctagtacgtagtctagaaatggcattttcgtctccaaatatccctcctgaaattcttgctacacttctgaacttggcggagtttatggaacacgatgagagaccccttcctattgatatccgtctgcttggtgctcttgcggagaagtgtcgagcatttgcaaaggccctacactacaaggaaatggaatttgaaggcgcactttcaaataggagggatgcaaatcctgttgctgtagttgaagctctaatccatataaataatcaattacatcaacatgaggcagctgttggaatattaacatatgctcagcagcatttgggggttcaattgaaggagtcatggtacgagaaattgcaacgctgggatgatgctcttaaagcatacactgctaaggcgtcacaagcttcgagtccacatcttgctttggatgctactttagggcgtatgcgatgccttgctgctctagctcggtgggaggagcttaacaatctttgtaaggaatactggacaccagctgagccagcagctcgactggaaatggcaccaatggctgctagtgcggcctggaacatgggtgagtgggatcagatggcagagtatgtttctcggcttgatgatggtgatgaaaccaaactgcgagtcttgggaaataccgctgccagtggcgatggaagtagtaatggcacctttttcagggctgttcttctagttcggcgagggaagtacgatgaagcacgtgaatatgttgaaagagcaaggaaatgtttggcgaccgagctcgctgcactggttcttgagagctatgaacgtgcttacagcaacatggtccgtgttcagcagctttctgaattagaagaggtgattgaatactgtactcttcctatgggaaaccctgttgctgaaggaagaagagctcttgttcgcaatatgtggaatgagcgcataaagggtacaaaaagaaatgttgaggtttggcaagtacttttagctgtgagggcacttgtattgcctcctacagaagacattgaaacatggatcaaatttgcatcactttgccggaagaatggcagaattagccaagctagatctacattggttaaacttttacagttcgatccagaatcaactcctgcaactgtgcggtatcatggtccccctcaggtgatgctagcatacttaaagtaccaatggtcacttggcgaggatcataagcgaaaggaagcctttgctaggttgcaggaccttgccatggacctctcaagaacagcagctcttcaaccagtattgcagaatggattagttgcttcttctggtgtgccacttgttgctcgtgtatatctcagactcggcacttggaagtgggcactttctcctggtttggatgatgattctattcaagaaattcttagtgcatttacaaatgctactcactgtgcaacgaagtggggaaaggcatggcatacctgggcacttttcaataccgcagtgatgtctcattacactctgagaggttttgcgaatattgcttcacagtttgttgttgctgccgtaactggttattttcactctatagcatgcggagcacatgctaagggtgttgatgatagtttacaggatattcttcgtcttcttactttgtggttcaaccatggagctacttcggatgtccaaatggcattgcagaaaggattcactcatgttaacatcaacacatggttggttgttttacctcagattattgcacggatacattcaaataaccatgctgtcagagaactgatacaatccttgctagtgcgaattggacagagtcatccacaggctcttatgtatccgcttcttgtggcatgtaagtcaattagcaatttgcgcagagctgcggctcaagaagtggttgataaagttagacagcacagcggcgtactcgttgatcaggcccaacttgtctcaaaggagcttatcagggttgcaatactgtggcatgaaatgtggcatgaggcactggaagaggccagccgtttatattttggcgaacacaacattgagggcatgctgaaggtgttagagcctctgcatgaaatgcttgaggaaggagcgatgaggaacaataccactataaaggagaaagcattcatccaggcataccgtcttgagttgttggaggcgtatgaatgttgtatgaagtatcggagaactggtaaagatgctgaattaacgcaggcttgggatctctattatcatgtattcaggcggatagataagcagcttcaaacactcacaaccctggatttgcagtctgtttcccccgagttactggagtgtcgaaatttggagctagctgttcctggaacttatatagcagatgcaccagtggtgacaattgcatcatttgcaccccaacttgttgtaattacatccaaacaacggcctcgaaaattgacaatccatgggagtgatggagaagactatgcttttttgctcaaagggcacgaagatctacgccaagatgaacgtgtcatgcagttgtttggtctggttaatactttgctcgagaattcaagaaagactgcagagaaagatttatcaattcaacgatatgctgtcattccattgtcccctaatagtggactgataggatgggttccaaattgcgacaccttgcaccagcttattcgagaatatagggatgcccggaagatcaccctaaatcaagagcataaattgatgctgagttttgcaccggattatgataatttgccacttattgctaaggtggaggtgtttgaatatgctttgcaaaatacagaagggaatgacttatcaagggttctttggttaaagagtcgtacttctgaagtctggctggacagaagaacaaattatacaagaagtttggctgtcatgagtatggttggatacctacttggtctgggtgatcgacatcctagtaacctcatgcttcaccgatacagtgggaagattctgcatattgactttggagattgctttgaagcttcaatgaatcgggagaagtttccagagaaggttccctttcgactcactagaatgcttgtaaaagcaatggaggttagtggtatagagggaaatttccggtcaacatgtgagaatgtaatgcaagttctccgactgcataaagatagtgttatggctatgatggaggcctttgttcacgatccacttataaattggcgtcttttcaacttcaatgaagttccgcaaatgtccgcacttgccagtgcacatgtccctcctgttgtgaacagtgaggaatcttcttcaaatagagagcttcttcagccacaaaggggtgcaagggagagagaactgcttcaggcggtcaatcaattaggtgatgccaatgaggttctaaatgaacgtgctgtggctgttatggctcgaatgagtaataaactcacaggacgtgattttgctgctacttctacatctgcgagctctctacaacatgcactggaccacagtacgttaatttctggagagacgcgtgaagctgatcatggtttatcagtgaaactacaagtccaaaaacttattcaacaagcgtcgtctcatgaaaatctttgccaaaattatgttgggtggtgtccattttggtag

SEQ ID NO: 106 - DNA, 1513 bp, synthetic construct
  misc_feature 1..1513: note = Chimeric DNA encoding a ledRNA construct targeting a TOR gene ofN. benthamiana
  source 1..1513: mol_type = other DNA
tgaatttaggtgacactatagaacaaagaagagctcaggaaggatgctgttgatgcactttgttgtctagctcatgctcttggagaggacttcaccatttttattcattctattcacaagcttatggttaaacataggctgcagcacaaggaatttgaagaaatccgaggacgactggaaaaacgtgagccactgattttggggagcaccgcagctcagagattaaatcggcggttcccagttgaggtcatcagtgatcctttgagtgatggagagaatgagcactacgaggttgggacggacatgcataagcagcttaaaagccatcaggttaatgatggtagattgcgtaccgctggtgaggcttctcaacgaagcactaaagaggattgggcagagtggatgaggcatttcagcattgaacttctgaaagaatcacctagtccagcattgcgaacttttaagctgcttatgcatgtccgtcccaacctcgtagtgctcattctctccatcactcaaaggatcactgatgacctcaactgggaaccgccgatttaatctctgagctgcggtgctccccaaaatcagtggctcacgtttttccagtcgtcctcggatttcttcaaattccttgtgctgcagcctatgtttaaccataagcttgtgaatagaatgaataaaaatggtgaagtcctctccaagagcatgagctagacaacaaagtgcatcaacagcatccttcctgagctcttctttgttcccgtccaagacaagcttcaagtgatgcacaagagaagatatgtgtccagtgacctgcacacgaggtatcaatcttgtgagagttttgattgcaccgcgtcttacttctactgaagcatccaatttaaacaaccgaataagtgcaggaagaagcagatgcatatgctcatctaatgtcccaccaaaaacttcaagtgtgtggagaataggaataacgtatgtgtagtcactaaacctctctgcatcagtaagaacttgaatacagcagggaagtatatcagcaaggtactttctaaattcacacatccgtaagtatctgccagagttactctctctgatatcagaaatatggtcatctttcagcttgcctgttgctaacagacctgttcacattgctcctattttgcatctcgtggagcaactttgcttggctctcaacgatgaatttagaaagtaccttgctgatatacttccctgctgtattcaagttcttactgatgcagagaggtttagtgactacacatacgttattcctattctccacacacttgaagtttttggtgggacattagatgagcatatgcatctgcttcttcctgcacttattcggttgtttaaattggatgcttcagtagaagtaagacgcggtgcaatcaaaactctcacaagattgatacctcgtgtgcaggtcactggacacatatcttctcttgtgcatcacttgaagcttgtcttggacggcccgggactcgaa

SEQ ID NO: 107 - DNA, 1941 bp, Hordeum vulgare
  source 1..1941: mol_type = other DNA
atggccgcagccacctccgccgccgtcgcattctcgggcgccgccgccgccgccgcggccttacccaagcccgccctccatcctctcccgcgccaccagcccgcctcgcgccgcgcgctccccgcccgcgtcgtcaggtgctgcgccgcgtcccccgccgccaccacggccgcgcctccccccacctctctccggccgtgggggccctccgagccccgcaagggcgccgacatcctcgtcgaggcgctcgagcgctgcggcatcgtcgacgtcttcgcctaccccggcggcgcgtccatggagatccaccaggcgctcacgcgctcgcccgtcatcaccaaccacctcttccgccacgagcagggggaggcgttcgcagcgtccgggtacgcacgcgcgtccggccgcgtcggcgtctgcgtcgccacctccggccccggggccaccaacctcgtctccgcgctcgccgacgctctcctcgactccatccccatggtcgccatcacgggccaggtcccacgccgcatgatcggcacggacgcgttccaggagacgcccatagtggaggtcacgcgctccatcaccaagcacaactacctggtccttgacgtggaggacatcccccgcgtcatccaggaagccttcttcctcgcgtcctctggccgcccggggcctgtgctggttgatatccccaaggacatccagcagcagatggccgtgcctgtttgggacacgccgatgagtttgccagggtacatcgcccgcctgcccaagccaccatctactgaatcgcttgagcaggtcctgcgcctggttggcgaggcacggcgcccgattctgtatgttggtggcggctgcgctgcatctggcgaggagttgcgccgctttgttgagctcactggaattccagttacaactactctgatgggccttggcaacttccccagtgacgacccactgtcactgcgcatgcttgggatgcatggtaccgtgtatgcaaattatgcagtagataaggctgacctgttgcttgcatttggtgtgcggtttgatgatcgcgtgactgggaaaattgaggcttttgcaagcaggtccaagattgtgcacattgacattgatccagctgagattggcaagaacaagcagccacatgtctccatttgtgcagatgttaagcttgctttacaggggttgaatggtctattaagtggcagcaaagcacaacagggtctagattttggtccatggcacaaggagttggatcagcagaagagggagtttcctctaggatacaagacttttggtgaggcaatcccaccgcagtatgctatccaggtactggatgagctgacaaaaggggaggcgattattgccacaggtgttgggcagcatcagatgtgggcggctcagtattacacttacaagcggccacgtcagtggctgtcttcgtctggtttgggggcaatgggatttgggttgccagctgcagctggcgcttctgtggccaacccaggtgtcacagttgttgacattgatggggatggtagtttcctcatgaacattcaggagttggcgttgatccgtattgagaacctcccagtgaaggtgatgatattgaacaaccagcacctgggaatggtggtgcagtgggaggataggttttacaaggccaaccgggcgcacacataccttggcaacccagaaaatgagagtgagatatatccagattttgtgacgattgctaaaggattcaacgttccggcagttcgtgtgacaaagaagagtgaagtcagtgcagctatcaagaagatgcttgagaccccagggccgtacctgctggatatcattgtcccgcatcaggagcacgtgctgcctatgatcccaagcggtggtgctttcaaggacatgatcatggagggtgatggcaggacctcgtattaa

SEQ ID NO: 108 - DNA, 1505 bp, synthetic construct
  misc_feature 1..1505: note = Chimeric DNA encoding a ledRNA targeting the ALS gene of barley(H. vulgare)
  source 1..1505: mol_type = other DNA
agatttaggtgacactatagaatggtggtgcagtgggaggataggttttacaaggccaaccgggcgcacacataccttggcaacccagaaaatgagagtgagatatatccagattttgtgacgattgctaaaggattcaacgttccggcagttcgtgtgacaaagaagagtgaagtcagtgcagctatcaagaagatgcttgagaccccagggccgtacctgctggatatcattgtcccgcatcaggagcacgtgctgcctatgatcccaaacggtggtgctttcaaggacatgatcatggagggtgatggcaggacctcgtattaatctgaatttcgacctacaagacctacaagtgtgacatgcgcaatcagcatgatgcctgcgtgttgtatcaactactaggggttcgactgtgaaccatgcgtttttctagtttacttgtttcattcatataagaggtcctgccatcaccctccatgatcatgtccttgaaagcaccaccgtttgggatcataggcagcacgtgctcctgatgcgggacaatgatatccagcaggtacggccctggggtctcaagcatcttcttgatagctgcactgacttcactcttctttgtcacacgaactgccggaacgttgaatcctttagcaatcgtcacaaaatctggatatatctcactctcattttctgggttgccaaggtatgtgtgcgcccggttggccttgtaaaacctatcctcccactgcaccaccattcccaggtgctggttgttcaatatcatcaccttcactgggaggttctcaatacggatcaacgccaactcctgaatgttcatgaggaaactaccatccccatcaatgtcaacaactgtgacacctgggttggccacagaagcgccagctgcagctggcaacccaaatcccattgcccccaaaccagacgaagacagccactgacgtggccgcttgtaagtgtaatactgagccgcccacatctgatgctgcccaacacctgtggcaataatcgcctccccttttgtcagctcatccagtacctgaatggtctattaagtggcagcaaagcacaacagggtctagattttggtccatggcacaaggagttggatcagcagaagagggagtttcctctaggatacaagacttttggtgaggcaatcccaccgcagtatgctatccaggtactggatgagctgacaaaaggggaggcgattattgccacaggtgttgggcagcatcagatgtgggcggctcagtattacacttacaagcggccacgtcagtggctgtcttcgtctggtttgggggcaatgggatttgggttgccagctgcagctggcgcttctgtggccaacccaggtgtcacagttgttgacattgatggggatggtagtttcctcatgaacattcaggagttggcgttgatccgtattgagaacctcccagtgaaggtgatgatattgaacaaccagcacctggcccggg

SEQ ID NO: 109 - DNA, 1824 bp, Hordeum vulgare
  source 1..1824: mol_type = other DNA
atgcagactctgtcggcgcagcccctcgcctcctcctcttcgatacagcgccaccatgggcgccgacgcggccccggctccgtccggttcgctccccgcgcggccgccgcggctgccgccacgtccaccagcacggcccgctcgccggcgtacgtctcgtcgccgtccacgaggaaggtgcccgggtacgagcagtcgtcgccgcctgccattgcctcgccgcagaagcaggggagcagcggcggcgagggcgagcagagcctcaacttcttccagcgcgcggcggccgcggcgctcgacgcgttcgaggaggggttcatcaacaatgtcctggagcggccccacgcgctgccgcgcacggccgacccggccgtgcagatcgccggcaacttcgcccccgtcggcgagcagccccccgtgcgcgccctcacggtctccggccgcatcccgcccttcatcaacggcgtctacgcccgcaacggcgccaacccctgcttcgagcccacggccggccaccacctcttcgacggcgacggcatggtccacgccatccgcatccgaaacggcgccgccgagtcctacgcctgccgcttcaccgagaccgcccgcctctcccaggagcgcgccgcggggaggcccgtcttccctaagaccatcggcgagctccacggccactctggcatcgcgaggctggccctcttctacgcgcgcggcgcctgcggcctcgtcgacccgtcccacggcactggtgttgccaacgccggcctcgtctacttcaacggccgcctcctcgccatgtccgaggacgacctcccgtaccaggtccgcgtcaccgccggtggcgacctcgagaccgtcggccgctacgacttcgacggccagctcgactgcgccatgatcgcgcaccccaagctcgaccctgtctccggcgagctcttcgcgctcagctacgatgtcatcaagaagccgtacctcaagtacttctacttccacgccgacggcaccaagtccgccgacgtcgagatcgagctcgaccagcccaccatgatccacgacttcgccatcaccgagaacttcgtcgtcgtgcccgaccaccagatggtgttcaagctcgccgagatgttccgcggcggctcgccggtgatgctcgacaaggagaagacctcccgcttcggcgtcctcccaaagtacgccaaggactcgtcggagatgatgtgggtggacgtgccggactgcttctgtttccacctctggaactcgtgggaggagccggagacggacgaggtggtggtgatcggctcctgcacgacccccgcagactccatcttcaacgacacggacgaccacctcgagagcgtgctcaccgagatccggctcaacacgcgcaccggcgagtccacgcggcgggccatcctgccgctggagagccaggtgaacctcgaggtcggcatggtgaaccgcaacatgctgggccggaagacgaggtacgcctacctggccgtggccgagccgtggcccaaggtgtccgggttcgccaaggtggacctggtgaccggcgagctgaccaagttcgagtacggcgagggccggttcggcggcgagccgtgcttcgtgcccatggacggcgagcacgcgcgccccggcgccgaggacgacggctacgtgctctccttcgtgcgcgacgaggacgccggcacatccgagctcctggtcgtcaacgccgccgacatgcggctcgaggccaccgtgcagctgccgtcccgggtcccctatggcttccacggcacattcatcggcgacgccgacctcgacgcccagcactaa

SEQ ID NO: 110 - DNA, 1779 bp, Hordeum vulgare
  source 1..1779: mol_type = other DNA
atgcagacactcacagcgtccagctcggtctcctccatacagcggcaccggccgcaccccgcgggccgccggtccagctcggtcaccttctccgcccgcgccgtcagctccgcgccgcgcgcgccggcaccgtcccggttcgtgcgcggcgccgacgcggcgcccgccaagcccctcattgccgtccccaagccgcccgccgtggagaggcaggagaagaagctcaacttcttccagcgcgccgcggtcacggcgctcgacgcgttcgaggaaggatttgtggccaacgtgctcgagcgcccgcacggcctctccaggacggtcgaccccgcggtgcagatcgccggcaacttcgcgcctgtcggggagacacctcctgtgcaggcgctgcccgtgaccgaccgcatccccccgttcatcaacggcgtgtacgcccgcaacggcgccaacccgcacttcgaccccgtcgccgggcaccacctgttcgacggcgacggcatggtgcacgctctgcgcatccgcaacggcgtcgccgagacctacgcctcccgcttcaccgagacggagcgcctgcagcaggagcgcgcgctggggcgcccgatgttccccaaggccattggtgagctccatggccactctgggatcgcgcgccttgctctgttctacgcgcgcgcggcctgcggcctcatcgacccctcgcgcggcaccggcgtggccaacgccggcctggtctacttcaacggccacctcctcgccatgtccgaggacgacatcccgtaccacgtccgcgtcaccgacgacggcgacctccagaccgtcggccgctacgacttcgacgggcagctcgagtgccccatgatcgcgcaccccaaactcgaccccgccaccggggagctccacgcgctcagctacgacgtcatcaagaagccttacctgaagtacttctacttcgcggccgacggcaccaagtcggccgacgtcgagatcccgctggaccagcccaccatgatccacgacttcgccatcaccgagaattacgtggtcgtgcccgaccaccaggtggtgttcaagctgcaggagatgctgcgcggcggctcgcccgtggtgctcgacaaggagaagacgtcccgcttcggcgtgctgcccaagtgcgccgccgacgcgtcggagatggtgtgggtggacgtgccggactgcttctgcttccacctctggaacgcgtgggaggaggaggagaccgacgaggtggtggtgatcggctcctgcatgacccccgccgactccatcttcaacgagtcggacgagtgcctcgagagcgtgctcacggagatccgcctcaacacccgcaccggcgagtccacgcggcgccccatcctggcgctgtcagagcaggtgaacctggaggtcggcatggtgaactccaacctgctgggccgcaagacgcggtacgcctacctggccgtggccgagccgtggcccaaggtgtccggcttcgccaaggtcgacctggccacgggcgagctcaccaaattcgagtacggcgagggccggttcggcggcgagccctgcttcgtgcccatggacccggccacgtcccgcggcgaggacgacgggtacattctcaccttcgtgcacgacgaggccgccggcacgtcggagctgctggtggtcaatgccgccgacatgcggctggaggcgaccatccagctgccgtcccgcgtgccatacgggttccacggcaccttcatcaccggcaaggagctcgaatcccaggcctga

SEQ ID NO: 111 - DNA, 1500 bp, synthetic construct
  misc_feature 1..1500: note = Chimeric DNA encoding a ledRNA construct targeting the NCED1genes of barley Hordeum vulgare and wheat Triticum aestivum
  source 1..1500: mol_type = other DNA
taatacgactcactatagggtcgacgaggccgcaggcgccgcgcgcgtagaagagggccagcctcgcgatgccagagtggccgtggagctcgccgatggtcttagggaagacgggcctccccgcggcgcgctcctgggagaggcgggcggtctcggtgaagcggcaggcgtaggactcggcggcgccgtttcggatgcggatggcgtggaccatgccgtcgccgtcgaagaggtggtggccggccgtgggctcgaagcaggggttggcgccgttgcgggcgtagacgccgttgatgaagggcgggatgcggccggagaccgtgagggcgcgcacggggggctgctcgccgacgggggcgaagttgccggcgatctgcacggccgggtcggccgtgcgcggcagcgcgtggggccgctccaggacattgttgatgaacccctcctcgaacgcgtcgagctccggccgcatcccgcccttcatcaacggcgtctacgcccgcaacggcgccaacccctgcttcgagcccacggccggccaccacctcttcgacggcgacggcatggtccacgccatccgcatccgaaacggcgccgccgagtcctacgcctgccgcttcaccgagaccgcccgcctctcccaggagcgcgccgcggggaggcccgtcttccctaagaccatcggcgagctccacggccactctggcatcgcgaggctggccctcttctacgcgcgcggcgcctgcggcctcgtcgacccgtaccacggcactggtgttgccaacgccggcctcgtctacttcaacggccgcctcctcgccatgtccgaggacgacctcccgtaccaggtccgcgtcaccgccggtggcgacctcgagaccgtcggccgctacgacttcgacggccagctcgactgcgccatgatcgcgcaccccaagctcgaccctgtctccggcgagctcttcgcgctcagctacgatgtcatcaagaagccgtacctcaagtacttctacttcacgcccgacggcaccaagtccgccgacgtcgagatcgagctcgacgaagcgggaggtcttctccttgtcgagcatcaccggcgagccgccgcggaacatctcggcgagcttgaacaccatctggtggtcgggcacgacgacgaagttctcggtgatggcgaagtcgtggatcatggtgggctggtcgagctcgatctcgacgtcggcggacttggtgccgtcgggcgtgaagtagaagtacttgaggtacggcttcttgatgacatcgtagctgagcgcgaagagctcgccggagacagggtcgagcttggggtgcgcgatcatggcgcagtcgagctggccgtcgaagtcgtagcggccgacggtctcgaggtcgccaccggcggtgacgcggacctggtacgggaggtcgtcctcggacatggcgaggaggcggccgttgaagtagacgaggccggcgttggcaacaccagtgccgtggtacgta

SEQ ID NO: 112 - DNA, 1500 bp, synthetic construct
  misc_feature 1..1500: note = Chimeric DNA encoding a ledRNA construct targeting the NCED2genes of barley Hordeum vulgare and wheat Triticum aestivum
  source 1..1500: mol_type = other DNA
taatacgactcactatagggttggcgccgttgcgggcgtacacgccgttgatgaacggggggatgcggtcggtcacgggcagcgcctgcacaggaggtgtctccccgacaggcgcgaagttgccggcgatctgcaccgcggggtcgaccgtcctggagaggccgtgcgggcgctcgagcacgttggccacaaatccttcctcgaacgcgtcgagcgccgtgaccgcggcgcgctggaagaagttgagcttcttctcctgcctctccacggcgggcggcttggggacggcaatgaggggcttggcgggcgccgcgtcggcgccgcgcacgaaccgggacggtgccggcgcgcgcggcgcggagctgacggcgcgggcggagaaggtgaccgagctggaccggcggcccgcggggtgcggccggtgccgctgtatggaggagaccgagctggacgctgtcgacgcggcgcccgccaagcccctcattgccgtccccaagccgcccgccgtggagaggcaggagaagaagctcaacttcttccagcgcgccgcggtcacggcgctcgacgcgttcgaggaaggatttgtggccaacgtgctcgagcgcccgcacggcctctccaggacggtcgaccccgcggtgcagatcgccggcaacttcgcgcctgtcggggagacacctcctgtgcaggcgctgcccgtgaccgaccgcatccccccgttcatcaacggcgtgtacgcccgcaacggcgccaacccgtacttcgaccccgtcgccgggcaccacctgttcgacggcgacggcatggtgcacgctctgcgcatccgcaacggcgtcgccgagacctacgcctcccgcttcaccgagacggagcgcctgcagcaggagcgcgcgctggggcgcccgatgttccccaaggccattggtgagctccatggccactctgggatcgcgcgccttgctctgttctacgcgcgcgcggcctgcggcctcatcgacccctcgcgcggcaccggcgtggccaacgccggcctggtctacttcaacggccacctcctccccggtggcggggtcgagtttggggtgcgcgatcatggggcactcgagctgcccgtcgaagtcgtagcggccgacggtctggaggtcgccgtcgtcggtgacgcggacgtggtacgggatgtcgtcctcggacatggcgaggaggtggccgttgaagtagaccaggccggcgttggccacgccggtgccgcgcgaggggtcgatgaggccgcaggccgcgcgcgcgtagaacagagcaaggcgcgcgatcccagagtggccatggagctcaccaatggccttggggaacatcgggcgccccagcgcgcgctcctgctgcaggcgctccgtctcggtgaagcgggaggcgtaggtctcggcgacgccgttgcggatgcgcagagcgtgcaccatgccgtcgccgtcgaacaggtggtgcccggcgacggggtcgaagtacgta

SEQ ID NO: 113 - DNA, 1521 bp, Hordeum vulgare
  source 1..1521: mol_type = other DNA
atggccttcttcctcctcctgtgcatcctcgtctctgtggccatcgtgtcctacgcccaccacgcaatccggcggaggcgccagggctgcgctcatggccgtcatgagcaggccgccctcaagctgccccccggctccatgggcctgccttacgtcggcgagaccctgcagctctactcccaggaccccagcgtcttcctctcctccaagcagaagcggtacggcgagatcttcaagacgcacctcctggggtgcccgtgcgtgatgctggcgagcccggaggcggcgcgcttcgtgctggtgtcgcgggcccacctcttcaagccgacgtacccgcggagcaaggagcgcctcatcggcccgtcggcgctcttcttccaccagggcgactaccacctccgcctccgccggctcgtccagggcccgctcggccccgaggccctgcgcaagctcgtgccggacatcgaggccgccgttcgctccacgctcgccgcctgggcggacggcgacgtcgccagcactttccacgccatgaagaggctctcgttcgacgtcggcatcgtgacgatcttcggcgggcggctggacgagcggcggaaggaggagctcaggcggaactacgccgtcgtggagaaaggctacaactccttccccaacagcttccccgggacgctatactacaaggcgatccaggcgaggcggcggctgaacggcgtgctgagcgacgtcgtgcacgagcgtagggagcggggcgagcacggcgacgacctcctcggctgcctcatgcggtcgcgggccggcggcgacgacgccgacgacgagggcgcgctgctgacggacgagcaggtcgccgacaacgtcatcggcgtgctgttcgcggcgcaggacacgacggccagcgtgctcacctggatcgtcaagtacctccacgaccgcccgaagctgctcgaggccgtcagggcggagcacgcggcgatccacgaggccaacgacggcgggaggcggccgctgacatgggcgcagacgaggagcatgacgctgacgcacagggtgattttggagagcctaaggatggccagcatcatctccttcacgttcagggaggccgtggccgacgtggagtacaaagggtttcttatccccaaggggtggaaggtgatgccgctcttcaggaacatccatcacagcccggactacttccaggatccacacaagttcgacccttcgcgattcaaggtggcgccgcggccgaacaccttcacsccgttcgggagcggggtgcacgcgtgcccggggaacgagctggccaagctcgagatgctggtgctcatccaccacctggtcaccggctacaggtgggaggttgttggatcgagcgacgacgtcgagtacagcccattccccgttccccgccatggcctgctcgccagggtacggcgagatgacggcgtctgcgcgggtaggaaggggtgcccgactgatgaagatgacaactacgacgacgacgaagtgatagtgtga

SEQ ID NO: 114 - DNA, 1506 bp, synthetic construct
  misc_feature 1..1506: note = Chimeric DNA encoding a ledRNA construct targeting the ABA-OH-2genes of barley Hordeum vulgare and wheat Triticum aestivum
  source 1..1506: mol_type = other DNA
taatacgactcactatagggcggtcgtggaggtacttgacgatccaggtgagcacgctggccgtcgtgtcctgcgccgcgaacagcacgccgatgacgttgtcggcgacctgctcgtccgtcagcagcgcgccctcgtcgtcggcgtcgtcgccgccggcccgcgaccgcatgaggcagccgaggaggtcgtcgccgtgctcgccccgctccctacgctcgtgcacgacgtcgctcagcacgccgttcagccgccgcctcgcctggatcgccttgtagtatagcgtcccggggaagctgttggggaaggagttgtagcctttctccacgacggcgtagttccgcctgagctcctccttccgccgctcgtccagccgcccgccgaagatcgtcacgatgccgacgtcgaacgagagcctcttcatggcgtggaaagtgctggcgacgtcgccgtccgcctacaactccttccccaacagcttccccgggacgctatactacaaggcgatccaggcgaggcggcggctgaacggcgtgctgagcgacgtcgtgcacgagcgtagggagcggggcgagcacggcgacgacctcctcggctgcctcatgcggtcgcgggccggcggcgacgacgccgacgacgagggcgcgctgctgacggacgagcaggtcgccgacaacgtcatcggcgtgctgttcgcggcgcaggacacgacggccagcgtgctcacctggatcgtcaagtacctccacgaccgcccgtagctgctcgaggccgtcagggcggagcacgcggcgatccacgaggccaacgacggcgggaggcggccgctgacatgggcgcagacgaggagcatgacgctgacgcacagggtgattttggagagcctaaggatggccagcatcatctccttcacgttcagggaggccgtggccgacgtggagtacaaagggtttcttatccccaaggggtggaaggtgatgccgctcttcaggaacatccatcacagcccggactacttccaggatccacacaagttcgacccttcgcgattcaaggtcgctcgatccaacaacctcccacctgtagccggtgaccaggtggtggatgagcaccagcatctcgagcttggccagctcgttccccgggcacgcgtgcaccccgctcccgaacggagtgaaggtgttcggccgcggcgccaccttgaatcgcgaagggtcgaacttgtgtggatcctggaagtagtccgggctgtgatggatgttcctgaagagcggcatcaccttccaccccttggggataagaaaccctttgtactccacgtcggccacggcctccctgaacgtgaaggagatgatgctggccatccttaggctctccaaaatcaccctgtgcgtcagcgtcatgctcctcgtctgcgcccatgtcagcggccgcctcccgccgtcgttggcctcgtggatcgccgcgtgctccgccctgacggcctcgagcagctacgtaggtacc

SEQ ID NO: 115 - DNA, 3885 bp, Arabidopsis thaliana
  source 1..3885: mol_type = other DNA
atggaagctgaaattgtgaatgtgagacctcagctagggtttatccagagaatggttcctgctctacttcctgtccttttggtttctgtcggatatattgatcccgggaaatgggttgcaaatatcgaaggaggtgctcgtttcgggtatgacttggtggcaattactctgcttttcaattttgccgccatcttatgccaatatgttgcagctcgcataagcgttgtgactggtaaacacttggctcagatctgcaatgaagaatatgacaagtggacgtgcatgttcttgggcattcaggcggagttctcagcaattctgctcgaccttaccatggttgtgggagttgcgcatgcacttaaccttttgtttggggtggagttatccactggagtgtttttggccgccatggatgcgtttttatttcctgttttcgcctctttccttgaaaatggtatggcaaatacagtatccatttactctgcaggcctggtattacttctctatgtatctggcgtcttgctgagtcagtctgagatcccactctctatgaatggagtgttaactcggttaaatggagagagcgcattcgcactgatgggtcttcttggcgcaagcatcgtccctcacaatttttatatccattcttattttgctggggaaagtacatcttcgtctgatgtcgacaagagcagcttgtgtcaagaccatttgttcgccatctttggtgtcttcagcggactgtcacttgtaaattatgtattgatgaatgcagcagctaatgtgtttcacagtactggccttgtggtactgacttttcacgatgccttgtcactaatggagcaggtatttatgagtccgctcattccagtggtctttttgatgctcttgttcttctctagtcaaattaccgcactagcttgggctttcggtggagaggtcgtcctgcatgacttcctgaagatagaaatacccgcttggcttcatcgtgctacaatcagaattcttgcagttgctcctgcgctttattgtgtatggacatctggtgcagacggaatataccagttacttatattcacccaggtcttggtggcaatgatgcttccttgctcggtaataccgcttttccgcattgcttcgtcgagacaaatcatgggtgtccataaaatccctcaggttggcgagttcctcgcacttacaacgtttttgggatttctggggttgaatgttgtttttgttgttgagatggtatttgggagcagtgactgggctggtggtttgagatggaataccgtgatgggcacctcgattcagtacaccactctgcttgtatcgtcatgtgcatccttatgcctgatactctggctggcagccacgccgctgaaatctgcgagtaacagagcggaagctcaaatatggaacatggatgctcaaaatgctttatcttatccatctgttcaagaagaggaaattgaaagaacagaaacaaggaggaacgaagacgaatcaatagtgcggttggaaagcagggtaaaggatcagttggatactacgtctgttactagctcggtctatgatttgccagagaacattctaatgacggatcaagaaatccgttcgagccctccagaggaaagagagttggatgtaaagtactctacctctcaagttagtagtcttaaggaagactctgatgtaaaggaacagtctgtattgcagtcaacagtggttaatgaggtcagtgataaggatctgattgttgaaacaaagatggcgaaaattgaaccaatgagtcctgtggagaagattgttagcatggagaataacagcaagtttattgaaaaggatgttgaaggggtttcatgggaaacagaagaagctaccaaagctgctcctacaagcaactttactgtcggatctgatggtcctccttcattccgcagcttaagtggggaagggggaagtgggactggaagcctttcacggttgcaaggtttgggacgtgctgcccggagacacttatctgcgatccttgatgaattttggggacatttatatgattttcatgggcaattggttgctgaagccagggcaaagaaactagatcagctgtttggcactgatcaaaagtcagcctcttctatgaaagcagattcgtttggaaaagacattagcagtggatattgcatgtcaccaactgcgaagggaatggattcacagatgacttcaagtttatatgattcactgaagcagcagaggacaccgggaagtatcgattcgttgtatggattacaaagaggttcgtcaccgtcaccgttggtcaaccgtatgcagatgttgggtgcatatggtaacaccactaataataataatgcttacgaattgagtgagagaagatactctagcctgcgtgctccatcatcttcagagggttgggaacaccaacaaccagctacagttcacggataccagatgaagtcatatgtagacaatttggcaaaagaaaggcttgaagccttacaatcccgtggagagatcccgacatcgagatctatggcgcttggtacattgagctatacacagcaacttgctttagccttgaaacagaagtcccagaatggtctaacccctggaccagctcctgggtttgagaattttgctgggtctagaagcatatcgcgacaatctgaaagatcttattacggtgttccatcttctggcaatactgatactgttggcgcagcagtagccaatgagaaaaaatatagtagcatgccagatatctcaggattgtctatgtccgcaaggaacatgcatttaccaaacaacaagagtggatactgggatccgtcaagtggaggaggagggtatggtgcgtcttatggtcggttaagcaatgaatcatcgttatattctaatttggggtcacgggtgggagtaccctcgacttatgatgacatttctcaatcaagaggaggctacagagatgcctacagtttgccacagagtgcaacaacagggaccggatcgctttggtccagacagccctttgagcagtttggtgtagcggagaggaatggtgctgttggtgaggagctcaggaatagatcgaatccgatcaatatagacaacaacgcttcttctaatgttgatgcagaggctaagcttcttcagtcgttcaggcactgtattctaaagcttattaaacttgaaggatccgagtggttgtttggacaaagcgatggagttgatgaagaactgattgaccgggtagctgcacgagagaagtttatctatgaagctgaagctcgagaaataaaccaggtgggtcacatgggggagccactaatttcatcggttcctaactgtggagatggttgcgtttggagagctgatttgattgtgagctttggagtttggtgcattcaccgtgtccttgacttgtctctcatggagagtcggcctgagctttggggaaagtacacttacgttctcaaccgcctacagggagtgattgatccggcgttctcaaagctgcggacaccaatgacaccgtgcttttgccttcagattccagcgagccaccagagagcgagtccgacttcagctaacggaatgttacctccggctgcaaaaccggctaaaggcaaatgcacaaccgcagtcacacttcttgatctaatcaaagacgttgaaatggcaatctcttgtagaaaaggccgaaccggtacagctgcaggtgatgtggctttcccaaaggggaaagagaatttggcttcggttttgaagcggtataaacgtcggttatcgaataaaccagtaggtatgaatcaggatggacccggttcaagaaaaaacgtgactgcgtacggatcattgggttga

SEQ ID NO: 116 - DNA, 1080 bp, synthetic construct
  misc_feature 1..1080: note = Chimeric DNA encoding a ledRNA construct targeting the EIN2 geneof A. thaliana
  source 1..1080: mol_type = other DNA
taatacgactcactatagggagctgcaacatattggcataagatggcggcaaaattgaaaagcagagtaattgccaccaagtcatacccgaaacgagcacctccttcgatatttgcaacccatttcccgggatcaatatatccgacagaaaccaaaaggacaggaagtagagcaggaaccattctctggataaaccctagctgaggtctcacattcacaatttcagcttccatcctaaatctatctgataatataattactcagagtaggattcaaggtaaactctacagtcgtggttcactaaaagcctcagttgagtaaaattcacagatttgatctaaaacacctgaatgtgagacctcagctagggtttatccagagaatggttcctgctctacttcctgtccttttggtttctgtcggatatattgatcccgggaaatgggttgcaaatatcgaaggaggtgctcgtttcgggtatgacttggtggcaattactctgcttttcaattttgccgccatcttatgccaatatgttgcagctcccaacagcgttgtgactggtaaacacttggctcagatctgcaatgaagaatatgacaagtggacgtgcatgttcttgggcattcaggcggagttctcagcaattctgctcgaccttaccatggttgtgggagttgcgcatgcacttaaccttttgtttggggtggagttatccactggagtgtttttggccgccatggatgcgagtgggatctcagactgactcagcaagacgccagatacatagagaagtaataccaggcctgcagagtaaatggatactgtatttgccataccattttcaaggaaagaggcgaaaacaggaaataaaaacgcatccatggcggccaaaaacactccagtggataactccaccccaaacaaaaggttaagtgcatgcgcaactcccacaaccatggtaaggtcgagcagaattgctgagaactccgcctgaatgcccaagaacatgcacgtccacttgtcatattcttcattgcagatctgagccaagtgtttaccagtcacaacgctgttgac

SEQ ID NO: 117 - DNA, 1188 bp, Arabidopsis thaliana
  source 1..1188: mol_type = other DNA
atggtgatggctggtgcttcttctttggatgagatcagacaggctcagagagctgatggacctgcaggcatcttggctattggcactgctaaccctgagaaccatgtgcttcaggcggagtatcctgactactacttccgcatcaccaacagtgaacacatgaccgacctcaaggagaagttcaagcgcatgtgcgacaagtcgacaattcggaaacgtcacatgcatctgacggaggaattcctcaaggaaaacccacacatgtgtgcttacatggctccttctctggacaccagacaggacatcgtggtggtcgaagtccctaagctaggcaaagaagcggcagtgaaggccatcaaggagtggggccagcccaagtcaaagatcactcatgtcgtcttctgcactacctccggcgtcgacatgcctggtgctgactaccagctcaccaagcttcttggtctccgtccttccgtcaagcgtctcatgatgtaccagcaaggttgcttcgccggcggtactgtcctccgtatcgctaaggatctcgccgagaacaatcgtggagcacgtgtcctcgttgtctgctctgagatcacagccgttaccttccgtggtccctctgacacccaccttgactccctcgtcggtcaggctcttttcagtgatggcgccgccgcactcattgtggggtcggaccctgacacatctgtcggagagaaacccatctttgagatggtgtctgccgctcagaccatccttccagactctgatggtgccatagacggacatttgagggaagttggtctcaccttccatctcctcaaggatgttcccggcctcatctccaagaacattgtgaagagtctagacgaagcgtttaaacctttggggataagtgactggaactccctcttctggatagcccaccctggaggtccagcgatcctagaccaggtggagataaagctaggactaaaggaagagaagatgagggcgacacgtcacgtgttgagcgagtatggaaacatgtcgagcgcgtgcgttctcttcatactagacgagatgaggaggaagtcagctaaggatggtgtggccacgacaggagaagggttggagtggggtgtcttgtttggtttcggaccaggtctcactgttgagacagtcgtcttgcacagcgttcctctctaa

SEQ ID NO: 118 - DNA, 1080 bp, synthetic construct
  misc_feature 1..1080: note = Chimeric DNA encoding a ledRNA construct targeting the CHS geneof A. thaliana
  source 1..1080: mol_type = other DNA
taatacgactcactataggggccacaccatccttagctgacttcctcctcatctcgtctagtatgaagagaacgcacgcgctcgacatgtttccatactcgctcaacacgtgacgtgtcgccctcatcttctcttcctttagtcctagctttatctccacctggtctaggatcgctggacctccagggtgggctatccagaagagggagttccagtcacttatccccaaaggtttaaacgcttcgtctagactcttcacaatgttcttggagatgaggccgggaacatccttgaggagatggaaggtgagaccaacttccctcaaatgtccgtctatggcaccatcagactggaactccctcttctggatagcccaccctggaggtccagcgatcctagaccaggtggagataaagctaggactaaaggaagagaagatgagggcgacacgtcacgtgttgagcgagtatggaaacatgtcgagcgcgtgcgttctcttcatactagacgagatgaggaggaagtcagctaaggatggtgtggccccgacaggagaagggttggagtggggtgtcttgtttggtttcggaccaggtctcactgttgagacagtcgtcttgcacagcgttcctctctaaacagaacgcttgccttctatctgcctacctacctacgcaaaactttaatcctgtcttatgttttatataatataatcattatatgtttacgcaataattaaggaagaatgacatttccaaacaaagatttgatgtcattcaagacccatagatttaatattgtaaaaagacacaaaaaagagagtacaaaaacagtcgaatagacctgtccagcacatatcacatatcacatcaaatgcattcttccttaattattgcgtaaacatataatgattatattatataaaacataagacaggattaaagttttgcgtaggtaggtaggcagatagaaggcaagcgttctgtttagagaggaacgctgtgcaagacgactgtctcaacagtgagacctggtccgaaaccaaacaagacaccccactccaacccttctcctgtcaac

SEQ ID NO: 119 - DNA, 3456 bp, Lupinus angustifolius
  source 1..3456: mol_type = other DNA
atgttgactcttcaacccacacatgagtcaagtagtcaataccctcctcatacacttatagctgagacctgtcactttgattatctgtactatactaatcaaagttctctaattatgtcacttggagaatcatccctgcaatggaaataccatgttttcttgagttttaggggaggtgacacccgcttaagcttcactaatcacttatatgctgcgttggtgcgaaaaggaatcattactttccgagatgacaaacaacttcacaaaggagatgccatttctcaacatctgcatcaatcaatccaacagtctctagctgccattgttgttatctcggagaactatgcttcttccacttggtgtttggatgagctaaaactaattcttgaatcgagaatagatgtttttccagtcttttatggtgtcactccttctgatgttcgataccagaaaaatagttttgctgaggctttcaataaacatgttgtaagatttgaacaagatgaagagaaagtgcaaaaatggagagattgcttgaaagaagttgctgatttttctggatgggagtccaaggacatggctgaagcagaactcattgaagatgttattgaaaaggtatggataaaactacaaccaaaattgccatcctacaatgaaggagtggttggatttgattcaagggtgaagaaaatgatttcacttttaagcataggatcacaagatattcggtttatcgggatatggggtatggctggaactggaaaaacaattcttgctagagtaatctacgaaacaataagtagccaatttgagattaaatgtttccttcttaatgttagagaggtttctcaaacatctgatggattggtttccttacaaagaaaacttctttctacccttaagataagcaacctagaaattgatgatttgtatgatggaaagaagaaaattatgaaccttttgtgcaacaaaagtgttcttcttgtccttgatgacattagtcatttaagtcagctagagaatttggctaaaactaaaggttggtttggtccatgcagcagagtgataataacaaccaaagatatgcacttactagtatcacatggtgcgtgtgagaagtatgagatgagaatcttaaatgaaagttcttcctttcaactcttcagccagaaagcattcagaagagataaacctccagagggttatttagaaataactaaaagtatggtcaaatatgctggaggtcttcctttggcacttaaagtgttgggttcttttgtttgtggaagaagtctcagtcagtggaaggatgctttggataagataaaacaagttctgccgaaagacattttgaacacactaataataggttatgatggactagaagatgcagaaaagactttgtttttagatattgctttcttctttacaggacggtcgaaaattgaagtgatacaggtattggcagattgtggccttaatccaacaattggaataagtcttcttattgaaagatctctagtaagttgttgtggaggaattttggaaatgcatgatttacttcaagaaatgggtagaaatattgtatatcaagaatctccggatgatgcaagcagacgcagtaggttatgctctttagaagatattaaccgagtattcagaaaaaacaagggaaccaatatcattcaaggaatagttctgaaatcaagtgacccatgtgaagcatattggcatcctgaagccttctcaaaaatggataatcttagagtactcatcattttgtgtgatttgcaccttcccctcggcctcaaatgtctctctagttcattaaaacttcttgaatggaagggatatcctttggaatatctaccatttggcctgcaactgctagaacttgttcacttgaaaatgcattgcagcaaacttaaacaactttggaatggaactcaaattttcagagagctaaaatcaattgatctcagtgattccagagatctaattcaaactccagatatttctgaggttccatgtcttgagagtttagttttgaaaggttgtaaaaaccttgttgaggttcatcaatctgttgcaaagcacaagaatgttgctatactagacctggaaggttgcatcagtcttaagaccctgccaagaaaattggagatgaatgctttggaaaagttcattctctccggctgctcacaaattaaaaaccttcccgaatttggggagagtatggaatgtctatctatgcttaatttaagagattgcacaagtcttgtttctcttccacagagtgttcgaaacatgaaatcctttagagatctcaatatccatggttgctcaaaattgtttaagctgacaaacaattcaaatgaaaataatgtcgtggaagaaattgatgagactgaaacaggtaggagagaagtgcattcatcatggagcttttctctccttactgagaaagtgtttgatttcgtaaagtatccagttagcatggactcgaagttgccttctctctcaagtttccctcggttgaagaaattagatatgggcaactgtaatctcagtgatggaccaattatagatcatattggacatttaacatcactggaagtgttatatttagctgggaacaactttgttgaccttacagcaagcattggtaacctttctcggctacaacgccttggtttatataaatgccgaagacttaggacattgcctgagcttccacccagtgtatgccagttacttatgaacgactgcactcaactggaacctatgttatttgacacacaaataattttgaaaatatttgaggcaaatagatggagcctgacacgcgaattgtggttcctgattccagggagtgaaatcccagcatggtttgagcatcaagattattttagcctgaaaccaagtttagcgcctttcgattatcacgaggagtatgcttttattgtttcaacaatagtaaacatccctgactattgcctttcaagtgattggataggaattattgtatgctttttactggaaagtggtttaaaggcagacctacacagacatattcgtagaagtccggtcacgatcggatggtcttttaaagatcccgatgcagaaacggtttaccccttacgcttcactaaacgtcgttggacacatttcaaaggcaatcacctattgattactacttttggaagtgatcatagaatatacaagcactacttaacttgtggcaaaagcaaagtgcaattgatattttgtggtgagaatatttgcaagtgcgggaagctaaagctgaaaaactgtgggatccgtgtgatttgtaaggaagatggtgtatcgcgtagaggcgaggaaacgagtgaagttgaggtgccttccacttcagttgaatctgatgttcacaaacaatcacgaataactgaaattacagatgaatatgaataa

SEQ ID NO: 120 - DNA, 1280 bp, synthetic construct
  misc_feature 1..1280: note = Chimeric DNA encoding a ledRNA construct targeting the L.angustifolius N-like gene
  source 1..1280: mol_type = other DNA
taatacgactcactatagggtatcgaacatcagaaggagtgacaccataaaagactggaaaaacatctattctcgattcaagaattagttttagctcatccaaacaccaagtggaagagtatttccattgcagggatgattctccaagtgacataattagagaactttgattagtatagtacagataatcaaagtgacaggtctcagctataagtgtatgaggagggtattgactacttgactcatgtgtgggttgaagagtcaacatatagcttcacggatcccacagtttttcagctttagcttcccgcacttgcaaatattctcaccacaaaatatcaattgcactttgcttttgccacaagttaagtagtgcttgtatattctatgatcaccctgttgactcttcaacccacacatgagtcaagtagtcaataccctcctcatacacttatagctgagacctgtcactttgattatctgtactatactaatcaaagttctctaattatgtcacttggagaatcatccctgcaatggaaatactcttccacttggtgtttggatgagctaaaactaattcttgaatcgagaatagatgtttttccagtcttttatggtgtcactccttctgatgttcgatacccgtagttttgctgaggctttcaataaacatgttgtaagatttgaacaagatgaagagaaagtgcagtttgagcatcaagattattttagcctgaaaccaagtttagcgcctttcgattatcacgaggagtatgcttttattgtttcaacaatagtaaacatccctgactattgcctttcaagtgattggataggaattattgtatgctttttactggaaagtggtttaaaggcagacctacacagacatattccaaaagtagtaatcaataggtgattgcctttgaaatgtgtccaacgacgtttagtgaagcgtaaggggtaaaccgtttctgcatcgggatctttaaaagaccatccgatcgtgaccggacttctacgaatatgtctgtgtaggtctgcctttaaaccactttccagtaaaaagcatacaataattcctatccaatcacttgaaaggcaatagtcagggatgtttactattgttgaaacaataaaagcatactcctcgtgataatcgaaaggcgctaaacttggtttcaggctaaaataatcttgatgctcaaactgcactttctcttcatcttgttcaaatcttacaacatgtttattgaaagcctcagcaaaactacgta

SEQ ID NO: 121 - DNA, 1527 bp, Vitis pseudoreticulata
  source 1..1527: mol_type = other DNA
atggctggcgacgaggagacgacgacgacggcagcaacacttgaaacaacgtccacttgggctgttgcctctgtttgctttattttgattgcactctccatacttattgagcatgccctccatctcttagccaagtacttcaacaagaagcggaggaggtctctcattcatgctcttaacaacgtcaaatcggagttgatgctcttggggttcgtctctttgttgctgactgtgtgccaaaagtatattgcgaagatttgtatcccaaggagcgtaggtgaaacttttcttccctgcaagaccttgacagaaagtgattcagaagaagaaaccaaatgcgaagagcagggaaagatgtctttgctgtctagacaaggcgtggaggaactacaatacttaattttcgtgctggccttcttccattccctctactgcgtcctcacattcggtcttgggatggccaagatgaagaaatgggagtcctgggaggcagaaacaagaacactggaatatcagtttacaaatgatccacggaggttcaggctcatccatcagacatcatttggaaagcaacatctgagatattggagtgagcatcagatacttcgttggccggcttgttttattcggcagttctatccatccgtctccaaagtggattacttgactcttagacatgggttcattatggcccattttgcagaaggaagcaactatgacttccaaaagtatataaaaagagctttggaaaaagactttggagtggtggtgggaggaagtttctgggtttggagtttctccatgctttttgtgttcttcaatgctcaagtattttacaactatttatggctaccctttattccattggtgatgctgttgttggttggaacaaagctacagggcattataactaagatgtgcttagatagccatgataaagctctcgttgttagaggaactttgcttgtcaggcccagtgatcacttcttctggtttggaaaaccggaattgctcctacatcttatgcactttatattgtttcagaactcttttcaactggcgttctttacatggacttggtacaaatttggattcagatcatgcttccatgatacaactgaggatatcgtcataaggcttgtcatgggtgtgttagtacaactcctttgtggctacgtgacactgcctctgtatgccctggtcacgcagatggggacatcaatgaggacaattgtctttactgagggagtcgttgaaggtctgaacagatggagaaggaaagccaagaaaaacatagcacgcaggaacaaccactcagctcgtccctccctggatgcttcactcgacaattcaccttcttttaacactctggatacttctttctctgtagacctcgatcagccatcatcagatgctggttatttgactgttgaaatatcagatgaagagacggtcgctactaaacggccagaaccgcgtcagaagttgggatcttttgagggtttcgactcgtgcaaaacatcataa

SEQ ID NO: 122 - DNA, 1480 bp, synthetic construct
  misc_feature 1..1480: note = Chimeric DNA encoding a first ledRNA construct targeting a VitisMLO gene
  source 1..1480: mol_type = other DNA
taatacgactcactatagggtagccataaatagttgtaaaatacttgagcattgaagaacacaaaaagcatggagaaactccaaacccagaaacttcctcccaccaccactccaaagtctttttccaaagctctttttatatacttttggaagtcatagttgcttccttctgcaaaatgggccataatgaacccatgtctaagagtcaagtaatccactttggagacggatggatagaactgctgaataaaacaaaccggccaacgaagtatccgatgctcactccaatatctcagatgttgctttccaaatgatgtctgatggatgagcgtgaacctccgtggatcatttgtaaactgatattccagtgttcttgtttctgcctcccaggactcccatttcttcatcttggccatcccaagaccgaatgtgaggacgcagtagagcacatcatttggaaagcaacatctgagatattggagtgagcatcggatacttcgttggccggtttgttttattcagcagttctatccatccgtctccaaagtggattacttgactcttagacatgggttcattatggcccattttgcagaaggaagcaactatgacttccaaaagtatataaaaagagctttggaaaaagactttggagtggtggtgggaggaagtttctgggtttggagtttctccatgctttttgtgttcttcaatgctcaagtattttacaactatttatggctacccgtaattccattggtgatgctgttgttggttggaacaaagctacagggcattataactaagatgtgcctagatagccatgataaagctctcgttgttagaggaactttgcttgtcaggcccagtgatcacttcttctggtttggaaaaccggaattgctcctacatcttatgcactttatattgtttcagaactcttttcaactggcgttctttacatggacttggtacaaatttggattcagatcatgcttccatgatacaactgaggatatcgtcataaggcttgtcatgggtgtgttatggctttccttctccatctgttcagaccttcaacgactccctcagtaaagacaattgtcctcattgatgtccccatctgcgtgaccagggcatacagaggcagtgtcacgtagccacaaaggagttgtactaacacacccatgacaagccttatgacgatatcctcagttgtatcatggaagcatgatctgaatccaaatttgtaccaagtccatgtaaagaacgccagttgaaaagagttctgaaacaatataaagtgcataagatgtaggagcaattccggttttccaaaccagaagaagtgatcactgggcctgacaagcaaagttcctctaacaacgagagctttatcatggctatctaggcacatcttagttataatgccctgtagctttgttccaaccaacaacagcatcaccaatggaattacgta

SEQ ID NO: 123 - DNA, 894 bp, Myzus persicae
  source 1..894: mol_type = other DNA
atgttcaaacacttgtgcaataccgtttcacaaagtataaaacctagtagttttttatcaaaagtttgttcaaacaaatatctcgtcgtgccgtaccggatagcgatttttaacaacatgggaagttacaaattgtacctggccgtcatggcaatagctgtcatagctgcagttcaggaaattagttgcaaggttcagacttccgaacaggacgatgatcaggaaggatattacgatgatgagggaggagtgaacgataatcagggagaagagaacgataatcagggagaagagaacgataatcagggagaagagaacgataatcagggagaagagaaggaagaagtttccgaaccagagatggagcaccatcagtgcgaagaatacaaatcgaagatctggaacgatgcatttagcaacccgaaggctatgaacctgatgaaactgacgtttaatacagctaaggaattgggctccaacgaagtgtgctcggacacgacccgggccttatttaacttcgtcgatgtgatggccaccagcccgtacgcccacttctcgctaggtatgtttaacaagatggtggcgtttattttgagggaggtggacacgacatcggacaaatttaaagagacgaagcaggtggtcgaccgtatctcgaaaactccagagatccgtgactatatcaggaactcggccgccaagaccgtcgacttgctcaaggaacccaagattagagcacgactgttcagagtgatgaaagccttcgagagtctgataaaaccaaacgaaaacgaagcattaatcaaacagaagattaaggggttaaccaatgctcccgtcaagttagccaagggtgccatgaaaacggttggacgtttctttagacatttttaa

SEQ ID NO: 124 - DNA, 960 bp, Myzus persicae
  source 1..960: mol_type = other DNA
atgactgagacaatgcaactccgtggtacccttcgtgggcataatggttgggttacgcagatcgccaccaatccgatccacactgacatgattctgtcttgttcacgagacaagaccttgattgtttgggatctgacacgtgatgagctcaactatggtatccccaagaaacgtttgtacggacattcgcacttcgtcagcgacgtcgttctttcatcagatggtaactacgctctttccggttcttgggataagactcttcgtctgtgggatttggctgctggacgtaccactcgtcgttttgaagaccacaccaaggatgtattgagcgttgccttctctgctgacaaccgtcaaatcgtttctggaagtcgggacaagactatcaagttgtggaatactttggctgagtgcaaatacactattcaggatgatggacatagcgattgggtatcatgtgtacggttctctcctaatatccataacccaatcattgtgagtgctggttgggacaaggttgtcaaggtatggaacttaactaactgccgcatcaagaccaaccattatggacacactggataccttaacaccgttactgtttcacctgatggttctttgtgtgcttcaggaggaaaagattgcaaagctatgttatgggatcttaatgacggcaaacacttgcacacactggaccataacgatatcattgaagctttgtgctttagccccaaccgttactggttgtgcgctgcatttggaccatcaatcaaaatttgggatttggaaagcaaagaaatggttgaggaacttcgcccagaagttgtatctcaatcacagaatagcaataccgaaccacccagatgtctgtcacttgcatggtcaactgatggacaaacattgtttgctggatactcagacaataacattagagtttggcaagtgtctgtcagtgctcgttaa

SEQ ID NO: 125 - DNA, 1401 bp, synthetic construct
  misc_feature 1..1401: note = Chimeric construct encoding the ledRNA targeting M.persicae C002gene
  source 1..1401: mol_type = other DNA
gaattctaatacgactcactatagggtgctccatctctggttcggaaacttcttccttctcttctccctgattatcgttctcttctccctgattatcgttctcttctccctgattatcgttctcttctccctgattatcgttcactcctccctcatcatcgtaatatccttcctgatcatcgtcctgttcggaagtctgaaccttgcaactaatttcctgaactgcagctatgacagctattgccatgacggccaggtacaatttgtaacttcccatgttgttaaaaatcgctatccggtacggcacgacgagatatttgtttgaacaaacttttgataaaaaactactaggttttatactttgtgaaacggtattgcacaagtgtttgaacataagagagttggaagttacaaattgtacctggccgtcatggcaatagctgtcatagctgcagttcaggaaattagttgcaaggttcagacttccgaacaggacgatgatcaggaaggatattacgatgatgagggaggagtgaacgataatcagggagaagagaacgataatcagggagaagagaacgataatcagggagaagagaacgataatcagggagaagagaaggaagaagtttccgaaccagagatggagcacccaacagtgcgaagaatacaaatcgaagatctggaacgatgcatttagcaacccgaaggctatgaacctgatgaaactgacgtttaatacagctaaggaattgggctccaacgaagtgtgctcggacacgacccgggccttatttaacttcgtcgatgtgatggccaccagcccgtacgcccacttctcgctaggtatgtttaacaagatggtggcgtttattttgagggaggtggacacgacatcggacaatctgaacagtcgtgctctaatcttgggttccttgagcaagtggacggtcttggcggccgagttcctgatatagtcacggatctctggagttttcgagatacgggcgaccacctgcttcgtctctttaaatttgtccgatgtcgtgtccacctccctcaaaataaacgccaccatcttgttaaacatacctagcgagaagtgggcgtacgggctggtggccatcacatcgacgaagttaaataaggcccgggtcgtgtccgagcacacttcgttggagcccaattccttagctgtattaaacgtcagtttcatcaggttcatagccttcgggttgctaaatgcatcgttccagatcttcgatttgtattcttcgcactgttaacaagcttagcatatccatgatatctgttagtttttttcctgaaagagcggccgccctagcataaccccgcggggcctcttcgggggtctcgcggggttttttgctgaaaggatcc

SEQ ID NO: 126 - DNA, 1401 bp, synthetic construct
  misc_feature 1..1401: note = Chimeric construct encoding the ledRNA targeting M.persicaeRack-1 gene
  source 1..1401: mol_type = other DNA
gaattctaatacgactcactatagggttatggatattaggagagaaccgtacacatgatacccaatcgctatgtccatcatcctgaatagtgtatttgcactcagccaaagtattccacaacttgatagtcttgtcccgacttccagaaacgatttgacggttgtcagcagagaaggcaacgctcaatacatccttggtgtggtcttcaaaacgacgagtggtacgtccagcagccaaatcccacagacgaagagtcttatcccaagaaccggaaagagcgtagttaccatctgatgaaagaacgacgtcgctgacgaagtgcgaatgtccgtacaaacgtttcttggggataccatagttgagctcatcacgtgtcagatcccaaacaatcaaggtcttgtcccggttcttgggataagactcttcgtctgtgggatttggctgctggacgtaccactcgtcgttttgaagaccacaccaaggatgtattgagcgttgccttctctgctgacaaccgtcaaatcgtttctggaagtcgggacaagactatcaagttgtggaatactttggctgagtgcaaatacactattcaggatgatggacatagcgattgggtatcatgtgtacggttctctcctaatatccataacccaaccattgtgagtgctggttgggacaaggttgtcaaggtatggaacttaactaactgccgcatcaagaccaaccattatggacacactggataccttaacaccgttactgtttcacctgatggttctttgtgtgcttcaggaggaaaagattgcaaagctatgttatgggatcttaatgacggcaaacacttgcacacactggaccataacgatatcattgaagctttgtgctttagccccaaccgttacacagacatctgggtggttcggtattgctattctgtgattgagatacaacttctgggcgaagttcctcaaccatttctttgctttccaaatcccaaattttgattgatggtccaaatgcagcgcacaaccagtaacggttggggctaaagcacaaagcttcaatgatatcgttatggtccagtgtgtgcaagtgtttgccgtcattaagatcccataacatagctttgcaatcttttcctcctgaagcacacaaagaaccatcaggtgaaacagtaacggtgttaaggtatccagtgtgtccataatggttggtcttgatgcggcagttagttaagttccataccttgacaaccttgtcccaaccagcactcacaatggttaacccgggtagcatatccatgatatctgttagtttttttcctgaaagagcggccgccctagcataaccccgcggggcctcttcgggggtctcgcggggttttttgctgaaaggatccc

SEQ ID NO: 127 - DNA, 2396 bp, Helicoverpa armigera
  source 1..2396: mol_type = other DNA
agacattgattagtgagctccaaactccgtacgtacgttcttagtttagtttgttcgttcgtattgtcgcagtcacatcgctccggtgcccgcttcgacatttcccgccaaaagtgacgtaacatatccgtgatctgtgtgaatatgtcagtgacttttttaaattaattttttaatagcaaaattgtgatcgaaggaatttttacaagatgacggctgggaatgaagagcatgagcctctaattacatcgtctgtcgacaatcagcgtgtggcctacagtaattcaccaccggatgaccgcacaccagaatcttcttccccacgcggcagtggcggagaagtaacgctagccataccatcacaccgcaactatggagccatcggaggcgtggagaaggtcacatacacctgggcagacatcaatgcctttgctactgaatccaggtctaggtcccgaaggatttggaacttctggaagccctccgccagtggcatgttccagcaaaggaaacagttgttgaggaatgtaaatggagccgcctacccaggcgaactgctcgccatcatgggatcctccggtgccgggaagaccacactcctcaacactctgaccttccgcactccaagcggggtgctgtccagtggcactcgagcactgaacggccagcctgctacccctgaggcgttatcagcactgtctgcgtatgttcagcagcaggatctgttcattggcacgctgactgtgaaggagcatttagtattccaggctatggtgcggatggaccgacatataccgtatgcgcagcgcatgaggagagttcaagaggttattactgagttggcgctaacaaaatgccagaacacagtgataggcatccctgggcggctgaagggtatctccggcggggagatgaagaggctgtccttcgccagcgaggtgctcacggatccaccgctcatgttctgcgatgaacccacctctggactcgattcttttatggcgcagaatgttatacaggtactgaaaggtctcgcacaaaaaggcaagacagtcgtatgcacgatccaccagccgtcttcggagctgtacgcgatgttcgataagctgctcatcatggcagacgggaaggtcgccttcctcggctcccctgatcaggctaatgatttctttaaagacctaggagcagcgtgtcctcctaactacaacccagcggaccacttcatccaactcctggcgggagtgccgggcagggaggagaccacgcgcaccactatcgatactgtctgcacggcattcgcgcgctctgaggtcggctgcaagattgctgcagaagctgaaaatgcactctactttgagcgcaagatatcgcagggctgggcggacccggcgtggtctgaagccacggctatccgcgcgcgccgctcgccgtacaaggcgtcgtggtgcgcgcagttccgcgcggtgctgtggcgctcgtggctgtccgtcactaaggagcccatgctcatcaaagtgcgcttcctacagactattatggtatcgatcctgatcggcgtgatctacttcgggcagcacctggaccaggacggcgtgatgaacatcaacggcgccatcttcatgttcctcaccaacatgaccttccagaacatcttcgctgttattaacgtattctgctcagaactgccaatattcatacgagaacaccactccgggatgtatcgagctgacgtgtacttcctatcgaagacgttagccgaagcacctgtgttcgccaccataccacttgtgttcaccaccatagcatactacatgatagggctgaaccctgaacctaagcggttctttatagcgtccggtttggctgccctgattactaacgttgctacgtcgtttggctacctgatatcgtgtgccagcaacagcgtgagcatggcagcgtcagtgggacctcccatcatcatccccttcatgttgttcggaggcttcttcctcaacactggctccgtaccaccatacctgggctggatatcgtacctgtcctggttccactacggcaacgaagcgctgctggtcaaccagtggtctggagtggaaaccatcgcctgcacccgggagaacttcacctgtcccgcctctgggcaggtcgtcttggatactcttagcttttctgaggatgacttcacaatggacgtggtgaacatgatcctacttttcatcggcttcagatttttggcgtatctcgctctcttgtaccgcgctcgccgaggcaagtgagtcttaggtacaaaatgctgcgagaatgggccatatgaaggaagaatgttgaataaatagtgtaattatttaggatgtaaggagtcaatggagatttgataaataaaacaatttataccg

SEQ ID NO: 128 - DNA, 1480 bp, synthetic construct
  misc_feature 1..1480: note = Chimeric DNA encoding a ledRNA construct targeting a ABCtransporter white gene of Helicoverpa armigera
  source 1..1480: mol_type = other DNA
taatacgactcactatagggtatatgtcggtccatccgcaccatagcctggaatactaaatgctccttcacagtcagcgtgccaatgaacagatcctgctgctgaacatacgcagacagtgctgataacgcctcaggggtagcaggctggccgttcagtgctcgagtgccactggacagcaccccgcttggagtgcggaaggtcagagtgttgaggagtgtggtcttcccggcaccggaggatcccatgatggcgagcagttcgcctgggtaggcggctccatttacattcctcaacaactgtttcctttgctggaacatgccactggcggagggcttccagaagttccaaatccttcgggacctagacctggattcagtagcaaaggcattgatgtctgcccaggtgtatgtgaccttctccacgcctccgatggctccatagttgttccagcaaaggaaacagttgttgaggaatgtaaatggagccgcctacccaggcgaactgctcgccatcatgggatcctccggtgccgggaagaccacactcctcaacactctgaccttccgcactccaagcggggtgctgtccagtggcactcgagcactgaacggccagcctgctacccctgaggcgttatcagcactgtctgcgtatgttcagcagcaggatctgttcattggcacgctgactgtgaaggagcatttagtattccaggctatggtgcggatggaccgacatatacccgtatgcgcagcgcatgaggagagttcaagaggttattactgagttggcgctaacaaaatgccagaacacagtgataggcatccctgggcggctgaagggtatctccggcggggagatgaagaggctgtccttcgccagcgaggtgctcacggatccaccgctcatgttctgcgatgaacccacctctggactcgattcttttatggcgcagaatgttatacaggtactgaaaggtctcgcacaaaaaggcaagacagtcgtatgcacgatccaccagccgtcttcggagctgtacgcgatgatgaagtggtccgctgggttgtagttaggaggacacgctgctcctaggtctttaaagaaatcattagcctgatcaggggagccgaggaaggcgaccttcccgtctgccatgatgagcagcttatcgaacatcgcgtacagctccgaagacggctggtggatcgtgcatacgactgtcttgcctttttgtgcgagacctttcagtacctgtataacattctgcgccataaaagaatcgagtccagaggtgggttcatcgcagaacatgagcggtggatccgtgagcacctcgctggcgaaggacagcctcttcatctccccgccggagatacccttcagccgcccagggatgcctatcactgtgttctggcattttgttagcgccaactcagtaataacctcttgaactctcctcatgcgctgcgcatacgta

SEQ ID NO: 129 - DNA, 811 bp, Linepithema humile
  source 1..811: mol_type = other DNA
agagagaacgatgaggacaatgagatggaaaaaacaacaacgtcccaacgtcccttcgacgacgccattccaccagccctataaaaccccgaggatcatcggcgtcccaacattactcggtcagagtctcgaggaacgccgtgtccgagatgatcatcaccaggaaccgcatcaaccgcgcaactctaatctgcgttctggcgtcgtggctttgcttggcgtctcgcgcttccgccgaatacgaatcgcgggagatgtcgaacggcggaccgggcgtcgacgcctcgtgcatcgagggcaagtgcatgaagcgcaccgccacgcaggatgctaccgccagcatgtggttcggcccgcgtttgggaagacggcgcagatcggacgagaagcaggaagtgaattccgagatacaggctctggcggaagccttggatagcgggcgtttggccctatttgccattccagctaacgacaagagacaaccgactcaatttacaccgcgactggggcgaggatcagacgaggacctatcctcctacggagacgcgattgagaggaacgagatcgacgatcgtatattacccgcgttattcgcgccgcgtttaggacgacgaattccttggtcaccgtcgccgagacttggacgccaattacgcagcattttgcgaaaaatgtaggcgccgtcgaaagattattatcaaaagttacaaatgaagagtgatctcgtagacctgcgcgtgaagatgaaataacaactaaaattatagcactattaagacataaagaaataaagtactgatgtttatttgta

SEQ ID NO: 130 - DNA, 1360 bp, synthetic construct
  misc_feature 1..1360: note = Chimeric DNA encoding a ledRNA construct targeting a PBAN gene inArgentine ants
  source 1..1360: mol_type = other DNA
taatacgactcactatagggaattcacttcctgcttctcgtccgatctgcgccgtcttcccaaacgcgggccgaaccacatgctggcggtagcatcctgcgtggcggtgcgcttcatgcacttgccctcgatgcacgaggcgtcgacgcccggtccgccgttcgacatctcccgcgattcgtattcggcggaagcgcgagacgccaagcaaagccacgacgccagaacgcagattagagttgcgcggttgatgcggttcctggtgatgatcatctcggacacggcgttcctcgagactctgaccgagtaatgttgggacgccgatgatcctcggggttttatagggctggtggaatggcgtcgtcgaagggacgttgggacgttgttgttttttccatctcattgtcctcatcgttcacgccgtgtccgagatgatcatcaccaggaaccgcatcaaccgcgcaactctaatctgcgttctggcgtcgtggctttgcttggcgtctcgcgcttccgccgaatacgaatcgcgggagatgtcgaacggcggaccgggcgtcgacgcctcgtgcatcgagggcaagtgcatgaagcgcaccgccacgcaggatgctaccgccagcatgtggttcggcccgcgtttgggaagacggcgcagatcggacgagaagcaggaagtgaattcccgtaatacaggctctggcggaagccttggatagcgggcgtttggccctatttgccattccagctaacgacaagagacaaccgactcaatttacaccgcgactggggcgaggatcagacgaggacctatcctcctacggagacgcgattgagaggaacgagatcgacgatcgtatattacccgcgttattcgcgccgcgtttaggacgacgaattccttggtcaccgtcgccgagacttggacgccaattacgcagcattttgcgaaaaatgaaacatcagtactttatttctttatgtcttaatagtgctataattttagttgttatttcatcttcacgcgcaggtctacgagatcactcttcatttgtaacttttgataataatctttcgacggcgcctacatttttcgcaaaatgctgcgtaattggcgtccaagtctcggcgacggtgaccaaggaattcgtcgtcctaaacgcggcgcgaataacgcgggtaatatacgatcgtcgatctcgttcctctcaatcgcgtctccgtaggaggataggtcctcgtctgatcctcgccccagtcgcggtgtaaattgagtcggttgtctcttgtcgttagctggaatggcaaatagggccaaacgcccgctatccaaggcttccgccagagcctgtattacgta

SEQ ID NO: 131 - DNA, 1480 bp, synthetic construct
  misc_feature 1..1480: note = Chimeric DNA encoding a ledRNA construct targeting a geneencoding V-type proton ATPase catalytic subunit A of L. cuprina
  source 1..1480: mol_type = other DNA
taatacgactcactatagggaagttcttgtcatagaaatcatccaaagcacgcatgtatttggagtaggaaatcaaccaattgatggaggggaaatgtttacgttgggccaatttcttgtccaaaccccagaacacttgtacgatacccaaagtggcagaagtaacgggatcagagaaatcaccaccaggaggagatacagcaccgacaatggaaacggaaccttcacgttcagggttacccaaacacttgacacgaccagcacgttcgtagaaggaggccaaacgggcacccaagtaggctgggtaaccggaatcggcaggcatttcagccaaacgaccagaaatttcacgaagagcttcggcccaacgggaggtagaatcagccatcatagatacgttgtaacccatatcacggaagtattcagacaaggtaataccggtataaacgattccggttacccagcctacttgggtgcccgtttggcctccttctacgaacgtgctggtcgtgtcaagtgtttgggtaaccctgaacgtgaaggttccgtttccattgtcggtgctgtatctcctcctggtggtgatttctctgatcccgttacttctgccactttgggtatcgtacaagtgttctggggtttggacaagaaattggcccaacgtaaacatttcccctccatcaattggttgatttcctactccaaatacatgcgtgctttggatgatttctatgacaagaacttcccgtaattcgtaccattgcgtaccaaggtcaaggaaatcttgcaagaagaagaagatttgtccgaaattgtacaattggtcggtaaggcttcattggccgaaactgacaagatcaccttggaagtcgccaaattgcttaaggacgatttcttgcaacagaactcctactcatcatacgacagattctgccccttctacaagagtgtgggtatgttgaagaacatcattgccttctacgacttggctcgtcactccgtcgaatccaccgctcaatctgaaaacaaaatcacctggaatgtcattctgaaagcctgttgtaaatcttcgtgtaattgttcaaagtcagccttgatcttggcttcaccgtccttaacgggatccttgaatttcatggaagacaattggtacataatgttacccatagcttcacggatgacattccaggtgattttgttttcagattgagcggtggattcgacggagtgacgagccaagtcgtagaaggcaatgatgttcttcaacatacccacactcttgtagaaggggcagaatctgtcgtatgatgagtaggagttctgttgcaagaaatcgtccttaagcaatttggcgacttccaaggtgatcttgtcagtttcggccaatgaagccttaccgaccaattgtacaatttcggacaaatcttcttcttcttgcaagatttccttgaccttggtacgcaatggtacgaattacgta

SEQ ID NO: 132 - DNA, 1480 bp, synthetic construct
  misc_feature 1..1480: note = Chimeric DNA encoding a ledRNA construct targeting a geneencoding RNAse 1/2 of L. cuprina
  source 1..1480: mol_type = other DNA
taatacgactcactatagggaataatttgtggtagacatagcgggttacttcctcatgttcgttaaagcagacctggtattgtctcatgaaacgggaagatgacaattcaaaaccaacattgacgagagtagtgccaccattgcagctgctgcccgacttcttagctacaaaagcaggccaactggtgcaaacaagactgggcagggagtgctgaacaccattgactttaaaagtggtgccactaacacaggtagcgatatgggtcttgcctgataaaggatgagcaaagccactggtacaatggatctcaatattcttgccagcagccacatcaattcttccagtatcagaaaaagggtagagttcagtagtgccaggtttgatatacaagggttgtttagccttaagaccaccgcgaatgggtatggaacaaccaccactgcgtggaatattgagatccattgtaccagtggctttgctcatcctttatcaggcaagacccatatcgctacctgtgttagtggcaccacttttaaagtcaatggtgttcagcactccctgcccagtcttgtttgcaccagttggcctgcttttgtagctaagaagtcgggcagcagctgcaatggtggcactactctcgtcaatgttggttttgaattgtcatcttcccgtttcatgagacaataccaggtctgctttaacgaacatgaggaagtaacccgctatgtctaccacaaattattcccgtacccaacagcgtgccactttcctattcattaatgcagctccccagtggcaagttttcaatgccggtaattgggctcgtgtagaggatggtgtacgcgcctgggtgtccaaaaataaaatcaatgttcgatgctataccggtgtttatggtgtcaccactctacccaacaaagagggacgtgagactcctctatatttgtctcgtgatgccaataataatggtttgattcctgttcccaaattatacttccgtgtggttatacaacctgccaccaataagggtattgttttcgttggtgtcacaggcataagaataaccagcagtgatatcagttttcttccaactaatatagttaaccttatcactgacatctttgcaaataatatagtcctttttgatttgttccaaagtcaaatggggattgttgacaccaacgaaaacaatacccttattggtggcaggttgtataaccacacggaagtataatttgggaacaggaatcaaaccattattattggcatcacgagacaaatatagaggagtctcacgtccctctttgttgggtagagtggtgacaccataaacaccggtatagcatcgaacattgattttatttttggacacccaggcgcgtacaccatcctctacacgagcccaattaccggcattgaaaacttgccactggggagctgcattaatgaataggaaagtggcacgctgttgggtacgta

SEQ ID NO: 133 - DNA, 1480 bp, synthetic construct
  misc_feature 1..1480: note = Chimeric DNA encoding a ledRNA construct targeting a geneencoding chitin synthase of L. cuprina
  source 1..1480: mol_type = other DNA
taatacgactcactatagggtggcttgatttttatattaacaccatacacctcaaatgcagctttttcaatgctggtaatcaatatttttacatattcattgagaggaggatttttcggattctccacatatttttcatccagtataaaggcatcatcaaagaaaatattagctaaaatgtaaaaaaaagaaaacttaattgatagatactcttcattccaatccacaattcgtctatttaatgttatacattgttcaaccaaaaaaccacaataccacggacaaatgaacagtttttcggtgggtaaatttttatcattttttggacgccatatatgatttgttatccacaattgtgacagccaccacaacaaccatatccaaagataatctttagcgaccacattaaaaaaactccatgtatcgtgaccgaagcccagtgacgttgataaaaatttacccaccgaaaaactgttcatttgtccgtggtattgtggttttttggttgaacaatgtataacattaaatagacgaattgtggattggaatgaagagtatctatcaattaagttttcttttttttacattttagctaatattttctttgatgatgcctttatactggatgaaaaatatgtggagaatccgaaaaatcctcctctcaatgaatatgtaaaaatattgattaccagcattgaaaaagctgcatttgaggtgtatggtgttaatataaaaatcaagccacccgtaaaaattgaaacaccttatggcggtcgtttggtgtggacactgcctggtcgctcaaagatgattgcccatttaaaaaacaaagataaaatacgacataagaaacgctggtcacaggttatgtacatgtactatttgttgggttttcgtataatggaattggaatcagtatcggccaagcgtaaggcagtgatagcagaaaatacatttttgctggctcttgatggtgatattgactttcaaccgcaggcagtgcaactgttaatagaccgtatgaaggccatagatgaattaggtgctagccaggactacataaaacacaaccaataacatgctctgttgctttttgcaaccaatgacctatagcgtattcgaagatttgataccaaaccatagggcctctaccaactggatgaatacgaccacaggcagcacctaattcatctatggccttcatacggtctattaacagttgcactgcctgcggttgaaagtcaatatcaccatcaagagccagcaaaaatgtattttctgctatcactgccttacgcttggccgatactgattccaattccattatacgaaaacccaacaaatagtacatgtacataacctgtgaccagcgtttcttatgtcgtattttatctttgttttttaaatgggcaatcatctttgagcgaccaggcagtgtccacaccaaacgaccgccataaggtgtttcaatttttacgta

SEQ ID NO: 134 - DNA, 1481 bp, synthetic construct
  misc_feature 1..1481: note = Chimeric DNA encoding a ledRNA construct targeting a geneencoding ecdysone receptor (EcR) of L. cuprina
  source 1..1481: mol_type = other DNA
taatacgactcactatagggtgaaagatcatcacgacctgatgatatggaattagctgattcagatctgggtgtatgatgcatcatactactgttattcatatgatgatggtgatgatgatttaatccattactgatgatactgtgaatgccaatattggcatgaataacttggccattttgtgaggcctgtaacgagtttaactgttgggcattgctaacgatattgggaccattaatattgaccgataagccaccaccaccgccgccaatacccatgtggccatttgtgtggtgggaactgctattactgtgattactgttgctgttgtggtgtaaatgattgtggctgtgattgtgattattcacttgactgccaccaccaccacccagaccattgagtgaagtcataccgggtacaccaccaccacctccacctcctccaacaaatcacagtaatagcagttcccaccacacaaatggccacatgggtattggcggcggtggtggtggcttatcggtcaatattaatggtcccaatatcgttagcaatgcccaacagttaaactcgttacaggcctcacaaaatggccaagttattcatgccaatattggcattcacagtatcatcagtaatggattaaatcatcatcaccatcatcatatgaataacagtagtatgatgcatcatacacccagatctgaatcagctaattccatatcatcaggtcgtgatgatctttcacccgtatccaccaaatcacccccttagtggttcgaaacacttgtgttccatttgtggagaccgcgccagtggaaaacattatggggtctacagttgtgagggttgtaaagggttcttcaaacgtaccgtacgcaaggacttgacatatgcttgtcgtgaggacagaaattgcattatagataaacgacaaagaaatcgttgccagtattgtcgttatcaaaagtgtttagcttgtggcatgaaacgcgaagcggtccaagaggaacgacaacgtggtactcgtgctgctaacgctagagctgccttttgctcggcttcaatgatgcgttctatagtgagatcacgtaatgaactgctgggtttaaagtcttctccgccagcaccaaccacattgcttaccccaccaccacctcctccaccaccgccagcaccagcagctctagcgttagcagcacgagtaccacgttgtcgttcctcttggaccgcttcgcgtttcatgccacaagctaaacacttttgataacgacaatactggcaacgatttctttgtcgtttatctataatgcaatttctgtcctcacgacaagcatatgtcaagtccttgcgtacggtacgtttgaagaaccctttacaaccctcacaactgtagaccccataatgttttccactggcgcggtctccacaaatggaacacaagtgtttcgaaccactaagggggtgatttggtggatacgtag

SEQ ID NO: 135 - DNA, 1481 bp, synthetic construct
  misc_feature 1..1481: note = Chimeric DNA encoding a ledRNA construct targeting a geneencoding gamma-tubulin 1/1-like of L. cuprina
  source 1..1481: mol_type = other DNA
taatacgactcactatagggaaaacgctgtaggtttgtataagtttcttaggaaaacgatcagacaaacgttccataatataagagcccatgccggaaccagtaccaccggctatagaatggcatagaacaaatccctccaaggaatcactgccatctgcctcacgatcaataatgtcaaaaatttcctcttgtaatttttcaccttgactatagccggaagcccaattgttgccggcaccaccaccatgtttagacaagtaaacattttcgggattatataacttggcatagggtgaactcataatggtgtgtataactcgcggctccaaatccaaaagtacggcacgtggtatatagtgatcatcgtcagcctgataaaagaatacatccttgcgatctactccatctgtagcaaaatcctctaacactccactaggtgaaatgctatacacaccattatgagttcaccctatgccaagttatataatcccgaaaatgtttacttgtctaaacatggtggtggtgccggcaacaattgggcttccggctatagtcaaggtgaaaaattacaagaggaaatttttgacattattgatcgtgaggcagatggcagtgattccttggagggatttgttctatgccattctatagccggtggtactggttccggcatgggctcttatattatggaacgtttgtctgatcgttttcctaagaaacttatacaaacctacagcgttttcccgtaccacaaccctacgttatccttcatatatgaataataatttgataggattgacggcacctttgatacctaccccccaattacattttctaatgaccggttatactcctctaactacagatagtgatcccaatttgaatatacgcaaaactacggtactagatgttatgagacgtttattgcaacccaaaaatatgatggtttcatcgggtccggataaagcaaatattcattgttatatttccatattaaatattatacagggtgaagtagatcccactcaagtccacaaatctctactgattggccatcattaggcccgaaactttatgattactttgtatatatggagaacttctggacaaggctacttgtatactggccggaccccagggtatgaattgagctaatttgcgttcacgtatacgttgtagagatttgtggacttgagtgggatctacttcaccctgtataatatttaatatggaaatataacaatgaatatttgctttatccggacccgatgaaaccatcatatttttgggttgcaataaacgtctcataacatctagtaccgtagttttgcgtatattcaaattgggatcactatctgtagttagaggagtataaccggtcattagaaaatgtaattggggggtaggtatcaaaggtgccgtcaatcctatcaaattattattcatatatgaaggataacgtagggttgtggtacgtag

SEQ ID NO: 136 - DNA, 1605 bp, Triticum aestivum
  source 1..1605: mol_type = other DNA
atggcaaaggacgacgggtaccccccggcgcggacgctgccggagacgccgtcctgggcggtggcgctggtcttcgccgtcatgatcatcgtctccgtcctcctggagcacgcgctccacaagctcggccattggttccacaagcggcacaagaacgcgctggcggaggcgctggagaagatgaaggcggagctgatgctggtgggattcatctcgctgctgctcgccgtcacgcaggacccaatctccgggatatgcatctcccagaaggccgccagcatcatgcgcccctgcaaggtggaacccggttccgtcaagagcaagtacaaggactactactgcgccaaagagggcaaggtggcgctcatgtccacgggcagcctgcaccagctccacatattcatcttcgtgctagccgtcttccatgtcacctacagcgtcatcatcatggctctaagccgtctcaagatgagaacatggaagaaatgggagacagagaccgcctccttggaataccagttcgcaaatgatcctgcgcggttccgcttcacgcaccagacgtcgttcgtgaagcggcacctgggcctgtccagcacccccggcgtcagatgggtggtggccttcttcaggcagttcttcaggtcggtcaccaaggtggactacctcatcttgagggcaggcttcatcaacgcgcacttgtcgcagaacagcaagttcgacttccacaagtacatcaagaggtccatggaggacgacttcaaagtcgtcgttggcatcagcctcccgctgtgggctgtggcgatcctcaccctcttccttgatatcgacgggatcggcacactcacctgggtttctttcatccctctcatcatcctcttgtgtgttggaaccaagctagagatgatcatcatggagatggccctggagatccaggaccggtcgagcgtcatcaagggggcacccgtggtcgagcccagcaacaagttcttctggttccaccgccccgactgggtcctcttcttcatacacctgacgctgttccagaacgcgtttcagatggcacatttcgtgtggacagtggccacgcccggcttgaaggactgcttccatatgaacatcgggctgagcatcatgaaggtcgtgctggggctggctctccagttcctgtgcagctacatcaccttccccctctacgcgctagtcacacagatgggatcaaacatgaagaggtccatctttgacgagcagacagccaaggcgctgaccaactggcggaacacggccaaggagaagaagaaggtccgagacacggacatgctgatggcgcagatgatcggcgacgcaacacccagccgaggcacgtccccgatgcctagccggggctcatcgccggtgcacctgcttcagaagggcatgggacggtctgacgatccccagagcgcaccgacctcgccaaggaccatggaggaggctagggacatgtacccggttgtggtggcgcatcctgtacacagactaaatcctgctgacaggcggaggtcggtctcttcatcagccctcgatgccgacatccccagcgcagatttttccttcagccagggatga

SEQ ID NO: 137 - DNA, 1277 bp, synthetic construct
  misc_feature 1..1277: note = Chimeric DNA encoding a ledRNA construct, targeting Mlo fromTriticum aestivum
  source 1..1277: mol_type = other DNA
taatacgactcactatagggtgcccccttgatgacgctcgaccggtcctggatctccagggccatctccatgatgatcatctctagcttggttccaacacacaagaggatgatgagagggatgaaagaaacccaggtgagtgtgccgatcccgtcgatatcaaggaagagggtgaggatcgccacagcccacagcgggaggctgatgccaacgacgactttgaagtcgtcctccatggacctcttgatgtacttgtggaagtcgaacttgctgttatgcgacaaatgcgcgttgatgaagcctgccctcaaggtgaggtagtccaccttggtgaccgacctgaagaactgcctgaagaaggccaccacccatctgacgccgggggtgctggagaggggttcgacttccacaagtacatcaagaggtccatggaggacgacttcaaagtcgtcgttggcatcagcctcccgctgtgggctgtggcgatcctcaccctcttccttgatatcgacgggatcggcacactcacctgggtttctttcatccctctcatcatcctcttgtgtgttggaaccaagctagagatgatcatcatggagatggccctggagatccaggaccggtcgagcgtcatcaagggggcacccgacgtcgagcccagcaacaagttcttctggttccaccgccccgactgggtcctcttcttcatacacctgacgctgttccagaagtcacacagatgggatcaaacatgaagaggtccatcttcgacgagcagacagccaaggcgctgaccaactggcggaacacggccaaggagaagaagaaggtccgagacacggacatgctgatggcgcagatgatcggcgacgcgacgcccagccgaggcacgtcccccaccacaaccgggtacatgtccctagcctcctccatggtccttggcgaggtcggtgcgctctggggatcgtcagaccgtcccatgcccttctgaagcaggtgcaccggcgatgagccccggctaggcatcggggacgtgcctcggctgggcgtcgcgtcgccgatcatctgcgccatcagcatgtccgtgtctcggaccttcttcttctccttggccgtgttccgccagttggtcagcgccttggctgtctgctcgtcgaagatggacctcttcatgtttgatcccatctgtgtgacttctggaacagcgtcaggtgtatgaagaagaggacccagtcggggcggtggaaccagaagaacttgttgctgggctcgacgtc

SEQ ID NO: 138 - DNA, 1527 bp, Vitis pseudoreticulata; identical to SEQ ID NO: 121
  source 1..1527: mol_type = other DNA

SEQ ID NO: 139 - DNA, 1480 bp, synthetic construct; identical to SEQ ID NO: 122
  misc_feature 1..1480: note = Chimeric DNA encoding a ledRNA construct targeting a Vitis MLOgene
  source 1..1480: mol_type = other DNA

(19) United States
(12) Patent Application Publication
(10) Pub. No.: US 2026/0125676 A1
(43) Pub. Date: May 7, 2026

(54) RNA MOLECULES

(71) Applicant: Commonwealth Scientific and Industrial Research Organisation, Acton (AU)

(72) Inventors: Neil Andrew SMITH, Cook, Australian Capital Territory (AU); Ming-Bo WANG, McKellar, Australian Capital Territory (AU); Timothy James DORAN, Ocean Grove, Victoria (AU); Mark TIZARD, Highton, Victoria (AU); Annapurna Devi ALLU, Mangalam, Tirupati, Andhra Pradesh (IN); Ian Kevin GREAVES, Richardson, Australian Capital Territory (AU); Lingling GAO, Floreat WA (AU); Jonathan Paul ANDERSON, Floreat WA (AU); Daai ZHANG, Bruce Act (AU); Robert DE FEYTER, Acton, Australian Capital Territory (AU)

(73) Assignee: Commonwealth Scientific and Industrial Research Organisation, Acton (AU)

(21) Appl. No.: 19/330,166

(22) Filed: Sep. 16, 2025

Related U.S. Application Data
(63) Continuation of application No. 16/647,241, now Pat. No. 12,428,641.

(30) Foreign Application Priority Data
Sep. 15, 2017 (AU) 2017903773
Aug. 3, 2018 (AU) 2018902840
Aug. 8, 2018 (AU) 2018902896

(51) Int. Cl. C12N 15/113; A01N 63/60; C12N 15/86

(52) U.S. Cl. CPC C12N 15/113; A01N 63/60; C12N 15/86; C12N 2310/11; C12N 2310/531

(57) ABSTRACT

The present invention relates to new double stranded RNA (dsRNA) structures and their use in gene silencing.

### CROSS REFERENCE TO RELATED APPLICATION

This application is a continuation of U.S. application Ser. No. 16/647,241, filed Mar. 13, 2020, which is a § 371 national stage of PCT International Application No. PCT/AU2018/051015, filed Sep. 17, 2018, claiming priority of Australian Patent Application No. 2018902896, filed Aug. 8, 2018, Australian Patent Application No. 2018902840, filed Aug. 3, 2018, and Australian Patent Application No. 2017903773, filed Sep. 15, 2017, the contents of each of which are hereby incorporated by reference into the subject application.

### REFERENCE TO SEQUENCE LISTING

This application incorporates by reference nucleotide sequences which are present in the file named “250916_91340-A_SequenceListing_AD.xml”, which is 312,093 bytes in size, and which was created on Sep. 16, 2025 in the IBM-PC machine format, having an operating system compatibility with MS-Windows, which is contained in the xml file filed Sep. 16, 2025 as part of this application.

### FIELD OF THE INVENTION

The present invention relates to new double stranded RNA (dsRNA) structures and their use in gene silencing.

### BACKGROUND OF THE INVENTION

Conventional dsRNA molecules used to induce RNA interference (RNAi) in animal cells are formed by annealing single-stranded sense and antisense RNAs or from self-complementary RNAs which have regions of complementarity that allow for formation of stem-loop structures, also called hairpin RNA (hpRNA). In plants, transgenes expressing long hairpin RNAs have proven to be highly effective at inducing RNAi. Some studies of long hpRNA have shown that the processed RNA products of dsRNA, called short interfering RNAs (siRNA), that are formed from the sequences nearer the loop end of the hpRNA are more abundant relative to open end of the hpRNA structure, implying that Dicer processing of the dsRNA stem of the hpRNA appears to start from the loop end. The entire loop can be excised precisely from the hpRNA structure, which may provide a dsRNA terminus for the RNAase enzyme Dicer to bind and start siRNA processing.

Whilst dsRNA induced gene silencing has proven to be a valuable tool in altering the phenotype of an organism, there is a need for alternate, preferably improved, dsRNA molecules which can be used for RNAi.

### SUMMARY OF THE INVENTION

The inventors conceived of a new design of RNAi-inducing molecule, herein called loop-ended dsRNA (ledRNA) that has one or more of the following features; is easily synthesized, more readily forms a dsRNA structure and induces efficient silencing of target genes in eukaryotic cells. The ledRNA is also effective when topically applied to plant leaves.

In a first aspect, the present invention provides a ribonucleic acid (RNA) molecule comprising a first RNA component, a second RNA component which is covalently linked to the first RNA component and, optionally, one or more or all of (i) a linking ribonucleotide sequence which covalently links the first and second RNA components, (ii) a 5′ leader sequence and (iii) a 3′ trailer sequence,
wherein the first RNA component consists of, in 5′ to 3′ order, a first 5′ ribonucleotide, a first RNA sequence and a first 3′ ribonucleotide, wherein the first 5′ and 3′ ribonucleotides basepair to each other in the RNA molecule, wherein the first RNA sequence comprises a first sense ribonucleotide sequence of at least 20 contiguous ribonucleotides, a first loop sequence of at least 4 ribonucleotides and a first antisense ribonucleotide sequence of at least 20 contiguous ribonucleotides, wherein the first antisense ribonucleotide sequence hybridises with the first sense ribonucleotide sequence in the RNA molecule, wherein the first antisense ribonucleotide sequence is capable of hybridising to a first region of a target RNA molecule,
wherein the second RNA component is covalently linked, via the linking ribonucleotide sequence if present or directly if the linking ribonucleotide sequence is not present, to the first 5′ ribonucleotide or the first 3′ ribonucleotide,
wherein the second RNA component consists of, in 5′ to 3′ order, a second 5′ ribonucleotide, a second RNA sequence and a second 3′ ribonucleotide, wherein the second 5′ and 3′ ribonucleotides basepair to each other in the RNA molecule, wherein the second RNA sequence comprises a second sense ribonucleotide sequence, a second loop sequence of at least 4 ribonucleotides and a second antisense ribonucleotide sequence, wherein the second sense ribonucleotide sequence hybridises with the second antisense ribonucleotide sequence in the RNA molecule,
wherein the 5′ leader sequence, if present, consists of a sequence of ribonucleotides which is covalently linked to the first 5′ ribonucleotide if the second RNA component is linked to the first 3′ ribonucleotide or to the second 5′ ribonucleotide if the second RNA component is linked to the first 5′ ribonucleotide, and
wherein the 3′ trailer sequence, if present, consists of a sequence of ribonucleotides which is covalently linked to the second 3′ ribonucleotide if the second RNA component is linked to the first 3′ ribonucleotide or to the first 3′ ribonucleotide if the second RNA component is linked to the first 5′ ribonucleotide.

In a second aspect, the present invention provides an RNA molecule comprising a first RNA component, a second RNA component which is covalently linked to the first RNA component and, optionally, one or more or all of (i) a linking ribonucleotide sequence which covalently links the first and second RNA components, (ii) a 5′ leader sequence and (iii) a 3′ trailer sequence,
wherein the first RNA component consists of, in 5′ to 3′ order, a first 5′ ribonucleotide, a first RNA sequence and a first 3′ ribonucleotide, wherein the first 5′ and 3′ ribonucleotides basepair, wherein the first RNA sequence comprises a first sense ribonucleotide sequence, a first loop sequence of at least 4 ribonucleotides and a first antisense ribonucleotide sequence,
wherein the first sense ribonucleotide sequence and first antisense ribonucleotide sequence each consist of at least 20 contiguous ribonucleotides whereby the at least 20 contiguous ribonucleotides of the first sense ribonucleotide sequence fully basepair with the at least 20 contiguous ribonucleotides of the first antisense ribonucleotide sequence, wherein the at least 20 contiguous ribonucleotides of the first sense ribonucleotide sequence or the at least 20 contiguous ribonucleotides of the first antisense ribonucleotide sequence are identical in sequence to a first region of a target RNA molecule or its complement, respectively, or both,
wherein the second RNA component is covalently linked, via the linking ribonucleotide sequence if present, to the first 5′ ribonucleotide or the first 3′ ribonucleotide,
wherein the second RNA component consists of, in 5′ to 3′ order, a second 5′ ribonucleotide, a second RNA sequence and a second 3′ ribonucleotide, wherein the second 5′ and 3′ ribonucleotides basepair, wherein the second RNA sequence comprises a second sense ribonucleotide sequence, a second loop sequence of at least 4 ribonucleotides and a second antisense ribonucleotide sequence, wherein the second sense ribonucleotide sequence basepairs with the second antisense ribonucleotide sequence,
wherein the 5′ leader sequence, if present, consists of a sequence of ribonucleotides which is covalently linked to the first 5′ ribonucleotide if the second RNA component is linked to the first 3′ ribonucleotide or to the second 5′ ribonucleotide if the second RNA component is linked to the first 5′ ribonucleotide, and
wherein the 3′ trailer sequence, if present, consists of a sequence of ribonucleotides which is covalently linked to the second 3′ ribonucleotide if the second RNA component is linked to the first 3′ ribonucleotide or to the first 3′ ribonucleotide if the second RNA component is linked to the first 5′ ribonucleotide.

In a preferred embodiment, the RNA molecule of the invention is a chimeric RNA molecule.

In a third aspect, the present invention provides a chimeric ribonucleic acid (RNA) molecule, comprising a double-stranded RNA (dsRNA) region which comprises a sense ribonucleotide sequence and an antisense ribonucleotide sequence which are capable of hybridising to each other to form the dsRNA region, wherein
i) the sense ribonucleotide sequence consists of, covalently linked in 5′ to 3′ order, a first 5′ ribonucleotide, a first RNA sequence and a first 3′ ribonucleotide,
ii) the antisense ribonucleotide sequence consists of, covalently linked in 5′ to 3′ order, a second 5′ ribonucleotide, a second RNA sequence and a second 3′ ribonucleotide,
iii) the first 5′ ribonucleotide basepairs with the second 3′ ribonucleotide to form a terminal basepair of the dsRNA region,
iv) the second 5′ ribonucleotide basepairs with the first 3′ ribonucleotide to form a terminal basepair of the dsRNA region,
v) between about 5% and about 40% of the ribonucleotides of the sense ribonucleotide sequence and the antisense ribonucleotide sequence, in total, are either basepaired in a non-canonical basepair or are not basepaired,
vi) the dsRNA region does not comprise 20 contiguous canonical basepairs,
vii) the RNA molecule is capable of being processed in a eukaryotic cell or in vitro whereby the antisense ribonucleotide sequence is cleaved to produce short antisense RNA (asRNA) molecules of 20-24 ribonucleotides in length,
viii) the RNA molecule or at least some of the asRNA molecules, or both, are capable of reducing the expression or activity of a target RNA molecule in the eukaryotic cell, and
ix) the RNA molecule is capable of being made enzymatically by transcription in vitro or in a cell, or both.

In a fourth aspect, the present invention provides a chimeric RNA molecule comprising a first RNA component and a second RNA component which is covalently linked to the first RNA component,
wherein the first RNA component comprises a first double-stranded RNA (dsRNA) region, which comprises a first sense ribonucleotide sequence and a first antisense ribonucleotide sequence which are capable of hybridising to each other to form the first dsRNA region, and a first intervening ribonucleotide sequence of at least 4 nucleotides which covalently links the first sense ribonucleotide sequence and the first antisense ribonucleotide sequence,
wherein the second RNA component comprises a second sense ribonucleotide sequence, a second antisense ribonucleotide sequence and a second intervening ribonucleotide sequence of at least 4 ribonucleotides which covalently links the second sense ribonucleotide sequence and the second antisense ribonucleotide sequence, wherein the second sense ribonucleotide sequence hybridises with the second antisense ribonucleotide sequence in the RNA molecule,
wherein in the first RNA component,
   i) the first sense ribonucleotide sequence consists of at least 20 contiguous ribonucleotides covalently linked, in 5′ to 3′ order, a first 5′ ribonucleotide, a first RNA sequence and a first 3′ ribonucleotide,
   ii) the first antisense ribonucleotide sequence consists of at least 20 contiguous ribonucleotides covalently linked, in 5′ to 3′ order, a second 5′ ribonucleotide, a second RNA sequence and a second 3′ ribonucleotide,
   iii) the first 5′ ribonucleotide basepairs with the second 3′ ribonucleotide,
   iv) the second 5′ ribonucleotide basepairs with the first 3′ ribonucleotide,
   v) between 5% and 40% of the ribonucleotides of the first sense ribonucleotide sequence and the first antisense ribonucleotide sequence, in total, are either basepaired in a non-canonical basepair or are not basepaired, and
   vi) the first dsRNA region does not comprise 20 contiguous canonical basepairs,
wherein the chimeric RNA molecule is capable of being processed in a eukaryotic cell or in vitro whereby the first antisense ribonucleotide sequence is cleaved to produce short antisense RNA (asRNA) molecules of 20-24 ribonucleotides in length, and wherein
   (a) the chimeric RNA molecule or at least some of the asRNA molecules, or both, are capable of reducing the expression or activity of a target RNA molecule in the eukaryotic cell, or
   (b) the first antisense ribonucleotide sequence comprises a sequence of at least 20 contiguous ribonucleotides which is at least 50% identical in sequence to a region of the complement of the target RNA molecule, or
   (c) both (a) and (b).

In the first, second and fourth aspects, the first 5′ ribonucleotide and first 3′ ribonucleotide of the first RNA component basepair to form a basepair. That basepair is defined herein as the terminal basepair of the dsRNA region formed by self-hybridisation of the first RNA component. In the embodiment where the first sense ribonucleotide sequence is linked covalently to the first 5′ ribonucleotide without any intervening nucleotides and the first antisense ribonucleotide sequence is linked covalently to the first 3′ ribonucleotide without any intervening nucleotides, the first 5′ ribonucleotide is directly linked to one of the sense sequence and antisense sequence and the first 3′ ribonucleotide is directly linked to the other of the sense sequence and antisense sequence.

In a preferred embodiment, the at least 20 contiguous ribonucleotides of the first antisense ribonucleotide sequence are all capable of basepairing to nucleotides of the first region of the target RNA molecule. In an embodiment, the first sense ribonucleotide sequence is linked covalently to the first 5′ ribonucleotide without any intervening nucleotides, or the first antisense ribonucleotide sequence is linked covalently to the first 3′ ribonucleotide without any intervening nucleotides, or both.

In an embodiment, the RNA molecule comprises the linking ribonucleotide sequence, wherein the linking ribonucleotide sequence is related in sequence to the target gene, either identical at least in part to a region of the target RNA molecule or to its complement. In a preferred embodiment, the linking ribonucleotide sequence together with sense sequences in the first and second RNA components form part of one contiguous sense sequence, or together with antisense sequences in the first and second RNA components form part of one contiguous antisense sequence. In an embodiment, the RNA molecule comprises the linking ribonucleotide sequence, wherein the linking ribonucleotide sequence is less than 20 ribonucleotides. In an embodiment, the linking ribonucleotide sequence hybridizes to the target RNA molecule. In an embodiment, the linking ribonucleotide sequence is identical to a portion of the complement of the target RNA molecule. In an embodiment, the linking ribonucleotide sequence is between 1 and 10 ribonucleotides.

In embodiments of the first, second or fourth aspects, the RNA molecule comprises one or more or all of (i) a linking ribonucleotide sequence which covalently links the first and second RNA components, (ii) a 5′ extension sequence and (iii) a 3′ extension sequence, wherein the 5′ extension sequence, if present, consists of a sequence of ribonucleotides which is covalently linked to the first RNA component or to the second RNA component, and wherein the 3′ extension sequence, if present, consists of a sequence of ribonucleotides which is covalently linked to the second RNA component or to the first RNA component, respectively. In an embodiment, the first RNA component and the second RNA component are covalently linked via a linking ribonucleotide sequence. In an alternative embodiment, the first RNA component and the second RNA component are directly linked, without any linking ribonucleotide sequence present.

In embodiments of the first to fourth aspects, the RNA molecule comprises two or more sense ribonucleotide sequences which are each identical in sequence to a region of a target RNA molecule, and the RNA molecule comprises one or more antisense ribonucleotide sequences based paired to the sense ribonucleotide sequences, wherein the one or more antisense sequences are complementary, preferably fully complementary, to the regions of the target molecule. In an embodiment, the two or more sense ribonucleotide sequences are identical in sequence to different regions of the same target RNA molecule, which may or may not be contiguous in the target RNA molecule. In an embodiment, the two or more sense ribonucleotide sequences are identical in sequence to a region of different target RNA molecules. In an embodiment, the two or more sense ribonucleotide sequences have no intervening loop sequences, i.e. they are contiguous relative to the target RNA molecule.

In preferred embodiments of the first to fourth aspects, the RNA molecule comprises two or more antisense ribonucleotide sequences, and sense ribonucleotide sequences based paired thereto, which antisense sequences are each complementary to a region of a target RNA molecule. The regions of the target RNA molecule to which they are complementary may or may not be contiguous in the target RNA molecule. In an embodiment, the two or more antisense ribonucleotide sequences are complementary to different regions of the same target RNA molecule. In an embodiment, the second of the two or more antisense ribonucleotide sequences is complementary to a region of a different target RNA molecule than the first of the two or more antisense ribonucleotide sequences. In a preferred embodiment, the two or more antisense ribonucleotide sequences have no intervening loop sequences, i.e. they are contiguous relative to the complement of the target RNA molecule. In a preferred embodiment, one or both of the two or more antisense ribonucleotide sequences and sense ribonucleotide sequences basepair along their full length through canonical basepairs, or through some canonical and some non-canonical basepairs, preferably G:U basepairs.

In a preferred embodiment of the first to fourth aspects, the RNA molecule is a single strand of ribonucleotides. For example, the RNA molecule can comprise a single strand of ribonucleotides having a 5′ end, at least one sense ribonucleotide sequence which is at least 21 nucleotides in length, an antisense ribonucleotide sequence which is fully base paired with each sense ribonucleotide sequence over at least 21 contiguous nucleotides, at least two loop sequences and a 3′ end. The order 5′ to 3′ may be the sense ribonucleotide sequence and then the antisense ribonucleotide sequence, or vice versa. In an embodiment, the ribonucleotide at the 5′ end and the ribonucleotide at the 3′ end are adjacent, each base paired and are not directly covalently bonded, see for example FIG. 1.

In another embodiment of the first to fourth aspects, the RNA molecule comprises a first antisense ribonucleotide sequence which hybridizes to a first region of a target RNA, a second antisense ribonucleotide sequence which hybridizes to a second region of a target RNA, the second region of the target RNA being different to the first region of the target RNA, and the RNA molecule comprising only one sense ribonucleotide sequence which hybridizes to the target RNA, wherein the two antisense sequences are not contiguous in the RNA molecule. In an embodiment, the first and second regions of the target RNA are contiguous in the target RNA. Alternatively, they are not contiguous.

In another embodiment of the first to fourth aspects, the RNA molecule comprises a first sense ribonucleotide sequence which is at least 60% identical to a first region of a target RNA, a second sense ribonucleotide sequence which is at least 60% identical to a second region of a target RNA, the second region of the target RNA being different to the first region of the target RNA, and the RNA molecule comprising only one antisense ribonucleotide sequence which hybridizes to the target RNA, wherein the two sense sequences are not contiguous in the RNA molecule. In an embodiment, the first and second regions of the target RNA are contiguous in the target RNA molecule. Alternatively, they are not contiguous. In preferred embodiments, the first and second sense ribonucleotide sequences are each, independently, at least 70%, at least 80%, at least 90%, at least 95%, or at least 99% identical to the respective region of target RNA i.e. the first sense sequence may be at least 70% identical to its target region and the second sequence at least 80% identical to its target sequence, etc.

In a preferred embodiment of the first to fourth aspects, the RNA molecule is a single strand of ribonucleotides having a 5′ end, at least one sense ribonucleotide sequence which is at least 21 nucleotides in length, an antisense ribonucleotide sequence which is fully base paired with each sense ribonucleotide sequence over at least 21 contiguous nucleotides, at least two loop sequences and a 3′ end. In a more preferred embodiment, the basepairing in the RNA molecule is comprised in a double-stranded region which is at least 21 contiguous basepairs in length which includes some non-canonical basepairs, most preferably some G:U basepairs, the double-stranded region comprising the at least one sense ribonucleotide sequence which is at least 21 nucleotides in length.

In preferred embodiments of the first and second aspects, the second RNA component is characterised in that:
i) the second sense ribonucleotide sequence consists of at least 20 contiguous ribonucleotides covalently linked, in 5′ to 3′ order, the second 5′ ribonucleotide, a third RNA sequence and a third 3′ ribonucleotide,
ii) the second antisense ribonucleotide sequence consists of at least 20 contiguous ribonucleotides covalently linked, in 5′ to 3′ order, a third 5′ ribonucleotide, a fourth RNA sequence and the second 3′ ribonucleotide,
iii) the second 5′ ribonucleotide basepairs with the second 3′ ribonucleotide,
iv) the third 3′ ribonucleotide basepairs with the third 5′ ribonucleotide, wherein the chimeric RNA molecule is capable of being processed in a eukaryotic cell or in vitro whereby the second antisense ribonucleotide sequence is cleaved to produce short antisense RNA (asRNA) molecules of 20-24 ribonucleotides in length. Most preferably, the asRNA molecules produced from the second antisense sequence are capable of reducing expression of the target RNA, either without or in combination with asRNAs produced from the first antisense sequence of the first RNA component.

In a preferred embodiment of the fourth aspect, the second RNA component is characterised in that:
i) the second sense ribonucleotide sequence consists of at least 20 contiguous ribonucleotides covalently linked, in 5′ to 3′ order, a third 5′ ribonucleotide, a third RNA sequence and a third 3′ ribonucleotide,
ii) the second antisense ribonucleotide sequence consists of at least 20 contiguous ribonucleotides covalently linked, in 5′ to 3′ order, a fourth 5′ ribonucleotide, a fourth RNA sequence and the fourth 3′ ribonucleotide,
iii) the third 5′ ribonucleotide basepairs with the fourth 3′ ribonucleotide,
iv) the third 3′ ribonucleotide basepairs with the third 5′ ribonucleotide, wherein the chimeric RNA molecule is capable of being processed in a eukaryotic cell or in vitro whereby the second antisense ribonucleotide sequence is cleaved to produce short antisense RNA (asRNA) molecules of 20-24 ribonucleotides in length.

In these preferred embodiments, it is more preferred that between 5% and 40% of the ribonucleotides of the second sense ribonucleotide sequence and the second antisense ribonucleotide sequence, in total, are either basepaired in a non-canonical basepair or are not basepaired, and/or the second dsRNA region does not comprise 20 contiguous canonical basepairs. More preferably, about 12%, about 15%, about 18%, about 21%, about 24%, or between 15% and 30%, or even more preferably between 16% and 25%, of the ribonucleotides of the second sense ribonucleotide sequence and the second antisense ribonucleotide sequence, in total, are either basepaired in a non-canonical basepair or are not basepaired. In preferred embodiments, at least 50%, at least 60%, at least 70%, at least 80%, at least 90%, at least 95%, at least 97%, or 100% of the non-canonical basepairs in the second dsRNA region are G:U basepairs. Most preferably, in these embodiments,
(a) the chimeric RNA molecule or at least some of the asRNA molecules, or both, are capable of reducing the expression or activity of a target RNA molecule in an eukaryotic cell, or
(b) the second antisense ribonucleotide sequence comprises a sequence of at least 20 contiguous ribonucleotides which is at least 50% identical in sequence to a region of the complement of the target RNA molecule, preferably at least 60% identical, more preferably at least 70% identical, even more preferably at least 80% identical, most preferably at least 90% identical or 100% identical to the region of the complement of the target RNA molecule, or
   both (a) and (b).

In an embodiment of the first to fourth aspects, the RNA molecule comprises a 5′ leader sequence or 5′ extension sequence. In an embodiment, the RNA molecule comprises a 3′ trailer sequence or 3′ extension sequence. In a preferred embodiment, the RNA molecule comprises both the 5′ leader/extension sequence and the 3′ trailer/extension sequence.

In an embodiment of the first to fourth aspects, each ribonucleotide of the RNA molecule is covalently linked to two other nucleotides, i.e. it is a covalently closed circle. Alternatively, the RNA molecule may be represented as a dumbbell shape (FIG. 1) but have a gap or nick in one part of the double-stranded structure.

In an embodiment of the first to fourth aspects, at least one or all of the loop sequences of the RNA molecule are longer than 20 nucleotides. In a preferred embodiment, at least one of the loops of the RNA molecule is between 4 and 1,000 ribonucleotides in length. In a more preferred embodiment, all of the loops are between 4 and 1,000 ribonucleotides in length. In a more preferred embodiment, at least one of the loops of the RNA molecule is between 4 and 200 ribonucleotides in length. In an even more preferred embodiment, all of the loops are between 4 and 200 ribonucleotides in length. In an even more preferred embodiment, at least one of the loops of the RNA molecule is between 4 and 50 ribonucleotides in length. In a most preferred embodiment, all of the loops are between 4 and 50 ribonucleotides in length. In an embodiment, the eukaryotic cell is a vertebrate cell, and each loop of the RNA molecule is between 20 and 50, or between 20 and 30, ribonucleotides in length.

In embodiments of the first to fourth aspects, the RNA molecule has none, or one, or two or more bulges in a double-stranded region. In this context, a bulge is a nucleotide, or two or more contiguous nucleotides, in the sense or antisense ribonucleotide sequence which is not basepaired in the dsRNA region and which does not have a mismatched nucleotide at the corresponding position in the complementary sequence in the dsRNA region. The dsRNA region of the RNA molecule may comprise a sequence of more than 2 or 3 nucleotides within the sense or antisense sequence, or both, which loops out from the dsRNA region when the dsRNA structure forms. The sequence which loops out may itself form some internal basepairing, for example it may itself form a stem-loop structure.

In embodiments of the first to fourth aspects, the RNA molecule has none, or one, or two or more bulges in a double-stranded region. In this context, a bulge is a nucleotide, or two or more contiguous nucleotides, in the sense or antisense ribonucleotide sequence which is not basepaired in the dsRNA region and which does not have a mismatched nucleotide at the corresponding position in the complementary sequence in the dsRNA region. The dsRNA region of the RNA molecule may comprise a sequence of more than 2 or 3 nucleotides within the sense or antisense sequence, or both, which loops out from the dsRNA region when the dsRNA structure forms. The sequence which loops out may itself form some internal basepairing, for example it may itself form a stem-loop structure.

In an embodiment, the RNA molecule has three, four or more loops. In a preferred embodiment, the RNA molecule has only two loops. In an embodiment, the first double-stranded region, or the first and second dsRNA region, of the RNA molecule comprises one, or two, or more nucleotides which are not basepaired in the double-stranded region, or up to 2%, 3%, 4%, 5%, 6%, 7%, 8%, 9% or 10% of the nucleotides in the double-stranded region which are not basepaired.

In preferred embodiments of the first to fourth aspects, the target RNA molecule or the RNA molecule of the invention, or both, is in a eukaryotic cell. For example, the eukaryotic cell may be a plant cell, animal cell or fungal cell. In an embodiment, the eukaryotic cell is a fungal cell. In an embodiment, the RNA molecule of the invention is produced in a cell, such as for example a bacterial cell or other microbial cell, which is different to the cell comprising the target RNA. Similarly, in an embodiment the RNA molecule of the invention is produced in a eukaryotic cell which does not comprise the target RNA when the RNA molecule of the invention is produced, but the eukaryotic cell comprising the RNA molecule of the invention and/or its processed RNA products, may become a host for the target RNA, for example if the target RNA is a viral RNA or other introduced RNA. Such cells may be protected prophylactically against the viral or other introduced RNA.

In preferred embodiments of the first to fourth aspects, the RNA molecule is capable of being made enzymatically by transcription in vitro or in a cell, or both. In an embodiment, an RNA molecule of the present invention is expressed in a cell i.e. produced in the cell by transcription from one or more nucleic acids encoding the RNA molecule. The one or more nucleic acids encoding the RNA molecule is preferably a DNA molecule, which may be present on a vector in the cell or integrated into the genome of the cell, either the nuclear genome of the cell or in the plastid DNA of the cell. The one or more nucleic acids encoding the RNA molecule may also be an RNA molecule such as a viral vector.

Accordingly, in an embodiment, the present invention provides a cell comprising an RNA molecule described herein. In a preferred embodiment, the present invention provides an RNA molecule described herein which was expressed in a cell and which has been isolated and/or purified from the cell. The present invention therefore provides a preparation of isolated RNA molecules according to one or more of the first to fourth aspects, which is suitable for administration to a cell comprising the target RNA or potentially comprising the target RNA.

In an embodiment, one or more of the target RNAs encodes a protein. Alternatively, one or more of the target RNAs do not encode a protein, such as a rRNA, tRNA, snoRNA or miRNA.

In embodiments of the first to fourth aspects, about 12%, about 15%, about 18%, about 21%, about 24%, or between about 15% and about 30%, or preferably between about 16% and about 25%, of the ribonucleotides of the sense ribonucleotide sequence and the antisense ribonucleotide sequence, in total, that form a dsRNA region are either basepaired in a non-canonical basepair or are not basepaired. In a preferred embodiment, at least 50%, at least 60%, at least 70%, at least 80%, at least 90%, at least 95%, at least 97%, or 100% of the non-canonical basepairs in a dsRNA region, or in all dsRNA regions in the RNA molecule, are G:U basepairs. The G nucleotide in each G:U basepair may independently be in the sense ribonucleotide sequence or preferably in the antisense ribonucleotide sequence. Regarding the G nucleotides in the G:U basepairs of a dsRNA region, preferably at least 50% are in the antisense ribonucleotide sequence, more preferably at least 60% or 70%, even more preferably at least 80% or 90%, and most preferably at least 95% of them are in the antisense ribonucleotide sequence in the dsRNA region. This feature may apply to all of the dsRNA regions in the RNA molecule. In an embodiment, less than 25%, less than 20%, less than 15%, less than 10%, preferably less than 5%, more preferably less than 1% or most preferably none, of the ribonucleotides in the dsRNA region, or in all of the dsRNA regions in the RNA molecule in total, are not basepaired. In a preferred embodiment, every one in four to every one in six ribonucleotides in the dsRNA region, or in the dsRNA regions in total, form a non-canonical basepair or are not basepaired within the RNA molecule. In a preferred embodiment, the dsRNA region, or in the dsRNA regions in total, do not comprise 8 contiguous canonical basepairs. In an alternative embodiment, the dsRNA region comprises at least 8 contiguous canonical basepairs, for example 8 to 12 or 8 to 14 contiguous canonical basepairs. In a preferred embodiment, all of the ribonucleotides in the dsRNA region are base-paired with a canonical basepair or a non-canonical basepair. In an embodiment, one or more ribonucleotides of the sense ribonucleotide sequence or one or more ribonucleotides of the antisense ribonucleotide sequence, or both, are not basepaired. In an embodiment, one or more ribonucleotides of each sense ribonucleotide sequence and one or more ribonucleotides of each antisense ribonucleotide sequence are not basepaired in the RNA molecule of the invention.

In an embodiment, the antisense RNA sequence of the first RNA component or of the second RNA component, or both, is less than 100% identical, or between about 80% and 99.9% identical, or between about 90% and 98% identical, or between about 95% and 98% identical, preferably between 98% and 99.9% identical, in sequence to the complement of a region of the target RNA molecule or to two such regions, which may or may not be contiguous in the target RNA molecule. In a preferred embodiment, the antisense RNA sequence is 100% identical in sequence to a region of the complement of the target RNA molecule, for example to a region comprising 21, 23, 25, 27, 30, or 32 contiguous nucleotides. In an embodiment, the sense or antisense ribonucleotide sequence, or both, is at least 40, at least 50, at least about 100, at least about 200, at least about 300, at least about 400, at least about 500, at least about 600, at least about 700, at least about 800, at least about 900, at least about 1,000, or about 100 to about 1,000, contiguous nucleotides in length. The lengths of at least 100 nucleotides are preferred when using the RNA molecule in plant cells or fungal cells, or for non-vertebrate animal cells. Lengths for the sense and antisense ribonucleotide sequence in the dsRNA of 50 nucleotides or less, for example 31 to 50 nucleotides, are preferred when using the RNA molecule in vertebrate animal cells. In an embodiment, the number of ribonucleotides in the sense ribonucleotide sequence is between about 90% and about 110%, preferably between 95% and 105%, more preferably between 98% and 102%, even more preferably between 99% and 101%, of the number of ribonucleotides in the antisense ribonucleotide sequence. In a most preferred embodiment, the number of ribonucleotides in the sense ribonucleotide sequence is the same as the number of ribonucleotides in the antisense ribonucleotide sequence. These features can be applied to each dsRNA region in the RNA molecule.

In embodiments of the first to fourth aspects, the first 3′ ribonucleotide and the second 5′ ribonucleotide in the RNA molecule are covalently joined by a loop sequence consisting of at least 4 ribonucleotides, or between 4 and 1,000 ribonucleotides, or preferably between 4 and 200 ribonucleotides, more preferably between 4 and 50 ribonucleotides. In an embodiment, the RNA molecule further comprises a 5′ extension sequence which is covalently linked to the first 5′ ribonucleotide or a 3′ extension sequence which is covalently linked to the second 3′ ribonucleotide, or both. In an embodiment, the chimeric RNA molecule further comprises a 5′ extension sequence which is covalently linked to the second 5′ ribonucleotide or a 3′ extension sequence which is covalently linked to the first 3′ ribonucleotide, or both. In this embodiment, the RNA molecule comprises two separate strands of RNA which hybridise to form the RNA molecule, although it may have been produced by transcription from a nucleic acid molecule as a single RNA transcript and subsequently been processed to comprise the two RNA strands.

The overall length of the RNA molecule of the invention, produced as a single strand of RNA, after splicing out of any introns but before any processing of the RNA molecule by Dicer enzymes or other RNAses, is typically between 50 and 2000 ribonucleotides, preferably between 60 or 70 and 2000 ribonucleotides, more preferably between 80 or 90 and 2000 ribonucleotides, even more preferably between 100 or 110 and 2000 ribonucleotides. In preferred embodiments, the minimum length of the RNA molecule is 120, 130, 140, 150, 160, 180, or 200 nucleotides, and the maximum length is 400, 500, 600, 700, 800, 900, 1000, 1200, 1400, 1500 or 2000 ribonucleotides. Each combination of the mentioned minimum and maximum lengths is contemplated. Production of RNA molecules of such lengths by transcription in vitro or in cells such as bacterial or other microbial cells, or in the eukaryotic cell where the target gene is to be down-regulated, is readily achieved.

In an embodiment of the first to fourth aspects, the chimeric RNA molecule comprises two or more dsRNA regions which are the same or preferably different.

In preferred embodiments of the first to fourth aspects, the RNA molecule is expressed in a eukaryotic cell i.e. produced by transcription in the cell. In these embodiments, a greater proportion of dsRNA molecules are formed by processing of the RNA molecule that are 22 and/or 20 ribonucleotides in length when compared to processing of an analogous RNA molecule which has a corresponding dsRNA region which is fully basepaired with canonical basepairs. That is, the RNA molecules of these embodiments are more readily processed to provide 22- and/or 20-ribonucleotide short antisense RNAs than the analogous RNA molecule whose dsRNA region is fully basepaired with canonical basepairs, as a proportion of the total number of 20-24 nucleotide asRNAs produced from the RNA molecule.

In an embodiment, the RNA molecule of the invention comprises a combination of two or more features of an RNA molecule described herein.

In another aspect, the present invention provides a polynucleotide encoding an RNA molecule described herein, preferably a chimeric RNA molecule described herein. In an embodiment, the polynucleotide is a DNA construct which may be integrated into a larger DNA molecule such as a chromosome. In an embodiment, the polynucleotide is operably linked to a promoter capable of directing expression of the RNA molecule in a host cell. The host cell may be a bacterial cell such as E. coli, a fungal cell such as a yeast cell, or a eukaryotic cell such as a plant cell or an animal cell. In an embodiment, the promoter is heterologous relative to the polynucleotide. The polynucleotide encoding the RNA molecule may be a chimeric or recombinant polynucleotide, or an isolated and/or exogenous polynucleotide. In an embodiment, the promoter can function in vitro, for example a bacteriophage promoter such as a T7 RNA polymerase promoter or SP6 RNA polymerase promoter. In an embodiment, the promoter is an RNA polymerase III promoter such as a U6 promoter or an H1 promoter. In an embodiment, the promoter is an RNA polymerase II promoter, which may be a constitutive promoter, a tissue-specific promoter, a developmentally regulated promoter or an inducible promoter. In an embodiment, the polynucleotide encodes an RNA precursor molecule comprising an intron in at least one loop sequence which is capable of being spliced out during or after transcription of the polynucleotide in a host cell. In an embodiment, the present invention provides a vector comprising a polynucleotide described herein. In an embodiment, the vector is a viral vector. In an embodiment, the vector is a plasmid vector such as a binary vector suitable for use with Agrobacterium tumefaciens.

In an embodiment where the polynucleotide or vector of the invention is in a eukaryotic host cell, preferably in a plant, the promoter region of the polynucleotide or vector, which is operably linked to the region which encodes an RNA molecule of the invention, has a lower level of methylation when compared to the promoter of a corresponding polynucleotide or vector encoding an RNA molecule which has a corresponding dsRNA region which is fully basepaired with canonical basepairs. In an embodiment, the lower level of methylation is less than 50%, less than 40%, less than 30% or less than 20%, when compared to the promoter of the corresponding polynucleotide or vector. In an embodiment, the host cell comprises at least two copies of the polynucleotide or vector encoding an RNA molecule of the invention. In this embodiment:
i) the level of reduction in the expression and/or activity of the target RNA molecule in the eukaryotic cell is at least the same relative to a corresponding eukaryotic cell having a single copy of the polynucleotide or vector, and/or
ii) the level of reduction in the expression and/or activity of the target RNA molecule in the eukaryotic cell is lower when compared to a corresponding cell comprising an RNA molecule which has a corresponding dsRNA region which is fully basepaired with canonical basepairs.

In another aspect, the present invention provides a host cell comprising an RNA molecule described herein, a polynucleotide described herein or a vector comprising the same. In an embodiment, the host cell is a non-human cell such as bacterial cell, a fungal cell, a plant cell or a non-human animal cell. In an embodiment, the cell is a non-human cell or a human cell in cell culture. In an embodiment, the cell is a eukaryotic cell such as a cell other than an animal cell. In an embodiment, the cell is a microbial cell such as a prokaryotic cell. In an embodiment, the host cell is alive. In an alternative embodiment, the host cell is dead.

In another aspect, the present invention provides a non-human organism comprising an RNA molecule of the invention, preferably a chimeric RNA molecule described herein, a polynucleotide or vector of the invention comprising the same or a host cell comprising the same. In an embodiment, the non-human organism is transgenic insofar as it comprises a polynucleotide of the invention. In an embodiment, the polynucleotide is stably integrated into the genome of the non-human organism.

In another aspect, the present invention provides a method of producing an RNA molecule of the invention, the method comprising expressing the polynucleotide of the invention in a host cell or cell-free expression system. In this embodiment, the method may further comprise at least partially purifying the RNA molecule, or not.

In another aspect, the present invention provides a method of producing a cell or non-human organism, preferably a plant or fungus, the method comprising introducing a polynucleotide or vector of the invention into a cell, preferably a plant cell or fungus, preferably so that the polynucleotide or vector or part thereof encoding the RNA molecule is stably integrated into the genome of the cell. In an embodiment, the non-human organism is generated from the cell or a progeny cell, for example by regenerating a plant. In an embodiment, the non-human organism is generated by introducing the cell or one or more progeny cells into a non-human organism. Alternatively to the stable integration of the polynucleotide or vector into the genome of the cell, the polynucleotide or vector may be introduced into the cell without integration of the polynucleotide or vector into the genome, for example to express the RNA molecule transiently in the cell or organism.

In another aspect, the present invention provides an extract of a host cell or organism or part thereof of the invention, wherein the extract comprises an RNA molecule of the invention, small RNA molecules (20-24 nt in length) produced by processing of the RNA molecule, or both, and/or the polynucleotide or vector of the invention. In an embodiment, the present invention provides a composition comprising one or more of an RNA molecule of the invention, small RNA molecules (20-24 nt in length) produced by processing of the RNA molecule, or both, a polynucleotide of the invention, a vector of the invention, a host cell of the invention, or an extract produced by a method of the invention, and one or more suitable carriers. In an embodiment, the composition is a pharmaceutical composition, such as a composition suitable for administration to a human or other animal. The pharmaceutical composition may be suitable for prophylaxis or treatment of a disease, or for topical application such as a cosmetic application. In an embodiment, the composition is suitable for application to a plant, preferably a plant or population of plants in a field, or to an insect or population of insects. In an embodiment, the composition is suitable for application to a crop, for example by spraying on crop plants in a field.

In an embodiment, the extract or a composition comprising the RNA molecule of the invention or small RNA molecules (20-24 nt in length) produced by processing of the RNA molecule, or both, further comprises at least one compound which enhances the stability of the RNA molecule, or the polynucleotide and/or vector, whereby the at least one compound assists in the RNA molecule, polynucleotide or vector being taken up by a cell, such as for example a cell of an organism. In an embodiment, the compound is a transfection promoting agent, for example a lipid-containing compound.

In another aspect, the present invention provides a method for reducing or down-regulating the level and/or activity of a target RNA molecule in a cell or an organism, the method comprising delivering to the cell or organism one or more RNA molecule(s) of the invention or small RNA molecules (20-24 nt in length) produced by processing of the RNA molecule, or both, a polynucleotide of the invention, a vector of the invention, or a composition of the invention. In an embodiment, the target RNA molecule encodes a protein. In an embodiment, the method reduces the level and/or activity of more than one target RNA molecule, the target RNA molecules being different, for example two or more target RNAs are reduced in level and/or activity which are related in sequence such as from a gene family.

In another aspect, the present invention provides a method of controlling a non-human organism, the method comprising delivering to the non-human organism one or more RNA molecule(s) of the invention or small RNA molecules (20-24 nt in length) produced by processing of the RNA molecule, or both, or a polynucleotide or vector of the invention, a host cell of the invention, an extract produced by a method of the invention, or a composition of the invention, wherein the RNA molecule or small RNA molecules have a deleterious effect on the non-human organism. In an embodiment, the non-human organism is an arthropod, such as for example an insect, or a plant such as for example a weed. In an embodiment, the non-human organism is a plant, and the arthropod eats the plant or a portion thereof, whereby the arthropod is controlled.

In an embodiment, the present invention provides a method of preventing or treating a disease in a subject, the method comprising administering to the subject one or more RNA molecule(s) of the invention or small RNA molecules (20-24 nt in length) produced by processing of the RNA molecule, or both, a polynucleotide or vector of the invention, a host cell of the invention, an extract produced by a method of the invention, or a composition of the invention, wherein the RNA molecule or small RNA molecules has a beneficial effect on at least one symptom of the disease. In an embodiment, the RNA molecule or small RNA molecules, polynucleotide, vector or composition are administered topically, orally or injected. In an embodiment, the subject is a vertebrate animal. In an embodiment, the vertebrate animal is a mammal such as a human, a livestock animal such as cattle or sheep, or birds such as chickens and other poultry.

In another aspect, the present invention provides an RNA molecule of the invention, a polynucleotide or vector of the invention, a host cell of the invention, an extract produced by a method of the invention, or a composition of the invention for use in treating a disease in a subject, wherein the RNA molecule or small RNA molecules has a beneficial effect on at least one symptom of the disease. In an embodiment, the present invention provides a use of an RNA molecule of the invention or small RNA molecules produced therefrom, a polynucleotide or vector of the invention, a host cell of the invention, an extract produced by a method of the invention, or a composition of the invention for the manufacture of a medicament for preventing or treating a disease in a subject, wherein the RNA molecule or small RNA molecules produced therefrom have a beneficial effect on at least one symptom of the disease.

In another aspect, the present invention provides a kit comprising one or more of an RNA molecule(s) of the invention or small RNA molecules produced therefrom, a polynucleotide or vector of the invention, a host cell of the invention, an extract produced by a method of the invention, or a composition of the invention.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. Schematic designs of two ledRNA molecules. (A) This ledRNA molecule comprises a sense sequence which can be considered to be two adjacent sense sequences, covalently linked without an intervening spacer sequence and having identity to the target RNA, an antisense sequence which is complementary to the sense sequence and which is divided into two regions, a 5′ region and a 3′ region, and two loops that separate the sense from the antisense sequences. (B) This ledRNA molecule comprises an antisense sequence which can be considered to be two adjacent antisense sequences, covalently linked without an intervening spacer sequence and having identity to the complement of a target RNA, a sense sequence which is complementary to the antisense sequence and which is divided into two regions, and two loops that separate the sense from the antisense sequences. The RNA molecule produced by transcription, for example by in vitro transcription from a promoter such as a T7 or Sp6 promoter, self-anneals by basepairing between the complementary sense and antisense sequences to form a double-stranded region with a loop at each end and having a “nick” in either the antisense or sense sequence. Additional sequences may be linked to the 5′ and/or 3′ ends as 5′- or 3′-extensions.

FIG. 2. ledRNA is more efficient in forming dsRNA than sense/antisense annealing or hairpin RNA. Schematic representations of three forms of double-stranded RNA molecules are shown: A, conventional dsRNA formed by annealing of two separate strands; B, a hairpin RNA having a 5′- and a 3′-extension; and C, ledRNA molecule. The lower panel shows a photograph after gel electrophoresis of the RNA transcripts for the three types of RNA molecules targeting either a GUS gene or a GFP gene.

FIG. 3. Northern blot hybridization of treated (A and B) and untreated distal (C and D) tissues shows that ledRNA is more stable than dsRNA and spread through tobacco leaf tissue. In the distal tissues (C and D, top panel) the dsRNA signal could not be detected, in contrast to strong ledRNA signals.

FIG. 4. ledRNA treatment induced downregulation of GUS in both the treated area (1) and the untreated area above (3).

FIG. 5. ledRNA induces silencing of the FAD2.1 gene in N. benthamiana leaves.

FIG. 6. Northern blot hybridization confirms strong downregulation of FAD2.1 mRNA by treatment with ledFAD2.1 at 6 and 24 hours.

FIG. 7. Alignment of the nucleotide sequences of a region of the GUS target gene (SEQ ID NO:14) and the sense sequence of the hpGUS[G:U] construct (nucleotides 9 to 208 of SEQ ID NO:11). 52 cytidine (C) nucleotides were substituted with thymidine (T) nucleotides. Conserved nucleotides are asterisked, substituted C's are not asterisked.

FIG. 8. Alignment of the nucleotide sequences of a region of the GUS target gene (SEQ ID NO:14) and the sense sequence of the hpGUS[1:4] construct (nucleotides 9 to 208 of SEQ ID NO:12). Every 4th nucleotide in hpGUS[1:4] was substituted relative to the corresponding wild-type sense sequence, whereby for every 4th nucleotide, C was changed to G, G was changed to C, A was changed to T, and T was changed to A. Conserved nucleotides are asterisked, substituted G's and C's are not asterisked, substituted A's and T's are shown with semi-colons.

FIG. 9. Alignment of the nucleotide sequences of a region of the GUS target gene (SEQ ID NO:14) and the sense sequence of the hpGUS[2:10] construct (nucleotides 9 to 208 of SEQ ID NO:13). Every 9th and 10th nucleotide in each block of 10 nucleotides in hpGUS[2:10] was substituted relative to the corresponding wild-type sense sequence, whereby for every 9th and 10th nucleotide, C was changed to G, G was changed to C, A was changed to T and T was changed to A. Conserved nucleotides are asterisked, substituted G's and C's are not asterisked, substituted A's and T's are shown with semi-colons.

FIG. 10. Schematic diagram showing structures of the genetic constructs encoding modified hairpin RNAs targeting GUS mRNA.

FIG. 11. Schematic diagram of vector pWBPPGH used to transform tobacco plants, providing a GUS target gene. The T-DNA extends from the right border (RB) to the left border (LB) of the vector. The selectable marker gene on the T-DNA is the 35S-HPT-tm1′ gene encoding hygromycin resistance.

FIG. 12. GUS activity in plants transformed with constructs encoding modified hairpin RNAs for reducing expression of a GUS target gene. No hp: control PPGH11 and PPGH24 plants with no hpGUS constructs. The number of plants showing less than 10% GUS activity compared to the corresponding control PPGH11 or PPGH24 plants and the percentage of such plants relative to the number of plants tested are given in brackets.

FIG. 13. (A) Average GUS activity of all transgenic plants: 59 plants for hpGUS[wt], 74 for hpGUS[G:U], 33 for hpGUS[1:4] and 41 for hpGUS[2:10]. (B) Average GUS activity of all silenced plants (32 for hpGUS[wt], 71 for hpGUS[G:U], 33 for hpGUS[1:4] and 28 for hpGUS[2:10].

FIG. 14. GUS activity of transgenic progeny plants containing hpGUS[wt], hpGUS[G:U] or hpGUS[1:4].

FIG. 15. Autoradiograph of a Southern blot of DNA from 16 plants transformed with the hpGUS[G:U] construct. DNAs were digested with HindIII prior to gel electrophoresis and probed with an OCS-T probe. Lane 1: size markers (HindIII-digested lambda DNA); Lanes 2 and 3, DNA from parental plants PPGH11 and PPGH24; Lanes 4-19: DNAs from 16 different transgenic plants.

FIG. 16. Autoradiogram of a Northern blot hybridisation experiment to detect sense (upper panel) and antisense (lower panel) sRNAs derived from hairpin RNAs expressed in transgenic tobacco plants. Lanes 1 and 2 contained RNA obtained from the parental plants PPGH11 and PPGH24 lacking the hpGUS constructs. Lanes 3-11 contained RNA from hpGUS[wt] plants and lanes 12-20 contained RNA from hpGUS[G:U] plants.

FIG. 17. Autoradiograph of a Northern blot hybridisation to detect antisense sRNAs from transgenic plants. Lanes 1-10 were from hpGUS[wt] plants, lanes 11-19 were from hpGUS[G:U] plants. The antisense sRNAs have mobility corresponding to 20-24 nt in length. The blot was reprobed with antisense to U6 RNA as a lane-loading control.

FIG. 18. Autoradiograph of a repeat Northern blot hybridisation to detect antisense sRNAs from transgenic plants FIG. 19. DNA methylation analysis of the junction region of the 35S promoter and sense GUS region in hpGUS constructs in transgenic plants. The junction fragments were PCR-amplified either with (+) or without (−) prior treatment of plant DNA with McrBC enzyme.

FIG. 20. DNA methylation analysis of the 35S promoter region in hpGUS constructs in transgenic plants. The 35S fragments were PCR-amplified either with (+) or without (−) prior treatment of plant DNA with McrBC enzyme.

FIG. 21. Size distribution and abundance of processed RNA. (A) EIN2 constructs. (B) GUS constructs.

FIG. 22. Alignment of the sense sequence (upper sequence, nucleotides 17 to 216 of SEQ ID NO:22) of the hpEIN2[G:U] construct and the nucleotide sequence (lower sequence, SEQ ID NO:27) of a region of the cDNA corresponding to the A. thaliana EIN2 target gene. The sense sequence was made by replacing 43 cytidine (C) nucleotides in the wild-type sequence with thymidine (T) nucleotides. Conserved nucleotides are asterisked, substituted C's are not asterisked.

FIG. 23. Alignment of the sense sequence (upper sequence, nucleotides 13 to 212 of SEQ ID NO:24) of the hpCHS[G:U] construct with the nucleotide sequence of a region of the cDNA corresponding to the A. thaliana CHS target gene (SEQ ID NO:28, lower sequence). The sense sequence was made by replacing 65 cytidine (C) nucleotides in the wild-type sequence with thymidine (T) nucleotides. Conserved nucleotides are asterisked, substituted C's are not asterisked.

FIG. 24. Alignment of the antisense sequence (upper sequence, nucleotides 8 to 207 of SEQ ID NO:25) of the hpEIN2[G:U/U:G] construct and the nucleotide sequence (lower sequence, SEQ ID NO:29) of a region of the complement of the A. thaliana EIN2 target gene and the. The antisense sequence was made by replacing 49 cytidine (C) nucleotides in the wild-type sequence with thymidine (T) nucleotides. Conserved nucleotides are asterisked, substituted C's are not asterisked.

FIG. 25. Alignment of the antisense sequence (upper sequence, nucleotides 13 to 212 of SEQ ID NO:26) of the hpCHS[G:U/U:G] construct and the nucleotide sequence (lower sequence, SEQ ID NO:30) of a region of the complement of the A. thaliana CHS target gene. The antisense sequence was made by replacing 49 cytidine (C) nucleotides in the wild-type sequence with thymidine (T) nucleotides. Conserved nucleotides are asterisked, substituted C's are not asterisked.

FIG. 26. Schematic diagrams of the ethylene insensitive 2 (EIN2) and chalcone synthase (CHS) hpRNA constructs. 35S:CaMV 35S promoter; EIN2 and CHS regions are show either as wild-type sequence (wt) or the G:U modified sequence (G:U). The arrows indicate the orientation of the DNA fragments—right to left arrows indicate the antisense sequences. Restriction enzyme sites are also shown.

FIG. 27. Hypocotyl lengths of transgenic A. thaliana seedlings in the EIN2 assay, containing either the hpEIN2[wt] or hpEIN2[G:U]

FIG. 28. qRT-PCR for CHS mRNA in transgenic A. thaliana transgenic for the hpCHS[wt] or hpCHS[G:U] constructs, normalised to the levels of Actin2 RNA. Col-0 is the wild-type (nontransgenic) A. thaliana.

FIG. 29. Autoradiograph of Northern blot hybridisation of RNA from plants transformed with hpEIN2[wt] or hpEIN2[G:U]. Upper panel shows the hypocotyl length for the lines. The autoradiograph shows Northern blot probed with an EIN2 sense probe to detect antisense sRNAs. The same blot was re-probed with a U6 RNA probe as a loading control (U6 RNA).

FIG. 30. DNA methylation analysis of 35S promoter and 35S-sense EIN2 sequences in genomic DNA of transgenic A. thaliana plants.

FIG. 31. Levels of DNA methylation in the promoter and 5′ region of hairpin RNA constructs.

FIG. 32. 35S promoter in the least methylated lines of the hpEIN2[wt] population still shows significant methylation.

FIG. 33. 35S promoter in the G:U hpEIN2 lines shows only weak methylation (<10%).

FIG. 34. ledRNA and hpRNA with G:U gene silencing in CHO and Vero cells at 72 hrs.

FIG. 35. Dumbbell plasmids tested in Hela cells at 48 hrs.

FIG. 36. Examples of possible modifications of dsRNA molecules.

FIG. 37. Reduced aphid performance following feeding from artificial diet supplemented with ledRNA for down-regulating expression of the MpC002 or MpRack-1 genes in green peach aphid. Upper panel (A): the average number of nymphs per adult aphid after a ten day period with 100 μl of 50 ng/μl ledRNA. Lower panel (B): percentage of aphids surviving over a five day time course after feeding on 100 μl containing 200 ng/μl ledRNA of MpC002, MpRack-1 or the control ledGFP.

### KEY TO THE SEQUENCE LISTING

SEQ ID NO:1—Ribonucleotide sequence of GFP ledRNA.

SEQ ID NO:2—Ribonucleotide sequence of GUS ledRNA.

SEQ ID NO:3—Ribonucleotide sequence of N. benthamiana FAD2.1 ledRNA.

SEQ ID NO:4—Nucleotide sequence encoding GFP ledRNA.

SEQ ID NO:5—Nucleotide sequence encoding GUS ledRNA.

SEQ ID NO:6—Nucleotide sequence encoding N. benthamiana FAD2.1 ledRNA.

SEQ ID NO:7—Nucleotide sequence encoding GFP.

SEQ ID NO:8—Nucleotide sequence encoding GUS.

SEQ ID NO:9—Nucleotide sequence encoding N. benthamiana FAD2.1.

SEQ ID NO: 10—Nucleotide sequence used to provide the GUS sense region for constructs encoding hairpin RNA molecules targeting the GUS mRNA.

SEQ ID NO: 11—Nucleotide sequence used to provide the GUS sense region for the construct encoding the hairpin RNA molecule hpGUS[G:U].

SEQ ID NO: 12—Nucleotide sequence used to provide the GUS sense region for constructs encoding the hairpin RNA molecule hpGUS[1:4].

SEQ ID NO: 13—Nucleotide sequence used to provide the GUS sense region for constructs encoding the hairpin RNA molecule hpGUS[2:10].

SEQ ID NO:14—Nucleotide sequence of nucleotides 781-1020 of the protein coding region of the GUS gene.

SEQ ID NO:15—Ribonucleotide sequence of the hairpin structure (including its loop) of the hpGUS[wt] RNA.

SEQ ID NO: 16—Ribonucleotide of the hairpin structure (including its loop) of the hpGUS[G:U] RNA.

SEQ ID NO: 17—Ribonucleotide of the hairpin structure (including its loop) of the hpGUS[1:4] RNA.

SEQ ID NO:18—Ribonucleotide of the hairpin structure (including its loop) of the hpGUS[2:10] RNA.

SEQ ID NO:19—Nucleotide sequence of the cDNA corresponding to the A. thaliana EIN2 gene, Accession No. NM_120406.

SEQ ID NO:20—Nucleotide sequence of the cDNA corresponding to A. thaliana CHS gene, Accession No. NM_121396, 1703 nt.

SEQ ID NO:21—Nucleotide sequence of a DNA fragment comprising a 200 nt sense sequence from the cDNA corresponding to the A. thaliana EIN2 gene flanked by restriction enzyme sites.

SEQ ID NO:22—Nucleotide sequence of a DNA fragment comprising the 200 nt sense sequence of EIN2 as for SEQ ID NO:21 except that 43 C's were replaced with T's, used in constructing hpEIN2[G:U].

SEQ ID NO:23—Nucleotide sequence of a DNA fragment comprising a 200 nt sense sequence from the cDNA corresponding to A. thaliana CHS gene flanked by restriction enzyme sites.

SEQ ID NO:24—Nucleotide sequence of a DNA fragment comprising the 200 nt sense sequence of CHS as for SEQ ID NO:23 except that 65 C's were replaced with T's, used in constructing hpCHS[G:U].

SEQ ID NO:25—Nucleotide sequence of a DNA fragment comprising the 200 nt antisense sequence of EIN2 with 50 C's replaced with T's, used in constructing hpEIN2[G:U/U:G].

SEQ ID NO:26—Nucleotide sequence of a DNA fragment comprising the 200 nt antisense sequence of CHS with 49 C's replaced with T's, used in constructing hpCHS[G:U/U:G].

SEQ ID NO:27—Nucleotide sequence of nucleotides 601-900 of the cDNA corresponding to the EIN2 gene from A. thaliana (Accession No. NM_120406).

SEQ ID NO:28—Nucleotide sequence of nucleotides 813-1112 of the cDNA corresponding to the CHS gene from A. thaliana (Accession No. NM_121396).

SEQ ID NO:29—Nucleotide sequence of the complement of nucleotides 652-891 of the cDNA corresponding to the EIN2 gene from A. thaliana (Accession No. NM_120406).

SEQ ID NO:30—Nucleotide sequence of the complement of nucleotides 804-1103 of the cDNA corresponding to the CHS gene from A. thaliana.

SEQ ID NO:31—FANCM I protein coding region of the cDNA of Arabidopsis thaliana, Accession No NM_001333162. Target region nucleotides 675-1174 (500 nucleotides)

SEQ ID NO:32—FANCM I protein coding region of a cDNA of Brassica napus. Target region nucleotides 896-1395 (500 bp)

SEQ ID NO:33—Nucleotide sequence encoding hpFANCM-At[wt] targeting the FANCM I protein coding region of A. thaliana. FANCM sense sequence, nucleotides 38-537; loop sequence, nucleotides 538-1306; FANCM antisense sequence, nucleotides 1307-1806.

SEQ ID NO:34—Nucleotide sequence encoding hpFANCM-At[G:U] targeting the FANCM I protein coding region of A. thaliana. FANCM sense sequence, nucleotides 38-537; loop sequence, nucleotides 538-1306; FANCM antisense sequence, nucleotides 1307-1806.

SEQ ID NO:35—Nucleotide sequence encoding hpFANCM-Bn[wt] targeting the FANCM I protein coding region of B. napus. FANCM sense sequence, nucleotides 34-533; loop sequence, nucleotides 534-1300; FANCM antisense sequence, nucleotides 1301-1800.

SEQ ID NO:36—Nucleotide sequence encoding hpFANCM-Bn[G:U] targeting the FANCM I protein coding region of B. napus. FANCM sense sequence, nucleotides 34-533; loop sequence, nucleotides 534-1300; FANCM antisense sequence, nucleotides 1301-1800.

SEQ ID NO:37—Nucleotide sequence of the protein coding region of the cDNA corresponding to the B. napus DDM1 gene; Accession No. XR_001278527.

SEQ ID NO:38—Nucleotide sequence of DNA encoding hpDDM1-Bn[wt] targeting the DDM1 protein coding region of B. napus.

SEQ ID NO:39—Nucleotide sequence encoding hpDDM1-Bn[G:U] targeting the DDM1 protein coding region of B. napus. DDM1 sense sequence, nucleotides 35-536; loop sequence, nucleotides 537-1304; DDM1 antisense sequence, nucleotides 1305-1805.

SEQ ID NO:40—EGFP cDNA.

SEQ ID NO:41—Nucleotide sequence of the coding region of hpEGFP[wt], with the order antisense/loop/sense with respect to the promoter.

SEQ ID NO:42—Nucleotide sequence of the coding region of hpEGFP[G:U] which has 157 C to T substitutions in the EGFP sense sequence.

SEQ ID NO:43—Nucleotide sequence of the coding region of ledEGFP[wt] which has no C to T substitutions in the EGFP sense sequence.

SEQ ID NO:44—Nucleotide sequence of the coding region of ledEGFP[G:U] which has 162 C to T substitutions in the EGFP sense sequence.

SEQ ID NO:45—Nucleotide sequence used to provide the GUS sense region for the construct encoding the hairpin RNA molecule hpGUS[G:U] without flanking restriction enzyme sites.

SEQ ID NO:46—Nucleotide sequence used to provide the GUS sense region for constructs encoding the hairpin RNA molecule hpGUS[1:4] without flanking restriction enzyme sites.

SEQ ID NO:47—Nucleotide sequence used to provide the GUS sense region for constructs encoding the hairpin RNA molecule hpGUS[2:10] without flanking restriction enzyme sites.

SEQ ID NO:48—Nucleotide sequence of a DNA fragment comprising the 200 nt sense sequence of EIN2 as for SEQ ID NO:21 except that 43 C's were replaced with T's, used in constructing hpEIN2[G:U] without flanking sequences.

SEQ ID NO:49—Nucleotide sequence of a DNA fragment comprising the 200 nt sense sequence of CHS as for SEQ ID NO:23 except that 65 C's were replaced with T's, used in constructing hpCHS[G:U] without flanking sequences.

SEQ ID NO:50—Nucleotide sequence of a DNA fragment comprising the 200 nt antisense sequence of EIN2 with 50 C's replaced with T's, used in constructing hpEIN2[G:U/U:G] without flanking sequences

SEQ ID NO:51—Nucleotide sequence of a DNA fragment comprising the 200 nt antisense sequence of CHS with 49 C's replaced with T's, used in constructing hpCHS[G:U/U:G] without flanking sequences.

SEQ ID NO:52—Oligonucleotide primer used for amplifying the 200 bp GUS sense sequence (GUS-WT-F)

SEQ ID NO:53—Oligonucleotide primer used for amplifying the 200 bp GUS sense sequence (GUS-WT-R)

SEQ ID NO:54—Oligonucleotide primer (forward) used for producing the hpGUS[G:U] fragment with every C replaced with T (GUS-GU-F)

SEQ ID NO:55—Oligonucleotide primer (reverse) used for producing the hpGUS[G:U] fragment with every C replaced with T (GUS-GU-R)

SEQ ID NO:56—Oligonucleotide primer (forward) used for producing the hpGUS[1:4] fragment with every 4th nucleotide substituted (GUS-4M-F)

SEQ ID NO:57—Oligonucleotide primer (reverse) used for producing the hpGUS[1:4] fragment with every 4th nucleotide substituted (GUS-4M-R)

SEQ ID NO:58—Oligonucleotide primer (forward) used for producing the hpGUS[2:10] fragment with every 9th and 10th nucleotide substituted (GUS-10M-F)

SEQ ID NO:59—Oligonucleotide primer (reverse) used for producing the hpGUS[2:10] fragment with every 9th and 10th nucleotide substituted (GUS-10M-R)

SEQ ID NO:60—Nucleotide sequence encoding forward primer (35S-F3)

SEQ ID NO:61—Nucleotide sequence encoding reverse primer (GUSwt-R2)

SEQ ID NO:62—Nucleotide sequence encoding forward primer (GUSgu-R2)

SEQ ID NO:63—Nucleotide sequence encoding reverse primer (GUS4m-R2)

SEQ ID NO:64—Nucleotide sequence encoding forward primer (35S-F2)

SEQ ID NO:65—Nucleotide sequence encoding reverse primer (35S-R1)

SEQ ID NO:66—Oligonucleotide primer used for amplifying the wild-type 200 bp EIN2 sense sequence (EIN2 wt-F)

SEQ ID NO:67—Oligonucleotide primer used for amplifying the wild-type 200 bp EIN2 sense sequence (EIN2 wt-R)

SEQ ID NO:68—Oligonucleotide primer used for amplifying the wild-type 200 bp CHS sense sequence (CHSwt-F)

SEQ ID NO:69—Oligonucleotide primer used for amplifying the wild-type 200 bp CHS sense sequence (CHSwt-R)

SEQ ID NO:70—Oligonucleotide primer (forward) used for producing the hpEIN2[G:U] fragment, with every C replaced with T (EIN2gu-F)

SEQ ID NO:71—Oligonucleotide primer (reverse) used for producing the hpEIN2[G:U] fragment, with every C replaced with T (EIN2gu-R)

SEQ ID NO:72—Oligonucleotide primer (forward) used for producing the hpCHS[G:U] fragment, with every C replaced with T (CHSgu-F)

SEQ ID NO:73—Oligonucleotide primer (reverse) used for producing the hpCHS[G:U] fragment, with every C replaced with T (CHSgu-R)

SEQ ID NO:74—Oligonucleotide primer (forward) used for producing the hpEIN2[G:U/U:G] fragment, with every C replaced with T (asEIN2gu-F)

SEQ ID NO:75—Oligonucleotide primer (reverse) used for producing the hpEIN2[G:U/U:G] fragment with every C replaced with T (asEIN2gu-R)

SEQ ID NO:76—Oligonucleotide primer (forward) used for producing the hpCHS[G:U/U:G] fragment, with every C replaced with T (asCHSgu-F)

SEQ ID NO:77—Oligonucleotide primer (reverse) used for producing the hpCHS[G:U/U:G] fragment, with every C replaced with T (asCHSgu-R)

SEQ ID NO:78—Nucleotide sequence encoding forward primer (CHS-200-F2)

SEQ ID NO:79—Nucleotide sequence encoding reverse primer (CHS-200-R2)

SEQ ID NO:80—Nucleotide sequence encoding forward primer (Actin2-For)

SEQ ID NO:81—Nucleotide sequence encoding reverse primer (Actin2-Rev)

SEQ ID NO:82—Nucleotide sequence encoding forward primer (Top-35S-F2)

SEQ ID NO:83—Nucleotide sequence encoding reverse primer (Top-35S-R2)

SEQ ID NO:84—Nucleotide sequence encoding forward primer (Link-35S-F2)

SEQ ID NO:85—Nucleotide sequence encoding reverse primer (Link-EIN2-R2)

SEQ ID NO:86—Ribonucleotide sequence of sense si22

SEQ ID NO:87—Ribonucleotide sequence of antisense si22

SEQ ID NO:88—Ribonucleotide sequence of forward primer

SEQ ID NO:89—Ribonucleotide sequence of reverse primer

SEQ ID NO:90—Ribonucleotide sequence of forward primer

SEQ ID NO:91—Ribonucleotide sequence of reverse primer

SEQ ID NO:92—Possible modifications of dsRNA molecules

SEQ ID NO:93—Nucleotide sequence of a cDNA corresponding to the Brassica napus DDM1 gene (Accession No. XR_001278527).

SEQ ID NO:94—Nucleotide sequence of a chimeric DNA encoding a hairpin RNAi (hpRNA) construct targeting a DDM1 gene of B. napus.

SEQ ID NO:95—Nucleotide sequence of a chimeric DNA encoding a hairpin RNAi (hpRNA) construct with G:U basepairs, targeting a DDM1 gene of B. napus.

SEQ ID NO:96—Nucleotide sequence of a chimeric DNA encoding a ledRNA construct, targeting a DDM1 gene of B. napus.

SEQ ID NO:97—Nucleotide sequence of cDNA corresponding to A. thaliana FANCM gene (Accession No. NM_001333162).

SEQ ID NO:98—Nucleotide sequence of a chimeric DNA encoding a hairpin RNAi (hpRNA) construct targeting a FANCM gene of A. thaliana.

SEQ ID NO:99—Nucleotide sequence of a chimeric DNA encoding a hairpin RNAi (hpRNA) construct with G:U basepairs, targeting a FANCM gene of A. thaliana.

SEQ ID NO:100—Nucleotide sequence of a chimeric DNA encoding a ledRNA construct, targeting a FANCM gene of A. thaliana.

SEQ ID NO:101—Nucleotide sequence of cDNA corresponding to B. napus FANCM gene (Accession No. XM_022719486.1).

SEQ ID NO: 102—Nucleotide sequence of a chimeric DNA encoding a hairpin RNAi (hpRNA) construct targeting a FANCM gene of B. napus.

SEQ ID NO: 103—Nucleotide sequence of a chimeric DNA encoding a hairpin RNAi (hpRNA) construct with G:U basepairs, targeting a FANCM gene of B. napus.

SEQ ID NO:104—Nucleotide sequence of a chimeric DNA encoding a ledRNA construct, targeting a FANCM gene of B. napus.

SEQ ID NO:105—Nucleotide sequence of the protein coding region of the cDNA corresponding to the Nicotiana benthamiana TOR gene.

SEQ ID NO:106—Nucleotide sequence of a chimeric DNA encoding a ledRNA construct targeting a TOR gene of N. benthamiana.

SEQ ID NO:107—Nucleotide sequence of the protein coding region of the cDNA corresponding to the acetolactate synthase (ALS) gene of barley, Hordeum vulgare (Accession No. LT601589).

SEQ ID NO:108—Nucleotide sequence of a chimeric DNA encoding a ledRNA targeting the ALS gene of barley (H. vulgare).

SEQ ID NO:109—Nucleotide sequence of the protein coding region of the cDNA corresponding to the HvNCED1 gene of barley Hordeum vulgare (Accession No. AK361999).

SEQ ID NO:110—Nucleotide sequence the protein coding region of the cDNA corresponding to the HvNCED2 gene of barley Hordeum vulgare (Accession No. DQ145931).

SEQ ID NO:111—Nucleotide sequence of a chimeric DNA encoding a ledRNA construct targeting the NCED1 genes of barley Hordeum vulgare and wheat Triticum aestivum.

SEQ ID NO:112—Nucleotide sequence of a chimeric DNA encoding a ledRNA construct targeting the NCED2 genes of barley Hordeum vulgare and wheat Triticum aestivum.

SEQ ID NO:113—Nucleotide sequence of the protein coding region of a cDNA corresponding to the barley gene encoding ABA-OH-2 (Accession No. DQ145933).

SEQ ID NO:114—Nucleotide sequence of a chimeric DNA encoding a ledRNA construct targeting the ABA-OH-2 genes of barley Hordeum vulgare and wheat Triticum aestivum.

SEQ ID NO:115—Nucleotide sequence of the protein coding region of a cDNA corresponding to the A. thaliana gene encoding EIN2 (At5g03280).

SEQ ID NO:116—Nucleotide sequence of a chimeric DNA encoding a ledRNA construct targeting the EIN2 gene of A. thaliana.

SEQ ID NO:117—Nucleotide sequence of the protein coding region of a cDNA corresponding to the A. thaliana gene encoding CHS (Accession No. NM-121396).

SEQ ID NO:118—Nucleotide sequence of a chimeric DNA encoding a ledRNA construct targeting the CHS gene of A. thaliana.

SEQ ID NO:119—Nucleotide sequence of the protein coding region of a cDNA corresponding to the L. angustifolius N-like gene (Accession No. XM_019604347).

SEQ ID NO:120—Nucleotide sequence of a chimeric DNA encoding a ledRNA construct targeting the L. angustifolius N-like gene.

SEQ ID NO:121—Nucleotide sequence of the protein coding region of a cDNA corresponding to a Vitis pseudoreticulata MLO gene (Accession No. KR362912).

SEQ ID NO: 122—Nucleotide sequence of a chimeric DNA encoding a first ledRNA construct targeting a Vitis MLO gene.

SEQ ID NO:123—Nucleotide sequence of the protein coding region of the cDNA corresponding to the MpC002 gene of Myzus persicae.

SEQ ID NO:124—Nucleotide sequence of the protein coding region of the cDNA corresponding to the MpRack-1 gene of Myzus persicae.

SEQ ID NO:125—Nucleotide sequence of the chimeric construct encoding the ledRNA targeting M. persicae C002 gene.

SEQ ID NO:126—Nucleotide sequence of the chimeric construct encoding the ledRNA targeting M. persicae Rack-1 gene.

SEQ ID NO:127—Nucleotide sequence of the cDNA corresponding to the Helicoverpa armigera ABCwhite gene.

SEQ ID NO:128—Nucleotide sequence of a chimeric DNA encoding a ledRNA construct targeting a ABC transporter white gene of Helicoverpa armigera.

SEQ ID NO:129—Nucleotide sequence of the cDNA corresponding to the Linepithema humile PBAN-type neuropeptides-like (XM_012368710).

SEQ ID NO:130—Nucleotide sequence of a chimeric DNA encoding a ledRNA construct targeting a PBAN gene in Argentine ants (Accession No. XM_012368710).

SEQ ID NO:131—Nucleotide sequence of a chimeric DNA encoding a ledRNA construct targeting a gene encoding V-type proton ATPase catalytic subunit A (Accession No. XM_023443547) of L. cuprina.

SEQ ID NO:132—Nucleotide sequence of a chimeric DNA encoding a ledRNA construct targeting a gene encoding RNAse 1/2 of L. cuprina.

SEQ ID NO:133—Nucleotide sequence of a chimeric DNA encoding a ledRNA construct targeting a gene encoding chitin synthase of L. cuprina.

SEQ ID NO:134—Nucleotide sequence of a chimeric DNA encoding a ledRNA construct targeting a gene encoding ecdysone receptor (EcR) of L. cuprina.

SEQ ID NO:135—Nucleotide sequence of a chimeric DNA encoding a ledRNA construct targeting a gene encoding gamma-tubulin 1/1-like of L. cuprina.

SEQ ID NO: 136—TaMlo target gene (AF384144).

SEQ ID NO:137—Nucleotide sequence of a chimeric DNA encoding a ledRNA construct targeting a gene encoding TaMlo.

SEQ ID NO:138—Nucleotide sequence of the protein coding region of a cDNA corresponding to a Vitis pseudoreticulata MLO gene (Accession No. KR362912).

SEQ ID NO: 139—Nucleotide sequence of a chimeric DNA encoding a first ledRNA construct targeting a Vitis MLO gene.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, gene silencing, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “antisense regulatory element” or “antisense ribonucleic acid sequence” or “antisense RNA sequence” as used herein means an RNA sequence that is at least partially complementary to at least a part of a target RNA molecule to which it hybridizes. In certain embodiments, an antisense RNA sequence modulates (increases or decreases) the expression or amount of a target RNA molecule or its activity, for example through reducing translation of the target RNA molecule. In certain embodiments, an antisense RNA sequence alters splicing of a target pre-mRNA resulting in a different splice variant. Exemplary components of antisense sequences include, but are not limited to, oligonucleotides, oligonucleosides, oligonucleotide analogues, oligonucleotide mimetics, and chimeric combinations of these.

The term “antisense activity” is used in the context of the present disclosure to refer to any detectable and/or measurable activity attributable to the hybridization of an antisense RNA sequence to its target RNA molecule. Such detection and/or measuring may be direct or indirect. In an example, antisense activity is assessed by detecting and or measuring the amount of target RNA molecule transcript. Antisense activity may also be detected as a change in a phenotype associated with the target RNA molecule. As used herein, the term “target RNA molecule” refers to a gene transcript that is modulated by an antisense RNA sequence according to the present disclosure. Accordingly, “target RNA molecule” can be any RNA molecule the expression or activity of which is capable of being modulated by an antisense RNA sequence. Exemplary target RNA molecules include, but are not limited to, RNA (including, but not limited to pre-mRNA and mRNA or portions thereof) transcribed from DNA encoding a target protein, rRNA, tRNA, small nuclear RNA, and miRNA, including their precursor forms. The target RNA may be the genomic RNA of a pathogen or pest such as a virus, or an RNA molecule derived therefrom such as a replicative form of a viral pathogen, or transcript therefrom. For example, the target RNA molecule can be an RNA from an endogenous gene (or mRNA transcribed from the gene) or a gene which is introduced or may be introduced into the eukaryotic cell whose expression is associated with a particular phenotype, trait, disorder or disease state, or a nucleic acid molecule from an infectious agent. In an example, the target RNA molecule is in a eukaryotic cell. In another example, the target RNA molecule encodes a protein. In this context, antisense activity can be assessed by detecting and or measuring the amount of target protein, for example through its activity such as enzyme activity, or a function other than as an enzyme, or through a phenotype associated with its function. As used herein, the term “target protein” refers to a protein that is modulated by an antisense RNA sequence according to the present disclosure.

In certain embodiments, antisense activity is assessed by detecting and/or measuring the amount of target RNA molecules and/or cleaved target RNA molecules and/or alternatively spliced target RNA molecules.

Antisense activity can be detected or measured using various methods. For example, antisense activity can be detected or assessed by comparing activity in a particular sample and comparing the activity to that of a control sample.

The term “targeting” is used in the context of the present disclosure to refer to the association of an antisense RNA sequence to a particular target RNA molecule or a particular region of nucleotides within a target RNA molecule. In an example, an antisense RNA sequence according to the present disclosure shares complementarity with at least a region of a target RNA molecule. In this context, the term “complementarity” refers to a sequence of ribonucleotides that is capable of base pairing with a sequence of ribonucleotides on a target RNA molecule, through hydrogen bonding between bases on the ribonucleotides. For example, in RNA, adenine (A) is complementary to uracil (U) and guanine (G) to cytosine (C).

In certain embodiments, “complementary base” refers to a ribonucleotide of an antisense RNA sequence that is capable of base pairing with a ribonucleotide of a sense RNA sequence in an RNA molecule of the invention or of its target RNA molecule. For example, if a ribonucleotide at a certain position of an antisense RNA sequence is capable of hydrogen bonding with a ribonucleotide at a certain position of a target RNA molecule, then the position of hydrogen bonding between the antisense RNA sequence and the target RNA molecule is considered to be complementary at that ribonucleotide. In contrast, the term “non-complementary” refers to a pair of ribonucleotides that do not form hydrogen bonds with one another or otherwise support hybridization. The term “complementary” can also be used to refer to the capacity of an antisense RNA sequence to hybridize to another nucleic acid through complementarity. In certain embodiments, an RNA sequence and its target are complementary to each other when a sufficient number of corresponding positions in each molecule are occupied by ribonucleotides that can bond with each other to allow stable association between the antisense RNA sequence and a sense RNA sequence in the RNA molecule of the invention and/or the target RNA molecule. One skilled in the art recognizes that the inclusion of mismatches is possible without eliminating the ability of the antisense RNA sequence and target to remain in association. Therefore, described herein are antisense RNA sequence that may comprise up to about 20% nucleotides that are mismatched (i.e., are not complementary to the corresponding nucleotides of the target). Preferably the antisense compounds contain no more than about 15%, more preferably not more than about 10%, most preferably not more than 5% or no mismatches. The remaining ribonucleotides are complementary or otherwise do not disrupt hybridization (e.g., G:U or A:G pairs) between the antisense RNA sequence and the sense RNA sequence or the target RNA molecule. One of ordinary skill in the art would recognise the antisense RNA sequence s described herein are at least 80%, at least 85%, at least 90%, at least 95%, at least 96%, at least 97%, at least 98%, at least 99% or 100% (fully) complementary to at least a region of a target RNA molecule.

As used herein, “chimeric RNA molecule” refers to any RNA molecule that is not naturally found in nature. In an example, chimeric RNA molecules disclosed herein have been modified to create mismatches in region(s) of dsRNA. For example, chimeric RNA molecules may be modified to convert cytosines to uracils. In an example, chimeric RNA molecules have been modified via treatment with bisulfite for a time and under conditions sufficient to convert non-methylated cytosines to uracils.

One of skill in the art would appreciate that various ribonucleotide combinations can base pair. Both canonical and non-canonical base pairings are contemplated by the present disclosure. In an example, abase pairing can comprise A:T or G:C in a DNA molecule or U:A or G:C in an RNA molecule. In another example, a base pairing may comprise A:G or G:T or U:G.

The term “canonical base pairing” as used in the present disclosure means base pairing between two nucleotides which are A:T or G:C for deoxyribonucleotides or A:U or G:C for ribonucleotides.

The term “non-canonical base pairing” as used in the present disclosure means an interaction between the bases of two nucleotides other than canonical base pairings, in the context of two DNA or two RNA sequences. For example, non-canonical base pairing includes pairing between G and U (G:U) or between A and G (A:G). Examples of non-canonical base pairing include purine-purine or pyrimidine-pyrimidine. Most commonly in the context of this disclosure, the non-canonical base pairing is G:U. Other examples of non-canonical base pairs, less preferred, are A:C, G:T, G:G and A:A.

The present disclosure refers to RNA components that “hybridize” across a series of ribonucleotides. Those of skill in the art will appreciate that terms such as “hybridize” and “hybridizing” are used to describe molecules that anneal based on complementary nucleic acid sequences. Such molecules need not be 100% complementary in order to hybridize (i.e. they need not “fully base pair”). For example, there may be one or more mismatches in sequence complementarity. In an example, RNA components defined herein hybridise under stringent hybridization conditions. The term “stringent hybridization conditions” refers to parameters with which the art is familiar, including the variation of the hybridization temperature with length of an RNA molecule. Ribonucleotide hybridization parameters may be found in references which compile such methods, Sambrook, et al. (supra), and Ausubel, et al. (supra). For example, stringent hybridization conditions, as used herein, can refer to hybridization at 65° C. in hybridization buffer (3.5×SSC, 0.02% Ficoll, 0.02% polyvinyl pyrrolidone, 0.02% Bovine Serum Albumin (BSA), 2.5 mM NaH₂PO₄ (pH7), 0.5% SDS, 2 mM EDTA), followed by one or more washes in 0.2×SSC, 0.01% BSA at 50° C. Shorter RNA components such as RNA sequences of 20-24 nucleotides in length hybridise under lower stringency conditions. The term “low stringency hybridization conditions” refers to parameters with which the art is familiar, including the variation of the hybridization temperature with length of an RNA molecule. For example, low stringency hybridization conditions, as used herein, can refer to hybridization at 42° C. in hybridization buffer (3.5×SSC, 0.02% Ficoll, 0.02% polyvinyl pyrrolidone, 0.02% Bovine Serum Albumin (BSA), 2.5 mM NaH₂PO₄ (pH7), 0.5% SDS, 2 mM EDTA), followed by one or more washes in 0.2×SSC, 0.01% BSA at 30° C.

The present invention also encompasses RNA components that “fully base pair” across contiguous ribonucleotides. The term “fully base pair” is used in the context of the present disclosure to refer to a series of contiguous ribonucleotide base pairings. A fully base paired series of contiguous ribonucleotides does not comprise gaps or non-basepaired nucleotides within the series. The term “contiguous” is used to refer to a series of ribonucleotides. Ribonucleotides comprising a contiguous series will be joined by a continuous series of phosphodiester bonds, each ribonucleotide being directly bonded to the next.

RNA molecules of the present invention comprise a sense sequence and a corresponding antisense sequence. The relationship between these sequences is defined herein. The sequence relationship and activity of the antisense sequence in relation to a target RNA molecule is also defined herein.

The term “covalently linked” is used in the context of the present disclosure to refer to the link between the first and second RNA components or any RNA sequences or ribonucleotides. As one of skill in the art would appreciate, a covalent link or bond is a chemical bond that involves the sharing of electron pairs between atoms. In an example, the first and second RNA components or the sense RNA sequence and the antisense RNA sequence are covalently linked as part of a single RNA strand which may fold back on itself through self-complementarity. In this example, the components are covalently linked across one or more ribonucleotides by phosphodiester bonds.

In the context of the present disclosure, the term “hybridization” means the pairing of complementary polynucleotides through basepairing of complementary bases. While not limited to a particular mechanism, the most common mechanism of pairing involves hydrogen bonding, which may be Watson-Crick hydrogen bonding, between complementary ribonucleotides.

As used herein, the phrase “the RNA molecule has a deleterious effect on the non-human organism” or similar phrases means that the target RNA molecule of the molecule is present in the non-human organism and exposure of cells expressing the target RNA molecule to the target RNA molecule results in reduced levels and/or activity of the target RNA molecule when compared to the same cells lacking the RNA molecule. In an embodiment, the target RNA molecule encodes a protein important for growth, reproduction or survival. As an example, if the non-human organism is a crop pest or pathogen, or a pest or pathogen of an animal, the RNA molecule can have a deleterious effect on feeding by the pest or pathogen, cell apoptosis, cell differentiation and development, capacity or desire for sexual reproduction, muscle formation, muscle twitching, muscle contraction, juvenile hormone formation, juvenile hormone regulation, ion regulation and transport, maintenance of cell membrane potential, amino acid biosynthesis, amino acid degradation, sperm formation, pheromone synthesis, pheromone sensing, antennae formation, wing formation, leg formation, egg formation, larval maturation, digestive enzyme formation, haemolymph synthesis, haemolymph maintenance, neurotransmission, larval stage transition, pupation, emergence from pupation, cell division, energy metabolism, respiration, chitin metabolism, formation of cytoskeletal structure. In another example, the non-human organism is a weed and the RNA molecule has a deleterious effect on amino acid biosynthesis, photosynthesis, fatty acid synthesis, cell membrane integrity, pigment synthesis or growth.

As used herein, the phrase “the RNA molecule has a beneficial effect on at least one symptom of the disease” or similar phrases means that the target RNA of the molecule is present in the subject and exposure of cells expressing the target RNA to the RNA molecule results in reduced levels and/or activity of the target RNA when compared to the same cells lacking the RNA molecule. In an embodiment, the target RNA is encodes a protein which plays a role in the presence of the disease. In an embodiment, the disease is cancer or cancerous disease, an infectious disease, a cardiovascular disease, a neurological disease, a prion disease, an inflammatory disease, an autoimmune disease, a pulmonary disease, a renal disease, liver disease, mitochondrial disease, endocrine disease, reproduction related diseases and conditions, and any other indications that can respond to the level of an expressed gene product in a cell or organism.

RNA molecules according to the present disclosure and compositions comprising the same can be administered to a subject. Terms such as “subject”, “patient” or “individual” are terms that can, in context, be used interchangeably in the present disclosure. In an example, the subject is a mammal. The mammal may be a companion animal such as a dog or cat, or a livestock animal such as a horse or cow. In one example, the subject is a human. For example, the subject can be an adult. In another example, the subject can be a child. In another example, the subject can be an adolescent. In another example, RNA molecules according to the present disclosure and compositions comprising the same can be administered to an insect. In another example, RNA molecules according to the present disclosure and compositions comprising the same can be administered to a plant. In another example, RNA molecules according to the present disclosure and compositions comprising the same can be administered to a fungal cell or population.

The term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−20%, more preferably +/−10%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### RNA Molecule

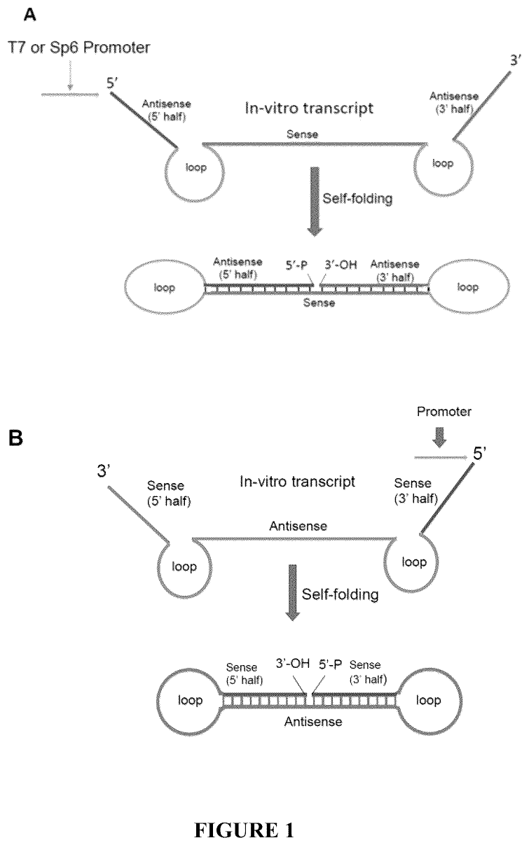

In certain embodiments, RNA molecules of the present invention comprise a first RNA component which is covalently linked to a second RNA component. In preferred embodiments, the RNA molecule self-hybridizes or folds to form a “dumbbell” or ledRNA structure, for example see FIG. 1. In an embodiment, the molecule further comprises one or more of the following:
a linking ribonucleotide sequence which covalently links the first and second RNA components;
a 5′ leader sequence; and,
a 3′ trailer sequence.

In an embodiment, the first RNA component consists of, in 5′ to 3′ order, a first 5′ ribonucleotide, a first RNA sequence and a first 3′ ribonucleotide, wherein the first 5′ and 3′ ribonucleotides basepair to each other in the RNA molecule, wherein the first RNA sequence comprises a first sense ribonucleotide sequence of at least 20 contiguous ribonucleotides, a first loop sequence of at least 4 ribonucleotides and a first antisense ribonucleotide sequence of at least 20 contiguous ribonucleotides, wherein the first antisense ribonucleotide sequence hybridises with the first sense ribonucleotide sequence in the RNA molecule, wherein the first antisense ribonucleotide sequence is capable of hybridising to a first region of a target RNA molecule.

In another embodiment, the first RNA component consists of, in 5′ to 3′ order, a first 5′ ribonucleotide, a first RNA sequence and a first 3′ ribonucleotide, wherein the first 5′ and 3′ ribonucleotides basepair to each other in the RNA molecule, wherein the first RNA sequence comprises a first sense ribonucleotide sequence of at least 20 contiguous ribonucleotides, a first loop sequence of at least 4 ribonucleotides and a first antisense ribonucleotide sequence of at least 20 contiguous ribonucleotides, wherein the first antisense ribonucleotide sequence fully basepairs with the first sense ribonucleotide sequence in the RNA molecule, wherein the first antisense ribonucleotide sequence is identical in sequence to the complement of a first region of a target RNA molecule. An example of this first RNA component of these two embodiments is shown schematically in the left-hand half of FIG. 1A or the right-hand half of FIG. 1B.

In another embodiment, the first RNA component consists of a first 5′ ribonucleotide, a first RNA sequence and a first 3′ ribonucleotide, wherein the first 5′ and 3′ ribonucleotides basepair with each other in the first RNA component, wherein the first RNA sequence comprises a first sense ribonucleotide sequence, a first loop sequence of at least 4 ribonucleotides and a first antisense ribonucleotide sequence, wherein the first sense ribonucleotide sequence and first antisense ribonucleotide sequence each of at least 20 contiguous ribonucleotides whereby the at least 20 contiguous ribonucleotides of the first sense ribonucleotide sequence fully basepair with the at least 20 contiguous ribonucleotides of the first antisense ribonucleotide sequence, wherein the at least 20 contiguous ribonucleotides of the first sense ribonucleotide sequence are substantially identical in sequence to a first region of a target RNA molecule.

In these embodiments, the basepair formed between the first 5′ ribonucleotide and the first 3′ ribonucleotide is considered to be the terminal basepair of the dsRNA region formed by self-hybridization of the first RNA component, i.e it defines the end of the dsRNA region.

In an embodiment, the first sense sequence has substantial sequence identity to a region of the target RNA, which identity may be to a sequence of less than 20 nucleotides in length. In an embodiment at least 15, at least 16, at least 17, at least 18, or at least 19 contiguous ribonucleotides, preferably at least 20 contiguous ribonucleotides, of the first sense ribonucleotide sequence and a first region of a target RNA molecule are at least 60%, at least 65%, at least 70%, at least 75%, at least 80%, at least 85%, at least 90%, at least 95%, at least 96%, at least 97%, at least 98%, or 99% identical in sequence. In another embodiment, the at least 15, at least 16, at least 17, at least 18, at least 19 contiguous ribonucleotides of the first sense ribonucleotide sequence and a first region of a target RNA molecule are 100% identical. In an embodiment, the first 3, first 4, first 5, first 6, or first 7 ribonucleotides from the 5′ end of the first sense ribonucleotide sequence are 100% identical to the region of the target RNA molecule, with the remaining ribonucleotides being at least 60%, at least 65%, at least 70%, at least 75%, at least 80%, at least 85%, at least 90%, at least 95%, at least 96%, at least 97%, at least 98%, at least 99% identical to the target RNA molecule.

In an embodiment the at least 20 contiguous ribonucleotides of the first sense ribonucleotide sequence and a first region of a target RNA molecule are at least 60%, at least 65%, at least 70%, at least 75%, at least 80%, at least 85%, at least 90%, at least 95%, at least 96%, at least 97%, at least 98%, or at least 99% identical. Again, in this embodiment, the first 3, first 4, first 5, first 6, or first 7 ribonucleotides can be 100% identical to the region of the target RNA molecule, with the remaining ribonucleotides being at least 60%, at least 65%, at least 70%, at least 75%, at least 80%, at least 85%, at least 90%, at least 95%, at least 96%, at least 97%, at least 98%, or at least 99% identical to target RNA molecule. In another embodiment, the at least 20 contiguous ribonucleotides of the first sense ribonucleotide sequence and a first region of a target RNA molecule are 100% identical.

In an embodiment, the first antisense sequence has substantial sequence identity to the complement of a region of the target RNA, which identity may be to a sequence of less than 20 nucleotides in length of the complement. In an embodiment at least 15, at least 16, at least 17, at least 18, or at least 19 contiguous ribonucleotides, preferably at least 20 contiguous ribonucleotides, of the first antisense ribonucleotide sequence and the complement of a first region of a target RNA molecule are at least 60%, at least 65%, at least 70%, at least 75%, at least 80%, at least 85%, at least 90%, at least 95%, at least 96%, at least 97%, at least 98%, or 99% identical in sequence. In another embodiment, the at least 15, at least 16, at least 17, at least 18, at least 19 contiguous ribonucleotides of the first antisense ribonucleotide sequence and the complement of the first region of the target RNA molecule are 100% identical. In an embodiment, the first 3, first 4, first 5, first 6, or first 7 ribonucleotides from the 5′ end of the first antisense ribonucleotide sequence are 100% identical to the complement of the region of the target RNA molecule, with the remaining ribonucleotides being at least 60%, at least 65%, at least 70%, at least 75%, at least 80%, at least 85%, at least 90%, at least 95%, at least 96%, at least 97%, at least 98%, at least 99% identical to the complement of the target RNA molecule.

In an embodiment the at least 20 contiguous ribonucleotides of the first antisense ribonucleotide sequence and the complement of a first region of the target RNA molecule are at least 60%, at least 65%, at least 70%, at least 75%, at least 80%, at least 85%, at least 90%, at least 95%, at least 96%, at least 97%, at least 98%, or at least 99% identical. Again, in this embodiment, the first 3, first 4, first 5, first 6, or first 7 ribonucleotides are 100% identical to the complement of the region of the target RNA molecule, with the remaining ribonucleotides being at least 60%, at least 65%, at least 70%, at least 75%, at least 80%, at least 85%, at least 90%, at least 95%, at least 96%, at least 97%, at least 98%, or at least 99% identical to the complement of the target RNA molecule. In another embodiment, the at least 20 contiguous ribonucleotides of the first antisense ribonucleotide sequence and a first region of a target RNA molecule are 100% identical.

In another embodiment, the second RNA component consists of, in 5′ to 3′ order, a second 5′ ribonucleotide, a second RNA sequence and a second 3′ ribonucleotide, wherein the second 5′ and 3′ ribonucleotides basepair, wherein the second RNA sequence comprises a second sense ribonucleotide sequence, a second loop sequence of at least 4 ribonucleotides and a second antisense ribonucleotide sequence, wherein the second sense ribonucleotide sequence basepairs with the second antisense ribonucleotide sequence. In this embodiment, the basepair formed between the second 5′ ribonucleotide and the second 3′ ribonucleotide is considered to be the terminal basepair of the dsRNA region formed by self-hybridization of the second RNA component.

In an embodiment, the RNA molecule comprises a 5′ leader sequence, or 5′ extension sequence, which may arise as a result of transcription from a promoter in the genetic construct, from the start site of transcription to the beginning of the polynucleotide encoding the remainder of the RNA molecule. It is preferred that this 5′ leader sequence or 5′ extension sequence is relatively short compared to the remainder of the molecule, and it may be removed from the RNA molecule post-transcriptionally, for embodiment by RNAse treatment. The 5′ leader sequence or 5′ extension sequence may be mostly non-basepaired, or it may contain one or more stem-loop structures. In this embodiment, the 5′ leader sequence can consist of a sequence of ribonucleotides which is covalently linked to the first 5′ ribonucleotide if the second RNA component is linked to the first 3′ ribonucleotide or to the second 5′ ribonucleotide if the second RNA component is linked to the first 5′ ribonucleotide. In an embodiment, the 5′ leader sequence is at least 10, at least 20, at least 30, at least 100, at least 200 ribonucleotides long, preferably to a maximum length of 250 ribonucleotides. In another embodiment, the 5′ leader sequence is at least 50 ribonucleotides long. In an embodiment, the 5′ leader sequence can act as an extension sequence for amplification of the RNA molecule via a suitable amplification reaction. For embodiment, the extension sequence may facilitate amplification via polymerase.

In another embodiment, the RNA molecule comprises a 3′ trailer sequence or 3′ extension sequence which may arise as a result of transcription continuing until a transcription termination or polyadenylation signal in the construct encoding the RNA molecule. The 3′ trailer sequence or 3′ extension sequence may comprise a polyA tail. It is preferred that this 3′ trailer sequence or 3′ extension sequence is relatively short compared to the remainder of the molecule, and it may be removed from the RNA molecule post-transcriptionally, for embodiment by RNAse treatment. The 3′ trailer sequence or 3′ extension sequence may be mostly non-basepaired, or it may contain one or more stem-loop structures. In this embodiment, the 3′ trailer sequence can consist of a sequence of ribonucleotides which is covalently linked to the second 3′ ribonucleotide if the second RNA component is linked to the first 3′ ribonucleotide or to the first 3′ ribonucleotide if the second RNA component is linked to the first 5′ ribonucleotide. In an embodiment, the 3′ leader sequence is at least 10, at least 20, at least 30, at least 100, at least 200 ribonucleotides long, preferably to a maximum length of 250 ribonucleotides. In another embodiment, the 3′ leader sequence is at least 50 ribonucleotides long. In an embodiment, the 3′ trailer sequence can act as an extension sequence for amplification of the RNA molecule via a suitable amplification reaction. For embodiment, the extension sequence may facilitate amplification via polymerase.

In an embodiment, all except for two of the ribonucleotides are covalently linked to two other nucleotides i.e. the RNA molecule consists of only one RNA strand which has self-complementary regions, and so has only one 5′ terminal nucleotide and one 3′ terminal nucleotide. In another embodiment, all except for four of the ribonucleotides are covalently linked to two other nucleotides i.e. the RNA molecule consists of two RNA strands which have complementary regions which hybridise, and so has only two 5′ terminal nucleotides and two 3′ terminal nucleotides. In another embodiment, each ribonucleotide is covalently linked to two other nucleotides i.e the RNA molecule is circular as well as having self-complementary regions, and so has no 5′ terminal nucleotide and no 3′ terminal nucleotide.

In an embodiment, the double-stranded region of the RNA molecule can comprise one or more bulges resulting from unpaired nucleotides in the sense RNA sequence or the antisense RNA sequence, or both. In an embodiment, the RNA molecule comprises a series of bulges. For embodiment, the double-stranded region of the RNA molecule may have 2, 3, 4, 5, 6, 7, 8, 9, 10 or more bulges. Each bulge may be, independently, one, two or more unpaired nucleotides, to as many as 10 nucleotides. Longer sequences may loop out of the sense or antisense sequences in the dsRNA region, which may basepair internally or remain unpaired. In another embodiment, the double-stranded region of the RNA molecule does not comprise a bulge i.e. is fully basepaired along the full length of the dsRNA region.

In another embodiment, the first sense ribonucleotide sequence is covalently linked to the first 5′ ribonucleotide without any intervening nucleotides, or the first antisense ribonucleotide sequence is covalently linked to the first 3′ ribonucleotide without any intervening nucleotides, or both. In another embodiment, there are at least one, at least two, at least three, at least four, at least five, at least six, at least seven, at least eight, at least nine, at least 10 intervening nucleotides. It is understood that such intervening nucleotides are unrelated in sequence to the target RNA molecule but may assist in stabilising the basepairing of adjacent sense and antisense sequences.

In another embodiment, the 20 consecutive nucleotides of the first sense ribonucleotide sequence are covalently linked to the first 5′ ribonucleotide without any intervening nucleotides, and the 20 consecutive nucleotides of the first antisense ribonucleotide sequence are covalently linked to the first 3′ ribonucleotide without any intervening nucleotides. In another embodiment, there are at least one, at least two, at least three, at least four, at least five, at least six, at least seven, at least eight, at least nine, at least 10 intervening nucleotides. The intervening nucleotides may be basepaired as part of the double-stranded region of the RNA molecule but are unrelated in sequence to the target RNA. They may assist in providing increased stability to the double-stranded region or to hold together two ends of the RNA molecule and not leave an unbasepaired 5′ or 3′ end, or both.

In an embodiment, the above referenced first and second RNA components comprise a linking ribonucleotide sequence. In an embodiment, the linking ribonucleotide sequence acts as a spacer between the first sense ribonucleotide sequence that is substantially identical in sequence to a first region of a target RNA molecule and the other components of the molecule. For example, the linking ribonucleotide sequence may act as a spacer between this region and a loop. In another embodiment, the RNA molecule comprises multiple sense ribonucleotide sequences that are substantially identical in sequence to a first region of a target RNA molecule and a linking ribonucleotide sequence which acts as a spacer between these sequences. In an embodiment, at least two, at least three, at least four, at least five, at least six, at least seven, at least eight, at least nine, at least 10 ribonucleotide sequences that are substantially identical in sequence to a first region of a target RNA molecule are provided in the RNA molecule, each being separated from the other(s) by a linking ribonucleotide sequence.

In an embodiment, the above referenced RNA molecules comprise a 5′ leader sequence. In an embodiment, the 5′ leader sequence consists of a sequence of ribonucleotides which is covalently linked to the first 5′ ribonucleotide if the second RNA component is linked to the first 3′ ribonucleotide or to the second 5′ ribonucleotide if the second RNA component is linked to the first 5′ ribonucleotide. In an embodiment, the RNA molecule has a modified 5′ or 3′ end, for embodiment by attachment of a lipid group such as cholesterol, or a vitamin such as biotin, or a polypeptide. Such modifications may assist in the uptake of the RNA molecule into the eukaryotic cell where the RNA is to function.

In an embodiment, the linking ribonucleotide sequence is less than 100 ribonucleotides in length. In an embodiment, the linking ribonucleotide sequence is less than 50 ribonucleotides in length. In an embodiment, the linking ribonucleotide sequence is less than 20 ribonucleotides in length. In an embodiment, the linking ribonucleotide sequence is less than 10 ribonucleotides in length. In an embodiment, the linking ribonucleotide sequence is less than 5 ribonucleotides in length. In an embodiment, the linking ribonucleotide sequence is between 1 and 100 ribonucleotides in length. In an embodiment, the linking ribonucleotide sequence is between 1 and 50 ribonucleotides in length. In an embodiment, the linking ribonucleotide sequence is between 1 and 20 ribonucleotides in length. In an embodiment, the linking ribonucleotide sequence is between 1 and 10 ribonucleotides in length. In an embodiment, the linking ribonucleotide sequence is between 1 and 5 ribonucleotides in length.

In an embodiment, the ribonucleotides of the linking ribonucleotide sequence are not basepaired. In a preferred embodiment, the ribonucleotides of the linking ribonucleotide sequence are all basepaired, or all except for 1, 2 or 3 of the ribonucleotides are basepaired.

In an embodiment, the first or second RNA component comprises a hairpin structure. In a preferred embodiment, the first and second RNA components each comprise a hairpin structure. In these embodiments, the hairpin structure can be a stem-loop. Accordingly, in an embodiment, the RNA molecule can comprise first and second RNA components which each comprise a hairpin structure, wherein the hairpins are covalently bound by a linker sequence. See, for example, FIG. 1. In an embodiment, the linker sequence is one or more unpaired ribonucleic acid(s). In an embodiment, the linker sequence is between 1 and 10 unpaired ribonucleotides.

In an embodiment, the RNA molecule has a double hairpin structure i.e. an “ledRNA structure” or “dumbbell structure”. In this embodiment, the first hairpin is the first RNA component and the second hairpin is the second RNA component. In these embodiments, either the first 3′ ribonucleotide and the second 5′ ribonucleotide, or the second 3′ ribonucleotide and the first 5′ ribonucleotide, but not both, are covalently joined. In this embodiment, the other 5′/3′ ribonucleotides can be separated by a nick (i.e. a discontinuity in the dsRNA molecule where there is no phosphodiester bond between the 5′/3′ ribonucleotides. An embodiment, of this type of arrangement is shown in FIG. 1B. In another embodiment, the respective 5′/3′ ribonucleotides can be separated by a loop. The lengths of the 5′ leader and 3′ trailer sequences may be the same or different. For embodiment, the 5′ leader may be around 5, 10, 15, 20, 25, 50, 100, 200, 500 ribonucleotides longer than the 3′ trailer sequence or vice versa.

In embodiments where the RNA molecule has a double hairpin structure, the second hairpin (in addition to the first hairpin structure) comprises a sense RNA sequence and an antisense RNA sequence that are substantially identical in sequence to a region of a target RNA molecule or its complement, respectively. In an embodiment, each hairpin has a series of ribonucleotides that are substantially identical in sequence to a region of the same target RNA molecule. In an embodiment, each hairpin has a series of ribonucleotides that are substantially identical in sequence to different regions of the same target RNA molecule. In an embodiment, each hairpin has a series of ribonucleotides that are substantially identical in sequence to a region of different target RNA molecules i.e. the RNA molecule can be used to reduce the expression and/or activity of two target RNA molecules which may be unrelated in sequence.

In each hairpin of the double hairpin structure of the RNA molecule, the order of the sense and antisense RNA sequences in each hairpin, in 5′ to 3′ order, may independently be either sense then antisense, or antisense then sense. In preferred embodiments, the order of the sense and antisense sequences in the double hairpin structure of the RNA molecule is either antisense-sense-sense-antisense where the two sense sequences are contiguous (FIG. 1A), or sense-antisense-antisense-sense where the two antisense sequences are contiguous (FIG. 1B).

In an embodiment, the RNA molecule can comprise, in 5′ to 3′ order, a 5′ leader sequence, a first loop, a sense RNA sequence, a second loop and a 3′ trailer sequence, wherein the 5′ and 3′ leader sequences covalently bond to the sense strand to form a dsRNA sequence. In an embodiment, the 5′ leader and 3′ trailer sequences are not covalently bound to each other. In an embodiment, the 5′ leader and 3′ trailer sequences are separated by a nick. In an embodiment, the 5′ leader and 3′ trailer sequences are ligated together to provide a RNA molecule with a closed structure. In another embodiment, the 5′ leader and 3′ trailer sequences are separated by a loop.

The term “loop” is used in the context of the present disclosure to refer to a loop structure in an RNA molecule disclosed herein that is formed by a series of non-complementary ribonucleotides. Loops generally follow a series of base-pairs between the first and second RNA components or join a sense RNA sequence and an antisense RNA sequence in one or both of the first and second RNA components. In an embodiment, all of the loop ribonucleotides are non-complementary, generally for shorter loops of 4-10 ribonucleotides. In other embodiments, some ribonucleotides in one or more of the loops are complementary and capable of basepairing within the loop sequence, so long as these basepairings enable a loop structure to form. For example, at least 5%, at least 10%, or at least 15% of the loop ribonucleotides are complementary. Embodiments of loops include stem loops or hairpins, pseudoknots and tetraloops.

In an embodiment, the RNA molecule comprises only two loops, In another embodiment, the RNA molecule comprises at least two, at least three, at least 4, at least 5, at least 6, at least 7, at least 8, at least 9, or at least 10 loops, preferably to a maximum of 10 loops. For example, the RNA molecule can comprise 4 loops.

Loops of various sizes are contemplated by the present disclosure. For example, loops can comprise 4, 5, 6, 7, 8, 9, 10, 11 or 12 ribonucleotides. In other embodiments, loops comprise 15, 20, 25 or 30 nucleotides. In an embodiment, one or all of the loop sequences are longer than 20 nucleotides. In other embodiments, loops are larger, for example comprising 50, 100, 150, 200 or 300 ribonucleotides. In an embodiment, loops comprise 160 ribonucleotides. In another embodiment, less preferred, loops comprise 200, 500, 700 or 1,000 ribonucleotides provided that the loops do not interfere with the hybridisation of the sense and antisense RNA sequences. In an embodiment, each of the loops have the same number of ribonucleotides. For example, loops can have between 100 and 1,000 ribonucleotides in length. For example, loops can have between 600 and 1,000 ribonucleotides in length. For example, loops can have between 4 and 1,000 ribonucleotides. For example, loops preferably have between 4 and 50 ribonucleotides. In another embodiment, loops comprise differing numbers of ribonucleotides.

In another embodiment, one or more loops comprise an intron which can be spliced out of the RNA molecule. In an embodiment, the intron is from a plant gene. Exemplary introns include intron 3 of the maize alcohol dehydrogenase 1 (Adh1) (GenBank: AF044293), intron 4 of the soya beta-conglycinin alpha subunit (GenBank: AB051865); one of the introns of the pea rbcS-3A gene for the ribulose-1,5-bisphosphate carboxylase (RBC) small subunit (GenBank: X04333). Other embodiments of suitable introns are discussed in (McCullough and Schuler, 1997; Smith et al., 2000).

In various embodiments, a loop may be at the end of at least 3, at least 4, at least 5, at least 6, at least 7, at least 8, at least 9, at least 10 consecutive basepairs, which may be canonical basepairs or may include one or more non-canonical basepairs. In other embodiments, less preferred, particularly for vertebrate animal cells, a loop may be at the end of at least 20, 30, 50, 100, 200, 500 or more consecutive basepairs.

In another embodiment, the RNA molecule comprises two or more sense ribonucleotide sequences, and an antisense ribonucleotide sequences fully based paired thereto, which are each identical in sequence to a region of a target RNA molecule. For example, the RNA molecule can comprise 2, 3, 4, 5, 6, 7, 8, 9, 10, 11, 12 or more sense ribonucleotide sequences, and antisense ribonucleotide sequences fully based paired thereto, which sense ribonucleotide sequences are each independently identical in sequence to a region of a target RNA molecule. In this embodiment, any one or more or all of the sequences can be separated by a linking ribonucleotide sequence(s). In this embodiment, any one or more or all of the sequences can be separated by a loop.

In an embodiment, the two or more sense ribonucleotide sequences are identical in sequence to different regions of the same target RNA molecule. For example, the sequences can be identical to at least 2, at least 3, at least 4, at least 5, at least 6 regions of the same target molecule. In another embodiment, the two or more sense ribonucleotide sequences are identical in sequence. In an embodiment, the two or more sense ribonucleotide sequences are identical in sequence to the same region of the same target RNA molecule. In another embodiment, the two or more sense ribonucleotide sequences are identical in sequence to different target RNA molecules. For embodiment, the sequences can be identical to at least 2, at least 3, at least 4, at least 5, at least 6 regions of different target molecules.

In another embodiment, the two or more sense ribonucleotide sequences have no intervening loop (spacer) sequences.

In an embodiment, the RNA molecule has a single strand of ribonucleotides having a 5′ end, at least one sense ribonucleotide sequence which is at least 21 nucleotides in length, an antisense ribonucleotide sequence which is fully basepaired with each sense ribonucleotide sequence over at least 21 contiguous nucleotides, at least two loop sequences and a 3′ end. In this embodiment, the ribonucleotide at the 5′ end and the ribonucleotide at the 3′ end are not directly covalently bonded but are rather positioned adjacent with each basepaired.

In another embodiment, consecutive basepairs of RNA components are interspaced by at least one gap. In an embodiment, the “gap” is provided by an unpaired ribonucleotide. In another embodiment, the “gap” is provided by un-ligated 5′ leader sequence and/or 3′ trailer sequence. In this embodiment, the gap can be referred to as an “unligated gap”. Mismatches and unligated gap(s) can be located at various position(s) of the RNA molecule. For embodiment, an unligated gap can immediately follow an antisense sequence. In another embodiment, an unligated gap can be close to a loop of the RNA molecule. In another embodiment, an unligated gap is positioned about equidistant between at least two loops.

In an embodiment, the RNA molecule is produced from a single strand of RNA. In an embodiment, the single strand is not circularly closed, for example, comprising an unligated gap. In another embodiment, the RNA molecule is a circularly closed molecule. Closed molecules can be produced by ligating an above referenced RNA molecule comprising an unligated gap, for example with an RNA ligase.

In another embodiment, the RNA molecule comprises a 5′- or 3′-, or both, extension sequence. For example, the RNA molecule can comprise a 5′ extension sequence which is covalently linked to the first 5′ ribonucleotide. In another embodiment, the RNA molecule comprises a 3′ extension sequence which is covalently linked to the second 3′ ribonucleotide.

In another embodiment, the RNA molecule comprises a 5′ extension sequence which is covalently linked to the first 5′ ribonucleotide and a 3′ extension sequence which is covalently linked to the second 3′ ribonucleotide.

In another embodiment, the RNA molecule comprises a 5′ extension sequence which is covalently linked to the second 5′ ribonucleotide. In another embodiment, the RNA molecule comprises a 3′ extension sequence which is covalently linked to the first 3′ ribonucleotide. In another embodiment, the RNA molecule comprises a 5′ extension sequence which is covalently linked to the second 5′ ribonucleotide and a 3′ extension sequence which is covalently linked to the first 3′ ribonucleotide.

In another embodiment, the RNA molecule can comprise one or more of the following:
5′ extension sequence which is covalently linked to the first 5′ ribonucleotide;
3′ extension sequence which is covalently linked to the second 3′ ribonucleotide;
5′ extension sequence which is covalently linked to the first 5′ ribonucleotide and a 3′ extension sequence which is covalently linked to the second 3′ ribonucleotide;
5′ extension sequence which is covalently linked to the second 5′ ribonucleotide;
3′ extension sequence which is covalently linked to the first 3′ ribonucleotide;
a 5′ extension sequence which is covalently linked to the second 5′ ribonucleotide and a 3′ extension sequence which is covalently linked to the first 3′ ribonucleotide.

### Non-Canonical Basepairing

In an embodiment, RNA molecules of the present invention comprise a sense ribonucleotide sequence and an antisense ribonucleotide sequence which are capable of hybridising to each other to form a double stranded (ds)RNA region with some non-canonical basepairing i.e. with a combination of canonical and non-canonical basepairing. In an embodiment, RNA molecules of the present invention comprise two or more sense ribonucleotide sequences which are each capable of hybridising to regions of one (contiguous) antisense ribonucleotide sequence to form a dsRNA region with some non-canonical basepairing. See for example, FIG. 1B. In an embodiment, RNA molecules of the present invention comprise two or more antisense sense ribonucleotide sequences which are each capable of hybridising to regions of one (contiguous) sense ribonucleotide sequence to form a dsRNA region with some non-canonical basepairing. See for example, FIG. 1A. In an embodiment, RNA molecules of the present invention comprise two or more antisense sense ribonucleotide sequences and two or more sense ribonucleotide sequences wherein each antisense ribonucleotide sequence is capable of hybridising to an antisense ribonucleotide sequence to form two or more dsRNA regions, one or both comprising some non-canonical basepairing.

In the following embodiments, the full length of the dsRNA region (i.e. the whole dsRNA region) of the RNA molecule of the invention is considered as the context for the feature if there is only one (contiguous) dsRNA region, or for each of the dsRNA regions of the RNA molecule if there are two or more dsRNA regions in the RNA molecule. In an embodiment, at least 5% of the basepairs in a dsRNA region are non-canonical basepairs. In an embodiment, at least 6% of the basepairs in a dsRNA region are non-canonical basepairs. In an embodiment, at least 7% of the basepairs in a dsRNA region are non-canonical basepairs. In an embodiment, at least 8% of the basepairs in a dsRNA region are non-canonical basepairs. In an embodiment, at least 9% or 10% of the basepairs in a dsRNA region are non-canonical basepairs. In an embodiment, at least 11% or 12% of the basepairs in a dsRNA region are non-canonical basepairs. In an embodiment, at least 15% or about 15% of the basepairs in a dsRNA region are non-canonical basepairs. In an embodiment, at least 20% or about 20% of the basepairs in a dsRNA region are non-canonical basepairs. In an embodiment, at least 25% or about 25% of the basepairs in a dsRNA region are non-canonical basepairs. In an embodiment, at least 30% or about 30% of the basepairs in a dsRNA region are non-canonical basepairs. In each of these embodiments, it is preferred that a maximum of 40% of the basepairs in the dsRNA region are non-canonical basepairs, more preferably a maximum of 35% of the basepairs in the dsRNA region are non-canonical basepairs, still more preferably a maximum of 30% of the basepairs in the dsRNA region are non-canonical basepairs. In an embodiment, less preferred, about 35% of the basepairs in a dsRNA region are non-canonical basepairs. In an embodiment, even less preferred, about 40% of the basepairs in a dsRNA region are non-canonical basepairs. In each of the above embodiments, the dsRNA region may or may not comprise one or more non-basepaired ribonucleotides, in either the sense sequence or the antisense sequence, or both.

In an embodiment, between 10% and 40% of the basepairs in a dsRNA region of the RNA molecule of the invention are non-canonical basepairs. In an embodiment, between 10% and 35% of the basepairs in a dsRNA region are non-canonical basepairs. In an embodiment, between 10% and 30% of the basepairs in a dsRNA region are non-canonical basepairs. In an embodiment, between 10% and 25% of the basepairs in a dsRNA region are non-canonical basepairs. In an embodiment, between 10% and 20% of the basepairs in a dsRNA region are non-canonical basepairs. In an embodiment, between 10% and 15% of the basepairs in a dsRNA region are non-canonical basepairs. In an embodiment, between 15% and 30% of the basepairs in a dsRNA region are non-canonical basepairs. In an embodiment, between 15% and 25% of the basepairs in a dsRNA region are non-canonical basepairs. In an embodiment, between 15% and 20% of the basepairs in a dsRNA region are non-canonical basepairs. In an embodiment, between 5% and 30% of the basepairs in a dsRNA region are non-canonical basepairs. In an embodiment, between 5% and 25% of the basepairs in a dsRNA region are non-canonical basepairs. In an embodiment, between 5% and 20% of the basepairs in a dsRNA region are non-canonical basepairs. In an embodiment, between 5% and 15% of the basepairs in a dsRNA region are non-canonical basepairs. In an embodiment, between 5% and 10% of the basepairs in a dsRNA region are non-canonical basepairs. In each of the above embodiments, the dsRNA region may or may not comprise one or more non-basepaired ribonucleotides, in either the sense sequence or the antisense sequence, or both.

In an embodiment, the dsRNA region of the RNA molecule of the invention comprises 20 contiguous basepairs, wherein at least one basepair of the 20 contiguous basepairs is a non-canonical basepair. In an embodiment, the dsRNA region comprises 20 contiguous basepairs, wherein at least 2 basepairs of the 20 contiguous basepairs are non-canonical basepairs. In an embodiment, the dsRNA region comprises 20 contiguous basepairs, wherein at least 3 basepairs of the 20 contiguous basepairs are non-canonical basepairs. In an embodiment, the dsRNA region comprises 20 contiguous basepairs, wherein at least 4 basepairs of the 20 contiguous basepairs are non-canonical basepairs. In an embodiment, the dsRNA region comprises 20 contiguous basepairs, wherein at least 5 basepairs of the 20 contiguous basepairs are non-canonical basepairs. In an embodiment, the dsRNA region comprises 20 contiguous basepairs, wherein at least 6 basepairs of the 20 contiguous basepairs are non-canonical basepairs. In an embodiment, the dsRNA region comprises 20 contiguous basepairs, wherein at least 7 basepairs of the 20 contiguous basepairs are non-canonical basepairs. In an embodiment, the dsRNA region comprises 20 contiguous basepairs, wherein at least 8 basepairs of the 20 contiguous basepairs are non-canonical basepairs. In an embodiment, the dsRNA region comprises 20 contiguous basepairs, wherein at least 9 basepairs of the 20 contiguous basepairs are non-canonical basepairs. In each of these embodiments, it is preferred that a maximum of 10 of the 20 contiguous basepairs in the dsRNA region are non-canonical basepairs, more preferably a maximum of 9 of the basepairs in the dsRNA region are non-canonical basepairs, still more preferably a maximum of 8 of the basepairs in the dsRNA region are non-canonical basepairs, even still more preferably a maximum of 7 of the basepairs in the dsRNA region are non-canonical basepairs, and most preferably a maximum of 6 of the basepairs in the dsRNA region are non-canonical basepairs. Preferably, in the above embodiments, the non-canonical basepairs comprise at least one G:U basepair, more preferably all of the non-canonical basepairs are G:U basepairs. Preferably, the features of the above embodiments apply to each and every one of the 20 contiguous basepairs that are present in the RNA molecule of the invention.

In an embodiment, the dsRNA region of the RNA molecule of the invention comprises 21 contiguous basepairs, wherein at least one basepair of the 21 contiguous basepairs is a non-canonical basepair. In an embodiment, the dsRNA region comprises 21 contiguous basepairs, wherein at least 2 basepairs of the 21 contiguous basepairs are non-canonical basepairs. In an embodiment, the dsRNA region comprises 21 contiguous basepairs, wherein at least 3 basepairs of the 21 contiguous basepairs are non-canonical basepairs. In an embodiment, the dsRNA region comprises 21 contiguous basepairs, wherein at least 4 basepairs of the 21 contiguous basepairs are non-canonical basepairs. In an embodiment, the dsRNA region comprises 21 contiguous basepairs, wherein at least 5 basepairs of the 21 contiguous basepairs are non-canonical basepairs. In an embodiment, the dsRNA region comprises 21 contiguous basepairs, wherein at least 6 basepairs of the 21 contiguous basepairs are non-canonical basepairs. In an embodiment, the dsRNA region comprises 21 contiguous basepairs, wherein at least 7 basepairs of the 21 contiguous basepairs are non-canonical basepairs. In an embodiment, the dsRNA region comprises 21 contiguous basepairs, wherein at least 8 basepairs of the 21 contiguous basepairs are non-canonical basepairs. In an embodiment, the dsRNA region comprises 21 contiguous basepairs, wherein at least 9 basepairs of the 21 contiguous basepairs are non-canonical basepairs. In each of these embodiments, it is preferred that a maximum of 10 of the 21 contiguous basepairs in the dsRNA region are non-canonical basepairs, more preferably a maximum of 9 of the basepairs in the dsRNA region are non-canonical basepairs, still more preferably a maximum of 8 of the basepairs in the dsRNA region are non-canonical basepairs, even still more preferably a maximum of 7 of the basepairs in the dsRNA region are non-canonical basepairs, and most preferably a maximum of 6 of the basepairs in the dsRNA region are non-canonical basepairs. Preferably, in the above embodiments, the non-canonical basepairs comprise at least one G:U basepair, more preferably all of the non-canonical basepairs are G:U basepairs. Preferably, the features of the above embodiments apply to each and every one of the 21 contiguous basepairs that are present in the RNA molecule of the invention.

In an embodiment, the dsRNA region of the RNA molecule of the invention comprises 22 contiguous basepairs, wherein at least one basepair of the 22 contiguous basepairs is a non-canonical basepair. In an embodiment, the dsRNA region comprises 22 contiguous basepairs, wherein at least 2 basepairs of the 22 contiguous basepairs are non-canonical basepairs. In an embodiment, the dsRNA region comprises 22 contiguous basepairs, wherein at least 3 basepairs of the 22 contiguous basepairs are non-canonical basepairs. In an embodiment, the dsRNA region comprises 22 contiguous basepairs, wherein at least 4 basepairs of the 22 contiguous basepairs are non-canonical basepairs. In an embodiment, the dsRNA region comprises 22 contiguous basepairs, wherein at least 5 basepairs of the 22 contiguous basepairs are non-canonical basepairs. In an embodiment, the dsRNA region comprises 22 contiguous basepairs, wherein at least 6 basepairs of the 22 contiguous basepairs are non-canonical basepairs. In an embodiment, the dsRNA region comprises 22 contiguous basepairs, wherein at least 7 basepairs of the 22 contiguous basepairs are non-canonical basepairs. In an embodiment, the dsRNA region comprises 22 contiguous basepairs, wherein at least 8 basepairs of the 22 contiguous basepairs are non-canonical basepairs. In an embodiment, the dsRNA region comprises 22 contiguous basepairs, wherein at least 9 basepairs of the 22 contiguous basepairs are non-canonical basepairs. In each of these embodiments, it is preferred that a maximum of 10 of the 22 contiguous basepairs in the dsRNA region are non-canonical basepairs, more preferably a maximum of 9 of the basepairs in the dsRNA region are non-canonical basepairs, still more preferably a maximum of 8 of the basepairs in the dsRNA region are non-canonical basepairs, even still more preferably a maximum of 7 of the basepairs in the dsRNA region are non-canonical basepairs, and most preferably a maximum of 6 of the basepairs in the dsRNA region are non-canonical basepairs. Preferably, in the above embodiments, the non-canonical basepairs comprise at least one G:U basepair, more preferably all of the non-canonical basepairs are G:U basepairs. Preferably, the features of the above embodiments apply to each and every one of the 22 contiguous basepairs that are present in the RNA molecule of the invention.

In an embodiment, the dsRNA region of the RNA molecule of the invention comprises 23 contiguous basepairs, wherein at least one basepair of the 23 contiguous basepairs is a non-canonical basepair. In an embodiment, the dsRNA region comprises 23 contiguous basepairs, wherein at least 2 basepairs of the 23 contiguous basepairs are non-canonical basepairs. In an embodiment, the dsRNA region comprises 23 contiguous basepairs, wherein at least 3 basepairs of the 23 contiguous basepairs are non-canonical basepairs. In an embodiment, the dsRNA region comprises 23 contiguous basepairs, wherein at least 4 basepairs of the 23 contiguous basepairs are non-canonical basepairs. In an embodiment, the dsRNA region comprises 23 contiguous basepairs, wherein at least 5 basepairs of the 23 contiguous basepairs are non-canonical basepairs. In an embodiment, the dsRNA region comprises 23 contiguous basepairs, wherein at least 6 basepairs of the 23 contiguous basepairs are non-canonical basepairs. In an embodiment, the dsRNA region comprises 23 contiguous basepairs, wherein at least 7 basepairs of the 23 contiguous basepairs are non-canonical basepairs. In an embodiment, the dsRNA region comprises 23 contiguous basepairs, wherein at least 8 basepairs of the 23 contiguous basepairs are non-canonical basepairs. In an embodiment, the dsRNA region comprises 23 contiguous basepairs, wherein at least 9 basepairs of the 23 contiguous basepairs are non-canonical basepairs. In each of these embodiments, it is preferred that a maximum of 10 of the 23 contiguous basepairs in the dsRNA region are non-canonical basepairs, more preferably a maximum of 9 of the basepairs in the dsRNA region are non-canonical basepairs, still more preferably a maximum of 8 of the basepairs in the dsRNA region are non-canonical basepairs, even still more preferably a maximum of 7 of the basepairs in the dsRNA region are non-canonical basepairs, and most preferably a maximum of 6 of the basepairs in the dsRNA region are non-canonical basepairs. Preferably, in the above embodiments, the non-canonical basepairs comprise at least one G:U basepair, more preferably all of the non-canonical basepairs are G:U basepairs. Preferably, the features of the above embodiments apply to each and every one of the 23 contiguous basepairs that are present in the RNA molecule of the invention.

In an embodiment, the dsRNA region of the RNA molecule of the invention comprises 24 contiguous basepairs, wherein at least one basepair of the 24 contiguous basepairs is a non-canonical basepair. In an embodiment, the dsRNA region comprises 24 contiguous basepairs, wherein at least 2 basepairs of the 24 contiguous basepairs are non-canonical basepairs. In an embodiment, the dsRNA region comprises 24 contiguous basepairs, wherein at least 3 basepairs of the 24 contiguous basepairs are non-canonical basepairs. In an embodiment, the dsRNA region comprises 24 contiguous basepairs, wherein at least 4 basepairs of the 24 contiguous basepairs are non-canonical basepairs. In an embodiment, the dsRNA region comprises 24 contiguous basepairs, wherein at least 5 basepairs of the 24 contiguous basepairs are non-canonical basepairs. In an embodiment, the dsRNA region comprises 24 contiguous basepairs, wherein at least 6 basepairs of the 24 contiguous basepairs are non-canonical basepairs. In an embodiment, the dsRNA region comprises 24 contiguous basepairs, wherein at least 7 basepairs of the 24 contiguous basepairs are non-canonical basepairs. In an embodiment, the dsRNA region comprises 24 contiguous basepairs, wherein at least 8 basepairs of the 24 contiguous basepairs are non-canonical basepairs. In an embodiment, the dsRNA region comprises 24 contiguous basepairs, wherein at least 9 basepairs of the 24 contiguous basepairs are non-canonical basepairs. In each of these embodiments, it is preferred that a maximum of 10 of the 24 contiguous basepairs in the dsRNA region are non-canonical basepairs, more preferably a maximum of 9 of the basepairs in the dsRNA region are non-canonical basepairs, still more preferably a maximum of 8 of the basepairs in the dsRNA region are non-canonical basepairs, even still more preferably a maximum of 7 of the basepairs in the dsRNA region are non-canonical basepairs, and most preferably a maximum of 6 of the basepairs in the dsRNA region are non-canonical basepairs. Preferably, in the above embodiments, the non-canonical basepairs comprise at least one G:U basepair, more preferably all of the non-canonical basepairs are G:U basepairs. Preferably, the features of the above embodiments apply to each and every one of the 24 contiguous basepairs that are present in the RNA molecule of the invention.

In the following embodiments, the full length of the dsRNA region (i.e. the whole dsRNA region) of the RNA molecule of the invention is considered as the context for the feature if there is only one (contiguous) dsRNA region, or for each of the dsRNA regions of the RNA molecule if there are two or more dsRNA regions in the RNA molecule. In an embodiment, the dsRNA region does not comprise 20 contiguous canonical basepairs i.e. every subregion of 20 contiguous basepairs includes at least one non-canonical basepair, preferably at least one G:U basepair. In an embodiment, the dsRNA region does not comprise 19 contiguous canonical basepairs. In an embodiment, the dsRNA region does not comprise 18 contiguous canonical basepairs. In an embodiment, the dsRNA region does not comprise 17 contiguous canonical basepairs. In an embodiment, the dsRNA region does not comprise 16 contiguous canonical basepairs. In an embodiment, the dsRNA region does not comprise 15 contiguous canonical basepairs. In an embodiment, the dsRNA region does not comprise 14 contiguous canonical basepairs. In an embodiment, the dsRNA region does not comprise 13 contiguous canonical basepairs. In an embodiment, the dsRNA region does not comprise 12 contiguous canonical basepairs. In an embodiment, the dsRNA region does not comprise 11 contiguous canonical basepairs. In an embodiment, the dsRNA region does not comprise 10 contiguous canonical basepairs. In an embodiment, the dsRNA region does not comprise 9 contiguous canonical basepairs. In an embodiment, the dsRNA region does not comprise 8 contiguous canonical basepairs. In an embodiment, the dsRNA region does not comprise 7 contiguous canonical basepairs. In the above embodiments, it is preferred that the longest subregion of contiguous canonical basepairing in the dsRNA region of the RNA molecule, or each and every dsRNA region in the RNA molecule, is 5, 6 or 7 contiguous canonical basepairs i.e. towards the shorter lengths mentioned. Each of the features of the above embodiments is preferably combined in the RNA molecule with the following features. In an embodiment, the dsRNA region comprises between 10 and 19 or 20 contiguous basepairs. In a preferred embodiment, the dsRNA region comprises between 12 and 19 or 20 contiguous basepairs. In an embodiment, the dsRNA region comprises between 14 and 19 or 20 contiguous basepairs. In these embodiments, the dsRNA region comprises 15 contiguous basepairs. In an embodiment, the dsRNA region comprises 16, 17, 18 or 19 contiguous basepairs. In an embodiment, the dsRNA region comprises 20 contiguous basepairs. Preferably, in the above embodiments, the contiguous basepairs comprise at least one non-canonical basepair which comprises at least one G:U basepair, more preferably all of the non-canonical basepairs in the region of contiguous basepairs are G:U basepairs.

In an embodiment, the dsRNA region comprises a subregion of 4 canonical basepairs flanked by non-canonical basepairs, i.e. at least one, preferably one or two (not more than 2), non-canonical basepairs adjacent to each end of the 4 canonical basepairs. In an embodiment, the dsRNA region comprises 2 subregions each of 4 canonical basepairs flanked by non-canonical basepairs. In an embodiment, the dsRNA region comprises 3 subregions each of 4 canonical basepairs flanked by non-canonical basepairs. In an embodiment, the dsRNA region comprises 4 or 5 subregions each of 4 canonical basepairs flanked by non-canonical basepairs. In an embodiment, the dsRNA region comprises 6 or 7 subregions each of 4 canonical basepairs flanked by non-canonical basepairs. In an embodiment, the dsRNA region comprises 8 to 10 subregions each of 4 canonical basepairs flanked by non-canonical basepairs. In an embodiment, the dsRNA region comprises 11 to 15 subregions each of 4 canonical basepairs flanked by non-canonical basepairs. In an embodiment, the dsRNA region comprises between 2 and 50 subregions each of 4 canonical basepairs flanked by non-canonical basepairs. In an embodiment, the dsRNA region comprises between 2 and 40 subregions each of 4 canonical basepairs flanked by non-canonical basepairs. In an embodiment, the dsRNA region comprises between 2 and 30 subregions each of 4 canonical basepairs flanked by non-canonical basepairs. In an embodiment, the dsRNA region comprises between 2 and 20 subregions each of 4 canonical basepairs flanked by non-canonical basepairs. Preferably, in the above embodiments, the non-canonical basepairs comprise at least one G:U basepair, more preferably all of the non-canonical basepairs flanking the contiguous canonical basepairs in the subregions are G:U basepairs. In variations of the above embodiments, one or both of the flanking non-canonical basepairs are replaced with a non-basepaired ribonucleotide in the sense sequence, the antisense sequence or in both sequences, for some or all of the subregions. It is readily understood that, in the above embodiments, the maximum number of subregions is determined by the length of the dsRNA region in the RNA molecule.

In an embodiment, the dsRNA region comprises a subregion of 5 canonical basepairs flanked by non-canonical basepairs. In an embodiment, the dsRNA region comprises 2 subregions each of 5 canonical basepairs flanked by non-canonical basepairs. In an embodiment, the dsRNA region comprises 3 subregions each of 5 canonical basepairs flanked by non-canonical basepairs. In an embodiment, the dsRNA region comprises 4 or 5 subregions each of 5 canonical basepairs flanked by non-canonical basepairs. In an embodiment, the dsRNA region comprises 6 or 7 subregions each of 5 canonical basepairs flanked by non-canonical basepairs. In an embodiment, the dsRNA region comprises 8 to 10 subregions each of 5 canonical basepairs flanked by non-canonical basepairs. In an embodiment, the dsRNA region comprises 11 to 15 subregions each of 5 canonical basepairs flanked by non-canonical basepairs. In an embodiment, the dsRNA region comprises between 2 and 50 subregions each of 5 canonical basepairs flanked by non-canonical basepairs. In an embodiment, the dsRNA region comprises between 2 and 50 subregions each of 5 canonical basepairs flanked by non-canonical basepairs. In an embodiment, the dsRNA region comprises between 2 and 30 subregions each of 5 canonical basepairs flanked by non-canonical basepairs. In an embodiment, the dsRNA region comprises between 2 and 20 subregions each of 5 canonical basepairs flanked by non-canonical basepairs. Preferably, in the above embodiments, the non-canonical basepairs comprise at least one G:U basepair, more preferably all of the non-canonical basepairs flanking the contiguous canonical basepairs in the subregions are G:U basepairs. In variations of the above embodiments, one or both of the flanking non-canonical basepairs are replaced with a non-basepaired ribonucleotide in the sense sequence, the antisense sequence or in both sequences, for some or all of the subregions. It is readily understood that, in the above embodiments, the maximum number of subregions is determined by the length of the dsRNA region in the RNA molecule.

In an embodiment, the dsRNA region comprises a subregion of 6 canonical basepairs flanked by non-canonical basepairs. In an embodiment, the dsRNA region comprises 2 subregions each of 6 canonical basepairs flanked by non-canonical basepairs. In an embodiment, the dsRNA region comprises 3 subregions each of 6 canonical basepairs flanked by non-canonical basepairs. In an embodiment, the dsRNA region comprises 4 or 5 subregions each of 6 canonical basepairs flanked by non-canonical basepairs. In an embodiment, the dsRNA region comprises 6 or 7 subregions each of 6 canonical basepairs flanked by non-canonical basepairs. In an embodiment, the dsRNA region comprises 8 to 10 subregions each of 6 canonical basepairs flanked by non-canonical basepairs. In an embodiment, the dsRNA region comprises 11 to 16 subregions each of 6 canonical basepairs flanked by non-canonical basepairs. In an embodiment, the dsRNA region comprises between 2 and 60 subregions each of 6 canonical basepairs flanked by non-canonical basepairs. In an embodiment, the dsRNA region comprises between 2 and 60 subregions each of 6 canonical basepairs flanked by non-canonical basepairs. In an embodiment, the dsRNA region comprises between 2 and 30 subregions each of 6 canonical basepairs flanked by non-canonical basepairs. In an embodiment, the dsRNA region comprises between 2 and 20 subregions each of 6 canonical basepairs flanked by non-canonical basepairs. Preferably, in the above embodiments, the non-canonical basepairs comprise at least one G:U basepair, more preferably all of the non-canonical basepairs flanking the contiguous canonical basepairs in the subregions are G:U basepairs. In variations of the above embodiments, one or both of the flanking non-canonical basepairs are replaced with a non-basepaired ribonucleotide in the sense sequence, the antisense sequence or in both sequences, for some or all of the subregions. It is readily understood that, in the above embodiments, the maximum number of subregions is determined by the length of the dsRNA region in the RNA molecule.

In an embodiment, the dsRNA region comprises a subregion of 10 contiguous basepairs wherein 2-4 of the basepairs are non-canonical basepairs. In an embodiment, the dsRNA region comprises 2 subregions each of 10 contiguous basepairs wherein 2-4 of the 10 contiguous basepairs are non-canonical basepairs. In an embodiment, the dsRNA region comprises 3 subregions each of 10 contiguous basepairs wherein 2-4 of the 10 contiguous basepairs are non-canonical basepairs. In an embodiment, the dsRNA region comprises 4 subregions each of 10 contiguous basepairs wherein 2-4 of the 10 contiguous basepairs are non-canonical basepairs. In an embodiment, the dsRNA region comprises 5 subregions each of 10 contiguous basepairs wherein 2-4 of the 10 contiguous basepairs are non-canonical basepairs. In an embodiment, the dsRNA region comprises 10 subregions each of 10 contiguous basepairs wherein 2-4 of the 10 contiguous basepairs are non-canonical basepairs. In an embodiment, the dsRNA region comprises 4 subregions each of 15 contiguous basepairs wherein 2-6 of the 15 contiguous basepairs are non-canonical basepairs. In an embodiment, the dsRNA region comprises between 2 and 50 subregions each of 10 contiguous basepairs wherein 2-4 of the 10 contiguous basepairs are non-canonical basepairs. In an embodiment, the dsRNA region comprises between 2 and 40 subregions each of 10 contiguous basepairs wherein 2-4 of the 10 contiguous basepairs are non-canonical basepairs. In an embodiment, the dsRNA region comprises between 2 and 30 subregions each of 10 contiguous basepairs wherein 2-4 of the 10 contiguous basepairs are non-canonical basepairs. In an embodiment, the dsRNA region comprises between 2 and 20 subregions each of 10 contiguous basepairs wherein 2-4 of the 10 contiguous basepairs are non-canonical basepairs. Preferably, in the above embodiments, the non-canonical basepairs comprise at least one G:U basepair, more preferably all of the non-canonical basepairs in the subregions are G:U basepairs. In variations of the above embodiments, one or more of the 2-4 or 2-6 non-canonical basepairs are replaced with a non-basepaired ribonucleotide in the sense sequence, the antisense sequence or in both sequences, for some or all of the subregions. It is readily understood that, in the above embodiments, the maximum number of subregions is determined by the length of the dsRNA region in the RNA molecule.

In an embodiment, the ratio of canonical to non-canonical basepairs in the dsRNA region is between 2.5:1 and 3.5:1, for example about 3:1. In an embodiment, the ratio of canonical to non-canonical basepairs in the dsRNA region is between 3.5:1 and 4.5:1, for example about 4:1. In an embodiment, the ratio of canonical to non-canonical basepairs in the dsRNA region is between 4.5:1 and 5.5:1, for example about 5:1. In an embodiment, the ratio of canonical to non-canonical basepairs in the dsRNA region is between 5.5:1 and 6.5:1, for example about 6:1. Different dsRNA regions in the RNA molecule may have different ratios.

In the above embodiments, the non-canonical basepairs in the dsRNA region(s) of the RNA molecule are preferably all G:U basepairs. In an embodiment, at least 99% of the non-canonical basepairs are G:U basepairs. In an embodiment, at least 98% of the non-canonical basepairs are G:U basepairs. In an embodiment, at least 97% of the non-canonical basepairs are G:U basepairs. In an embodiment, at least 95% of the non-canonical basepairs are G:U basepairs. In an embodiment, at least 90% of the non-canonical basepairs are G:U basepairs. In an embodiment, between 90 and 95% of the non-canonical basepairs are G:U basepairs. For example, if there are 10 non-canonical basepairs, at least 9 (90%) are G:U basepairs.

The dsRNA region comprising non-canonical basepairing(s) comprises an antisense sequence of 20 contiguous nucleotides which acts as an antisense regulatory element. In an embodiment, the antisense regulatory element is at least 80%, preferably at least 90%, more preferably at least 95% or most preferably 100% complementary to a target RNA molecule in a eukaryotic cell. In an embodiment, a dsRNA region comprises 2, 3, 4, or 5 antisense regulatory elements which either are complementary to the same target RNA molecule (i.e. to different regions of the same target RNA molecule) or are complementary to different target RNA molecules.

In an embodiment, one or more ribonucleotides of the sense ribonucleotide sequence or one or more ribonucleotides of the antisense ribonucleotide sequence, or both, are not basepaired in the dsRNA region when the sense and antisense sequences hybridize. In this embodiment, the dsRNA region does not include any loop sequence which covalently joins the sense and antisense sequences. One or more ribonucleotides of a dsRNA region or subregion may not be basepaired. Accordingly, in this embodiment, the sense strand of the dsRNA region does not fully basepair with its corresponding antisense strand.

Moreover, in an embodiment and optionally in combination with any of the features of the above embodiments, the total number of ribonucleotides in the sense sequence(s) and the total number of ribonucleotides in the antisense sequence(s) may not be identical, although preferably they are identical. In an embodiment, the total number of ribonucleotides in the sense ribonucleotide sequence(s) of the dsRNA region is between 90% and 110% of the total number of ribonucleotides in the antisense ribonucleotide sequence(s). In an embodiment, the total number of ribonucleotides in the sense ribonucleotide sequence(s) is between 95% and 105% of the total number of ribonucleotides in the antisense ribonucleotide sequence(s). In an embodiment, chimeric RNA molecules of the present disclosure can comprise one or more structural elements such as internal or terminal bulges or loops. Various embodiments of bulges and loops are discussed above. In an embodiment, dsRNA regions are separated by a structural element such as a bulge or loop. In an embodiment, dsRNA regions are separated by a intervening (spacer) sequence. Some of the ribonucleotides of the spacer sequence may be basepaired to other ribonucleotides in the RNA molecule, for example to other ribonucleotides within the spacer sequence, or they may not be basepaired in the RNA molecule, or some of each. In an embodiment, dsRNA regions are linked to a terminal loop. In an embodiment, dsRNA regions are flanked by terminal loops.

In an embodiment, where the dsRNA region of the RNA molecule of the invention has at least 3 non-canonical basepairs in any subregion of 5 contiguous basepairs, the non-canonical basepairs are not contiguous but are separated by one or more canonical basepairs i.e. the dsRNA region does not have 3 or more contiguous non-canonical basepairs. In an embodiment, the dsRNA region does not have 4 or more contiguous non-canonical basepairs. For example, in an embodiment, the dsRNA region comprises at least 3 non-canonical basepairs in a subregion of 10 basepairs, wherein each non-canonical basepair is separated by 4 canonical basepairs.

In an embodiment, an RNA molecule of the invention comprises more than one dsRNA region. For example, the RNA molecule comprises 2, 3, 4, 5, 6, 7, 8, 9, 10 or more dsRNA regions. In this example, one or more or all of the dsRNA regions can comprise above exemplified properties such as non-canonical basepairing and/or number of antisense regulatory elements.

### Silencing Activity

RNA molecules of the present disclosure have antisense activity as they comprise a sense ribonucleotide sequence that is essentially complementary to a region of a target RNA molecule. For example, the ribonucleotide sequence is essentially complementary to a region of a target RNA molecule in a eukaryotic cell. In an example, the target RNA molecule can be in a bacterial cell, fungal cell, plant cell, insect cell or animal cell. Such components of the RNA molecules defined herein can be referred to as an “antisense regulatory element”. “Essentially complementary” means that the sense ribonucleotide sequence may have insertions, deletions and individual point mutations in comparison with the complement of the target RNA molecule in the eukaryotic cell. Preferably, the homology is at least 80%, preferably at least 90%, preferably at least 95%, most preferably 100%, between the sense ribonucleotide sequence with antisense activity and the target RNA molecule. For example, the sense ribonucleotide sequence can comprise about 15, about 16, about 17, about 18, about 19 or more contiguous nucleotides that are identical in sequence to a first region of a target RNA molecule in a eukaryotic cell. In another example, the sense ribonucleotide sequence can comprise about 20 contiguous nucleotides that are identical in sequence to a first region of a target RNA molecule in a eukaryotic cell.

“Antisense activity” is used in the context of the present disclosure to refer to an antisense regulatory element from an RNA molecule defined herein that modulates (increase or decrease) expression of a target RNA molecule.

In various examples, antisense regulatory elements according to the present disclosure can comprise a plurality of monomeric subunits linked together by linking groups. Examples include primers, probes, antisense compounds, antisense oligonucleotides, external guide sequence (EGS) oligonucleotides, alternate splicers, gapmers, siRNAs and microRNAs. As such, RNA molecules according to the present disclosure can comprise antisense regulatory elements with single-stranded, double-stranded, circular, branched or hairpin structures. In an example, the antisense sequence can contain structural elements such as internal or terminal bulges or loops.

In an example, RNA molecules of the present disclosure comprise chimeric oligomeric components such as chimeric oligonucleotides. For example, an RNA molecule can comprise differently modified nucleotides, mixed-backbone antisense oligonucleotides or a combination thereof. In an example, chimeric oligomeric compounds can comprise at least one region modified so as to confer increased resistance to nuclease degradation, increased cellular uptake, and/or increased binding affinity for the target RNA molecule.

Antisense regulatory elements can have a variety of lengths. Across various examples, the present disclosure provides antisense regulatory elements consisting of X-Y linked bases, where X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 (provided that X<Y). For example, in certain embodiments, the present disclosure provides antisense regulatory elements comprising: 8-9, 8-10, 8-11, 8-12, 8-13, 8-14, 8-15, 8-16, 8-17, 8-18, 8-19, 8-20, 8-21, 8-22, 8-23, 8-24, 8-25, 8-26, 8-27, 8-28, 8-29, 8-30, 9-10, 9-11, 9-12, 9-13, 9-14, 9-15, 9-16, 9-17, 9-18, 9-19, 9-20, 9-21, 9-22, 9-23, 9-24, 9-25, 9-26, 9-27, 9-28, 9-29, 9-30, 10-11, 10-12, 10-13, 10-14, 10-15, 10-16, 10-17, 10-18, 10-19, 10-20, 10-21, 10-22, 10-23, 10-24, 10-25, 10-26, 10-27, 10-28, 10-29, 10-30, 11-12, 11-13, 11-14, 11-15, 11-16, 11-17, 11-18, 11-19, 11-20, 11-21, 11-22, 11-23, 11-24, 11-25, 11-26, 11-27, 11-28, 11-29, 11-30, 12-13, 12-14, 12-15, 12-16, 12-17, 12-18, 12-19, 12-20, 12-21, 12-22, 12-23, 12-24, 12-25, 12-26, 12-27, 12-28, 12-29, 12-30, 13-14, 13-15, 13-16, 13-17, 13-18, 13-19, 13-20, 13-21, 13-22, 13-23, 13-24, 13-25, 13-26, 13-27, 13-28, 13-29, 13-30, 14-15, 14-16, 14-17, 14-18, 14-19, 14-20, 14-21, 14-22, 14-23, 14-24, 14-25, 14-26, 14-27, 14-28, 14-29, 14-30, 15-16, 15-17, 15-18, 15-19, 15-20, 15-21, 15-22, 15-23, 15-24, 15-25, 15-26, 15-27, 15-28, 15-29, 15-30, 16-17, 16-18, 16-19, 16-20, 16-21, 16-22, 16-23, 16-24, 16-25, 16-26, 16-27, 16-28, 16-29, 16-30, 17-18, 17-19, 17-20, 17-21, 17-22, 17-23, 17-24, 17-25, 17-26, 17-27, 17-28, 17-29, 17-30, 18-19, 18-20, 18-21, 18-22, 18-23, 18-24, 18-25, 18-26, 18-27, 18-28, 18-29, 18-30, 19-20, 19-21, 19-22, 19-23, 19-24, 19-25, 19-26, 19-29, 19-28, 19-29, 19-30, 20-21, 20-22, 20-23, 20-24, 20-25, 20-26, 20-27, 20-28, 20-29, 20-30, 21-22, 21-23, 21-24, 21-25, 21-26, 21-27, 21-28, 21-29, 21-30, 22-23, 22-24, 22-25, 22-26, 22-27, 22-28, 22-29, 22-30, 23-24, 23-25, 23-26, 23-27, 23-28, 23-29, 23-30, 24-25, 24-26, 24-27, 24-28, 24-29, 24-30, 25-26, 25-27, 25-28, 25-29, 25-30, 26-27, 26-28, 26-29, 26-30, 27-28, 27-29, 27-30, 28-29, 28-30, or 29-30 linked bases.

RNA molecules according to the present disclosure can comprise multiple antisense regulatory elements. For example, RNA molecules can comprise at least 2, at least 3, at least 4, at least 5, at least 6, at least 7, at least 8, at least 9, at least 10 antisense regulatory elements. In an example, the antisense regulatory elements are the same. In this example, the RNA molecule can comprise at least 2, at least 3, at least 4, at least 5, at least 6, at least 7, at least 8, at least 9, at least 10 copies of an antisense regulatory element. In another example, RNA molecules according to the present disclosure can comprise different antisense regulatory elements. For example, antisense regulatory elements may be provided to target multiple genes in a pathway such as lipid biosynthesis. In this example, the RNA molecule can comprise at least 2, at least 3, at least 4, at least 5, at least 6, at least 7, at least 8, at least 9, at least 10 different antisense regulatory elements.

Antisense regulatory elements according to the present disclosure can modulate (increase or decrease) expression or amount of various target RNA molecules. In an example, the target RNA molecule is a fatty acid biosynthesis gene. Examples of such genes include genes encoding acetyl transacylases, acyl transport proteins (“acyl carrier protein”), desaturases such as stearyl desaturases or microsomal D12-desaturases, in particular Fad2-1 genes, malonyl transacylase, -ketoacyl-ACP synthetases, 3-keto-ACP reductases, enoyl-ACP hydrases, thioesterases such as acyl-ACP thioesterases, enoyl-ACP reductases. In an example, the target RNA molecule is FAD2 gene (for example those described by Genbank Acc. No.: AF124360 (Brassica carinata), AF042841 (Brassica rapa), L26296 (Arabidopsis thaliana), A65102 (Corylus avellana)). For example, the target RNA molecule can be FAD2.1 gene. In another example, the target RNA molecule can be FAD2.2 gene. In another example, the target RNA molecule can be FAD2.1 and FAD2.2 genes. Examples of other genes involved in modifying lipid composition that can be a target RNA molecule are known in the art (Shure M et al. (1983) Cell 35:225-233; Preiss et al. (1987) Tailoring Genes for Crop Improvement (Bruening et al., eds.), Plenum Press, S. 133-152; Gupta et al. (1988) Plant Mol. Biol. 10:215-224; Olive et al. (1989) Plant Mol Biol 12:525-538; Bhattacharyya et al. (1990) Cell 60:155-122; Dunwell J M (2000) J Exp Botany 51Spec No:487-96; Brar D S et al. (1996) Biotech Genet. Eng Rev 13:167-79; Kishore G M and Somerville C R (1993) Curr Opin Biotech 4(2):152-8; U.S. Pat. No. 5,530,192 and WO 94/18337).

In another example, the target RNA molecule is an arthropod gene such as an insect gene transcript. Examples of such genes include chitin synthase genes, such as CHS1 and/or CHS2 or other genes that control insect activity, behaviour, reproduction, growth and/or development. Various essential genes of a variety of pathogens are known to the those of skill in the art (for example nematode resistance genes are summarised in WO 93/10251, WO 94/17194).

In another example, the target RNA molecule is associated with a disease. For example, the target RNA molecule can be an oncogene or tumour suppressor gene transcript. Exemplary oncogenes include ABL1, BCL1, BCL2, BCL6, CBFA2, CBL, CSF1R, ERBA, ERBB, EBRB2, FGR, FOS, FYN, HRAS, JUN, LCK, LYN, MYB, MYC, NRAS, RET or SRC. Exemplary tumour suppressor genes include BRCA1 or BRCA2; adhesion molecules; cyclin kinases and their inhibitors.

In another example, the target RNA molecule is associated with delay of fruit maturation. Delayed fruit maturation can be achieved for example by reducing the gene expression of genes selected from the group consisting of polygalacturonases, pectin esterases, β-(1-4)glucanases (cellulases), β-galactanases (β-galactosidases), or genes of ethylene biosynthesis, such as 1-aminocyclopropane-1-carboxylate synthase, genes of carotenoid biosynthesis such as, for example, genes of prephytoene or phytoene biosynthesis, for example phytoene desaturases.

In another example, the target RNA molecule is associated with delay of senescence symptoms. Suitable target RNA molecules include cinnamoyl-CoA:NADPH reductases or cinnamoyl alcohol dehydrogenases. Further target RNA molecules are described (in WO 1995/07993).

In another example, the target RNA molecule is associated with modification of the fibre content in foodstuffs, preferably in seeds. For example, the RNA molecule can reduce expression of coffeic acid O-methyltransferase or of cinnamoyl alcohol dehydrogenase.

### Nucleic Acids Encoding RNA Molecules

One of skill in the art will appreciate from the foregoing description that the present disclosure also provides an isolated nucleic acid encoding RNA molecules disclosed herein and the component parts thereof. For example, a nucleic acid comprising a sequence set forth in any one or more of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9. The nucleic acid may be partially purified after expression in a host cell. The term “partially purified” is used to refer to an RNA molecule that has generally been separated from the lipids, nucleic acids, other peptides, and other contaminating molecules with which it is associated in a host cell. Preferably, the partially purified polynucleotide is at least 60% free, more preferably at least 75% free, and more preferably at least 90% free from other components with which it is associated.

In another example, a polynucleotide according to the present disclosure is a heterologous polynucleotide. The term “heterologous polynucleotide” is well understood in the art and refers to a polynucleotide which is not endogenous to a cell, or is a native polynucleotide in which the native sequence has been altered, or a native polypeptide whose expression is quantitatively altered as a result of a manipulation of the cell by recombinant DNA techniques.

In another example, a polynucleotide according to the present disclosure is a synthetic polynucleotide. For example, the polynucleotide may be produced using techniques that do not require pre-existing nucleic acid sequences such as DNA printing and oligonucleotide synthesis. In another example, the polynucleotide is produced from xeno nucleic acids.

In an example, a polynucleotide disclosed herein encodes an RNA precursor molecule comprising an intron in at least one loop sequence which is capable of being spliced out during transcription of the polynucleotide in a host cell. In another example, the loop sequence comprises two, three, four, five or more introns. The present disclosure also provides an expression construct such as a DNA construct comprising an isolated nucleic acid of the disclosure operably linked to a promoter. In an example, such isolated nucleic acids and/or expression constructs are provided in a cell or non-human organism. In an example isolated nucleic acids are stably integrated into the genome of the cell or non-human organism. Various examples of suitable expression constructs, promoters and cells comprising the same are discussed below.

Synthesis of RNA molecules according to the present disclosure can be achieved using various methods known in the art. The Examples section provides an example of in vitro synthesis. In this example, constructs comprising RNA molecules disclosed herein are restricted at the 3′ end, precipitated, purified and quantified. RNA synthesis can be achieved in bacterial culture following transformation of HT115 electro competent cells and induction of RNA synthesis using the T7, IPTG system.

### Recombinant Vectors

One embodiment of the present invention includes a recombinant vector, which comprises at least one RNA molecule defined herein and is capable of delivering the RNA molecule into a host cell. Recombinant vectors include expression vectors. Recombinant vectors contain heterologous polynucleotide sequences, that is, polynucleotide sequences that are not naturally found adjacent to an RNA molecule defined herein, that preferably, are derived from a different species. The vector can be either RNA or DNA, and typically is a viral vector, derived from a virus, or a plasmid.

Various viral vectors can be used to deliver and mediate expression of an RNA molecule according to the present disclosure. The choice of viral vector will generally depend on various parameters, such as the cell or tissue targeted for delivery, transduction efficiency of the vector and pathogenicity. In an example, the viral vector integrates into host cellular chromatin (e.g. lentiviruses). In another example, the viral vector persists in the cell nucleus predominantly as an extrachromosomal episome (e.g. adenoviruses). Examples of these types of viral vectors include oncoretroviruses, lentiviruses, adeno-associated virus, adenoviruses, herpes viruses and retroviruses.

Plasmid vectors typically include additional nucleic acid sequences that provide for easy selection, amplification, and transformation of the expression cassette in prokaryotic cells, e.g., pUC-derived vectors, pGEM-derived vectors or binary vectors containing one or more T-DNA regions. Additional nucleic acid sequences include origins of replication to provide for autonomous replication of the vector, selectable marker genes, preferably encoding antibiotic or herbicide resistance, unique multiple cloning sites providing for multiple sites to insert nucleic acid sequences or genes encoded in the nucleic acid construct, and sequences that enhance transformation of prokaryotic and eukaryotic (especially plant) cells.

“Operably linked” as used herein, refers to a functional relationship between two or more nucleic acid (e.g., DNA) segments. Typically, it refers to the functional relationship of a transcriptional regulatory element (promoter) to a transcribed sequence. For example, a promoter is operably linked to a coding sequence of an RNA molecule defined herein, if it stimulates or modulates the transcription of the coding sequence in an appropriate cell. Generally, promoter transcriptional regulatory elements that are operably linked to a transcribed sequence are physically contiguous to the transcribed sequence, i.e., they are cis-acting. However, some transcriptional regulatory elements such as enhancers need not be physically contiguous or located in close proximity to the coding sequences whose transcription they enhance.

When there are multiple promoters present, each promoter may independently be the same or different.

To facilitate identification of transformants, the recombinant vector desirably comprises a selectable or screenable marker gene. By “marker gene” is meant a gene that imparts a distinct phenotype to cells expressing the marker gene and thus, allows such transformed cells to be distinguished from cells that do not have the marker. A selectable marker gene confers a trait for which one can “select” based on resistance to a selective agent (e.g., a herbicide, antibiotic). A screenable marker gene (or reporter gene) confers a trait that one can identify through observation or testing, that is, by “screening” (e.g., β-glucuronidase, luciferase, GFP or other enzyme activity not present in untransformed cells). Exemplary selectable markers for selection of plant transformants include, but are not limited to, a hyg gene which encodes hygromycin B resistance; a neomycin phosphotransferase (nptII) gene conferring resistance to kanamycin, paromomycin; a glutathione-S-transferase gene from rat liver conferring resistance to glutathione derived herbicides as for example, described in EP 256223; a glutamine synthetase gene conferring, upon overexpression, resistance to glutamine synthetase inhibitors such as phosphinothricin as for example, described in WO 87/05327; an acetyltransferase gene from Streptomyces viridochromogenes conferring resistance to the selective agent phosphinothricin as for example, described in EP 275957; a gene encoding a 5-enolshikimate-3-phosphate synthase (EPSPS) conferring tolerance to N-phosphonomethylglycine as for example, described by Hinchee et al. (1988); a bar gene conferring resistance against bialaphos as for example, described in WO91/02071; anitrilase gene such as bxn fromKlebsiella ozaenae which confers resistance to bromoxynil (Stalker et al., 1988); a dihydrofolate reductase (DHFR) gene conferring resistance to methotrexate (Thillet et al., 1988); a mutant acetolactate synthase gene (ALS) which confers resistance to imidazolinone, sulfonylurea, or other ALS-inhibiting chemicals (EP 154,204); a mutated anthranilate synthase gene that confers resistance to 5-methyl tryptophan; or a dalapon dehalogenase gene that confers resistance to the herbicide.

Preferably, the recombinant vector is stably incorporated into the genome of the cell such as the plant cell. Accordingly, the recombinant vector may comprise appropriate elements which allow the vector to be incorporated into the genome, or into a chromosome of the cell.

### Expression Vector

As used herein, an “expression vector” is a DNA vector that is capable of transforming a host cell and of effecting expression of an RNA molecule defined herein. Expression vectors of the present invention contain regulatory sequences such as transcription control sequences, translation control sequences, origins of replication, and other regulatory sequences that are compatible with the host cell and that control the expression of RNA molecule according to the present disclosure. In particular, expression vectors of the present invention include transcription control sequences. Transcription control sequences are sequences which control the initiation, elongation, and termination of transcription. Particularly important transcription control sequences are those which control transcription initiation such as promoter, enhancer, operator and repressor sequences. The choice of the regulatory sequences used depends on the target organism such as a plant and/or target organ or tissue of interest. Such regulatory sequences may be obtained from any eukaryotic organism such as plants or plant viruses, or may be chemically synthesized.

Exemplary vectors suitable for stable transfection of plant cells or for the establishment of transgenic plants have been described in for example, Pouwels et al., Cloning Vectors: A Laboratory Manual, 1985, supp. 1987, Weissbach and Weissbach, Methods for Plant Molecular Biology, Academic Press, 1989, and Gelvin et al., Plant Molecular Biology Manual, Kluwer Academic Publishers, 1990. Typically, plant expression vectors include for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, a transcription termination site, and/or a polyadenylation signal.

Vectors of the invention can also be used to produce RNA molecules defined herein in a cell-free expression system, such systems are well known in the art.

In an example, a polynucleotide encoding an RNA molecule according to the present disclosure is operably linked to a promoter capable of directly expressing of the RNA molecule in a host cell. In an example, the promoter functions in vitro. In an example, the promoter is an RNA polymerase promoter. For example, the promoter can be an RNA polymerase III promoter. In another example, the promoter can be an RNA polymerase II promoter. However, the choice of promoter can depend on the target organism such as a plant, insect and/or target organ or tissue of interest. Exemplary mammalian promoters include CMV, EF1a, SV40, PGK1, Ubc, human beta actin, CAG, TRE, UAS, CaMKIIa, CAL1, 10, TEF1, GDS, ADH1, CaMV35S, Ubi, H1 and U6. Exemplary insect promoters include Ac5 and polyhedron. A number of constitutive promoters that are active in plant cells have also been described. Suitable promoters for constitutive expression in plants include, but are not limited to, the cauliflower mosaic virus (CaMV) 35S promoter, the Figwort mosaic virus (FMV) 35S, the light-inducible promoter from the small subunit (SSU) of the ribulose-1,5-bis-phosphate carboxylase, the rice cytosolic triosephosphate isomerase promoter, the adenine phosphoribosyltransferase promoter of Arabidopsis, the rice actin 1 gene promoter, the mannopine synthase and octopine synthase promoters, the Adh promoter, the sucrose synthase promoter, the R gene complex promoter, and the chlorophyll α/β binding protein gene promoter. These promoters have been used to create DNA vectors that have been expressed in plants, see for example, WO 84/02913. All of these promoters have been used to create various types of plant-expressible recombinant DNA vectors.

For the purpose of expression in source tissues of the plant such as the leaf, seed, root or stem, it is preferred that the promoters utilized in the present invention have relatively high expression in these specific tissues. For this purpose, one may choose from a number of promoters for genes with tissue- or cell-specific, or -enhanced expression. Examples of such promoters reported in the literature include, the chloroplast glutamine synthetase GS2 promoter from pea, the chloroplast fructose-1,6-biphosphatase promoter from wheat, the nuclear photosynthetic ST-LS1 promoter from potato, the serine/threonine kinase promoter and the glucoamylase (CHS) promoter from Arabidopsis thaliana. Also reported to be active in photosynthetically active tissues are the ribulose-1,5-bisphosphate carboxylase promoter from eastern larch (Larix laricina), the promoter for the Cab gene, Cab6, from pine, the promoter for the Cab-1 gene from wheat, the promoter for the Cab-1 gene from spinach, the promoter for the Cab 1R gene from rice, the pyruvate, orthophosphate dikinase (PPDK) promoter from Zea mays, the promoter for the tobacco Lhcb1*2 gene, the Arabidopsis thaliana Suc2 sucrose-H³⁰ symporter promoter, and the promoter for the thylakoid membrane protein genes from spinach (PsaD, PsaF, PsaE, PC, FNR, AtpC, AtpD, Cab, RbcS). Other promoters for the chlorophyll α/β-binding proteins may also be utilized in the present invention such as the promoters for LhcB gene and PsbP gene from white mustard (Sinapis alba).

A variety of plant gene promoters that are regulated in response to environmental, hormonal, chemical, and/or developmental signals, also can be used for expression of RNA-binding protein genes in plant cells, including promoters regulated by (1) heat, (2) light (e.g., pea RbcS-3A promoter, maize RbcS promoter), (3) hormones such as abscisic acid, (4) wounding (e.g., WunI), or (5) chemicals such as methyl jasmonate, salicylic acid, steroid hormones, alcohol, Safeners (WO 97/06269), or it may also be advantageous to employ (6) organ-specific promoters.

As used herein, the term “plant storage organ specific promoter” refers to a promoter that preferentially, when compared to other plant tissues, directs gene transcription in a storage organ of a plant. For the purpose of expression in sink tissues of the plant such as the tuber of the potato plant, the fruit of tomato, or the seed of soybean, canola, cotton, Zea mays, wheat, rice, and barley, it is preferred that the promoters utilized in the present invention have relatively high expression in these specific tissues. The promoter for β-conglycinin or other seed-specific promoters such as the napin, zein, linin and phaseolin promoters, can be used. Root specific promoters may also be used. An example of such a promoter is the promoter for the acid chitinase gene. Expression in root tissue could also be accomplished by utilizing the root specific subdomains of the CaMV 35S promoter that have been identified.

In a particularly preferred embodiment, the promoter directs expression in tissues and organs in which lipid biosynthesis take place. Such promoters may act in seed development at a suitable time for modifying lipid composition in seeds. Preferred promoters for seed-specific expression include: 1) promoters from genes encoding enzymes involved in lipid biosynthesis and accumulation in seeds such as desaturases and elongases, 2) promoters from genes encoding seed storage proteins, and 3) promoters from genes encoding enzymes involved in carbohydrate biosynthesis and accumulation in seeds. Seed specific promoters which are suitable are, the oilseed rape napin gene promoter (U.S. Pat. No. 5,608,152), the Viciafaba USP promoter (Baumlein et al., 1991), the Arabidopsis oleosin promoter (WO 98/45461), the Phaseolus vulgaris phaseolin promoter (U.S. Pat. No. 5,504,200), the Brassica Bce4 promoter (WO 91/13980), or the legumin B4 promoter (Baumlein et al., 1992), and promoters which lead to the seed-specific expression in monocots such as maize, barley, wheat, rye, rice and the like. Notable promoters which are suitable are the barley lpt2 or lpt1 gene promoter (WO 95/15389 and WO 95/23230), or the promoters described in WO 99/16890 (promoters from the barley hordein gene, the rice glutelin gene, the rice oryzin gene, the rice prolamin gene, the wheat gliadin gene, the wheat glutelin gene, the maize zein gene, the oat glutelin gene, the sorghum kasirin gene, the rye secalin gene). Other promoters include those described by Broun et al. (1998), Potenza et al. (2004), US 20070192902 and US 20030159173. In an embodiment, the seed specific promoter is preferentially expressed in defined parts of the seed such as the cotyledon(s) or the endosperm. Examples of cotyledon specific promoters include, but are not limited to, the FP1 promoter (Ellerstrom et al., 1996), the pea legumin promoter (Perrin et al., 2000), and the bean phytohemagglutnin promoter (Perrin et al., 2000). Examples of endosperm specific promoters include, but are not limited to, the maize zein-1 promoter (Chikwamba et al., 2003), the rice glutelin-1 promoter (Yang et al., 2003), the barley D-hordein promoter (Horvath et al., 2000) and wheat HMW glutenin promoters (Alvarez et al., 2000). In a further embodiment, the seed specific promoter is not expressed, or is only expressed at a low level, in the embryo and/or after the seed germinates.

In another embodiment, the plant storage organ specific promoter is a fruit specific promoter. Examples include, but are not limited to, the tomato polygalacturonase, E8 and Pds promoters, as well as the apple ACC oxidase promoter (for review, see Potenza et al., 2004). In a preferred embodiment, the promoter preferentially directs expression in the edible parts of the fruit, for example the pith of the fruit, relative to the skin of the fruit or the seeds within the fruit.

In an embodiment, the inducible promoter is the Aspergillus nidulans alc system. Examples of inducible expression systems which can be used instead of the Aspergillus nidulans alc system are described in a review by Padidam (2003) and Corrado and Karali (2009). In another embodiment, the inducible promoter is a safener inducible promoter such as, for example, the maize ln2-1 or ln2-2 promoter (Hershey and Stoner, 1991), the safener inducible promoter is the maize GST-27 promoter (Jepson et al., 1994), or the soybean GH2/4 promoter (Ulmasov et al., 1995).

In another embodiment, the inducible promoter is a senescence inducible promoter such as, for example, senescence-inducible promoter SAG (senescence associated gene) 12 and SAG 13 from Arabidopsis (Gan, 1995; Gan and Amasino, 1995) and LSC54 from Brassica napus (Buchanan-Wollaston, 1994). Such promoters show increased expression at about the onset of senescence of plant tissues, in particular the leaves.

For expression in vegetative tissue leaf-specific promoters, such as the ribulose biphosphate carboxylase (RBCS) promoters, can be used. For example, the tomato RBCS1, RBCS2 and RBCS3A genes are expressed in leaves and light grown seedlings (Meier et al., 1997). A ribulose bisphosphate carboxylase promoters expressed almost exclusively in mesophyll cells in leaf blades and leaf sheaths at high levels, described by Matsuoka et al. (1994), can be used. Another leaf-specific promoter is the light harvesting chlorophyll a/b binding protein gene promoter (see, Shiina et al., 1997). The Arabidopsis thaliana myb-related gene promoter (Atmyb5) described by Li et al. (1996), is leaf-specific. The Atmyb5 promoter is expressed in developing leaf trichomes, stipules, and epidermal cells on the margins of young rosette and cauline leaves, and in immature seeds. A leaf promoter identified in maize by Busk et al. (1997), can also be used.

In some instances, for example when LEC2 or BBM is recombinantly expressed, it may be desirable that the transgene is not expressed at high levels. An example of a promoter which can be used in such circumstances is a truncated napin A promoter which retains the seed-specific expression pattern but with a reduced expression level (Tan et al., 2011).

The 5′ non-translated leader sequence can be derived from the promoter selected to express the heterologous gene sequence of an RNA molecule of the present disclosure, or may be heterologous with respect to the coding region of the enzyme to be produced, and can be specifically modified if desired so as to increase translation of mRNA. For a review of optimizing expression of transgenes, see Koziel et al. (1996). The 5′ non-translated regions can also be obtained from plant viral RNAs (Tobacco mosaic virus, Tobacco etch virus, Maize dwarf mosaic virus, Alfalfa mosaic virus, among others) from suitable eukaryotic genes, plant genes (wheat and maize chlorophyll a/b binding protein gene leader), or from a synthetic gene sequence. The present invention is not limited to constructs wherein the non-translated region is derived from the 5′ non-translated sequence that accompanies the promoter sequence. The leader sequence could also be derived from an unrelated promoter or coding sequence. Leader sequences useful in context of the present invention comprise the maize Hsp70 leader (U.S. Pat. Nos. 5,362,865 and 5,859,347), and the TMV omega element.

The termination of transcription is accomplished by a 3′ non-translated DNA sequence operably linked in the expression vector to the RNA molecule of interest. The 3′ non-translated region of a recombinant DNA molecule contains a polyadenylation signal that functions in plants to cause the addition of adenylate nucleotides to the 3′ end of the RNA. The 3′ non-translated region can be obtained from various genes that are expressed in plant cells. The nopaline synthase 3′ untranslated region, the 3′ untranslated region from pea small subunit Rubisco gene, the 3′ untranslated region from soybean 7S seed storage protein gene are commonly used in this capacity. The 3′ transcribed, non-translated regions containing the polyadenylate signal of Agrobacterium tumor-inducing (Ti) plasmid genes are also suitable.

### Transfer Nucleic Acids

Transfer nucleic acids can be used to deliver an exogenous polynucleotide to a cell and comprise one, preferably two, border sequences and one or more RNA molecules of interest. The transfer nucleic acid may or may not encode a selectable marker. Preferably, the transfer nucleic acid forms part of a binary vector in a bacterium, where the binary vector further comprises elements which allow replication of the vector in the bacterium, selection, or maintenance of bacterial cells containing the binary vector. Upon transfer to a eukaryotic cell, the transfer nucleic acid component of the binary vector is capable of integration into the genome of the eukaryotic cell or, for transient expression experiments, merely of expression in the cell.

As used herein, the term “extrachromosomal transfer nucleic acid” refers to a nucleic acid molecule that is capable of being transferred from a bacterium such as Agrobacterium sp., to a eukaryotic cell such as a plant leaf cell. An extrachromosomal transfer nucleic acid is a genetic element that is well-known as an element capable of being transferred, with the subsequent integration of a nucleotide sequence contained within its borders into the genome of the recipient cell. In this respect, a transfer nucleic acid is flanked, typically, by two “border” sequences, although in some instances a single border at one end can be used and the second end of the transferred nucleic acid is generated randomly in the transfer process. An RNA molecule of interest is typically positioned between the left border-like sequence and the right border-like sequence of a transfer nucleic acid. The RNA molecule contained within the transfer nucleic acid may be operably linked to a variety of different promoter and terminator regulatory elements that facilitate its expression, that is, transcription and/or translation of the RNA molecule. Transfer DNAs (T-DNAs) from Agrobacterium sp. such as Agrobacterium tumefaciens or Agrobacterium rhizogenes, and man made variants/mutants thereof are probably the best characterized examples of transfer nucleic acids. Another example is P-DNA (“plant-DNA”) which comprises T-DNA border-like sequences from plants.

As used herein, “T-DNA” refers to a T-DNA of an Agrobacterium tumefaciens Ti plasmid or from an Agrobacterium rhizogenes Ri plasmid, or variants thereof which function for transfer of DNA into plant cells. The T-DNA may comprise an entire T-DNA including both right and left border sequences, but need only comprise the minimal sequences required in cis for transfer, that is, the right T-DNA border sequence. The T-DNAs of the invention have inserted into them, anywhere between the right and left border sequences (if present), the RNA molecule of interest. The sequences encoding factors required in trans for transfer of the T-DNA into a plant cell such as vir genes, may be inserted into the T-DNA, or may be present on the same replicon as the T-DNA, or preferably are in trans on a compatible replicon in the Agrobacterium host. Such “binary vector systems” are well known in the art. As used herein, “P-DNA” refers to a transfer nucleic acid isolated from a plant genome, or man made variants/mutants thereof, and comprises at each end, or at only one end, a T-DNA border-like sequence.

As used herein, a “border” sequence of a transfer nucleic acid can be isolated from a selected organism such as a plant or bacterium, or be a man made variant/mutant thereof. The border sequence promotes and facilitates the transfer of the RNA molecule to which it is linked and may facilitate its integration in the recipient cell genome. In an embodiment, a border-sequence is between 10-80 bp in length. Border sequences from T-DNA from Agrobacterium sp. are well known in the art and include those described in Lacroix et al. (2008).

Whilst traditionally only Agrobacterium sp. have been used to transfer genes to plants cells, there are now a large number of systems which have been identified/developed which act in a similar manner to Agrobacterium sp. Several non-Agrobacterium species have recently been genetically modified to be competent for gene transfer (Chung et al., 2006; Broothaerts et al., 2005). These include Rhizobium sp. NGR234, Sinorhizobium meliloti and Mezorhizobium loti.

Direct transfer of eukaryotic expression plasmids from bacteria to eukaryotic hosts was first achieved several decades ago by the fusion of mammalian cells and protoplasts of plasmid-carrying Escherichia coli (Schaffner, 1980). Since then, the number of bacteria capable of delivering genes into mammalian cells has steadily increased (Weiss, 2003), being discovered by four groups independently (Sizemore et al. 1995; Courvalin et al., 1995; Powell et al., 1996; Darji et al., 1997).

As used herein, the terms “transfection”, “transformation” and variations thereof are generally used interchangeably. “Transfected” or “transformed” cells may have been manipulated to introduce the RNA molecule(s) of interest, or may be progeny cells derived therefrom.

### Recombinant Cells

The invention also provides a recombinant cell, for example, a recombinant bacterial cell, fungal cell, plant cell, insect cell or animal cell, which is a host cell transformed with one or more RNA molecules or vectors defined herein, or combination thereof. Suitable cells of the invention include any cell that can be transformed with an RNA molecule or recombinant vector according to the present disclosure. In an example, the transformed host cell is dead.

The recombinant cell may be a cell in culture, a cell in vitro, or in an organism such as for example, a plant, or in an organ such as, for example, a seed or a leaf. Preferably, the cell is in a plant, more preferably in the seed of a plant. In one embodiment, the recombinant cell is a non-human cell. Accordingly, in an example, the present disclosure relates to a non-human organism comprising one or more or all of an RNA molecule disclosed herein.

In one example, the cells are insect cells. In one example, the insect cells are derived from Trichoplusia.

Another example of a suitable host cell is an electro competent HT115 cell.

Host cells into which the RNA molecules(s) are introduced can be either untransformed cells or cells that are already transformed with at least one nucleic acid. Such nucleic acids may be related to lipid synthesis, or unrelated. Host cells of the present invention either can be endogenously (i.e., naturally) capable of expressing RNA molecule(s) defined herein, in which case the recombinant cell derived therefrom has an enhanced capability of producing the RNA molecule(s), or can be capable of producing said RNA molecule(s) only after being transformed with at least one RNA molecule defined herein. In an example, the cell is a cell which is capable of being used for producing lipid. In an embodiment, a recombinant cell of the invention has an enhanced capacity to produce non-polar lipid such as TAG.

Host cells of the present disclosure can be any cell capable of expressing at least one RNA molecule described herein, and include bacterial, fungal (including yeast), parasite, arthropod, animal and plant cells. Examples of host cells include Salmonella, Escherichia, Bacillus, Listeria, Saccharomyces, Spodoptera, Mycobacteria, Trichoplusia, Agrobacterium, BHK (baby hamster kidney) cells, MDCK cells, CRFK cells, CV-1 cells, COS (e.g., COS-7) cells, and Vero cells. Further examples of host cells are E. coli, including E. coli K-12 derivatives; Salmonella typhi; Salmonella typhimurium, including attenuated strains; Spodoptera frugiperda; Trichoplusia ni; and non-tumorigenic mouse myoblast G8 cells (e.g., ATCC CRL 1246). Additional appropriate mammalian cell hosts include other kidney cell lines, other fibroblast cell lines (e.g., human, murine or chicken embryo fibroblast cell lines), myeloma cell lines, Chinese hamster ovary cells, mouse NIH/3T3 cells, LMTK cells and/or HeLa cells.

In a preferred embodiment, the plant cell is a seed cell, in particular, a cell in a cotyledon or endosperm of a seed. In one embodiment, the cell is an animal cell. The animal cell may be of any type of animal such as, for example, a non-human animal cell, a non-human vertebrate cell, a non-human mammalian cell, or cells of aquatic animals such as fish or crustacea, invertebrates, insects, etc. Examples of algal cells useful as host cells of the present invention include, for example, Chlamydomonas sp. (for example, Chlamydomonas reinhardtii), Dunaliella sp., Haematococcus sp., Chlorella sp., Thraustochytrium sp., Schizochytrium sp., and Volvox sp.

### Transgenic Plants

The invention also provides a plant comprising one or more exogenous RNA molecules defined herein, a cell of according to the present disclosure, a vector according to the present disclosure, or a combination thereof. The term “plant” when used as a noun refers to whole plants, whilst the term “part thereof” refers to plant organs (e.g., leaves, stems, roots, flowers, fruit), single cells (e.g., pollen), seed, seed parts such as an embryo, endosperm, scutellum or seed coat, plant tissue such as vascular tissue, plant cells and progeny of the same. As used herein, plant parts comprise plant cells.

As used herein, the terms “in a plant” and “in the plant” in the context of a modification to the plant means that the modification has occurred in at least one part of the plant, including where the modification has occurred throughout the plant, and does not exclude where the modification occurs in only one or more but not all parts of the plant. For example, a tissue-specific promoter is said to be expressed “in a plant”, even though it might be expressed only in certain parts of the plant. Analogously, “a transcription factor polypeptide that increases the expression of one or more glycolytic and/or fatty acid biosynthetic genes in the plant” means that the increased expression occurs in at least a part of the plant.

As used herein, the term “plant” is used in it broadest sense, including any organism in the Kingdom Plantae. It also includes red and brown algae as well as green algae. It includes, but is not limited to, any species of flowering plant, grass, crop or cereal (e.g., oilseed, maize, soybean), fodder or forage, fruit or vegetable plant, herb plant, woody plant or tree. It is not meant to limit a plant to any particular structure. It also refers to a unicellular plant (e.g., microalga). The term “part thereof” in reference to a plant refers to a plant cell and progeny of same, a plurality of plant cells, a structure that is present at any stage of a plant's development, or a plant tissue. Such structures include, but are not limited to, leaves, stems, flowers, fruits, nuts, roots, seed, seed coat, embryos. The term “plant tissue” includes differentiated and undifferentiated tissues of plants including those present in leaves, stems, flowers, fruits, nuts, roots, seed, for example, embryonic tissue, endosperm, dermal tissue (e.g., epidermis, periderm), vascular tissue (e.g., xylem, phloem), or ground tissue (comprising parenchyma, collenchyma, and/or sclerenchyma cells), as well as cells in culture (e.g., single cells, protoplasts, callus, embryos, etc.). Plant tissue may be in planta, in organ culture, tissue culture, or cell culture.

Different amounts of 18:3 and 16:3 fatty acids are found within the glycolipids of different plant species. This is used to distinguish between 18:3 plants whose fatty acids with 3 double bonds are generally always C₁₈ atoms long and the 16:3 plants that contain both C₁₆- and C₁₈-fatty acids. In 18:3 chloroplasts, enzymic activities catalyzing the conversion of phosphatidate to diacylglycerol and of diacyiglycerol to monogalactosyl diacylglycerol (MGD) are significantly less active than in 16:3 chloroplasts. In leaves of 18:3 plants, chloroplasts synthesize stearoyl-ACP2 in the stroma, introduce the first double bond into the saturated hydrocarbon chain, and then hydrolyze the thioester. Released oleate is exported across chloroplast envelopes into membranes of the eucaryotic part of the cell, probably the endoplasmic reticulum, where it is incorporated into PC. PC-linked oleoyl groups are desaturated in these membranes and subsequently move back into the chloroplast. The MGD-linked acyl groups are substrates for the introduction of the third double bond to yield MGD with two linolenoyl residues. This galactolipid is characteristic of 18:3 plants such as Asteraceae and Fabaceae, for example. In photosynthetically active cells of 16:3 plants which are represented, for example, by members of Apiaceae and Brassicaceae, two pathways operate in parallel to provide thylakoids with MGD. The cooperative ‘eucaryotic’ sequence is supplemented to various extents by a ‘procaryotic’ pathway. Its reactions are confined to the chloroplast and result in a typical arrangement of acyl groups as well as their complete desaturation once they are esterified to MGD. Procaryotic DAG backbones carry C16:0 and its desaturation products at C-2 from which position C18: fatty acids are excluded. The C-1 position is occupied by C18 fatty acids and to a small extent by C16 groups. The similarity in DAG backbones of lipids from blue-green algae with those synthesized by the chloroplast-confined pathway in 16:3 plants suggests a phylogenetic relation and justifies the term procaryotic.

As used herein, the term “vegetative tissue” or “vegetative plant part” is any plant tissue, organ or part other than organs for sexual reproduction of plants. The organs for sexual reproduction of plants are specifically seed bearing organs, flowers, pollen, fruits and seeds. Vegetative tissues and parts include at least plant leaves, stems (including bolts and tillers but excluding the heads), tubers and roots, but excludes flowers, pollen, seed including the seed coat, embryo and endosperm, fruit including mesocarp tissue, seed-bearing pods and seed-bearing heads. In one embodiment, the vegetative part of the plant is an aerial plant part. In another or further embodiment, the vegetative plant part is a green part such as a leaf or stem.

A “transgenic plant” or variations thereof refers to a plant that contains a transgene not found in a wild-type plant of the same species, variety or cultivar. Transgenic plants as defined in the context of the present invention include plants and their progeny which have been genetically modified using recombinant techniques to cause production of at least one polypeptide defined herein in the desired plant or part thereof. Transgenic plant parts has a corresponding meaning.

The terms “seed” and “grain” are used interchangeably herein. “Grain” refers to mature grain such as harvested grain or grain which is still on a plant but ready for harvesting, but can also refer to grain after imbibition or germination, according to the context. Mature grain commonly has a moisture content of less than about 18%. In a preferred embodiment, the moisture content of the grain is at a level which is generally regarded as safe for storage, preferably between 5% and 15%, between 6% and 8%, between 8% and 10%, or between 10% and 15%. “Developing seed” as used herein refers to a seed prior to maturity, typically found in the reproductive structures of the plant after fertilisation or anthesis, but can also refer to such seeds prior to maturity which are isolated from a plant. Mature seed commonly has a moisture content of less than about 12%.

As used herein, the term “plant storage organ” refers to a part of a plant specialized to store energy in the form of for example, proteins, carbohydrates, lipid. Examples of plant storage organs are seed, fruit, tuberous roots, and tubers. A preferred plant storage organ of the invention is seed.

As used herein, the term “phenotypically normal” refers to a genetically modified plant or part thereof, for example a transgenic plant, or a storage organ such as a seed, tuber or fruit of the invention not having a significantly reduced ability to grow and reproduce when compared to an unmodified plant or part thereof. Preferably, the biomass, growth rate, germination rate, storage organ size, seed size and/or the number of viable seeds produced is not less than 90% of that of a plant lacking said recombinant polynucleotide when grown under identical conditions. This term does not encompass features of the plant which may be different to the wild-type plant but which do not affect the usefulness of the plant for commercial purposes such as, for example, a ballerina phenotype of seedling leaves. In an embodiment, the genetically modified plant or part thereof which is phenotypically normal comprises a recombinant polynucleotide encoding a silencing suppressor operably linked to a plant storage organ specific promoter and has an ability to grow or reproduce which is essentially the same as a corresponding plant or part thereof not comprising said polynucleotide.

Plants provided by or contemplated for use in the practice of the present invention include both monocotyledons and dicotyledons. In preferred embodiments, the plants of the present invention are crop plants (for example, cereals and pulses, maize, wheat, potatoes, rice, sorghum, millet, cassava, barley) or legumes such as soybean, beans or peas. The plants may be grown for production of edible roots, tubers, leaves, stems, flowers or fruit. The plants may be vegetable plants whose vegetative parts are used as food. The plants of the invention may be: Acrocomia aculeata (macauba palm), Arabidopsis thaliana, Aracinis hypogaea (peanut), Astrocaryum murumuru (murumuru), Astrocaryum vulgare (tucumi), Attalea geraensis (Indaii-rateiro), Attalea humilis (American oil palm), Attalea oleifera (andaii), Attalea phalerata (uricuri), Attalea speciosa (babassu), Avena sativa (oats), Beta vulgaris (sugar beet), Brassica sp. such as Brassica carinata, Brassica juncea, Brassica napobrassica, Brassica napus (canola), Camelina sativa (false flax), Cannabis sativa (hemp), Carthamus tinctorius (safflower), Caryocar brasiliense (pequi), Cocos nucifera (Coconut), Crambe abyssinica (Abyssinian kale), Cucumis melo (melon), Elaeis guineensis (African palm), Glycine max (soybean), Gossypium hirsutum (cotton), Helianthus sp. such as Helianthus annuus (sunflower), Hordeum vulgare (barley), Jatropha curcas (physic nut), Joannesia princeps (arara nut-tree), Lemna sp. (duckweed) such as Lemna aequinoctialis, Lemna disperma, Lemna ecuadoriensis, Lemna gibba (swollen duckweed), Lemna japonica, Lemna minor, Lemna minuta, Lemna obscura, Lemna paucicostata, Lemna perpusilla, Lemna tenera, Lemna trisulca, Lemna turionifera, Lemna valdiviana, Lemna yungensis, Licania rigida (oiticica), Linum usitatissimum (flax), Lupinus angustifolius (lupin), Mauritia flexuosa (buriti palm), Maximiliana maripa (inaja palm), Miscanthus sp. such as Miscanthus x giganteus and Miscanthus sinensis, Nicotiana sp. (tabacco) such as Nicotiana tabacum or Nicotiana benthamiana, Oenocarpus bacaba (bacaba-do-azeite), Oenocarpus bataua (pataua), Oenocarpus distichus (bacaba-de-leque), Oryza sp. (rice) such as Oryza sativa and Oryza glaberrima, Panicum virgatum (switchgrass), Paraqueiba paraensis (mari), Persea amencana (avocado), Pongamia pinnata (Indian beech), Populus trichocarpa, Ricinus communis (castor), Saccharum sp. (sugarcane), Sesamum indicum (sesame), Solanum tuberosum (potato), Sorghum sp. such as Sorghum bicolor, Sorghum vulgare, Theobroma grandiforum (cupuassu), Trifolium sp., Trithrinax brasiliensis (Brazilian needle palm), Triticum sp. (wheat) such as Triticum aestivum, Zea mays (corn), alfalfa (Medicago sativa), rye (Secale cerale), sweet potato (Lopmoea batatus), cassava (Manihot esculenta), coffee (Cofea spp.), pineapple (Anana comosus), citris tree (Citrus spp.), cocoa (Theobroma cacao), tea (Camellia senensis), banana (Musa spp.), avocado (Persea americana), fig (Ficus casica), guava (Psidium guajava), mango (Mangifer indica), olive (Olea europaea), papaya (Carica papaya), cashew (Anacardium occidentale), macadamia (Macadamia intergrifolia) and almond (Prunus amygdalus). For example, plants of the disclosure may be Nicotiana benthamiana.

Other preferred plants include C4 grasses such as, in addition to those mentioned above, Andropogon gerardi, Bouteloua curtipendula, B. gracilis, Buchloe dactyloides, Schizachyrium scoparium, Sorghastrum nutans, Sporobolus cryptandrus; C3 grasses such as Elymus canadensis, the legumes Lespedeza capitata and Petalostemum villosum, the forb Aster azureus; and woody plants such as Quercus ellipsoidalis and Q. macrocarpa. Other preferred plants include C3 grasses.

In a preferred embodiment, the plant is an angiosperm.

In an embodiment, the plant is an oilseed plant, preferably an oilseed crop plant. As used herein, an “oilseed plant” is a plant species used for the commercial production of lipid from the seeds of the plant. The oilseed plant may be, for example, oil-seed rape (such as canola), maize, sunflower, safflower, soybean, sorghum, flax (linseed) or sugar beet. Furthermore, the oilseed plant may be other Brassicas, cotton, peanut, poppy, rutabaga, mustard, castor bean, sesame, safflower, Jatropha curcas or nut producing plants. The plant may produce high levels of lipid in its fruit such as olive, oil palm or coconut. Horticultural plants to which the present invention may be applied are lettuce, endive, or vegetable Brassicas including cabbage, broccoli, or cauliflower. The present invention may be applied in tobacco, cucurbits, carrot, strawberry, tomato, or pepper.

In a preferred embodiment, the transgenic plant is homozygous for each and every gene that has been introduced (transgene) so that its progeny do not segregate for the desired phenotype. The transgenic plant may also be heterozygous for the introduced transgene(s), preferably uniformly heterozygous for the transgene such as for example, in F1 progeny which have been grown from hybrid seed. Such plants may provide advantages such as hybrid vigour, well known in the art.

### Transformation

RNA molecules disclosed herein may be stably introduced to above referenced host cells and/or non-human organisms such as plants. For the avoidance of doubt, an example of the present disclosure encompasses an above referenced plant stably transformed with an RNA molecule disclosed herein. As used herein, the terms “stably transforming”, “stably transformed” and variations thereof refer to the integration of the RNA molecule or a nucleic acid encoding the same into the genome of the cell such that they are transferred to progeny cells during cell division without the need for positively selecting for their presence. Stable transformants, or progeny thereof, can be identified by any means known in the art such as Southern blots on chromosomal DNA, or in situ hybridization of genomic DNA, enabling their selection.

Transgenic plants can be produced using techniques known in the art, such as those generally described in Slater et al., Plant Biotechnology—The Genetic Manipulation of Plants, Oxford University Press (2003), and Christou and Klee, Handbook of Plant Biotechnology, John Wiley and Sons (2004).

In an embodiment, plants may be transformed by topically applying an RNA molecule according to the present disclosure to the plant or a part thereof. For example, the RNA molecule may be provided as a formulation with a suitable carrier and sprayed, dusted or otherwise applied to the surface of a plant or part thereof. Accordingly, in an example, the methods of the present disclosure encompass introducing an RNA molecule disclosed herein to a plant, the method comprising topically applying a composition comprising the RNA molecule to the plant or a part thereof.

Agrobacterium-mediated transfer is a widely applicable system for introducing genes into plant cells because DNA can be introduced into cells in whole plant tissues, plant organs, or explants in tissue culture, for either transient expression, or for stable integration of the DNA in the plant cell genome. For example, floral-dip (in planta) methods may be used. The use of Agrobacterium-mediated plant integrating vectors to introduce DNA into plant cells is well known in the art. The region of DNA to be transferred is defined by the border sequences, and the intervening DNA (T-DNA) is usually inserted into the plant genome. It is the method of choice because of the facile and defined nature of the gene transfer.

Acceleration methods that may be used include for example, microprojectile bombardment and the like. One example of a method for delivering transforming nucleic acid molecules to plant cells is microprojectile bombardment. This method has been reviewed by Yang et al., Particle Bombardment Technology for Gene Transfer, Oxford Press, Oxford, England (1994). Non-biological particles (microprojectiles) that may be coated with nucleic acids and delivered into cells, for example of immature embryos, by a propelling force. Exemplary particles include those comprised of tungsten, gold, platinum, and the like.

In another method, plastids can be stably transformed. Methods disclosed for plastid transformation in higher plants include particle gun delivery of DNA containing a selectable marker and targeting of the DNA to the plastid genome through homologous recombination (U.S. Pat. Nos. 5,451,513, 5,545,818, 5,877,402, 5,932,479, and WO 99/05265). Other methods of cell transformation can also be used and include but are not limited to the introduction of DNA into plants by direct DNA transfer into pollen, by direct injection of DNA into reproductive organs of a plant, or by direct injection of DNA into the cells of immature embryos followed by the rehydration of desiccated embryos.

The regeneration, development, and cultivation of plants from single plant protoplast transformants or from various transformed explants is well known in the art (Weissbach et al., In: Methods for Plant Molecular Biology, Academic Press, San Diego, Calif., (1988)). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene is well known in the art. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polynucleotide is cultivated using methods well known to one skilled in the art.

To confirm the presence of the transgenes in transgenic cells and plants, a polymerase chain reaction (PCR) amplification or Southern blot analysis can be performed using methods known to those skilled in the art. Expression products of the transgenes can be detected in any of a variety of ways, depending upon the nature of the product, and include Northern blot hybridisation, Western blot and enzyme assay. Once transgenic plants have been obtained, they may be grown to produce plant tissues or parts having the desired phenotype. The plant tissue or plant parts, may be harvested, and/or the seed collected. The seed may serve as a source for growing additional plants with tissues or parts having the desired characteristics. Preferably, the vegetative plant parts are harvested at a time when the yield of non-polar lipids are at their highest. In one embodiment, the vegetative plant parts are harvested about at the time of flowering, or after flowering has initiated. Preferably, the plant parts are harvested at about the time senescence begins, usually indicated by yellowing and drying of leaves.

Transgenic plants formed using Agrobacterium or other transformation methods typically contain a single genetic locus on one chromosome. Such transgenic plants can be referred to as being hemizygous for the added gene(s). More preferred is a transgenic plant that is homozygous for the added gene(s), that is, a transgenic plant that contains two added genes, one gene at the same locus on each chromosome of a chromosome pair. A homozygous transgenic plant can be obtained by self-fertilising a hemizygous transgenic plant, germinating some of the seed produced and analysing the resulting plants for the gene of interest.

It is also to be understood that two different transgenic plants that contain two independently segregating exogenous genes or loci can also be crossed (mated) to produce offspring that contain both sets of genes or loci. Selfing of appropriate F1 progeny can produce plants that are homozygous for both exogenous genes or loci. Back-crossing to a parental plant and out-crossing with a non-transgenic plant are also contemplated, as is vegetative propagation. Similarly, a transgenic plant can be crossed with a second plant comprising a genetic modification such as a mutant gene and progeny containing both of the transgene and the genetic modification identified. Descriptions of other breeding methods that are commonly used for different traits and crops can be found in Fehr, In: Breeding Methods for Cultivar Development, Wilcox J. ed., American Society of Agronomy, Madison Wis. (1987).

### Formulations

RNA molecules according to the present disclosure can be provided as various formulations. For example, RNA molecules may be in the form of a solid, ointment, gel, cream, powder, paste, suspension, colloid, foam or aerosol. Solid forms may include dusts, powders, granules, pellets, pills, pastilles, tablets, filled films (including seed coatings) and the like, which may be water-dispersible (“wettable”). In one example, the composition is in the form of a concentrate.

In an example, RNA molecules may be provided as a topical formulation. In an example, the formulation stabilises the RNA molecule in formulation and/or in-vivo. For example, RNA molecules may be provided in a lipid formulation. For example, RNA molecules may be provided in liposomes. In an example, the formulation comprises a transfection promoting agent.

In an example, RNA molecules can be incorporated into formulations suitable for application to a field. In an example, the field comprises plants. Suitable plants include crop plants (for example, cereals and pulses, maize, wheat, potatoes, tapioca, rice, sorghum, soybean millet, cassava, barley, or pea), or legumes. The plants may be grown for production of edible roots, tubers, leaves, stems, flowers or fruit. In an example, the crop plant is a cereal plant. Examples of cereal plants include, but are not limited to, wheat, barley, sorghum oats, and rye. In these examples, the RNA molecule may be formulated for administration to the plant, or to any part of the plant, in any suitable way. For example, the composition may be formulated for administration to the leaves, stem, roots, fruit vegetables, grains and/or pulses of the plant. In one example, the RNA molecule is formulated for administration to the leaves of the plant, and is sprayable onto the leaves of the plant.

Depending on the desired formulation, RNA molecules described herein may be formulated with a variety of other agents. Exemplary agents comprise one or more of suspension agents, agglomeration agents, bases, buffers, bittering agents, fragrances, preservatives, propellants, thixotropic agents, anti-freezing agents, and colouring agents.

In other examples, RNA molecule formulations can comprise an insecticide, a pesticide, a fungicide, an antibiotic, an insect repellent, an anti-parasitic agent, an anti-viral agent, or a nematicide.

In another example, RNA molecules can be incorporated into pharmaceutical compositions. Such compositions would typically include an RNA molecule described herein and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, inhalation, transdermal (topical), transmucosal, oral and rectal administration.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. For example, liposomal suspensions can also be used as pharmaceutically acceptable carriers. These can be prepared according to methods known to those skilled in the art, for example, as described in U.S. Pat. No. 4,522,811.

RNA molecules according to the present disclosure can be provided in a kit or pack. For example, RNA molecules disclosed herein may be packaged in a suitable container with written instructions for producing an above referenced cell or organism or treating a condition.

### Methods of Controlling Non-Human Organisms

In an example, the RNA molecules according to the present disclosure can be used to control non-human organisms such as insects. Such uses involve administering RNA molecules according to the present disclosure using various methods. In an example, RNA molecules according to the present disclosure can be provided as an insect bait for ingestion by insects. In another example, RNA molecules can be sprayed onto insects as required. In another example, RNA molecules can be sprayed onto a plant or crop to protect said plant or crop from insects. Exemplary crops include cotton, maize, tomato, chickpea, pigeon pea, alfalfa, rice, sorghum and cowpea.

In an example, RNA molecules can be provided to modify insect behaviour. In another example, RNA molecules can be provided to kill insects. In another example, RNA molecules can be provided to reduce insect fertility. Exemplary insect targets include household insects. Other exemplary insect targets include sap sucking insects such as aphids (e.g. Myzus persicae, Metopolophium dirhodum, Rhopalosiphum padi, Aphis glycines, Aphis fabae). Further exemplary insect targets include, arachnids, mosquitoes, ecto-parasites, flies, spider mites, thrips, ticks, red poultry mite, ants, cockroaches, termites, crickets including house-crickets, silverfish, booklice, beetles, earwigs, mosquitos and fleas. Other exemplary insect targets include agricultural pests. Examples include sap feeders such as stink bugs and aphids, chewing insects such as caterpillars, beetles, worms, rasping insects such as thrips and slugs, moths, fly's such as fruit fly, grain pests such as grain borer, weevils, and grain moths.

In an embodiment the insect is a sap sucking insect. In this example, the RNA molecule can have antisense activity for MpC002 and/or MpRack-1. In an embodiment the sap sucking insect is an aphid. In another embodiment, the aphid is Myzus persicae.

In an embodiment the insect target is an ant (e.g. Linepithema humile), cotton bollworm or corn ear worm (Helicoverpa armigera) or blowfly (e.g. Lucilia cuprina). In an embodiment the target insect is Helicoverpa armigera and the RNA molecule has antisense activity for the ABC transporter white gene (ABC white). In another embodiment the target insect is Linepithema humile and the RNA molecule has antisense activity for pheromone biosynthesis activating neuropeptide (PBAN). In another embodiment the target insect is Lucilia cuprina and the RNA molecule has antisense activity for one or more genes encoding proteins selected from the group consisting of V-type proton ATPase catalytic subunit A, RNAse 1/2, chitin synthase, ecdysone receptor and gamma-tubulin 1/1-like.

In the above embodiment, compositions and RNA molecules disclosed herein may be provided in a dispenser. In an example, the dispenser is a trap or a lure. In an embodiment, the trap and/or lure comprises a bait comprising an RNA molecule(s) disclosed herein.

In an embodiment, the present disclosure encompasses methods of controlling insect behaviour, the method comprising spraying, dusting or otherwise applying RNA molecules disclosed herein to the insects. In this embodiment, RNA molecules can be sprayed, dusted or otherwise applied directly to the insects. In another embodiment, RNA molecules can be sprayed, dusted or otherwise applied to plants or crops prior to insect infestation.

In one embodiment of the invention, the insect or arachnid may belong to the following orders: Acari, Arachnida, Anoplura, Blattodea, Coleoptera, Collembola, Dermaptera, Dictyoptera, Diplura, Diptera, Embioptera, Ephemeroptera, Grylloblatodea, Hem iptera, Heteroptera, Homoptera, Hymenoptera, Isoptera, Lepidoptera, Mallophaga, Mecoptera, Neuroptera, Odonata, Orthoptera, Phasmida, Phithiraptera, Plecoptera, Protura, Psocoptera, Siphonaptera, Siphunculata, Thysanura, Stemorrhyncha, Strepsiptera, Thysanoptera, Trichoptera, Zoraptera and Zygentoma.

In preferred, but non-limiting, embodiments of the invention the insect or arachnid is chosen from the group consisting of: (1) Acari: mites including Ixodida (ticks) (2) Arachnida:Araneae (spiders) and Opiliones (harvestman), examples include: Latrodectus mactans (black widow) and Loxosceles recluse (Brown Recluse Spider) (3) Anoplura: lice, such as Pediculus humanus (human body louse) (4) Blattodea: cockroaches including German cockroach (Blatella germanica), of the genus Periplaneta, including American cockroach (Periplaneta americana) and Australian cockroach (Periplaneta australiasiae), of the genus Blatta, including Oriental cockroach (Blatta orientalis) and of the genus Supella, including brown-banded cockroach (Supella longipalpa). A most preferred target is German cockroach (Blatella germanica). (5) Coleoptera: beetles, examples include: the family of Powderpost beetle (family of Bostrichoidea); Dendroctonus spp. (Black Turpentine Beetle, Southern Pine Beetle, IPS Engraver Beetle); Carpet Beetles (Anthrenus spp, Attagenus spp); Old House Borer (family of Cerambycidae: Hylotrupes bajulus); Anobium punctatum; Tribolium spp (flour beetle); Trogoderma granarium (Khapra Beetle); Oryzaephilus sarinamensis (Toothed Grain Beetle) etc. (Bookworm) (6) Dermaptera: family of earwigs (7) Diptera: mosquitoes (Culicidae) and flies (Brachycera), examples are: Anophelinae such as Anopheles spp. and Culicinae such as Aedes fulvus; Tabanidae such as Tabanus punctifer (Horse Fly), Glossina morsitans morsitans (tsetse fly), drain flies (Psychodidae) and Calyptratae such as Musca domestica (House fly), flesh flies (family of Sarcophagidae) etc. (8) Heteroptera: bugs, such as Cimex lectularius (bed bug) (9) Hymenoptera: wasps (Apocrita), including ants (Formicoidea), bees (Apoidea): Solenopsis invicta (Red Fire Ant), Monomorium pharaonis (Pharaoh Ant), Camponotus spp (Carpenter Ants), Iasius niger (Small Black Ant), tetramorium caespitum (Pavement Ant), Myrmica rubra (Red Ant), Formica spp (wood ants), Crematogaster lineolata (Acrobat Ant), Iridomyrmex humilis (Argentine Ant), Pheidole spp. (Big Headed Ants, Dasymutilla occidentalis (Velvet Ant) etc. (10) Isoptera: termites, examples include: Amitermes floridensis (Florida dark-winged subterranean termite), the eastern subterranean termite (Reticulitermes flavipes), the R. hesperus (Western Subterranean Termite), Coptotermes formosanus (Formosan Subterranean Termite), Incisitermes minor (Western Drywood Termite), Neotermes connexus (Forest Tree Termite) and Termitidae (11) Lepidoptera: moths, examples include: Tineidae & Oecophoridae such as Tineola bisselliella (Common Clothes Moth), and Pyralidae such as Pyralis farinalis (Meal Moth) etc (12) Psocoptera: booklice (Psocids) (13) Siphonaptera: fleas such as Pulex irritans (14) Stemorrhyncha: aphids (Aphididae) (15) Zygentoma: silverfish, examples are: Thermobia domestica and Lepisma saccharina

Other target insects or arachnids include household insects, ecto-parasites and insects and/or arachnids relevant for public health and hygiene such as, by way of example and not limitation, flies, spider mites, thrips, ticks, red poultry mite, ants (such as by targeting PBAN), cockroaches, termites, crickets including house-crickets, silverfish, booklice, beetles, earwigs, mosquitos and fleas. More preferred targets are cockroaches (Blattodea) such as but not limited to Blatella spp. (e.g. Blatella germanica (german cockroach)), Periplaneta spp. (e.g. Periplaneta americana (American cockroach) and Periplaneta australiasiae (Australian cockroach)), Blatta spp. (e.g. Blatta orientalis (Oriental cockroach)) and Supella spp. (e.g. Supella longipalpa (brown-banded cockroach); ants (Formicoidea), such as but not limited to Solenopsis spp. (e.g. Solenopsis invicta (Red Fire Ant)), Monomorium spp. (e.g. Monomorium pharaonis (Pharaoh Ant)), Camponotus spp. (e.g. Camponotus spp (Carpenter Ants)), Lasius spp. (e.g. Lasius niger (Small Black Ant)), Tetramorium spp. (e.g. Tetramorium caespitum (Pavement Ant)), Myrmica spp. (e.g. Myrmica rubra (Red Ant)), Formica spp (wood ants), Crematogaster spp. (e.g. Crematogaster lineolata (Acrobat Ant)), Iridomyrmex spp. (e.g. Iridomyrmex humilis (Argentine Ant)), Pheidole spp. (Big Headed Ants), and Dasymutilla spp. (e.g. Dasymutilla occidentalis (Velvet Ant)); termites (Isoptera and/or Termitidae) such as but not limited to Amitermes spp. (e.g. Amitermes floridensis (Florida dark-winged subterranean termite)), Reticulitermes spp. (e.g. Reticulitermes flavipes (the eastern subterranean termite), Reticulitermes hesperus (Western Subterranean Termite)), Coptotermes spp. (e.g. Coptotermes formosanus (Formosan Subterranean Termite)), Incisitermes spp. (e.g. Incisitermes minor (Western Drywood Termite)), Neotermes spp. (e.g. Neotermes connexus (Forest Tree Termite)).

In an embodiment, the target RNA encodes an insect acteolactate synthase.

The RNA molecules of the invention when delivered and/or expressed in a plant can have a wide range of desired properties which influence, for example, an agronomic trait, insect resistance (such as by targeting genes such as MpC002, MpRack-1 and an ABC transporter gene), disease resistance (such as by targeting genes such as LanR), herbicide resistance, sterility, grain characteristics, and the like. The target RNA molecule may be involved in metabolism of oil, starch, carbohydrates, nutrients, etc., or may be responsible for the synthesis of proteins, peptides, fatty acids, lipids, recombination frequency (by targeting genes such as DDM1 and FANCM), waxes, oils (by targeting genes such as TOR), starches, sugars, carbohydrates, flavors, odors, toxins, carotenoids, hormones (by targeting genes such as EIN2, NCED1 and NCED2), polymers, flavonoids (by targeting a gene such as chalcone synthase), storage proteins, phenolic acids, alkaloids, lignins, tannins, celluloses, glycoproteins, glycolipids, etc.

In a particular example, the plants produce increased levels of enzymes for oil production in plants such as Brassicas, for example oilseed rape or sunflower, safflower, flax, cotton, soybean or maize; enzymes involved in starch synthesis in plants such as potato, maize, and cereals such as wheat barley or rice; enzymes which synthesize, or proteins which are themselves, natural medicaments, such as pharmaceuticals or veterinary products.

In another embodiment, an RNA molecule of the present invention is directed to the prophylactic or therapeutic treatment of infection by a fungal pathogen selected from the group consisting of: Altemaria spp.; Armillaria mellae; Arthrobotrys oligosporus; Blumeria graminis (by taregting Mlo genes using an RNA molecule as described in Example 17), Boletus granulatus; Botritis cinerea; Botrytis fabae; Candida albicans; Claviceps purpurea; Cronartium ribicola; Epicoccum purpurescens; Epidermophyton floccosum; Fomes annosus; Fusarium oxysporum; Gaeumannomyces graminis var. tritici; Glomerella cingulata; Gymnosporangium juniperi-virginianae; Microsporum canis; Monilinia fructicola; Physoderma alfalfae; Phytopthera infestans; Pityrosporum orbiculare (Malassezia furfur); Polyporus sulphureus; Puccinia spp.; Saccharomyces cerevisiae; Septoria apiicola; Trichophyton rubrum; T. mentagrophytes; Ustilago spp.; Venturia inaequalis; and Verticillium dahliae.

### Exemplary Conditions to be Treated

RNA molecules according to the present disclosure may be used in methods of various conditions. In some examples, the present disclosure relates to a method of treating cancer comprising administering an RNA molecule disclosed herein. The term “cancer” refers to or describes the physiological condition in mammals that is typically characterized by unregulated cell growth/proliferation. Examples of cancer include but are not limited to, carcinoma, lymphoma, blastoma, sarcoma, and leukemia or lymphoid malignancies. More particular examples of such cancers include, but are not limited to, squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, multiple myeloma and B-cell lymphoma (including low grade/follicular non-Hodgkin's lymphoma (NHL); mantle cell lymphoma; AIDS-related lymphoma; and Waldenstrom's Macroglobulinemia); chronic lymphocytic leukemia (CLL); acute lymphoblastic leukemia (ALL); hairy cell leukemia; chronic myeloblastic leukemia; and post-transplant lymphoproliferative disorder (PTLD), as well as abnormal vascular proliferation associated with phakomatoses, edema (such as that associated with brain tumors), Meigs' syndrome, brain, as well as head and neck cancer, and associated metastases. Accordingly, in an example, the present disclosure relates to a method of treating breast, ovarian, colon, prostate, lung, brain, skin, liver, stomach, pancreatic or blood based cancer.

In other examples, a method described herein is used to treat cancers that are linked to mutations in BRCA1, BRCA2, PALB2, OR RAD51B, RAD51C, RAD51D or related genes.

In other examples, a method described herein is used to treat cancers that are linked to mutations in genes associated with DNA mismatch repair, such as MSH2, MLH1, PMS2, and related genes. In other examples, a method described herein is used to treat cancers with silenced DNA repair genes, such as BRCA1, MLH1, OR RAD51B, RAD51C, OR RAD51D.

In other examples of the disclosure, a method described herein is used to kill cells with impaired DNA repair processes. For example, cells with impaired DNA repair may aberrantly express a gene involved in DNA repair, DNA synthesis, or homologous recombination.

Exemplary genes include XRCC1, ADPRT (PARP-1), ADPRTL2, (PARP-2), POLYMERASE BETA, CTPS, MLH1, MSH2, FANCD2, PMS2, p53, p21, PTEN, RPA, RPA1, RPA2, RPA3, XPD, ERCC1, XPF, MMS19, RAD51, RAD51B, RAD51C, RAD51D, DMC1, XRCCR, XRCC3, BRCA1, BRCA2, PALB2, RAD52, RAD54, RAD50, MREU, NB51, WRN, BLM, KU70, KU80, ATM, ATR CPIK1, CHK2, FANCA, FANCB, FANCC, FANCD1, FANCD2, FANCE, FANCF, FANCG, FANCC, FANCD1, FANCD2, FANCE, FANCF, FANCG, RAD1, and RAD9. In an example, a method described herein is used to kill cells with a mutant tumour suppressor gene. For example, cells can have one or more mutations in BRCA1 or BRCA2.

In other examples of the disclosure, a method described herein is used to treat virally transformed cells. In other examples of the disclosure, a method described herein is used to kill cells transformed with a latent virus. Exemplary latent viruses include CMV, EBV, Herpes simplex virus (type 1 and 2), and Varicella zoster virus. In other examples of the disclosure, a method described herein is used to treat active viral infections due to viruses that give rise to cancer, immunodeficiency, hepatitis, encephalitis, pneumonitis or respiratory illness. Exemplary viruses include parvovirus, poxvirus, herpes virus.

In other examples of the disclosure, a method described herein is used to treat Zika Virus, Colorado Tick Fever (caused by Coltivirus, RNA virus), West Nile Fever (encephalitis, caused by a flavivirus that primarily occurs in the Middle East and Africa), Yellow Fever, Rabies (caused by a number of different strains of neurotropic viruses of the family Rhabdoviridae), viral hepatitis, gastroenteritis (viral)-acute viral gastroenteritis caused by Norwalk and Norwalk-like viruses, rotaviruses, caliciviruses, and astroviruses, poliomyelitis, influenza (flu), caused by orthomyxoviruses that can undergo frequent antigenic variation, measles (rubeola), paramyxoviridae, mumps, respiratory syndromes including viral pneumonia and acute respiratory syndromes including croup caused by a variety of viruses collectively referred to as acute respiratory viruses, and respiratory illness caused by the respiratory syncytial virus.

### EXAMPLES

### Example 1. Materials and Methods

### Synthesis of Genetic Constructs

To design a typical ledRNA construct, a region of the target RNA of about 100-1000 nucleotides in length, typically 400-600 nucleotides, was identified. In one example, the 5′ half of the sequence and approximately 130 nt of the flanking region and similarly the 3′ half and 130 nt of flanking region were orientated in an antisense orientation relative to a promoter. These sequences were interrupted with the 400-600 nucleotide sense target sequence (FIG. 1A). The 5′ end of the resultant construct was preceded with a promoter such as a T7 or SP6 RNA polymerase promoter and the 3′ end engineered to include a restriction enzyme cleavage site to allow for termination of transcription in vitro.

For transcription in cells such as bacterial cells, promoter and terminator sequences were incorporated to facilitate expression as a transgene, for example using an inducible promoter. The double-stranded region and loop sequence lengths can be varied. The constructs were made using standard cloning methods or ordered from commercial service providers.

### Synthesis of RNA

Following digestion with restriction enzyme to linearize the DNA at the 3′ end, transcription using RNA polymerase resulted in the 5′ and 3′ arms of the ledRNAi transcript annealing to the central target sequence, the molecule comprising a central stem or double-stranded region with a single nick and terminal loops. The central sequence can be orientated in sense or antisense orientation relative to the promoter (FIG. 1A, 1B respectively).

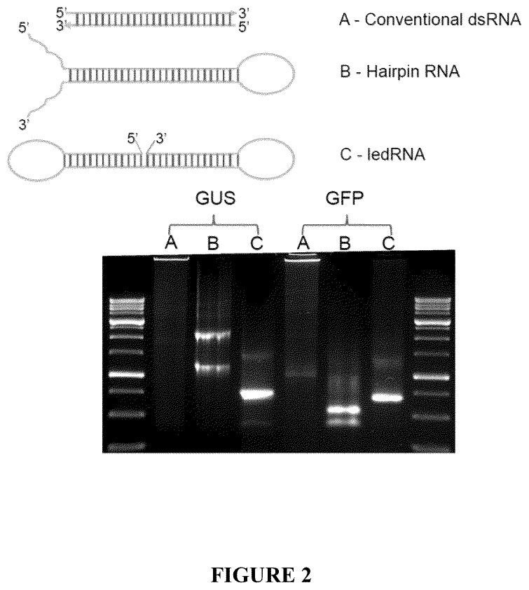

For in vitro synthesis, DNA of the construct was digested at the 3′ restriction site using the appropriate restriction enzyme, precipitated, purified and quantified. RNA synthesis was achieved using RNA polymerase according to the manufacturer's instructions. The ledRNA was resuspended in annealing buffer (25 mM Tris-HCL, pH 8.0, 10 mM MgCl₂) using DEPC-treated water to inactivate any traces of RNAse. The yield and integrity of the RNA produced by this method was determined by nano-drop analysis and gel electrophoresis (FIG. 2), respectively.

Synthesis of ledRNA was achieved in bacterial cells by introducing the constructs into E. coli strain HT115. Transformed cell cultures were induced with IPTG (0.4 mM) to express the T7 RNA polymerase, providing for transcription of the ledRNA constructs. RNA extraction from the bacterial cells and purification was performed essentially as described in Timmons et al. (2001).

For RNA transcription with Cy3 labelling, the ribonucleotide (rNTP) mix contained 10 mM each of ATP, GTP, CTP, 1.625 mM UTP and 8.74 mM Cy3-UTP. The transcription reactions were incubated at 37° C. for 2.5 hr. The transcription reactions (160 μl) were the transferred to Eppendorf tubes, 17.7 μl turbo DNase buffer and 1 μl turbo DNA added, and incubated at 37° C. for 10 minutes to digest the DNA. Then, 17.7 μl Turbo DNAse inactivation solution was added, mixed and incubated at room temperature for 5 min. The mixture was centrifuged for 2 min and the supernatant transferred to a new RNAse free Eppendorf tube. Samples of 1.5 μl of each transcription reaction were electrophoresed on gels to test the quality of the RNA product. Generally, one RNA band was observed of 500 bp to 1000 bp in size depending on the construct. The RNA was precipitated by adding to each tube: 88.5 μl 7.5M Ammonium acetate and 665 μl cold 100% ethanol. The tubes were cooled to −20° C. for several hours or overnight, then centrifuged at 4° C. for 30 min. The supernatant was removed carefully and the pellet of RNA washed with 1 ml 70% ethanol (made with nuclease free water) at −20° C. and centrifuged. The pellet was dried and the purified RNA resuspended in 50 μl 1×RNAi annealing buffer. The RNA concentration was measured using nanodrop method and stored at −80° C. until used.

### Example 2. Design of ledRNA

As shown schematically in FIG. 1A, a typical ledRNA molecule comprises a sense sequence which can be considered to be two adjacent sense sequences, covalently linked and having identity to the target RNA, an antisense sequence which is complementary to the sense sequence and which is divided into two regions, and two loops that separate the sense from the antisense sequences. A DNA construct which encodes this form of ledRNA therefore comprises, in 5′ to 3′ order, a promoter for transcription of the ledRNA coding region, a first antisense region having complementarity with a region towards the 5′ end of the target RNA, a first loop sequence, the sense sequence, a second loop sequence, then the second antisense region having complementarity with a region towards the 3′ end of the target RNA, and finally a means to terminate transcription. In this arrangement, the two antisense sequences flanked the sense sequence and loop sequences. When transcribed, the two regions of antisense sequence anneal with the sense sequence, forming a dsRNA stem with two flanking loops.

In another but related form of ledRNA, the sense sequence is split into two regions whilst the two antisense regions remain as a single sequence (FIG. 1B). A DNA construct which encodes this second form of ledRNA therefore comprises, in 5′ to 3′ order, a promoter for transcription of the ledRNA coding region, a first sense region having identity with a region towards the 3′ end of the target RNA, a first loop sequence, the antisense sequence, a second loop sequence, then the second sense region having identity towards the 5′ end of the target RNA, and finally a means to terminate transcription. In this arrangement, the two sense sequences flanked the antisense sequence and loop sequences.

Without wishing to be limited by theory, because of the closed loops at each end, these ledRNA structures would be more resistant to exonucleases than an open-ended dsRNA formed between single-stranded sense and antisense RNAs and not having loops, and also compared to a hairpin RNA having only a single loop. In addition, the inventors conceived that a loop at both ends of the dsRNA stem would allow Dicer to access both ends efficiently, thereby enhancing processing of the dsRNA into sRNAs and silencing efficiency.

As a first example, a genetic construct was made for in vitro transcription using T7 or SP6 RNA polymerase to form ledRNAs targeting genes encoding GFP or GUS. The ledGFP construct comprised the following regions in order: the first half of antisense sequence corresponded to nucleotides 358 to 131 of the GFP coding sequence (CDS) (SEQ ID NO:7), the first antisense loop corresponded to nucleotides 130 to 1 of GFP CDS, the sense sequence corresponded of nucleotides 131 to 591 of GFP CDS, the second antisense loop corresponding to nucleotides 731 to 592 of GFP CDS, and the second half of the antisense sequence corresponded to nucleotides 591 to 359 of the GFP CDS.

The ledGUS construct comprised the following regions in order: the first half of antisense sequence corresponded to nucleotides 609 to 357 of GUS CDS (SEQ ID NO:8); the first antisense loop corresponded to nucleotides 356 to 197 of GUS CDS, the sense sequence corresponded to nucleotides 357 to 860 of GUS CDS, the second antisense loop corresponding to nucleotides 1029 to 861 of GUS CDS; and the second half of antisense sequence corresponded to nucleotides 861 to 610 of GUS CDS.

For making the separate strand sense/antisense GUS dsRNA (conventional dsRNA), the same target sequence corresponding to nucleotides 357 to 860 of GUS CDS was ligated between the T7 and SP6 promoters in pGEM-T Easy vector. The sense and antisense strands were transcribed separately with T7 or SP6 polymerases, respectively, and annealed in annealing buffer after mixing the transcripts and heating the mixture to denature the RNA strands.

### Example 3. Stability of ledRNAs

The ability of ledRNA to form dsRNA structures was compared with open-ended dsRNA (i.e. no loops, formed by annealing of separate single-stranded sense and antisense RNA) and long hpRNA. ledRNA, long hpRNA, and the mixture of sense and antisense RNA, were denatured by boiling and allowed to anneal in annealing buffer (250 mM Tris-HCL, pH 8.0 and 100 mM MgCl2), and then subjected to electrophoresis in a 1.0% agarose gel under non-denaturing conditions.

As shown in FIG. 2, both the GUS ledRNA and the GFP ledRNA gave a dominant RNA band of the mobility expected for a double-stranded molecule, indicating the formation of the predicted ledRNA structure. This was in contrast to the mixture of sense and antisense RNA, which showed only a weak band for a dsRNA, indicating that most of the sense and antisense RNAs were not readily annealed to each other to form dsRNA. The hairpin RNA samples gave two prominent bands, indicating that only part of the transcript formed the predicted hairpin RNA structure. Thus, ledRNA was the most efficient in forming the predicted dsRNA structure.

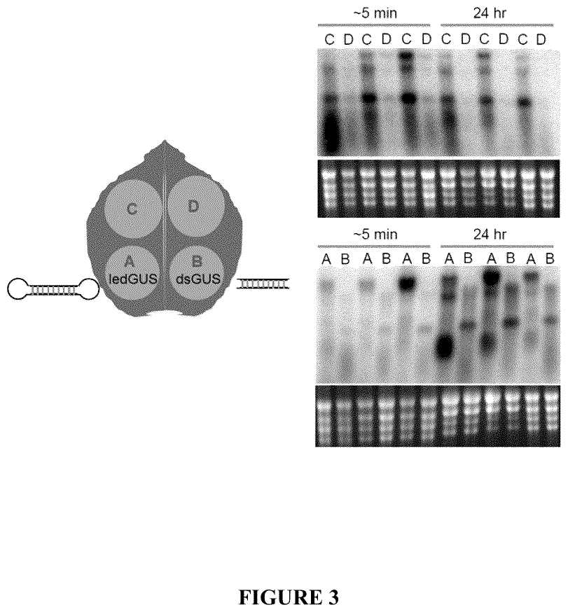

The ability of ledRNA to stay and spread on leaf surface was also compared with dsRNA. The GUS ledRNA (ledGUS), when applied to the lower part of tobacco leaf surface, could be readily detected in the untreated upper part of the leaf after 24 hrs (FIG. 3). However, the separate strand GUS dsRNA (dsGUS) could not be detected in the untreated upper part of the leaf (FIG. 3). This result indicates that the ledRNA is more resistant to degradation than dsRNA and therefore able to spread inside plant leaf tissues.

### Example 4. Testing of ledRNAs by Topical Delivery

The ability of the ledRNAs to induce RNAi after topical delivery was tested in Nicotiana benthamiana and Nicotiana tabacum plants expressing a GFP or GUS reporter gene, respectively. The sequences of the GFP and GUS target sequences and of the ledRNA encoding constructs are shown in SEQ ID NOs: 7, 8, 4 and 5, respectively. The ribonucleotide sequence of the encoded RNA molecules are provided as SEQ ID NO's 1 (GFP ledRNA) and 2 (GUS ledRNA).

To facilitate reproducible and uniform application of ledRNA onto leaf surfaces, ledRNA at a concentration of 75-100 μg/ml, in 25 mM Tris-HCL, pH 8.0, 10 mM MgCl₂ and Silwet 77 (0.05%), was applied to the adaxial surface of leaves using a soft paint brush. At 6 hours and 3 days following ledRNA application, leaf samples were taken for the analysis of targeted gene silencing.

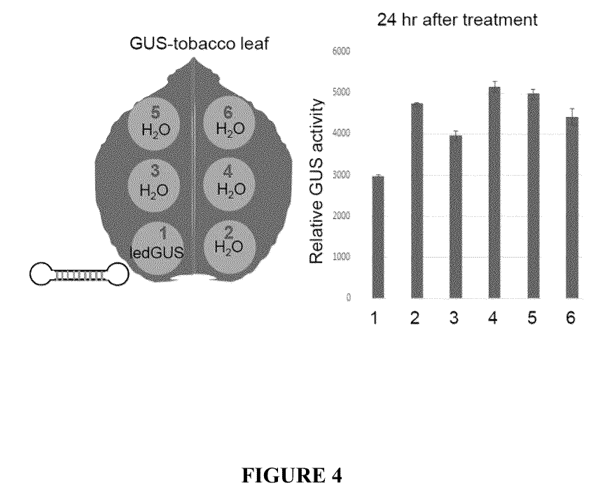

Application of ledRNA against GFP in N. benthamiana leaves and against GUS in N. tabacum leaves resulted in clear reductions of 20-40% and 40-50% of the respective target gene activity at the mRNA (GFP) or protein activity (GUS) level at 6 hours post treatment. However, in this experiment the reduction did not persist at 3 days post treatment. The inventors considered that the observation at 3 days was likely due to some nonspecific responses of transgenes to dsRNA treatment or dissipating amount of ledRNA. However, in a separate experiment, GUS silencing was detected in both the treated and distal untreated leaf areas at 24 hrs post ledRNA treatment (FIG. 4).

### Example 5. LedRNA-Induced Silencing of an Endogenous Target Gene

In a further example, a ledRNA was designed to target an mRNA encoded by an endogenous gene, namely the FAD2.1 gene of N. benthamiana. The sequence of the target FAD2.1 mRNA and of the ledFAD2.1 encoding construct are shown in SEQ ID NOs: 9 and 6, respectively. The ribonucleotide sequence of the encoded RNA molecule is provided as SEQ ID NO: 3 (N. benthamiana FAD2.1 ledRNA).

The FAD2.1 ledRNA construct was comprised of the following: the first half of antisense sequence corresponding to nucleotides 678 to 379 of FAD2.1 CDS (Niben101Scf09417g01008.1); the first antisense loop corresponding to nt. 378 to 242 of FAD2.1 CDS; the sense sequence corresponding of nt. 379 to 979; the second antisense loop corresponding to nt 1115 to 980; and the second half of antisense sequence corresponding to nt 979 to nt 679 of FAD2.1 CDS.

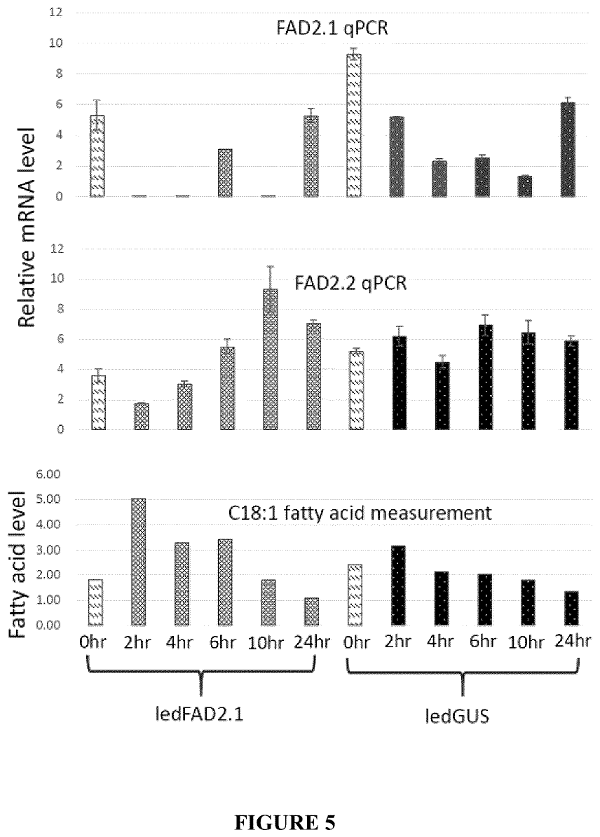

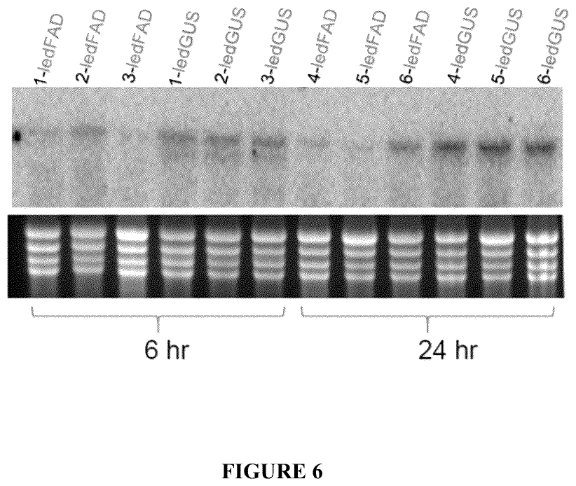

The ledGUS RNA from the previous example was used in parallel as a negative control. In the first experiment, target gene silencing was assayed for both the level of FAD2.1 mRNA and the accumulation C18:1 fatty acid (FIG. 5). The level of activity of a related gene, FAD2.2, was also assayed. For each sample approximately 3 μg of total RNA was DNase treated and reverse transcribed at 50° C. for 50 minutes using oligo dT primers. The reactions were terminated at 85° C. for 5 minutes and diluted to 120 μl with water. Using a rotor gene PCR machine, 5 μl of each sample, in triplicate, were analysed for their relative expression of FAD 2.1 and FAD 2.2 mRNA using gene specific primers with reference to the house keeping gene actin. In a subsequent experiment, northern blot hybridization was also used to confirm the silencing of the FAD2.1 gene by topically applied ledFAD2.1 RNA (FIG. 6).

The FAD2.1 mRNA was reduced significantly, to a level which was barely detectable in leaf tissues treated with the ledRNA at the 2, 4 and 10 hour time points (FIG. 5). In this experiment, it was unclear why the level of FAD2.1 mRNA was not reduced as much at the 6 hour time point. In the repeated experiment shown in FIG. 6, strong FAD2.1 downregulation occurred at both 6 and 24 hrs, particularly at the 24 hr time point. The related FAD2.2 gene, with sequence homology to FAD2.1, also showed downregulation at the 2 and 4 hour time points by the ledRNA (FIG. 5).

Since FAD2.1 and FAD2.2 encode fatty acid A12 desaturases which desaturate oleic acid to linoleic acid, the levels of these fatty acids were assayed in leaf tissues treated with the ledRNAs. There was a clear increase in oleic acid (18:1) accumulation in ledRNA-treated leaf tissues at the 2, 4 and 6 hour time points, which indicated a reduced amount of the FAD2 enzyme (FIG. 5). Thus, both qRT-PCR and the fatty acid composition assay showed that the ledRNA induced silencing of the FAD2.1 gene.

### Example 6. Design and Testing of Hairpin RNAs Comprising G:U Basepairs or Mismatched Nucleotides

### Modified Hairpin RNAs Targeting GUS RNA

Reporter genes such as the gene encoding the enzyme β-glucuronidase (GUS) provide a simple and convenient assay system that can be used to measure gene silencing efficiency in a eukaryotic cell including in plant cells (Jefferson et al., 1987). The inventors therefore designed, produced and tested some modified hairpin RNAs for their ability to reduce the expression of a GUS gene as a target gene, using a gene-delivered approach to provide the hairpin RNAs to the cells, and compared the modified hairpins to a conventional hairpin RNA. The conventional hairpin RNA used as the control in the experiment had a double-stranded region of 200 contiguous basepairs in length in which all of the basepairs were canonical basepairs, i.e. G:C and A:U basepairs without any G:U basepairs, and without any non-basepaired nucleotides (mismatches) in the double-stranded region, targeting the same 200 nt region of the GUS mRNA molecule as the modified hairpin RNAs. The sense and antisense sequences that formed the double-stranded region were covalently linked by a spacer sequence included a PDK intron (Helliwell et al., 2005; Smith et al., 2000), providing for an RNA loop of 39 or 45 nucleotides in length (depending on the cloning strategy used) after splicing of the intron from the primary transcript. The DNA fragment used for the antisense sequence was flanked by XhoI-BamHI restriction sites at the 5′ end and HindIII-KpnI restriction sites at the 3′ end for easy cloning into an expression cassette, and each sense sequence was flanked by XhoI and KpnI restriction sites. The 200 bp dsRNA region of each hairpin RNA, both for the control hairpin and the modified hairpins, included an antisense sequence of 200 nucleotides which was fully complementary to a wild-type GUS sequence from within the protein coding region. This antisense sequence, corresponding to nucleotides 13-212 of SEQ ID NO:10, was the complement of nucleotides 804-1003 of the GUS open reading frame (ORF) (cDNA sequence provided as SEQ ID NO:8). The GUS target mRNA was therefore more than 1900 nt long. The length of 200 nucleotides for the sense and antisense sequences was chosen as small enough to be reasonably convenient for synthesis of the DNA fragments using synthetic oligonucleotides, but also long enough to provide multiple sRNA molecules upon processing by Dicer. Being part of an ORF, the sequence was unlikely to contain cryptic splice sites or transcription termination sites.

### Preparation of Genetic Constructs

The 200 bp GUS ORF sequence was PCR-amplified using the oligonucleotide primer pair GUS-WT-F (SEQ ID NO:52) and GUS-WT-R (SEQ ID NO:53), containing XhoI and BamHI sites or HindIII and KpnI sites, respectively, to introduce these restriction enzyme sites 5′ and 3′ of the GUS sequence. The amplified fragment was inserted into the vector pGEM-T Easy and the correct nucleotide sequence confirmed by sequencing. The GUS fragment was excised by digestion with BamHI and HindIII and inserted into the BamHI/HindIII site of pKannibal (Helliwell and Waterhouse, 2005), which inserted the GUS sequence in the antisense orientation relative to the operably linked CaMV e35S promoter (Grave, 1992) and ocs gene polyadenylation/transcription terminator (Ocs-T). The resultant vector was designated pMBW606 and contained, in order 5′ to 3′, a 35S::PDK Intron::antisense GUS::Ocs-T expression cassette. This vector was the intermediate vector used as the base vector for assembling four hpRNA constructs.

Construct hpGUS[Wt] Having Only Canonical Basepairs

To prepare the vector designated hpGUS[wt] encoding the hairpin RNA molecule used as a control in the experiment, having only canonical basepairs, the 200 bp GUS PCR fragment was excised from the pGEM-T Easy plasmid with XhoI and KpnI, and inserted into the XhoI/KpnI sites between the 35S promoter and the PDK intron in pMBW606. This produced the vector designated pMBW607, containing the 35S::Sense GUS[wt]::PDK Intron::antisense GUS::OCS-T expression cassette. This cassette was excised by digestion with NotI and inserted into the NotI site of pART27 (Gleave, 1992), resulting in the vector designated hpGUS[wt], encoding the canonically basepaired hairpin RNA targeting the GUS mRNA.

When self-annealed by hybridisation of the 200 nt sense and antisense sequences, this hairpin had a double-stranded region of 200 consecutive basepairs corresponding to GUS sequences. The sense and antisense sequences in the expression cassette were each flanked by BamHI and HindIII restrictions sites present at the 5′ and 3′ ends, respectively, relative to the GUS sense sequence. When transcribed, the nucleotides corresponding to these sites were also capable of hybridising, extending the double-stranded region by 6 bp at each end. After transcription of the expression cassette and splicing of the PDK intron from the primary transcript, the hairpin RNA structure prior to any processing by Dicer or other RNAses was predicted to have a loop structure of 39 nucleotides. The nucleotide sequence of the hairpin RNA structure including its loop is provided as SEQ ID NO: 15, and its free energy of folding was predicted to be −471.73 kcal/mol. This was therefore an energetically stable hairpin structure. The free energy was calculated using “RNAfold” (http://ma.tbi.univie.ac.at/cgi-bin/RNAWebSuite/RNAfold.cgi) based on the nucleotide sequences after the splicing out of the PDK intron sequence.

When transcribed from the expression cassette having the 35S promoter and OCS-T terminator, the resultant hairpin RNAs were embedded in a larger RNA molecule with 8 nucleotides added to the 5′ end and approximately 178 nucleotides added at the 3′ end, without considering addition of any poly-A tail at the 3′ end. Since the same promoter-terminator design was used for the modified hairpin RNAs, those molecules also had these extensions at the 5′ and 3′ ends. The length of the hairpin RNA molecules after splicing of the PDH intron was therefore approximately 630 nucleotides.

Construct hpGUS[G:U] Comprising G:U Basepairs

A DNA fragment comprising the same 200 nucleotide sense sequence, but in which all 52 cytidine nucleotides (C) of the corresponding wild-type GUS region were substituted with thymidine nucleotides (T), was assembled by annealing the overlapping oligonucleotides GUS-GU-F (SEQ ID NO:54) and GUS-GU-R (SEQ ID NO:55) and PCR extension of the 3′ ends using the high-fidelity LongAmp Taq polymerase (New England Biolabs, catalogue number M0323). The amplified DNA fragment was inserted into the pGEM-T Easy vector and the correct nucleotide sequence (SEQ ID NO:11) was confirmed by sequencing. A DNA fragment comprising the modified sequence was then excised by digestion with XhoI and KpnI and inserted into the XhoI/KpnI sites of the base vector pMBW606. This produced the construct designated pMBW608, containing the expression cassette 35S::sense GUS[G:U]::PDK Intron::antisense GUS::OCS-T. This expression cassette was excised with NotI digestion and inserted into the NotI site of pART27, resulting in the vector designated hpGUS[G:U], encoding the G:U basepaired hairpin RNA molecule.

This cassette encoded a hairpin RNA targeting the GUS mRNA and which, when self-annealed by hybridisation of the 200 nt sense and antisense sequences, had 52 G:U basepairs (instead of G:C basepairs in hpGUS[wt]) and 148 canonical basepairs, i.e. 26% of the nucleotides of the double-stranded region were involved in G:U basepairs. The 148 canonical basepairs in hpGUS[G:U] were the same as in the control hairpin RNA, in the corresponding positions, including 49 U:A basepairs, 45 A:U basepairs and 54 G:C basepairs. The longest stretches of contiguous canonical basepairing in the double-stranded region was 9 basepairs. The antisense nucleotide sequence of hpGUS[G:U] was thereby identical in length (200 nt) and sequence to the antisense sequence of the control hairpin RNA hpGUS[wt]. After transcription of the expression cassette and splicing of the PDK intron from the primary transcript, the hairpin RNA structure prior to any processing by Dicer or other RNAses was predicted to have a loop structure of 45 nucleotides. The nucleotide sequence of the hairpin structure including its loop is provided as SEQ ID NO:16, and its free energy of folding was predicted to be −331.73 kcal/mol. As for hpGUS[wt], this was therefore an energetically stable hairpin structure, despite the 52 G:U basepairs which individually are much weaker than the G:C basepairs in hpGUS[wt].

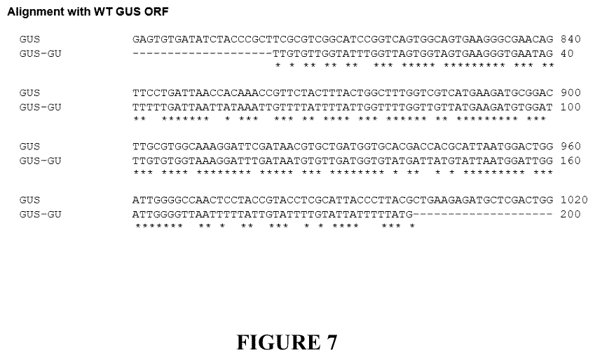

An alignment of the modified GUS sense sequence (nucleotides 9-208 of SEQ ID NO:11) with the corresponding region of the GUS target gene (SEQ ID NO:14) is shown in FIG. 7.

Construct hpGUS[1:4] Comprising Mismatched Nucleotides Every Fourth Nucleotide

A DNA fragment comprising the same 200 bp sense sequence, but in which every fourth nucleotide of the corresponding wild-type GUS sequence was substituted, was designed and assembled. Every 4th nucleotide in each block of 4 nucleotides (nucleotides at positions 4, 8, 12, 16, 20 etc) was substituted by changing C's to G's, G's to C's, A's to T's and T's to A's, leaving the other nucleotides unchanged. These substitutions were all transversion substitutions, which were expected to have a greater destabilising effect on the resultant hairpin RNA structure than transition substitutions. The DNA fragment was assembled by annealing the overlapping oligonucleotides GUS-4M-F (SEQ ID NO:56) and GUS-4M-R (SEQ ID NO:57) and PCR extension of 3′ ends using LongAmp Taq polymerase. The amplified DNA fragment was inserted into the pGEM-T Easy vector and the correct nucleotide sequence (SEQ ID NO: 12) was confirmed by sequencing. A DNA fragment comprising the modified sequence was then excised by digestion with XhoI and KpnI and inserted into the XhoI/KpnI sites of the base vector pMBW606. This produced the construct designated pMBW609, containing the expression cassette 35S::sense GUS[1:4]::PDK Intron::antisense GUS::OCS-T. This expression cassette was excised with NotI digestion and inserted into the NotI site of pART27, resulting in the vector designated hpGUS[1:4], encoding the 1:4 mismatched hairpin RNA molecule.

This cassette encoded a hairpin RNA targeting the GUS mRNA and which, when self-annealed by hybridisation of the sense and antisense sequences, had mismatches for 50 nucleotides of the 200 nt antisense sequence, including the mismatch for the nucleotide at position 200. Excluding position 200, the double-stranded region of the hairpin RNA had 150 canonical basepairs and 49 mismatched nucleotide pairs over a length of 199 nt sense and antisense sequences, i.e. 24.6% of the nucleotides of the double-stranded region were predicted to be mismatched (not involved in basepairs). After transcription of the expression cassette and splicing of the PDK intron from the primary transcript, the hairpin RNA structure prior to any processing by Dicer or other RNAses was predicted to have a loop structure of 45 nucleotides. The nucleotide sequence of the hairpin structure including its loop is provided as SEQ ID NO:17, and its free energy of folding was predicted to be −214.05 kcal/mol. As for hpGUS[wt], this was therefore an energetically stable hairpin structure, despite the mismatched nucleotides.

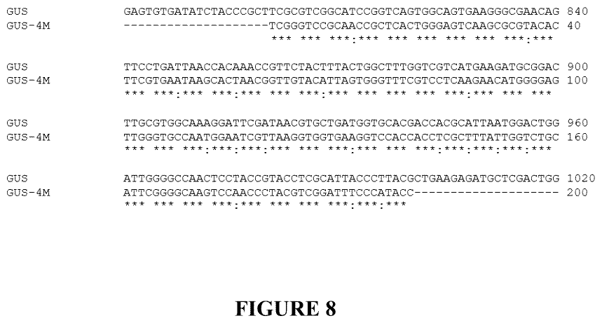

An alignment of the modified GUS sense sequence (nucleotides 9-208 of SEQ ID NO:12) with the corresponding region of the GUS target gene (SEQ ID NO:14) is shown in FIG. 8.

Construct hpGUS[2:10] in which Nucleotides 9 and 10 of 10 Nucleotides was Mismatched

A DNA fragment comprising the same 200 bp sense sequence, but in which every ninth and tenth nucleotide of the corresponding wild-type GUS sequence was substituted, was designed and assembled. Each 9th and 10^{th} nucleotide in each block of 10 nucleotides (nucleotides at positions 9, 10, 19, 20, 29, 30 etc) was substituted by changing C's to G's, G's to C's, A's to T's and T's to A's, leaving the other nucleotides unchanged. The DNA fragment was assembled by annealing the overlapping oligonucleotides GUS-10M-F (SEQ ID NO:58) and GUS-10M-R (SEQ ID NO:59) and PCR extension of 3′ ends using LongAmp Taq polymerase. The amplified DNA fragment was inserted into pGEM-T Easy and the correct nucleotide sequence (SEQ ID NO: 13) was confirmed by sequencing. A DNA fragment comprising the modified sequence was then excised by digestion with XhoI and KpnI and inserted into the XhoI/KpnI sites of the base vector pMBW606. This produced the construct designated pMBW610, containing the expression cassette 35S::sense GUS[2:10]::PDK Intron::antisense GUS::OCS-T. This expression cassette was excised with NotI digestion and inserted into the NotI site of pART27, resulting in the vector designated hpGUS[2:10], encoding the 2:10 mismatched hairpin RNA molecule.

This cassette encoded a hairpin RNA targeting the GUS mRNA which, when self-annealed by hybridisation of the sense and antisense sequences, had mismatches for 50 nucleotides of the 200 nt antisense sequence, including mismatches for the nucleotides at positions 199 and 200. Excluding positions 199 and 200, the double-stranded region of the hairpin RNA had 160 canonical basepairs and 19 di-nucleotide mismatches over a length of 198 nt sense and antisense sequences, i.e. 19.2% of the nucleotides of the double-stranded region were predicted to be mismatched (not involved in basepairs). The 160 basepairs in hpGUS[2:10] were the same as in the control hairpin RNA, in the corresponding positions, including 41 U:A basepairs, 34 A:U basepairs, 42 G:C and 43 C:G basepairs. After transcription of the expression cassette and splicing of the PDK intron from the primary transcript, the hairpin RNA structure prior to any processing by Dicer or other RNAses was predicted to have a loop structure of 45 nucleotides. The nucleotide sequence of the hairpin structure including its loop is provided as SEQ ID NO:18, and its free energy of folding was predicted to be −302.78 kcal/mol. As for hpGUS[wt], this was therefore an energetically stable hairpin structure, despite the mismatched nucleotides which were expected to bulge out of the stem of the hairpin structure.

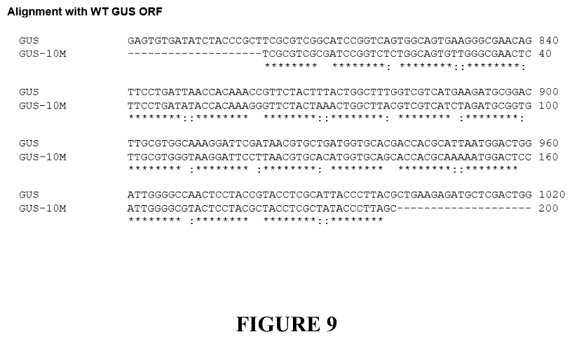

An alignment of the modified GUS sense sequence (nucleotides 9-208 of SEQ ID NO:13) with the corresponding region of the GUS target gene (SEQ ID NO:14) is shown in FIG. 9.

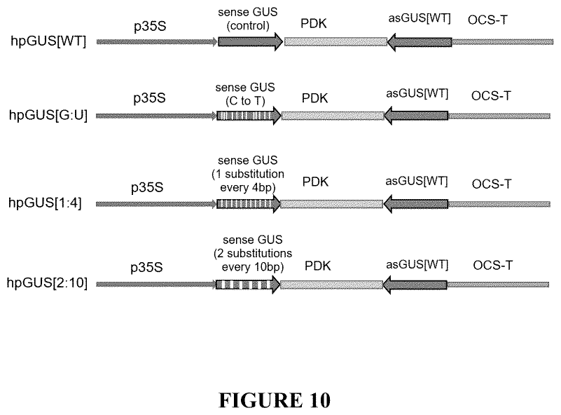

The four genetic constructs for expression of the control and modified hairpin RNAs are shown schematically in FIG. 10.

### Example 7. Testing the Modified Hairpin RNAs in Transgenic Plants

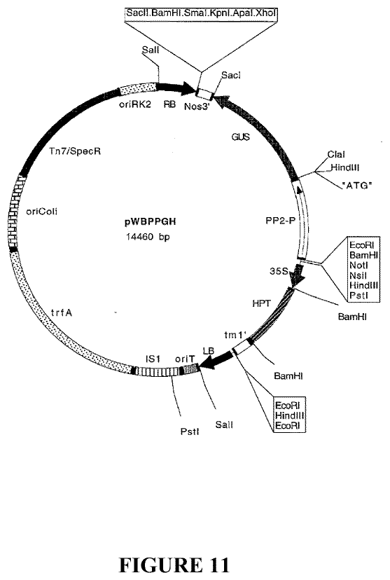

Plants of the species Nicotiana tabacum (tobacco) transformed with a GUS target gene were used to test the efficacy of the four hairpin RNA constructs described above. Specifically, the target plants were from two homozygous, independent transgenic lines, PPGH11 and PPGH24, each containing a single-copy insertion of a GUS transgene from a vector pWBPPGH which is shown schematically in FIG. 11. The GUS gene in the T-DNA of pWBPPGH had a GUS coding region (nucleotides 7-1812 of SEQ ID NO:8) operably linked to a 1.3 kb long promoter of the phloem protein 2 (PP2) gene from Cucurbita pepo L. cv. Autumn Gold (Wang et al., 1994; Wang, 1994). The construct pWBPPGH was made by excising the PP2 promoter plus the 5′ UTR and 54 nucleotides of the PP2 protein coding region, encoding the first 18 amino acids of PP2, from the lambda genomic clone CPP1.3 (Wang, 1994), and fusing this fragment with the GUS coding sequence starting with the nucleotides encoding the 3rd amino acid of GUS, generating an N-terminal fusion polypeptide having GUS activity. The pPP2::GUS:Nos-T cassette was inserted into pWBVec2a (Wang et al., 1998) to generate pWBPPGH, which was used to transform plants of Nicotiana tabacum cv. Wisconsin 38 using Agrobacterium tumefaciens-mediated leaf disk transformation (Ellis et al., 1987), selecting for resistance to hygromycin. GUS activities in homozygous progeny plants of two transgenic lines PPGH11 and PPGH24 were similar. GUS expression in both transgenic plants was not restricted to phloem but present in most tissues of the plants. GUS expression from the PP2 promoter in these plants therefore appeared to be constitutive. There were two reasons for choosing the PP2-GUS plants as the testing plants: i) they give constitutively high levels of GUS expression about the same as to a 35S-GUS plant; ii) the PP2 promoter is an endogenous PP2 gene promoter derived from Cucurbita pepo with a different sequence to the 35S promoter used to drive the expression of the hpRNA transgenes, which therefore would not be subject to transcriptional cosuppression by the incoming 35S promoter.

All four hairpin RNA constructs (Example 6) were used to transform PPGH11 and PPGH24 plants using the Agrobacterium-mediated leaf-disk method (Ellis et al., 1987), using 50 mg/L kanamycin as the selective agent. This selection system with kanamycin, a different agent to the previously used hygromycin used to introduce the T-DNA of pWBPPGH, was observed to yield only transformed plants, with no non-transformed plants being regenerated. Regenerated transgenic plants containing the T-DNAs from the hpGUS constructs were transferred to soil for growth in the greenhouse and maintained for about 4 weeks before assaying for GUS activity. When assayed, the transgenic plants were healthy and actively growing and in appearance were identical to non-transformed control plants and the parental PPGH11 and PPGH24 plants. In total, 59 transgenic plants were obtained that were transformed with the T-DNA encoding hpGUS[wt], 74 plants were obtained that were transformed with the T-DNA encoding hpGUS[G:U], 33 plants were obtained that were transformed with the T-DNA encoding hpGUS[1:4] and 41 plants were obtained that were transformed with the T-DNA encoding hpGUS[2:10].

GUS expression levels were measured using the fluorimetric 4-methylumbelliferyl β-D-glucuronide (MUG) assay (Jefferson et al., 1987) following the modified kinetic method described in Chen et al. (2005). Plants were assayed by taking leaf samples of about 1 cm diameter from three different leaves on each plant, choosing leaves which were well expanded, healthy and green. Care was taken that the test plants were at the same stage of growth and development as the control plants. Each assay used 5 μg protein extracted per leaf sample and measured the rate of cleavage of MUG as described in Chen et al. (2005).

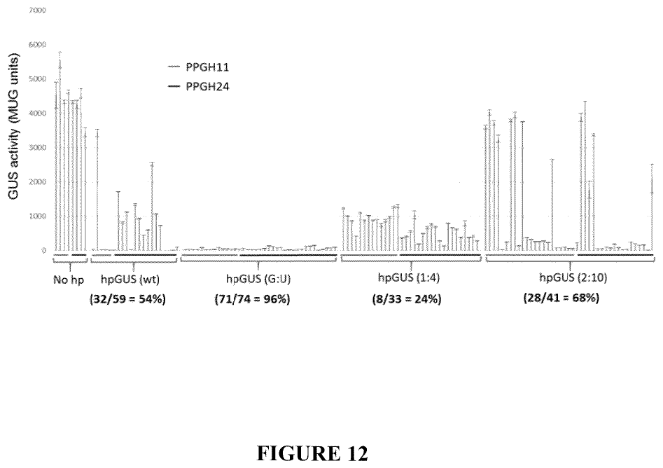

Representative data are shown in FIG. 12, showing GUS activity (MUG units in the assay) for each independent transgenic plant. Since the data for the hpGUS[wt] construct showed that some plants exhibited strong silencing with a reduction in activity of at least 90% and others weaker silencing, 10% GUS activity relative to the control plants was chosen, in this context, as an activity level for classifying the plants into two categories and comparing the different constructs.

The genetic construct encoding the canonically basepaired hpGUS[wt] induced strong GUS silencing, using the 10% activity level as the benchmark for strong silencing, in 32 of the 59 transgenic plants tested (54.2%). The other 27 plants all showed reduced GUS activity but retained more than 10% of the enzyme activity relative to the control plants, and so were considered to exhibit weak silencing in this context. The transgenic plants with this construct showed a wide range in the extent of GUS gene silencing (FIG. 12), from less than 1% to about 80% activity remaining, which was typical for conventional hairpin designs (Smith et al., 2000).

In clear contrast, the hpGUS[G:U] construct induced consistent and uniform silencing across the independent transgenic lines, with 71 of the 74 plants (95.9%) that were tested showing strong GUS silencing. Different again, all of the 33 hpGUS[1:4] plants tested showed reduced levels of GUS activity, with only 8 (24%) yielding <10% of the GUS activity relative to the control plants, and the other 25 classified as having weaker silencing. These results indicated that this construct induced weaker but more uniform levels of GUS down-regulation across the transgenic lines. The hpGUS[2:10] construct performed more like the hpGUS[wt] construct, inducing good levels of silencing in some lines (28 of 41, or 68.3%) and gave little or no GUS silencing in the remaining 13 plants.

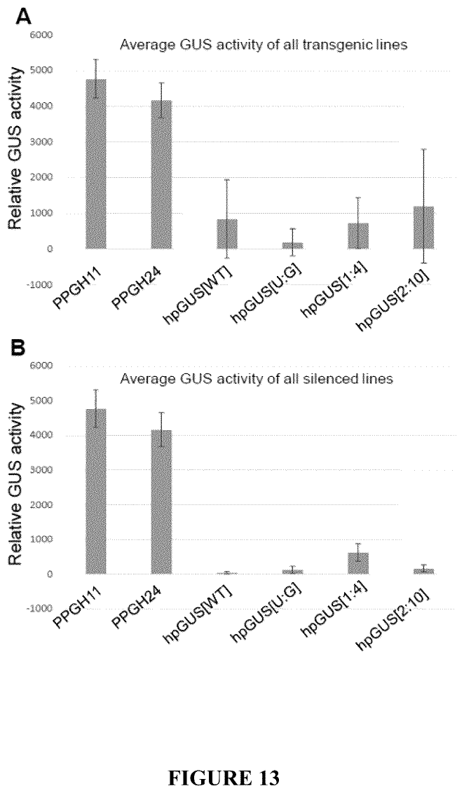

When only the silenced lines (<10% remaining activity) were used for comparison and average GUS activities calculated, the hpGUS[wt] plants showed the highest average extent of silencing, followed in order by the hpGUS[G:U] plants and the hpGUS[2:10] plants (FIG. 13). The hpGUS[1:4] plants showed the least average reduction in GUS activity. The extent of GUS silencing showed a good correlation with the thermodynamic stability of the predicted hpRNA structures derived from the four different hpRNA constructs (Example 6).

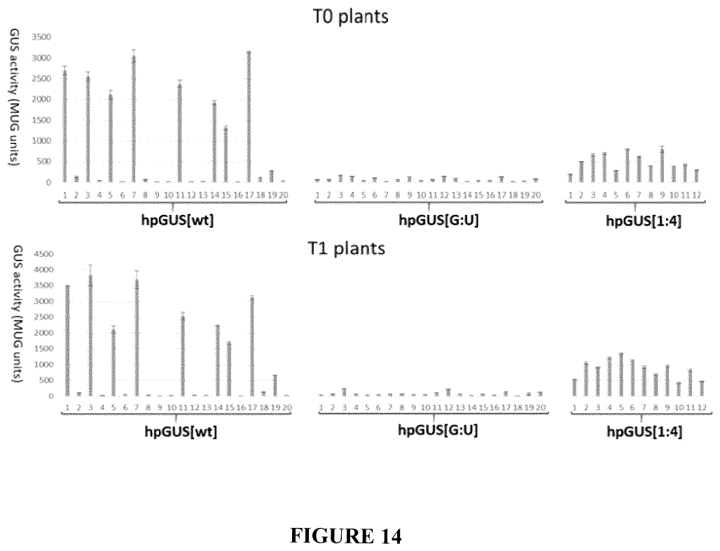

To test whether the differences would persist in progeny plants, representative transgenic plants containing both the target GUS gene, which was homozygous, and the hpGUS transgene (hemizygous) were self-fertilised. Kanamycin-resistant progeny plants from the hpGUS lines were selected, so discarding any null segregants lacking the hpGUS transgenes. This ensured that the hpGUS transgenes were present in all of the progeny, in either the homozygous or heterozygous state. The progeny plants were assayed for GUS activity and representative data are presented in FIG. 14. Progeny containing the hpGUS[wt] transgenes obviously fell into two categories, namely those that had strong GUS silencing and others that showed weak or no silencing. These classes correlated well with the phenotype of the previous generation, showing that the extent of target gene silencing was heritable. All of the plants in the hpGUS[G:U] lines tested consistently showed strong silencing, whilst the plants in the hpGUS[1:4] lines consistently showed weaker silencing. The inventors concluded that the phenotypes observed in the parental generation were generally maintained in the progeny plants.

### Southern Blot Hybridisation Experiments on Transformed Plants

The uniformity of the strong gene silencing observed in the large number of independent transgenic plants generated with the hpGUS[G:U] construct was striking as well as surprising and unexpected. The inventors sought to establish whether any explanation other than an effect caused by the hpGUS[G:U] RNA was causing the uniformity of the silencing. To test whether the multiple transgenic plants arose from independent transformation events as intended, Southern blot hybridisation experiments were carried out on DNA isolated from 18 representative transgenic plants containing the hpGUS[G:U] construct. DNA was isolated from leaf tissues using the hot-phenol method described by Wang et al. (2008). For Southern blot hybridization, approximately 10 μg of DNA from each plant sample was digested with HindIII enzyme, separated by gel electrophoresis in 1% agarose gels in TBE buffer, and blotted onto Hybond-N+ membrane using the capillary method (Sambrook et al., 1989). The membrane was hybridized overnight at 42° C. with a ³²P-labelled DNA fragment from the OCS-T terminator region. This probe was chosen as it hybridized to the hpGUS[G:U] transgene but not to the GUS target gene which did not have an OCS-T terminator sequence. The membrane was washed at high stringency and retained probe visualized with a PhosphoImager.

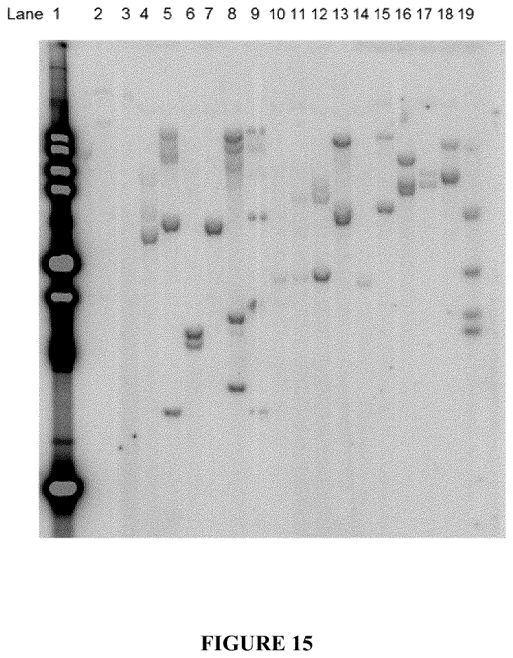

An autoradiograph of a hybridised blot is shown in FIG. 15. Each lane showed from one to five or six hybridising bands. No two lanes showed the same pattern i.e. the autoradiograph showed that the 16 representative hpGUS[G:U] plants each had different patterns of HindIII fragments that hybridized and therefore came from different transgene insertions. The inventors concluded that the uniform GUS silencing observed for hpGUS[G:U] lines was not due to similar transgene insertion patterns in the plants, and that the uniformity of silencing was caused by the structure of the hpGUS[G:U] RNA. The inventors also concluded that multiple copies of the hpGUS[G:U] transgene were not required in order to obtain strong gene silencing; a single copy of the transgene was sufficient.

### Northern Blot Hybridisation Experiments on Transformed Plants

To determine whether the hpGUS[G:U] RNA was processed in the same manner as the control hairpin RNA in the transgenic plants, Northern blot hybridisation experiments were carried out on RNA isolated from leaves of the transgenic plants. The Northern blot experiments were carried out to detect the shorter RNAs (sRNA, approx 21-24 nucleotides in length) which resulted from Dicer-processing of the hairpin RNAs. The experiment was carried out on small RNA isolated from transgenic hpGUS[wt] and hpGUS[G:U] plants which also containing the GUS target gene which was expressed as a (sense) mRNA. Nine plants for each construct were selected for sRNA analysis. For the hpGUS[wt]transgenic population, plants showing weak GUS silencing were included as well as some exhibiting strong GUS silencing. The small RNA samples were isolated using the hot-phenol method (Wang et al., 2008), and Northern blot hybridization was performed according to Wang et al. (2008), with gel electrophoresis of the RNA samples carried out under denaturing conditions. The probes used were 32P-labelled RNAs corresponding to either the sense sequence or the antisense sequence corresponding to nucleotides 804-1003 of SEQ ID NO:8.

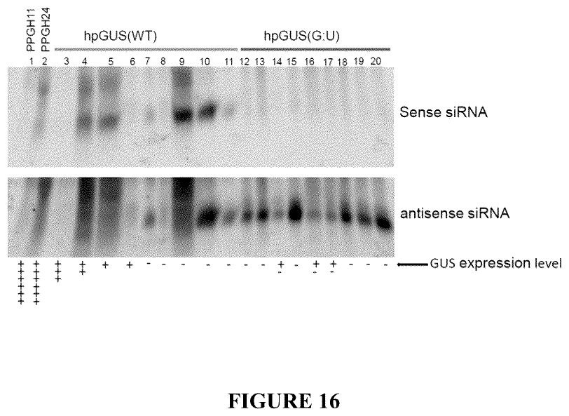

An autoradiograph of a Northern blot, hybridised with either the antisense probe (upper panel) to detect sense sRNA molecules derived from the hairpin RNAs, or hybridised with the sense probe to detect the antisense sRNAs (lower panel), is shown in FIG. 16. At the bottom, the Figure shows a qualitative score for the level of GUS expression relative to the control plants lacking the hpGUS constructs. Hybridisation to small RNAs of about 20-25 nucleotides was observed, based on the mobility of the sRNAs compared to RNAs of known length in other experiments. The hpGUS[wt] lines showed a range of variation in the amount of sRNA accumulation. This was observed for both the sense and antisense sRNAs, although the antisense sRNA bands were not as clear as the sense bands. Since the hpGUS[wt] plants contained both the hpGUS transgene, expressing both sense and antisense sequences corresponding to the 200 nt target region, and the GUS target gene expressing the full-length sense gene, the sense sRNAs could have been generated from either the hairpin RNA or the target mRNA. There appeared to be negative correlation between the level of sRNA and the degree of GUS silencing in the hpGUS[wt] plants. For example, the two plants represented in lanes 4 and 5 accumulated relatively more sRNA but showed only a moderate extent of GUS downregulation. In contrast, the two plants represented in lanes 7 and 8 had strong GUS silencing but accumulated relatively low levels of sRNA.

In contrast to the hpGUS[wt] plants and consistent with the relatively uniform extent of silencing by the hpGUS[G:U] construct, the hpGUS[G:U] plants accumulated uniform amounts of antisense sRNAs across the lines. Furthermore, the degree of GUS silencing appeared to show good correlation with the amount of antisense sRNA. Almost no sense sRNAs were detected in these plants. This was expected since the RNA probe used in the Northern blot hybridisation was transcribed from the wild-type GUS sequence and therefore had a lower level of complementarity to sense sRNAs from hpGUS[G:U] where all C nucleotides were replaced with U nucleotides, allowing only lower stringency hybridisation. However, this experiment did not exclude the possibility that the hpGUS[G:U] RNA was processed to produce less sense sRNAs or that they were degraded more quickly.

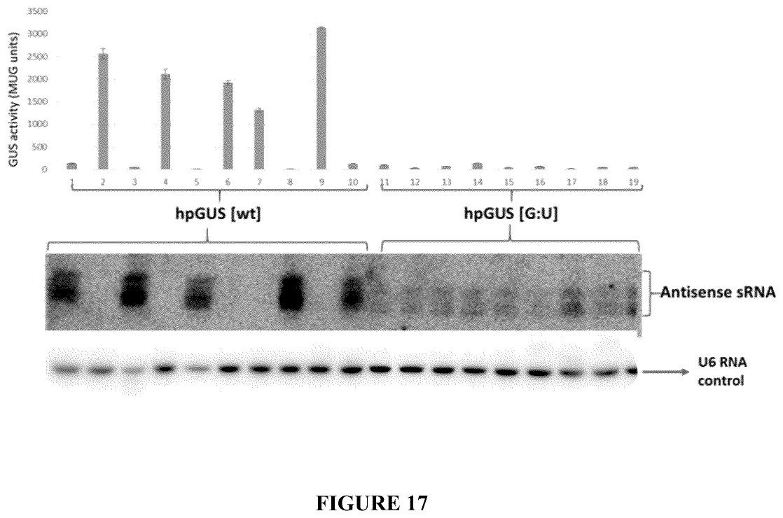

The Northern blot hybridisation experiment was repeated, this time using only a sense probe to detect antisense sRNAs; the autoradiograph is shown in FIG. 17. Once again, the production of antisense sRNAs from the hpGUS[wt] construct correlated negatively with the GUS activity (upper panel of FIG. 17). Plants which were strongly silenced yielded high levels of antisense sRNAs (lanes 1, 3, 5, 8 and 10) whereas plants that showed only weak or no silencing did not produce a hybridisation signal in this experiment (lanes 2, 4, 6, 7 and 9). In very clear contrast, the plants expressing hpGUS[G:U] produced a much lower, but consistent, amount of antisense sRNAs. The observation that the strongly silenced plants expressing hpGUS[G:U] accumulated much lower levels of sRNAs than the strongly silenced plants expressing hpGUS[wt] was intriguing and suggested to the inventors that the hpGUS [wt] was being processed by a different mechanism in the plants but was still about as effective as the hpGUS[wt] construct. A further observation in this experiment provided a clue in that the two, relatively faint antisense bands for the hpGUS[G:U] plants appeared to have the same mobility as the second and fourth bands observed for the antisense sRNA bands from hpGUS[wt]. This was confirmed in further experiments described below. The inventors postulated that the four bands for the sRNAs from hpGUS[wt] represent 24-, 22-, 21- and 20-mers, and that the hpGUS[G:U] RNA was processed primarily to produce 22- and 20-mers antisense sRNAs.

An important, definite conclusion from the data described above was that the hpGUS[G:U] RNA molecule was processed by one or more Dicer enzymes to produce sRNAs, in particular the production of antisense sRNAs which are thought to be mediators of RNA interference in the presence of various proteins such as Argonaute. The observed production of antisense sRNAs implied that the sense sRNAs were also produced, but the experiments did not distinguish between degradation/instability of the sense sRNAs or the lack of detection of sense sRNAs due to insufficient hybridisation with the probe that was used. From these experiments, the inventors also concluded that there were clear differences between the hpGUS[wt] and hpGUS[G:U] RNA molecules in their processing. This indicated that the molecules were recognised differently by one or more Dicers.

### Example 8. Analysis of sRNAs from Transgenic Plants Expressing Modified Hairpin RNAs

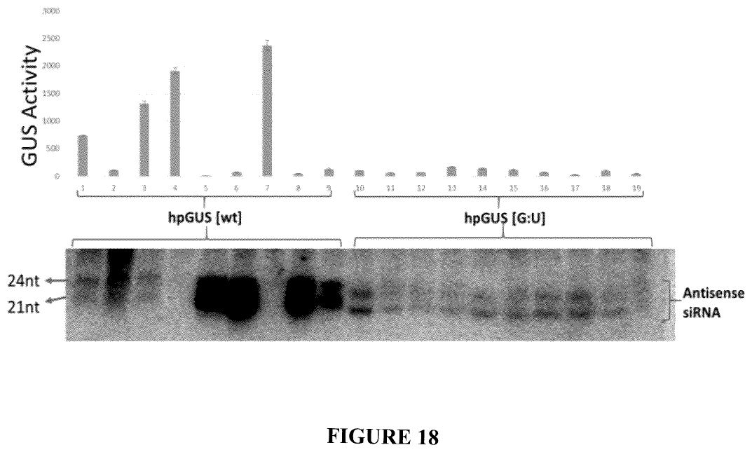

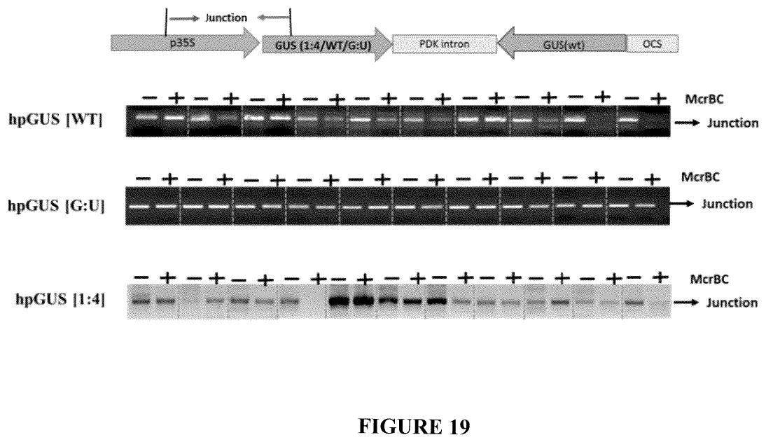

Another Northern blot hybridisation experiment was carried out to detect antisense sRNAs from hpGUS[G:U] plants and to compare their sizes to those produced from hpGUS[wt]. The autoradiograph is shown in FIG. 18. This time, the difference in size of the two antisense sRNA bands from hpGUS[G:U] compared to the main two bands from hpGUS[wt] was more distinct. This was best seen by comparing the mobility of the bands in adjacent lanes 9 and 10 of FIG. 18. This result confirmed that the two hairpin RNAs were processed differently by one or more Dicers in the plants.

To further investigate this, the small RNA populations from the hpGUS[wt] and hpGUS[G:U] were analysed by deep sequencing of the total, linker-amplifiable sRNAs isolated from the plants. The frequency of sRNAs which mapped to the double-stranded regions of the hairpin RNAs was determined. The length distribution of such sRNAs was also determined. The results showed that there was an increase in the frequency of 22-mer antisense RNAs from the hpGUS[G:U] construct relative to the hpGUS[wt] construct. The increase in the proportion of sRNAs of 22 nt in length indicated a shift in processing of the hpGUS[G:U] hairpin by Dicer-2 relative to hpGUS[wt].

### Example 9. DNA Methylation Analysis of Transgenes in Plants

The observations on the variability in the extent of GUS silencing conferred by hpGUS[wt] and that antisense 24-mer sRNAs were detected in the hpGUS[wt] plants but apparently not in the hpGUS[G:U] plants led the inventors to question whether the two populations of plants differed in their level of DNA methylation of the target GUS gene. Sequence-specific 24-mer sRNAs are thought to be involved in promoting DNA methylation of inverted repeat structures in plants (Dong et al., 2011). The inventors therefore tested the levels of DNA methylation of the GUS transgene in the hpGUS plants, in particular of the 35S promoter region of the hairpin encoding gene (silencing gene).

To do this, the DNA-methylation dependent endonuclease McrBC was used. McrBC is a commercially available endonuclease which cleaves DNA containing methylcytosine (^{m}C) bases on one or both strands of double-stranded DNA (Stewart et al., 2000). McrBC recognises sites on the DNA which consist of two half-sites of the form 5′ (G or A)^{m}C 3′, preferably G^{m}C. These half-sites may be separated by several hundred basepairs, but the optimal separation is from 55 to about 100 bp. Double-stranded DNA having such linked G^{m}C dinucleotides on both strands serve as the best substrate. McrBC activity is dependent on either one or both of the GC dinucleotides being methylated. Since plant DNA can be methylated at the C in CG, CHG or CHH sequences where H stands for A, C or T (Zhang et al., 2018), digestion of DNA using McrBC with subsequent PCR amplification of gene-specific sequences can be used to detect the presence or absence of ^{m}C in specific DNA sequences in plant genomes. In this assay, PCR amplification of McrBC-digested genomic DNA which is methylated yields reduced amounts of the amplification product compared to DNA which is not methylated, but will yield an equal amount of PCR product as untreated DNA if the DNA is not methylated.

Genomic DNA was isolated by standard methods from plants containing the hpGUS[wt], hpGUS[G:U] or hpGUS[1:4] construct in addition to the target GUS gene (Draper and Scott, 1988). Purified DNA samples were treated with McrBC (Catalog No. M0272; New England Biolabs, Massachusetts) according to the manufacturer's instructions, including the presence of Mg²⁺ ion and GTP required for endonuclease activity. In summary, approximately 1 μg of genomic DNA was digested with McrBC overnight in a 30 μl reaction volume. The digested DNA samples were diluted to 100 μl and regions of interest were PCR-amplified as follows.

The treated DNA samples were used in PCR reactions using the following primers. For the 35S-GUS junction sequence for hpGUS[wt]: Forward primer (35S-F3), 5′-TGGCTCCTACAAATGCCATC-3′ (SEQ ID NO:60); Reverse primer (GUSwt-R2), 5′-CARRAACTRTTCRCCCTTCAC-3′ (SEQ ID NO:61). For the 35S-GUS junction sequence for hpGUS[G:U]: Forward primer (GUSgu-R2), 5′-CAAAAACTATTCACCCTTCAC-3′ (SEQ ID NO:62); reverse primer (GUS4m-R2), CACRAARTRTACRCRCTTRAC (SEQ ID NO:63). For the 35S promoter sequence for both constructs: Forward primer (35S-F2), 5′-GAGGATCTAACAGAACTCGC-3′ (SEQ ID NO:64); reverse primer (35S-R1), 5′-CTCTCCAAATGAAATGAACTTCC-3′ (SEQ ID NO:65). In each case, R=A or G, Y=C or T. PCR reactions were performed with the following cycling conditions: 94° C. for 1 min, 35 cycles of 94° C. for 30 sec, 55° C. annealing for 45 sec, 68° C. extension for 1 min, and final extension at 68° C. for 5 min. PCR amplification products were electrophoresed and the intensity of the bands quantitated.

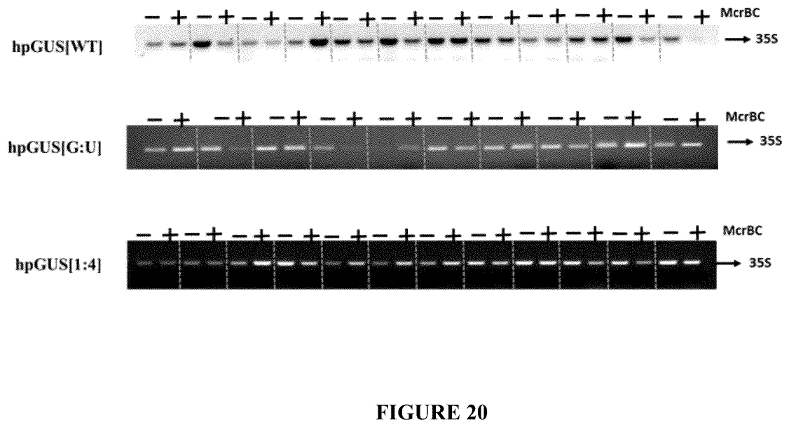

Representative results are shown in FIGS. 19 and 20. For the 35S-GUS junction region which included 200 bp of the 35S promoter sequence including the transcriptional start site, most of hpGUS[wt] plants showed significant levels of DNA methylation. Within the population of hpGUS[wt] plants, individual plants that retained higher levels of GUS activity i.e. less silencing, appeared to have more methylation of the promoter-GUS sense junction region. The results were similar for the 35S promoter region. In contrast, most of the hpGUS[G:U] and hpGUS[1:4] plants showed weaker DNA methylation at the 35S-GUS junction. The inventors considered that this proximal promoter sequence was important for expression of the transgene and methylation at this region would be likely to reduce expression of the silencing construct through transcriptional gene silencing (TGS) of the transgene. This is termed “self-silencing”.

### General Discussion in Relation to Examples 6 to 9

### Disruption of Inverted Repeat DNA Structure in a Transgene Enhances its Stability

Both of the populations of hpGUS[wt] and hpGUS[2:10]transgenic plants showed a wide range in the extent target gene silencing. In contrast, both of the populations containing hpGUS[G:U] and hpGUS[1:4] plants displayed relatively uniform GUS silencing in many independent lines, with strong silencing observed by the former construct and relatively weaker but still substantial reduction in gene activity by the latter construct. In the hairpin RNAs from the [G:U] and [1:4] constructs, about 25% of the nucleotides in the sense and antisense sequences were either involved in G:U basepairs or in a sequence mismatch that were evenly distributed across the 200 nucleotide sense/antisense sequences. Because of the sequence divergence between the sense and antisense sequences, the mismatches in the DNA constructs between the sense and antisense “arms” or the inverted request structure were considered to significantly disrupt that inverted-repeat DNA structure. Repetitive DNA structures may attract DNA methylation and silencing in various organisms (Hsieh and Fire, 2000). The hpGUS[2:10] construct also comprised mismatches between the sense and antisense region, but each of the 2 bp mismatches between the sense and antisense sequences were flanked by 8-bp consecutive matches, so the mismatches may not have disrupted the inverted repeat DNA structure as much as in the [G:U] and [1:4] transgenes. The uniformity of the GUS silencing induced by the hpGUS[G:U] and hpRNA[1:4] might therefore have been due, at least in part, to disruption of the inverted-repeat DNA structure that resulted in less methylation and therefore reduced the self-silencing of the two transgenes. Another benefit of the mismatches between the sense and antisense DNA regions was that cloning of the inverted repeat in E. coli was aided since the bacteria tend to delete or re-arrange perfect inverted repeats.

Thermodynamic Stability of hpRNA is Important for the Degree of Target Gene Silencing

When only the strongly-silenced transgenic lines were compared, the hpGUS[wt] plants had the greatest extent of target gene downregulation, followed in order by hpGUS[G:U], hpGUS[2:10] and hpGUS[1:4]. RNAFold analysis predicted that the hpGUS[wt] hairpin RNA structure had the lowest free energy, i.e. the greatest stability, followed by hpGUS[G:U], hpGUS[2:10] and hpGUS[1:4] hairpins. The inventors considered that the more stable the hairpin RNA structure, the greater the extent of target gene silencing it could induce. This also favoured longer double-stranded RNA structures rather than shorter ones. Stable double-stranded RNA formation was thought to be required for efficient Dicer processing. The results of the experiments described here indicated another important advantage of the G:U basepaired construct over the constructs comprising mostly simple mismatched nucleotides such as hpGUS[1:4]: while both types of constructs had disrupted inverted repeat DNA structures which reduced self-silencing, at the RNA level the hpGUS[G:U] RNA was more stable due to the ability of G and U to form basepairs. A combination of the two types of modifications was also considered beneficial, including both G:U basepairs and some mismatched nucleotides in the double-stranded RNA structure but with relatively more nucleotides involved in G:U basepairs than in mismatches, by a factor of at least 2, 3, 4 or even 5.

The hpGUS[G:U] RNA was Efficiently Processed by Dicer

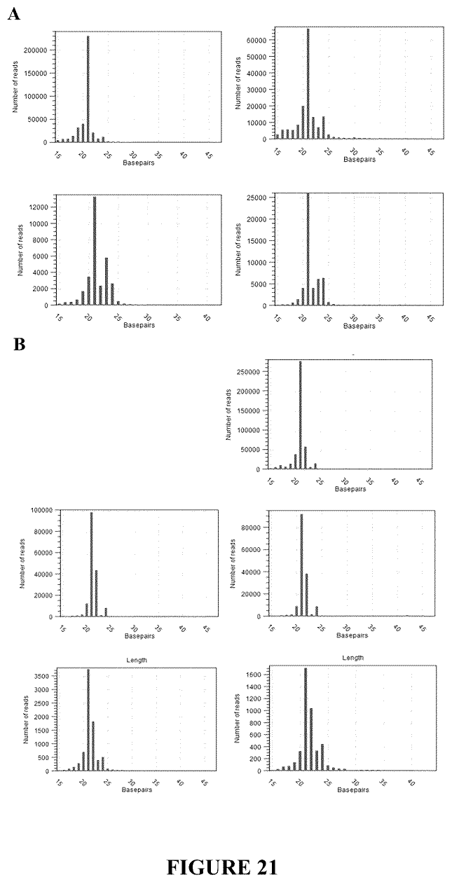

One important question that was answered in these experiments was whether the mismatched or G:U basepaired hpRNA could be processed by Dicer into small RNAs (sRNAs). The strong silencing in the hpGUS[G:U] plants and in the 1:4 and 2:10 mismatched hpRNA plants, implied that these hairpin RNA structures were processed by Dicer. This was confirmed for the [G:U] molecule by sRNA Northern blot hybridization, which readily detected antisense sRNAs. Furthermore, the degree of GUS silencing in the hpGUS[G:U] plants showed a good correlation with the amount of antisense sRNAs that accumulated. Small RNA deep sequencing analysis of two selected lines from each (only one for hpGUS[wt]) confirmed that hpGUS[G:U] plants, like the hpGUS[wt] plants, generated abundant sRNAs, whereas the hpGUS[1:4] plants also generated sRNAs but with a much lower abundance (FIG. 21). The lower level of sRNAs from the hpGUS[1:4] plants was consistent with the relatively low efficiency of GUS silencing and suggested that the low thermodynamic stability of the dsRNA stem in hpGUS[1:4] RNA reduced Dicer processing efficiency. It was noted that the extent of GUS silencing showed relatively poor correlation with the level of sRNA for the hpGUS[wt] construct, with some strongly silenced lines containing relatively low amounts of sRNA. This suggested that GUS silencing in some of the hpGUS[wt] lines was due at least in part to transcriptional silencing rather than sRNA-directed PTGS. The inventors recognised that the self-silencing of the hairpin-encoding gene, which involved methylation of the gene sequences such as the promoter region, was lessened by using the modified hairpin RNA constructs, particularly the G:U construct.

The G:U and 1:4 hpRNA Transgenes Showed Reduced DNA Methylation in the Proximal 35S Promoter Region

McrBC digestion-PCR analysis showed that DNA methylation levels in the 240 bp 35S sequence near the transcription start site (TSS) was reduced in the hpGUS[G:U] and hpGUS[1:4]transgenic populations relative to the hpGUS[wt] population. This result indicated to the inventors that the disruption of the perfect inverted-repeat structure, due to the C to T modifications (in hpGUS[G:U]) or 25% nucleotide mismatches (in hpGUS[1:4]) in the sense sequence, minimized transcriptional self-silencing of the hpRNA transgenes. This was consistent with the uniformity of GUS gene silencing observed in the hpGUS[G:U] and hpGUS[1:4] populations relative to the hpGUS[wt] population. The inventors recognised that the hpGUS[G:U] construct was more ideal than the hpGUS[1:4] construct in reducing promoter methylation hence transcriptional self-silencing at least because it had a reduced number, or even lacked, cytosine nucleotides in the sense sequence and therefore did not attract DNA methylation that could spread to the promoter.

### Example 10. Design and Testing of Hairpin RNAs Comprising G:U Basepairs Targeting Endogenous Genes

### Modified Hairpin RNAs Targeting EIN2 and CHS RNAs

Since the G:U modified hairpin RNA appeared to induce more consistent and uniform silencing of the target gene compared to the conventional hairpin RNA as described above, the inventors wanted to test whether the improved design would also reduce expression of endogenous genes. The inventors therefore designed, produced and tested several [G:U]-modified hairpin RNA constructs targeting either the EIN2 or CHS genes, or both, which were endogenous genes in Arabidopsis thaliana chosen as exemplary target genes for attempted silencing. The EIN2 gene (SEQ ID NO:19) encodes ethylene-insensitive protein 2 (EIN2) which is a central factor in signalling pathways regulated by the plant signalling molecule ethylene, i.e. a regulatory protein, and the CHS gene (SEQ ID NO:20) encodes the enzyme chalcone synthase (CHS) which is involved in anthocyanin production in the seedcoat in A. thaliana. Another G:U modified construct was produced which simultaneously targeted both of the EIN2 and CHS genes, in which the EIN2 and CHS sequences were transcriptionally fused to produce a single hairpin RNA. Furthermore, three additional constructs were made targeting either EIN2, CHS or both EIN2 and CHS, in which cytidine bases in both the sense and antisense sequences were replaced with thymidine bases (herein designated a G:U/U:G construct), rather than in just the sense sequence as done for the modified hairpins targeting GUS. The modified hairpin RNA constructs were tested for their ability to reduce the expression of the endogenous EIN2 gene or the EIN2 and CHS genes using a gene-delivered approach to provide the hairpin RNAs to the cells. The conventional hairpin RNAs used as the controls in the experiment had a double-stranded RNA region of 200 basepairs in length for targeting the EIN2 or CHS mRNAs, singly, or a chimeric double-stranded RNA region comprising 200 basepairs from each of the EIN2 and CHS genes which were fused together as a single hairpin molecule. In the fused RNA, the EIN2 double-stranded portion was adjacent to the loop of the hairpin and the CHS region was distal to the loop. All of the basepairs in the double-stranded region of the control hairpin RNAs were canonical basepairs.

### Construct Preparation

DNA fragments spanning the 200 bp regions of the wild-type EIN2 (SEQ ID NO:19) and CHS cDNAs (SEQ ID NO:20) were PCR-amplified from Arabidopsis thaliana Col-0 cDNA using the oligonucleotide primer pairs EIN2 wt-F (SEQ ID NO:66) and EIN2 wt-R (SEQ ID NO:67) or CHSwt-F (SEQ ID NO:68) and CHSwt-R (SEQ ID NO:69), respectively. The fragments were inserted into pGEMT-Easy as for the GUS hairpin constructs (Example 6). DNA fragments comprising the 200 bp modified sense EIN2[G:U] (SEQ ID NO:22) and CHS[G:U] (SEQ ID NO:24) fragments or the 200 bp modified antisense EIN2[G:U](SEQ ID NO:25) and modified antisense CHS[G:U] (SEQ ID NO:26) fragments, each flanked by restriction enzyme sites, were assembled by annealing of the respective pairs of oligonucleotides, EIN2gu-F+EIN2gu-R, CHSgu-F+CHSgu-R, asEIN2gu-F+asEIN2gu-R, and asCHSgu-F+asCHSgu-R (SEQ ID NOs:70-77), followed by PCR extension of 3′ ends using LongAmp Taq polymerase. All the G:U-modified PCR fragments were cloned into pGEM-T Easy vector and the intended nucleotide sequences confirmed by sequencing. The CHS[wt]::EIN2[wt], CHS[G:U]:EIN2[G:U], and asCHS[G:U]::asEIN2[G:U] fusion fragments were prepared by ligating the appropriate CHS and EIN2 DNA fragments at the common XbaI site in the pGEM-T Easy plasmid.

The 35S::sense fragment::PDK intron::antisense fragment::OCS-T cassettes were prepared in an analogous manner as for the hpGUS constructs. Essentially, the antisense fragments were excised from the respective pGEM-T Easy plasmids by digestion with HindIII and BamHI, and inserted into pKannibal between the BamHI and HindIII sites so they would be in the antisense orientation relative to the 35S promoter. The sense fragments were then excised from the respective pGEM-T Easy plasmid using XhoI and KpnI and inserted into the same sites of the appropriate antisense-containing clone. All of the cassettes in the pGEM-T Easy plasmids were then excised with Nod and inserted into pART27 to form the final binary vectors for plant transformation.

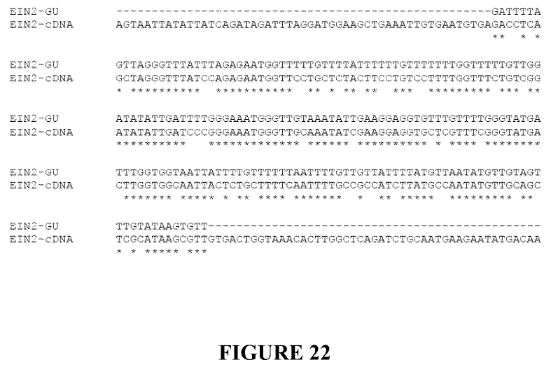

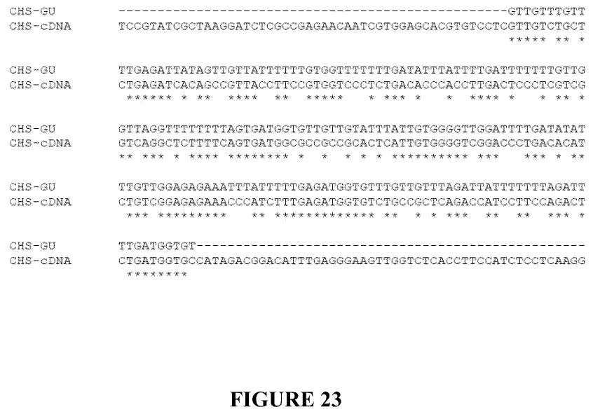

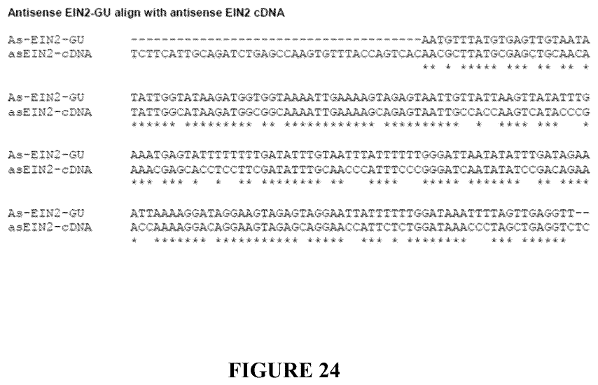

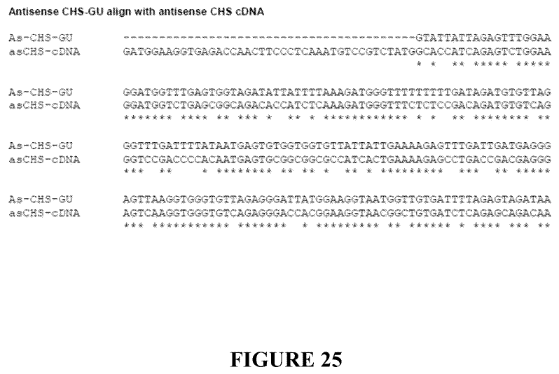

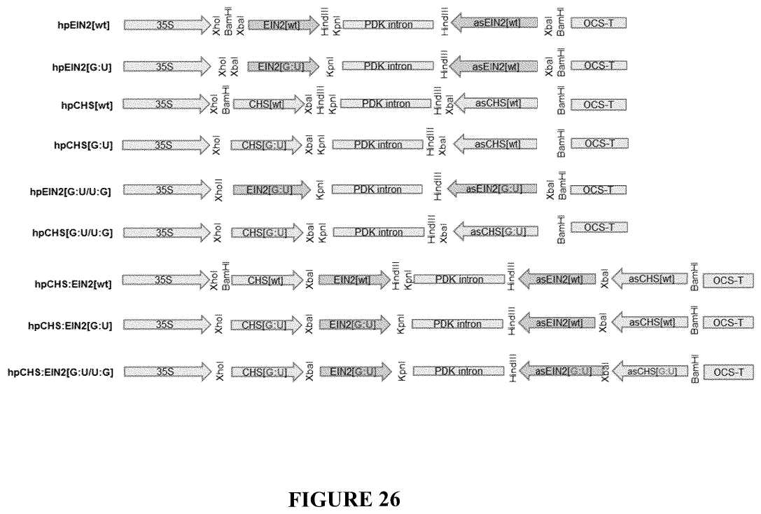

The alignments of the modified sense[G:U] and antisense[G:U] nucleotide sequences with the corresponding wild-type sequences, showing the positions of the substituted nucleotides, are shown in FIGS. 22 to 25. The designs of the expression cassettes for the hairpin RNAs are shown schematically in FIG. 26.

The predicted free energy of formation of the hairpin RNAs was estimated by using the FOLD program. These were calculated as (kcal/mol): hpEIN2[wt], −453.5; hpEIN2[G:U], −328.1; hpCHS[wt], −507.7; hpCHS[G:U]−328.5; hpEIN2[G:U/U:G], −173.5; hpCHS[G:Y/U:G], −186.0; hpCHS::EIN2[wt], −916.4; hpCHS::EIN2[G:U], −630.9; hpCHS::EIN2[G:U/U:G), −333.8.

### Plant Transformation

All of the EIN2, CHS and chimeric EIN2/CHS constructs were used to transform Arabidopsis thaliana race Col-0 plants using the floral dip method (Clough and Bent, 1998). To select for transgenic plants, seeds collected from the Agrobacterium-dipped flowers were sterilized with chlorine gas and plated on MS medium containing 50 mg/L kanamycin. Multiple transgenic lines were obtained for all nine constructs (Table 1). These primary transformants (T1 generation) were transferred to soil, self-fertilised and grown to maturity. Seed collected from these plants (T2 seed) was used to establish T2 plants and screened for lines that were homozygous for the transgene. These were used for analysing EIN2 and CHS silencing.

TABLE 1
Summary of transgenic plants
obtained in Col-0 background

| | Number of |
|---|---|
| | transgenic |
| Construct | lines obtained |
| hpEIN2[wt] | 46 |
| hpCHS[wt] | 34 |
| hpEIN2[G:U] | 23 |
| hpCHS[G:U] | 32 |
| hpEIN2[G:U/U:G] | 52 |
| hpCHS[G:U/U:G] | 13 |
| hpCHS::EIN2[wt] | 28 |
| hpCHS::EIN2[G:U] | 26 |
| hpCHS::EIN2[G:U/U:G] | 20 |

### Analysis of the Extent of EIN2 Silencing

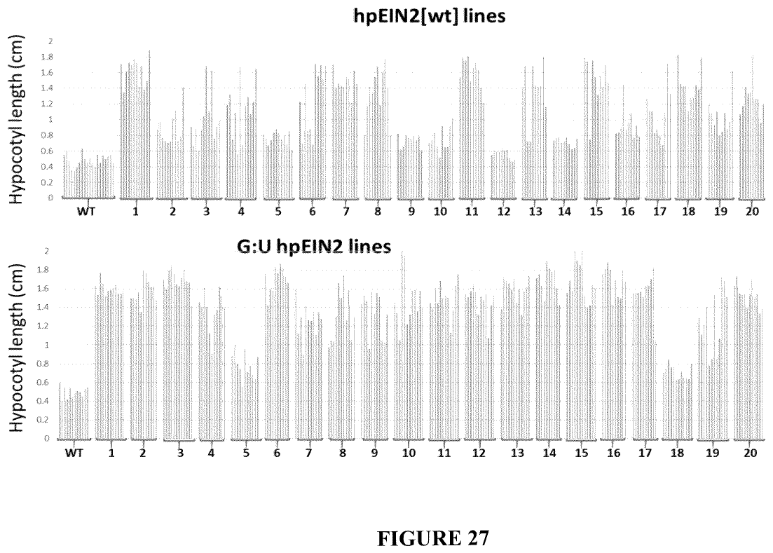

EIN2 is a gene in A. thaliana that encodes a receptor protein involved in ethylene perception. The gene is expressed in seedlings soon after germination of seeds as well as later in plant growth and development. EIN2 mutant seedlings exhibit hypocotyl elongation relative to isogenic wild-type seedlings when germinated in the dark in the presence of 1-aminocyclopropane-1-carboxylic acid (ACC), an intermediate in the synthesis of ethylene in plants. EIN2 gene expression and the extent of silencing in the transgenic plants was therefore assayed by germinating seed on MS medium containing 50 μg/L of ACC in total darkness and measuring their hypocotyl length, compared to the wild-type seedlings. The hypocotyl length was an easy phenotype to measure and was a good indicator of the extent of reduction in EIN2 gene expression, indicating different levels of EIN2 silencing. Plants with silenced EIN2 gene expression were expected to have various degrees of hypocotyl elongation depending on the level of EIN2 silencing, somewhere in the range between wild-type seedlings (short hypocotyls) and null-mutant seedlings (long hypocotyls). Seeds from 20 randomly selected, independently transformed plants for each construct were assayed. Seeds from one plant of the 20 containing the hpCHS::EIN2[G:U] construct did not germinate. The data for hypocotyl length are shown in FIG. 27.

The hpEIN2[wt] lines showed a considerable range in the extent of EIN2 silencing, with 7 lines (plant lines 2, 5, 9, 10, 12, 14, 16 in FIG. 27) clearly showing low levels of silencing or the same hypocotyl length relative to the wild-type, and the other 13 lines having moderate to strong EIN2 silencing. Individual plants within each independent line tended to exhibit a range in the extent of EIN2 silencing, as indicated by differences in hypocotyl length. In contrast, only two lines (plant lines 5, 18 in FIG. 27) comprising the hpEIN2[G:U]construct showed weak EIN2 silencing, with the remaining 18 showing uniform, strong EIN2 silencing. In addition, individual plants within each of the 18 lines appeared to have relatively uniform EIN2 silencing compared to the plants transformed with the hpEIN2[wt] construct. The inventors concluded that the G:U modified hairpin RNA construct was able to confer more consistent, less variable gene silencing of an endogenous gene which was more uniform and more predictable than the conventional hairpin RNA targeting the same region of the endogenous RNA.

The transgenic hpEIN2[wt] and hpEIN2[G:U] populations also differed in the relationship between the extent of EIN2 silencing and the transgene copy number. The transgene copy number was indicated by the segregation ratios for the kanamycin resistance marker gene in progeny plants—a 3:1 ratio of resistant:susceptible seedlings indicating a single locus insertion, whereas a ratio that was much higher indicated multi-loci transgene insertions. Several multiple copy-number lines transformed with the hpEIN2[wt] construct showed low levels of EIN2 silencing, but this was not the case for the hpEIN2[G:U] lines where both the single and multi-copy loci lines showed strong EIN2 silencing.

The EIN2 gene was also silencing in the seedlings transformed with the CHS::EIN2 fusion hairpin RNA. Similar to the plants containing the single hpEIN2[G:U] construct, the hpCHS::EIN2[G:U] seedlings clearly showed more uniform EIN2 silencing across the independent lines than the hpCHS::EIN2[wt] seedlings. The silencing among individual plants within an independent line also appeared to be more uniform for the hpCHS::EIN2[G:U] lines than the hpCHS::EIN2[wt] lines. At the same time, the extent of EIN2 silencing was slightly stronger for the highly silenced hpCHS::EIN2[wt] plants than for the hpCHS::EIN2[G:U] plants, similar to the comparison between plants transformed with hpGUS[wt] and hpGUS[G:U]. Comparison of the extent of silencing indicated that the fusion constructs did not induce stronger EIN2 silencing than the single hpEIN2[G:U] construct, indeed, the fusion G:U hairpin construct appeared to induce slightly weaker EIN2 silencing than the single gene-targeted hpEIN2[G:U] construct.

When the plants transformed with the G:U/U:G constructs were examined, where the cytidine (C) nucleotides of both the sense and antisense sequences were modified to thymidine (T) nucleotides, little to no increase in hypocotyl length was observed for all 20 independent lines analysed compared to wild-type plants. This was observed for both the hpEIN2[G:U/U:G] and hpCHS::EIN2[G:U/U:G] constructs. These results indicated to the inventors that the G:U/U:G basepaired hairpin RNA constructs having about 46% substitutions were not effective at inducing target gene silencing, perhaps because the basepairing of the hairpin RNAs had been destabilised too much. The inventors considered that two possible reasons might have contributed to the ineffectiveness. Firstly, the EIN2 double-stranded region of the hairpin RNAs had 92 G:U basepairs of the 200 potential basepairs between the sense and antisense sequences. Secondly, the alignment of the modified antisense sequence with the complement of the wild-type sense sequence showed that the 49 C to T replacements in the antisense sequence might have reduced the effectiveness of the antisense sequence to target the EIN2 mRNA. The inventors concluded from this experiment that, at least for the EIN2 target gene, there was an upper limit to the number of nucleotide substitutions that could be tolerated in the hairpin RNA and still maintain sufficient effectiveness for silencing. For instance, 92/200=46% substitutions was probably too high a percentage.

Analysis of the extent of CHS silencing Transgenic plants were assayed for the level of CHS gene expression by quantitative reverse transcription PCR (qRT-PCR) on RNA extracted from the whole plants, grown in vitro on tissue culture medium. The primers used for the CHS mRNA were: forward primer (CHS-200-F2), 5′-GACATGCCTGGTGCTGACTA-3′ (SEQ ID NO:78); reverse primer (CHS-200-R2) 5′-CCTTAGCGATACGGAGGACA-3′ (SEQ ID NO:79). The primers used for the reference gene Actin2 used as a standard were: Forward primer (Actin2-For) 5′-TCCCTCAGCACATTCCAGCA-3′ (SEQ ID NO:80) and reverse primer (Actin2-Rev) 5′-GATCCCATTCATAAAACCCCAG-3′ (SEQ ID NO:81).

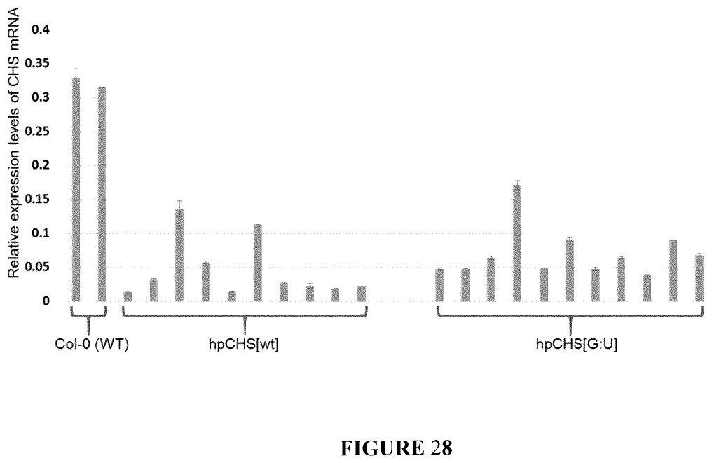

The data showed that the level of CHS mRNA the accumulated in the plants relative to the reference mRNA for the Actin2 gene was decreased in the range of 50-96% (FIG. 28).

A. thaliana seed completely lacking CHS activity have a pale seed coat colour compared to the brown colour of wild-type seeds. Therefore, seed of the transgenic plants were examined visually for their seedcoat colour. An obvious reduction of seed coat colour was observed in seeds from several plants but not in other plants, despite the reduction in CHS mRNA in the leaves of those plants. It was considered, however, that the seed coat colour phenotype was exhibited only when CHS activity was almost completely abolished in the developing seed coat during growth of the plants. Moreover, the 35S promoter may not have been sufficiently active in the developing seed coat to provide the level of reduction in CHS activity to provide for the pale seed phenotype seen in null mutants. Improvement in the visual seed coat colour phenotype could be gained by using a promoter that is more active in the seed coat of the seed.

### Example 11. Analysis of sRNAs from hairpin RNA constructs

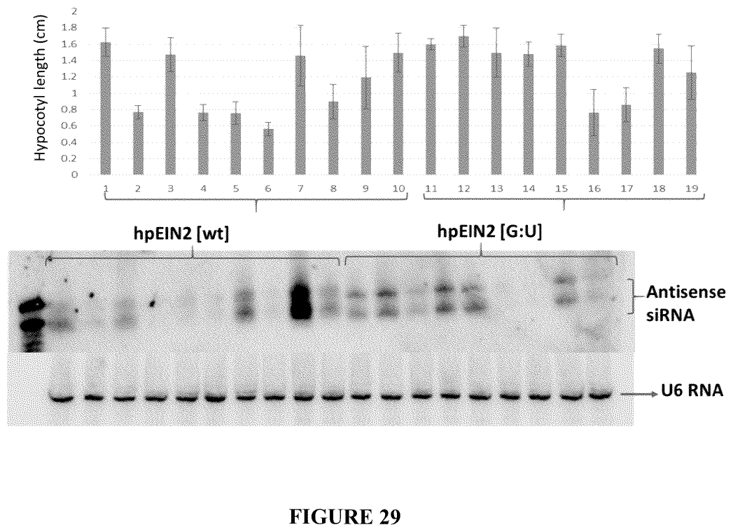

Northern blot hybridisation was carried out on RNA samples to detect antisense sRNAs from hpEIN2[G:U] plants and to compare their amount and their sizes to sRNAs produced from hpEIN2[wt]. The probe was a ³²P-labelled RNA probe corresponding to the 200 nucleotide sense sequence in the hpEIN2[wt] construct and hybridisation was carried out under low stringency conditions to allow for the detection of shorter (20-24 nucleotides) sequences. The autoradiograph from the probed Northern blot is shown in FIG. 29. This experiment showed that the hpEIN2[G:U] hairpin RNA was processed into sRNAs and the level of accumulation was relatively uniform across the 9 independently transformed hpEIN2[G:U] plants analysed compared to those of the hpEIN2[wt] lines. Similar to the analogous experiment for the GUS hairpin RNAs, a difference in size of the two antisense sRNA bands from hpEIN2[G:U] compared to the main two bands from hpEIN2[wt] was quite evident. This was best seen by comparing the mobility of the bands in adjacent lanes 10 and 11 of FIG. 28.

To further investigate this, the small RNA populations from the hpEIN2[wt] and hpEIN2[G:U] are analysed by deep sequencing of the total sRNA populations isolated from whole plants. The proportion of each population that mapped to the double-stranded regions of the hpEIN2[wt] and hpEIN2[G:U] was determined. From about 16 million reads in each population, about 50,000 sRNAs mapped to the hpEIN2[wt] double-stranded region, whereas only about 700 mapped to hpEIN2[G:U]. This indicated that many fewer sRNAs were generated from the [G:U] hairpin. An increase in the proportion of EIN2-specific 22-mers was also observed.

### Example 12. DNA Methylation Analysis of EIN2 Silenced Plants

Both the GUS and the EIN2 silencing results indicated that the hpRNA constructs having unmodified sense sequences induced highly variable levels of target gene silencing compared to the constructs having modified sense sequences providing for G:U basepairs. As described above, the promoter region of the hpGUS[G:U] construct appeared to have less methylation compared to the hpGUS[wt] construct. To test for DNA methylation and compare the hpEIN2[wt] and hpEIN2[G:U]transgenic plants, 12 plants from each population were analysed for DNA methylation at the 35S promoter and the 35S-promoter-sense EIN2 junction region using the McrBC method. The primers used for the 35S promoter region: Forward primer (Top-35S-F2), 5′-AGAAAATYTTYGTYAAYATGGTGG-3′ (SEQ ID NO:82), reverse primer (Top-35S-R2), 5′-TCARTRRARATRTCACATCAATCC-3′ (SEQ ID NO:83). The primers used for the 35S promoter-sense EIN2 junction region: Forward primer (Link-35S-F2), 5′-YYATYATTGYGATAAAGGAAAGG-3′ (SEQ ID NO:84) and reverse primer (Link-EIN2-R2), 5′-TAATTRCCACCAARTCATACCC-3′ (SEQ ID NO:85). In each ofthese primer sequences, Y=C or T and R=A or G.

Quantitation of the extent of DNA methylation was determined by carrying out Real-Time PCR assays. For each plant, the quotient was calculated: rate of amplification of the DNA fragment after treatment of the genomic DNA with McrBC/rate of amplification of the DNA fragment without treatment of the genomic DNA with McrBC.

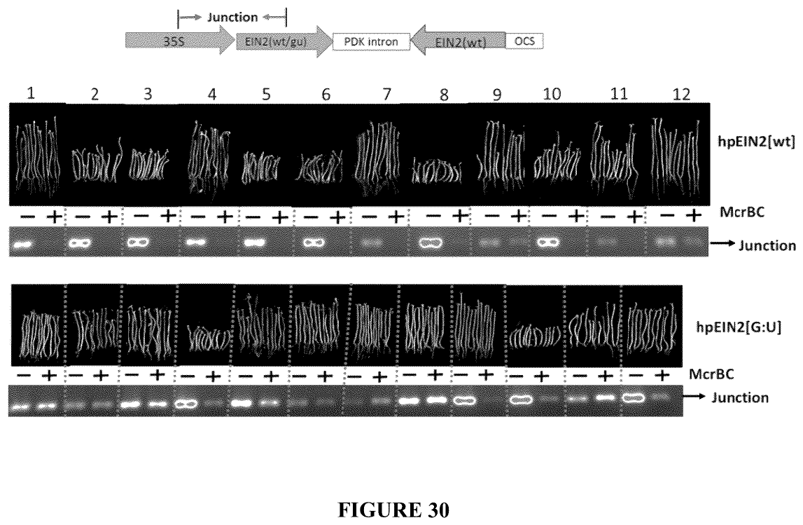

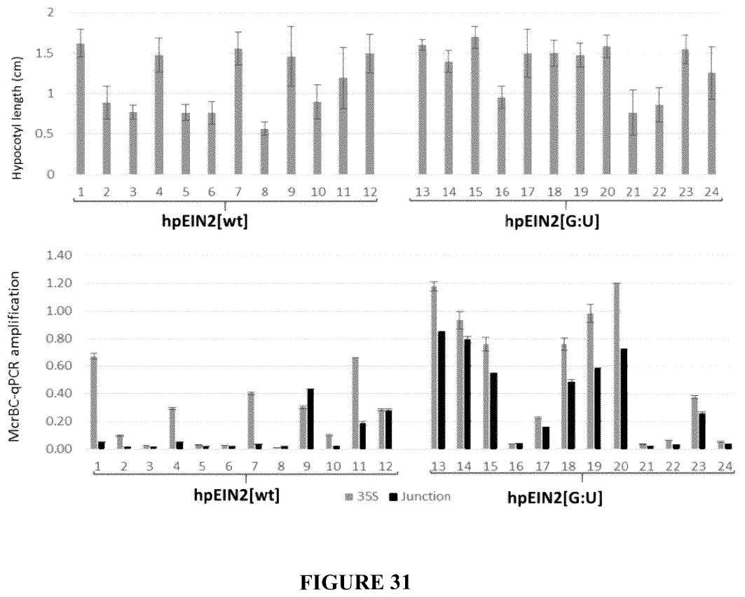

Almost every hpEIN2[wt] plant showed significant levels of DNA methylation at the 35S promoter, particularly at the 35S-EIN2 junction, but some more than others. As shown in FIGS. 30 and 31, the plant lines represented in lanes 1, 4, 7, 9, 11 and 12 all showed strong EIN2 silencing as shown by the longer hypocotyl lengths. In contrast, the other six lines represented in lanes 2, 3, 5, 6, 8, and 10 exhibited relatively weak EIN2 silencing, resulting in shorter hypocotyls. These weaker-silenced lines showed more DNA methylation at the promoter and junction sequences as indicated by much lower PCR band intensity when the genomic DNA was pre-digested with McrBC. The quantitative RealTime PCR (qPCR) assays confirmed these observations (FIG. 31). All 12 of the tested lines showed some extent of DNA methylation in both the 35S promoter region and in the 35S-sense junction region (“junction”). The greatest extent of methylation i.e. the lowest quotient in the qPCR assays, was for hpEIN2[wt] lines 2, 3, 5, 6, 8 and 10, correlating perfectly with the reduced extent of silencing as measured by hypocotyl length. These results confirmed that the reduced EIN2 silencing in some of the hpEIN2[wt] lines was associated with increased promoter methylation. Even in the hpEIN2[wt] plant lines which were silenced for EIN2, there was still considerable levels of DNA methylation, particularly of the 35S-sense EIN2 junction fragment region. When promoters are methylated, this is thought to cause transcriptional silencing. In the case of silencing constructs, as here, this is a form of “self-silencing”.

In contrast to the hpEIN2[wt] lines, the hpEIN2[G:U] lines showed less DNA methylation at both the 35S promoter and the 35S-EIN2 junction. Indeed, four of these 12 G:U lines, corresponding to lanes 1, 2, 3 and 7 in FIG. 30 (lanes 13, 14, 15 and 20 in FIG. 31), had no obvious DNA methylation as indicated by the equal strength of PCR bands between McrBC-treated and untreated samples. When these amplifications were quantitated by qPCR, six of the 12 lines showed little to no reduction in the fragment from the McrBC treatment and therefore little to no DNA methylation—see lower panel of FIG. 31, lines 13, 14, 15, 18, 19 and 20. These results indicated that the relatively uniform EIN2 silencing by the hpEIN2[G:U] construct, at least in some lines, was due to significantly less promoter methylation and therefore less transcriptional self-silencing compared to hpEIN2[wt].

These conclusions were further confirmed by analysis of the genomic DNA from the transgenic plant lines with bisulfite sequencing. This assay made use of the fact that treatment of DNA with bisulfite converted unmethylated cytosine bases in the DNA to uracil (U), but left 5-methylcytosine bases (^{m}C) unaffected. Following the bisulfite treatment, the defined segment of DNA of interest was amplified in PCR reactions in a way whereby only the sense strand of the treated DNA was amplified. The PCR product was then subjected to bulk sequencing, revealing the positions and extent of methylation of individual cytosine bases in the segment of DNA. Therefore, the assay yielded single-nucleotide resolution information about the methylation status of a segment of DNA.

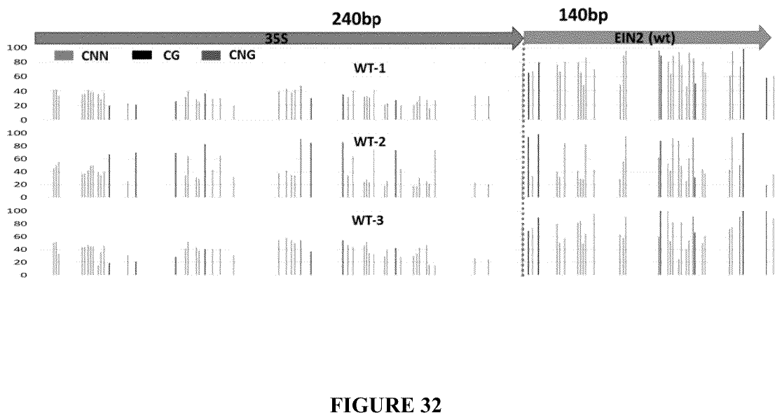

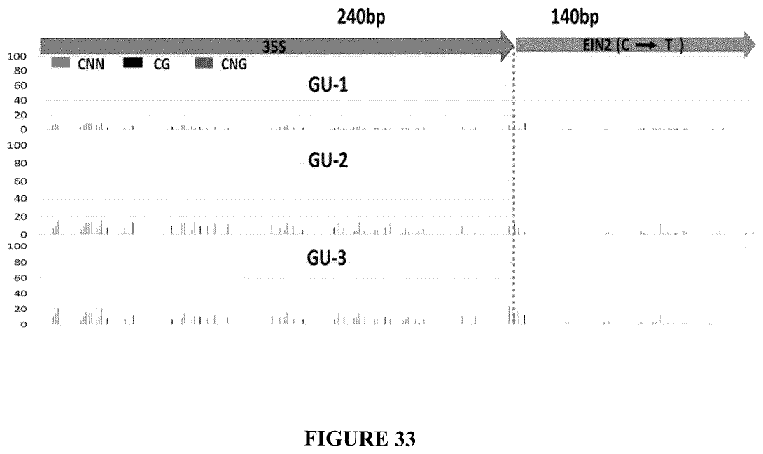

The three plant lines showing the strongest levels of EIN2 silencing for each of hpEIN2[wt] and hpEIN2[G:U] were analysed by bisulfite sequencing, corresponding to hpEIN2[wt] lines 1, 7 and 9 and hpEIN2[G:U] lines 13, 15 and 18 in FIG. 31. These plant lines showed the longest hypocotyl lengths and therefore were expected to have the lowest levels of DNA methylation out of the 20 lines for each construct. The results are presented in FIGS. 32 and 33 for hpEIN2[wt] and hpEIN2[G:U], respectively. When compared, it was clear that numerous cytosines in the 35S promoter region and the EIN2 sense region in the hpEIN2[wt] plants were extensively methylated. In clear contrast, the three hpEIN2[G:U] plant lines showed much lower levels of cytosine methylation in the 35S promoter region.

### Example 13. DNA Methylation Levels in Promoter of the hpGUS[1:4] Construct

When genomic DNA isolated from the hpGUS[1:4] plants was analysed for DNA methylation using the McrBC and bisulfite methods as described above, it was similarly observed that there was less methylation of cytosine bases in the 35S promoter and 35S promoter-GUS sense sequence regions relative to the hpGUS[wt] plants.

### General Discussion Relating to Examples 10 to 13

Double-Stranded RNA Having G:U Basepairs Induce More Uniform Gene Silencing than Conventional dsRNA

Like the GUS constructs, both hpEIN2[G:U] and hpCHS:EIN2[G:U] induced more consistent and uniform EIN2 silencing than the respective hpRNA[wt] constructs encoding a conventional hairpin RNA. The uniformity not only occurred across many independent transgenic lines, but also across sibling plants within a transgenic line each having the same transgenic insertion. In addition to the uniformity, the extent of EIN2 silencing induced by hpEIN2[G:U] was close to that of strongly silenced hpEIN2[wt] lines. Analysis of CHS gene silencing indicated that the hpCHS[G:U] construct was effective at reducing CHS mRNA levels by 50-97% but few plants showed a clearly visible phenotype in reduced seed coat colour. The likely explanation for not seeing more visible phenotypes in seed coat colour was that even low levels of CHS activity might be sufficient for producing the flavonoid pigments. Other possible explanations were that the 35S promoter was not sufficiently active in the developing seedcoat to produce the phenotype, or that the hpCHS[G:U] construct sequence contained 65 cytosine substitutions (32.5%), compared to only 43 (21.5%) for the EIN2 sequence and 52 (26%) for the GUS sequence. Furthermore, many of these cytosine bases in the CHS sequence occurred in sets of two or three consecutive cytosines, so not all of those need be substituted. When all of the cytosines in the sense strand were substituted, this resulted in more G:U basepairs in the hpCHS[G:U] RNA than in the hpEIN2[G:U] and hpGUS[G:U] RNAs, perhaps more than optimal. To verify this, another set of CHS constructs are made using a sequence containing a range of cytosine substitutions, from about 5%, 10%, 15%, 20% or 25% cytosine bases substituted. These constructs are tested and an optimal level determined.

The hpEIN2[G:U] Lines Express More Uniform Levels of siRNAs

Consistent with the relatively uniform EIN2 gene silencing, the hpEIN2[G:U] lines accumulated sRNAs with a more uniform level across the independent lines. This confirmed the conclusion with the hpGUS constructs that [G:U] modified hpRNA was efficiently processed by Dicer and capable of inducing effective target gene silencing.

### Fusion Constructs Also Provide for Gene Silencing

The purpose of including the CHS:EIN2 fusion constructs in the experiment was to test if two target genes could be silenced with a single hairpin-encoding construct. The GUS experiment suggested that the free energy and therefore stability of the hairpin RNA correlated positively with the extent of target gene silencing. The results showed that the CHS:EIN2 fusion construct did result in silencing of both genes—for CHS at least at the mRNA level.

The two hpRNA constructs, hpEIN2[G:U/U:G] and hpCHS:EIN2[G:U/U:G], in which both the sense and antisense sequences were modified from C to T so that 46% of basepairs were converted from canonical basepairs to G:U basepairs, induced only weak or no EIN2 silencing in most of the transgenic plants. Possible explanations include i) there were too many G:U basepairs which resulted in inefficient Dicer processing, and ii) sRNAs binding to target mRNA including too many G:U basepairs did not induce efficient mRNA cleavage, or a combination of factors.

Increased Uniformity in Target Gene Silencing by the G:U Basepaired Constructs is Associated with Reduced Promoter Methylation

DNA methylation analysis using both McrBC-digestion PCR and bisulfite sequencing showed that all hpEIN2[wt] plant lines showed DNA methylation at the promoter region, and the degree of methylation correlated negatively to the level of EIN2 silencing. Even the three least methylated lines, as judged by McrBC-digestion PCR, showed around 40% DNA methylation levels in the 35S promoter, relative to all cytosines being methylated. The widespread promoter methylation was thought to be due to sRNA-directed DNA methylation at the EIN2 repeat sequence that spread to the adjacent promoter region. In contrast to the hpRNA[wt] plant lines, a number of the hpEIN2[G:U] lines showed little to no promoter methylation and most of the plants analysed showed less methylated cytosines. As discussed for the hpGUS lines, several factors may contribute to the reduced methylation: i) the inverted-repeat DNA structure was disrupted by changing C bases to T bases in the sense sequence, and ii) the sense EIN2 sequence lacked cytosines so could not be methylated by sRNA-directed DNA methylation, and iii) a reduced level of production of 24-mer RNAs due to the change in the structure of the dsRNA region with the G:U basepairs, resulting in changes in the recognition by some Dicers and so a decrease in Dicer 3 and/or Dicer 4 activity and relatively more Dicer 2 activity. Thus, the hpEIN2[G:U] transgene may behave like a normal, non-RNAi transgene (such as an over-expression transgene) and the promoter methylation observed in some of the lines was due to T-DNA insertion patterns rather than the inherent inverted-repeat DNA structure of a hpRNA transgene.

### Example 14. Modified Hairpins for Reducing Expression of Another Endogenous Gene

Genetic constructs for production of modified silencing RNAs, either for hairpin RNAs or ledRNAs, targeting other endogenous genes were designed and synthesized. These included the following.

The FANCM gene in A. thaliana and in Brassica napus encodes a Fanconi Anemia Complementation Group M (FANCM) protein, which is a DEAD/DEAH box RNA helicase protein, Accession Nos and NM_001333162 and XM_018659358. The nucleotide sequence of the protein coding region of the cDNA corresponding to the FANCM gene of A. thaliana is provided in SEQ ID NO:31, and for B. napus in SEQ ID NO:32.

Genetic constructs were designed and made to express hairpin RNAs with or without C to T substitutions and an ledRNA targeting the FANCM gene in A. thaliana and in Brassica napus. A target region in the A. thaliana gene was selected: nucleotides 675-1174 (500 nucleotides) of SEQ ID NO:31. A target region in the B. napus gene was selected: nucleotides 896-1395 (500 bp) of SEQ ID NO:32. The constructs encoding the hairpin RNAs, using a wild-type sense sequence or a modified (G:U) sense sequence, were designed and assembled. Nucleotide sequences of the hpFANCM-At[wt], hpFANCM-At[G:U], hpFANCM-Bn[wt] and hpFANCM-Bn[G:U] constructs are provided in SEQ ID NOs:33-36. To make the G:U constructs, all cytosine bases in the sense sequences were replaced with thymine bases—102/500 (providing 20.4% G:U basepairs) in the A. thaliana construct and 109/500 (21.8% G:U basepairs) in the B. napus construct. The longest stretch of contiguous canonical basepairing in the double-stranded region of the B. napus G:U modified hairpin was 17 basepairs, and the second longest 16 contiguous basepairs.

The DDM1 gene in B. napus encodes a methyltransferase which methylates cytosine bases in DNA (Zhang et al., 2018). The nucleotide sequence of the protein coding region of the cDNA corresponding to the DDM1 gene of B. napus in SEQ ID NO:37.

Genetic constructs were designed and made to express hairpin RNAs with or without C to T substitutions and an ledRNA targeting the DDM1 gene in Brassica napus. Two non-contiguous target regions of the B. napus gene were selected: nucleotides 504-815 and 1885-2074 of SEQ ID NO:37, and were directly joined to make a chimeric sense sequence. The total length of the sense sequence was therefore 502 nucleotides. The constructs encoding the hairpin RNAs, using a wild-type sense sequence or a modified (G:U) sense sequence, were designed and assembled. Nucleotide sequences of the hpDDM1-Bn[wt] and hpDDM1-Bn[G:U] constructs are provided in SEQ ID NOs:38-39. To make the G:U construct, cytosines in the sense sequences were replaced with thymines—106/502 (21.1% G:U basepairs) in the B. napus construct. The longest stretch of contiguous canonical basepairing in the double-stranded region of the G:U modified hairpin was 20 basepairs, and the second longest 15 contiguous basepairs.

For another construct targeting an endogenous gene, a genetic construct was designed to express a hairpin RNA with 95 C to T substitutions in the sense sequence, out of 104 C's in the sense sequence of 350 nucleotides, providing for 95/350=27.1% G:U basepairs in the double-stranded region of the hairpin RNA. That is, not all of the C's in the sense sequence were replaced with T's. In particular, where a run of 3, 4 or 5 contiguous C's occurred in the sense sequence, only 1 or 2 of the three C's, or only 2 or 3 of four C's, or only 2, 3 or 4 of 5 contiguous C's, were replaced with T's. This provided for a more even distribution of G:U basepairs in the double-stranded RNA region. The longest stretch of contiguous canonical basepairing in the double-stranded region was 15 basepairs, and the second longest 13 contiguous basepairs.

A further construct was designed where one or two basepairs in every block of 4, 5, 6 or 7 nucleotides was modified with C to T or A to G substitutions. Where the wild-type sense sequence had a stretch of 8 or more nucleotides consisting of T's or G's, one or more nucleotides were substituted either in the sense strand to create a mismatched nucleotide within that block or a C to T or A to G substitution was made in the antisense strand, so as to avoid a double-stranded stretch of 8 or more contiguous canonical basepairs in the double-stranded region of the resultant hairpin RNA transcribed from the construct.

### Example 15. Modified Hairpins for Reducing Expression of Genes in Animal Cells

To test modified silencing RNAs in animal cells, of the G:U basepaired form, the ledRNA form or combining the two modifications, a gene encoding an enhanced green fluorescent protein (EGFP) was used in the following experiments as a model target gene. The nucleotide sequence of the coding region for EGFP is shown in SEQ ID NO:40. A target region of 460 nucleotides was selected, corresponding to nucleotides 131-591 of SEQ ID NO:40.

A genetic construct designated hpEGFP[wt] was designed and made which expressed a hairpin RNA comprising, in order 5′ to 3′ with respect to the promoter for expression, an antisense EGFP sequence of 460 nucleotides which was fully complementary to the corresponding region (nucleotides 131-590) of the EGFP coding region, a loop sequence of 312 nucleotides derived in part from a GUS coding region (corresponding to nucleotides 802-1042 of the GUS ORF), and a sense EGFP sequence of 460 nucleotides which was identical in sequence to nucleotides 131-590 of the EGFP coding region. The sequence of the DNA encoding the hairpin RNA hpEGFP[wt](SEQ ID NO:41) included a NheI restriction enzyme site at the 5′ end and a SalI site at the 3′ end to provide for cloning into the vector pCI (Promega Corporation). This vector was suitable for mammalian cell transfection experiments and would provide for expression from the strong CMV promoter/enhancer. The construct also had a T7 promoter sequence inserted between the NheI site and the beginning of the antisense sequence to provide for in vitro transcription to produce the hairpin RNA using T7 RNA polymerase. The hairpin encoding cassette was inserted into the NheI to SalI site in the expression vector pCI whereby the RNA coding region was operably linked to the CMV promoter and the SV40-late polyadenylation/transcription termination region.

A corresponding hairpin construct which had 157 C to T substitutions in the sense sequence and no substitutions in the antisense sequence was designed and made, designated hpEGFP[G:U](SEQ ID NO:42). The target region in the EGFP coding region was nucleotides 131-590. The percentage of C to T substitutions and therefore G:U basepairs in the stem of the hairpin RNA was 157/460=34.10%. The sense and antisense sequences were identical in length at 460 nucleotides. In the art of gene silencing, long double-stranded RNAs are generally avoided because of the potential for activating cellular response including interferon activation.

An ledRNA construct designated ledEGFP[wt] was designed and made to express an ledRNA comprising, in order 5′ to 3′ with respect to the promoter for expression, an antisense EGFP sequence of 228 nucleotides which was fully complementary to nucleotides 131-358 of the EGFP coding sequence, a loop sequence of 150 nucleotides, a sense EGFP sequence of 460 nucleotides which was identical in sequence to nucleotides 131-590 of the EGFP coding region (SEQ ID NO:40), a loop sequence of 144 nucleotides, and an antisense sequence of 232 nucleotides which was fully complementary to nucleotides 359-590 of the EGFP coding sequence, flanked by NheI and SalI restriction sites (SEQ ID NO:43). The encoded ledRNA was therefore of the type shown in FIG. 1A. The ledRNA structure, when self-annealed by basepairing between the one sense and two antisense sequences, had a double-stranded region of 460 basepairs corresponding to the EGFP target region, with the two antisense sequences not directly joined covalently to each other but having a “gap” or “nick” between the ends corresponding to nucleotides 358 and 359. The ledRNA structure was embedded in a larger RNA transcript including 5′- and 3′-regions coming from sequences in the CMV promoter and SV40-late poly adenylation/transcription termination regions.

A corresponding ledRNA construct which had 162 C to T substitutions in the sense sequence and no substitutions in the antisense sequence was also designed and made, designated ledEGFP[G:U](SEQ ID NO:44). In each case, the target region in the EGFP coding region was nucleotides 131-590 relative to the protein coding region starting with the ATG start codon (SEQ ID NO:40). The percentage of C to T substitutions and therefore G:U basepairs in the stem of the ledRNA was 162/460=35.2%.

Plasmids encoding the hpEGFP[wt], hpEGFP[G:U], ledEGFP[wt] and ledEGFP[G:U] silencing RNAs were tested for gene silencing activity in CHO, HeLa and VERO cells by transfection of the vectors into the cells. The assays were conducted by co-transfection of the test plasmids with a GFP expressing plasmid. All assays were conducted in triplicate. CHO cells (Chinese Hamster Ovary cells) and VERO cells (African Green monkey kidney cells) were seeded into 24 well plates at a density of 1×10⁵ cells per well. CHO cells were grown in MEMα modification (Sigma, USA), and HeLa and VERO cells were grown in DMEM (Invitrogen, USA). Both base media were supplemented with 10% foetal bovine serum, 2 mM glutamine, 10 mM Hepes, 1.5 g/L sodium bicarbonate, 0.01% penicillin and 0.01% streptomycin. Cells were grown at 37° C. with 5% CO₂. Cells were then transfected with 1 μg per well with plasmid DNA, or siRNA as a control for EGFP silencing, using Lipofectamine 2000. Briefly, the test siRNA or plasmid was combined with the GFP reporter plasmid (pGFP N1) and then mixed with 1 μl of Lipofectamine 2000, both diluted in 50 μl OPTI-MEM (Invitrogen, USA) and incubated at room temperature for 20 mins. The complex was then added to cells and incubated for 4 hr. Cell media was replaced and the cells incubated for 72 hr. Cells were next subjected to flow cytometry to measure GFP silencing. Briefly, cells to be analysed were trypsinized, washed in PBSA, resuspended in 200 L of 0.01% sodium azide and 2% FCS in PBSA and analysed using a FACScalibur (Becton Dickinson) flow cytometer. Data analysis was performed using CELLQuest software (Becton Dickinson) and reported as mean fluorescence intensity (MFI) as a percentage of control cells with reporter and non-related (negative control) shRNA.

The anti-GFP siRNA referred to as si22 was obtained from Qiagen (USA). The anti-GFP siRNA sequence of si22 was sense 5′-GCAAGCUGACCCUGAAGUUCAU-3′ (SEQ ID NO:86) and antisense 5′-GAACUUCAGGGUCAGCUUGCCG-3′(SEQ ID NO:87). A positive control genetic construct designated as pshGFP was created via a one-step PCR reaction using the mouse U6 sequence as the template. Forward primer was 5′-TTTTAGTATATGTGCTGCCG-3′ (SEQ ID NO:88) and reverse primer was 5′-CTCGAGTTCCAAAAAAGCTGACCCTGAAGTTCATCTCTCTTGAAGATGAACTTCA GGGTCAGCCAAACAAGGCTTTTCTCCAA-3′ (SEQ ID NO:89). An amplification product which included the full-length expression cassette was ligated into pGEM-T Easy. A non-related shRNA control plasmid was also constructed via the same PCR method. For that construction, the forward primer was 5′-TTTTAGTATATGTGCTGCCG-3′ (SEQ ID NO:90) and the reverse primer was 5′-ctcgagttccaaaaaaataagtcgcagcagtacaatctcttgaattgtactgctgcgacttatgaataccgcttcctcctgag-3′ (SEQ ID NO:91).

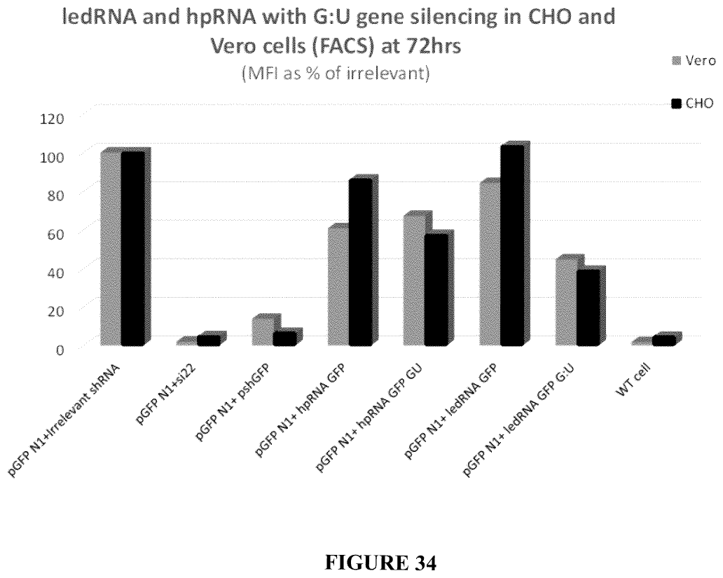

The resultant data from one experiment are shown in FIG. 34. Clear reduction in EGFP activity (RNA silencing) was observed in both VERO and CHO cells for both si22 and pshGFP positive controls when compared to the irrelevant shRNA control. These positive controls were a well validated small dsRNA molecule (si22) or encoded a shRNA (pshGFP) that were known to have very strong silencing activity in mammalian cells. The control RNA molecules have double-stranded regions of 20 contiguous basepairs and 21 contiguous basepairs, respectively, using only canonical basepairs and without any mismatched nucleotides in the double-stranded regions, and within the range of 20-30 basepairs long generally used for mammalian cells. In contrast, the hpRNA and ledRNA constructs express molecules having long dsRNA regions. All fours constructs were observed to specifically silence EGFP expression to significant extents in both cell types (FIG. 34). The inclusion of the G:U substitutions gave a pronounced improvement in silencing for both constructs in CHO cells. In VERO cells, a pronounced improvement in silencing was only observed with the ledEGFP[G:U] construct relative to ledEGFP[wt].

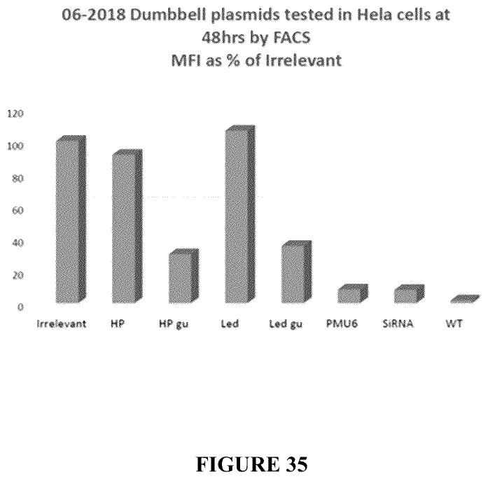

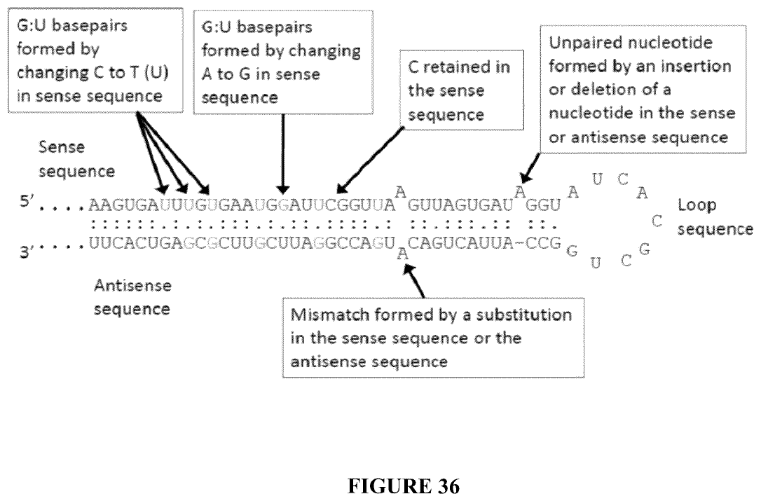

In a second experiment using HeLa (human) cells and assaying EGFP activity at 48 hr post-transfection, similar results were obtained (FIG. 35).

It was significant to note that the gene silencing was observed in mammalian cells using the hpRNA and ledRNA effector molecules given that they had longer double-stranded regions than the conventional 20 to 30 bp size range. It was also clear that the modification to substitute nucleotides to create the G:U basepairs significantly enhanced the gene silencing effect of these longer dsRNA molecules. This effect may be due to these structures more closely resembling endogenous priRNAs, the precursors of miRNAs, observed in eukaryotic cells and thus improving the processing of the longer dsRNA for loading into the RNA induced silencing complex (RISC) effector proteins.

### Example 16. RNA Constructs Targeting DDM1 and FANCM Genes in Plants

The inventors considered ways to increase the rate by which novel genetic profiles and diversity (genetic gain) could be generated and explored for desirable performance traits in plants. One way that was considered was to find a way to increase the rate of recombination that occurs during sexual reproduction of plants. Plant breeders rely on recombination events to create different genetic (allelic) combinations that they can search through for the desired genetic profile associated with performance gains. However, the number of recombination events in each breeding step is extremely low relative to the number of possible genetic profiles that could be explored. In addition, the elements that control where these events occur in the genome are not well understood. The inventors therefore considered whether ledRNA delivered either exogenously or endogenously through a transgenic approach could be used to modify recombination rates in plants to allow rapid increases in genetic diversity and make possible faster genetic gain within breeding populations.

The epigenome of plants is influenced by a range of different chemical modifications on the DNA and associated proteins that organize, package and stabilize the genome. These modifications also regulate where recombination takes place, with tight genome packaging being a strong inhibitor of recombination (Yelina et al, 2012; Melamed-Bessudo et al., 2012). DECREASED DNA METHYLATION 1 (DDM1) is an enzyme which regulates methylation of DNA and genome packaging. Mutation of this gene can alter the position of recombination events (Yelina et al, 2012; Melamed-Bessudo et al., 2012).

Recombination events during meiosis are tightly regulated with only 1-2 events occurring on each chromosome to ensure proper chromosome segregation at metaphase 1. Recombination events are initiated though double stranded breaks (DSB) of the DNA through the enzyme SPO11 (Wijnker et al, 2008). This results in hundreds of DSB along the chromosome. While a few of these DSB result in crossovers, the majority are repaired by DNA repair enzymes, before a recombination event can take place. Furthermore there are a number of negative regulators which inhibit DSB developing into crossovers. In an initial approach contemplated by the inventors, genetic constructs encoding ledRNA molecules or conventional hairpin RNA molecules as a comparison were to be introduced into A. thaliana plants, targeting a gene encoding a protein factor which could potentially impact recombination rates such as FANCONI ANEMIA COMPLEMENTATION GROUP M (FANCM).

The nucleotide sequence of the DDM1 gene of A. thaliana was provided by Accession No. AF143940 (Jeddeloh et al., 1999). Reduction of DDM1 gene expression has been shown to decrease DNA methylation and increase the number and position of cross over events in A. thaliana (Melamed-Bessudo and Levy, 2012).

Brassica napus is an allotetraploid species and has two DDM1 genes on each of the A and C subgenomes, on chromosomes A7, A9, C7 and C9, therefore having a total of four DDM1 genes. These genes are designated BnaA07g37430D-1, BnaC07g16550D-1, BnaA09g52610D-1 and BnaC09g07810D-1. The nucleotide sequence of the DDM1 gene BnaA07g37430D-1 of B. napus is provided by Accession No. XR_001278527 (SEQ ID NO:93). A hairpin RNA construct was designed and made targeting a 500 nucleotide region of the four genes, corresponding to nucleotides 650-959 and 2029-2218 of SEQ ID NO:93. The nucleotide region used to design the hpRNA and ledRNA constructs targeted all four of the DDM1 genes BnaA07g37430D-1, BnaC07g16550D-1, BnaA09g52610D-1 and BnaC09g07810D-1 present in B. napus, based on sequence conservation between the genes. The order of elements in the hpRNA construct was promoter-sense sequence-loop sequence comprising an intron from Hellsgate vector-antisense sequence-transcription terminator/polyadenylation region. The nucleotide sequence of the chimeric DNA encoding the hpRNA is provided as SEQ ID NO:94.

A second hairpin RNA construct was made encoding a hairpin RNA targeting the same 500 nucleotide region and having the same structure except that 97 cytosine nucleotides (C) of the sense sequence were replaced with thymidine nucleotides (T). When the chimeric DNA was transcribed and the G:U substituted hpRNA was self-annealed, this provided for 97/500=19.4% of the nucleotides in the dsRNA region being basepaired in a G:U basepair. The nucleotide sequence of the chimeric DNA encoding the G:U-modified hpRNA is provided as SEQ ID NO:95. Further, a chimeric DNA encoding a ledRNA targeting the same region of the DDM1 gene of B. napus was made. The nucleotide sequence of this chimeric DNA encoding the ledRNA is provided as SEQ ID NO:96.

For production of the RNAs by in vitro transcription, DNA preparations were cleaved with the restriction enzyme HincII which cleaved immediately after the coding region, transcribed in vitro with RNA polymerase T7, the RNA purified and then concentrated in an aqueous buffer solution. LedRNA was used to target endogenous DDM1 transcripts in B. napus (canola) cotyledons. Cotyledons from five-day-old seedlings grown aseptically on tissue culture medium were carefully excised and placed in a petri dish containing 2 ml MS liquid media, comprising 2% (w/v) sucrose, with 113 μg of ledRNA or 100 ul of aqueous buffer solution as a control. MS liquid media used for the treatments contained Silwett-77, a surfactant (0.5 μl in 60 ml). The petri dishes were incubated on a shaker with gentle shaking, so that the cotyledons soaked in the solution containing the ledRNA. Samples were harvested 5 hr and 7 hr after application of the ledRNA. In a parallel experiment, the upper surface of cotyledons was coated either with 10 μg of ledRNA or buffer solution and incubated on a wet tissue paper. Samples were collected 7 hr after ledRNA application.

Furthermore, in order to target the DDM1 endogenous transcripts in reproductive tissue of B. napus, canola floral buds were exposed to ledRNA either in the presence or absence of an aliquot of an Agrobacterium tumefaciens strain AGL1 cell suspension, i.e. living AGL1 cells. Aqueous buffer solution with or without the AGL1 cells served as respective controls. The AGL1 was grown in 10 ml of LB liquid media containing 25 mg/ml rifampicin for two days at 28° C. The cells were harvested by centrifugation at 3000 rpm for 5 minutes. The cell pellet was washed and the cells resuspended in 2 ml liquid MS media. Floral buds were incubated in a petri dish containing 2 ml of MS liquid media, including 0.5 μl of Silwett-77 in 50 ml of MS liquid media, with 62 μg of ledRNA or 62 μg+50 μl of AGL1 culture. As controls, 50 μl of buffer solution or 50 μl of buffer solution+50 μl of AGL1 culture was used. Samples were incubated on a shaker with gentle shaking for 7 hr. Three biological replicates were used for each of the treatments.

The treated and control cotyledons and floral buds were washed twice in sterile distilled water, the surface water removed using a tissue paper and flash frozen with liquid nitrogen. RNA was isolated from the treated and control tissues, treated with DNase to remove genomic DNA and quantified. First strand cDNA was synthesized using equal amounts of total RNA from ledRNA-treated samples and their respective controls. Expression of DDM1 was analysed using quantitative real-time PCR (qRT-PCR).

In the treated cotyledons that were soaked with the ledRNA, DDM1 transcript abundance was decreased by approximately 83-86% at 5 hr, which decreased further with a reduction of 91% at 7 hr compared to the controls. Similarly, a reduction of approximately 78-85% in the DDM1 mRNA level compared to the control was observed in cotyledons that were coated with ledRNA. No difference in DDM1 mRNA abundance was detected in the floral buds that were treated with ledRNA compared to control in the absence of Agrobacterium cells. However, a reduction of approximately 60-75% in DDM1 transcript levels was observed in floral buds that were treated with ledRNA in presence of Agrobacterium compared to its respective control. No significant difference in DDM1 transcript levels was detected when the control without Agrobacterium was compared with the control that had Agrobacterium showing that the Agrobacterium cells themselves were not causing the decrease in DDM1 transcript. Taken together, these results indicated that the ledRNA was able to reduce endogenous DDM1 transcript levels in both cotyledons and floral buds, while living Agrobacterium cells appeared to facilitate the ledRNA entry into the floral buds. Such accessibility of the ledRNA might also be achieved by physical means such as piercing the outer layers of the floral buds, centrifugation or vacuum infiltration, or a combination of such methods.

Certain Arabidopsis thaliana mutants such as zip4 mutants lack meiotic crossovers, causing mis-segregation of chromosome homologs and thus reduced fertility and leading to shorter siliques (fruit) that can be visually discriminated from that of the wild-type. The phenotype in zip4 mutants can be reversed by reducing FANCM gene expression.

The nucleotide sequence of the FANCM gene of A. thaliana was provided by Accession No. NM_001333162 (SEQ ID NO:97). A hairpin RNA construct was designed and made targeting a 500 nucleotide region of the gene, corresponding to nucleotides 853-1352 of SEQ ID NO:97. The order of elements in the construct was promoter-sense sequence-loop sequence comprising an intron from Hellsgate vector-antisense sequence-transcription terminator/polyadenylation region. The nucleotide sequence of the chimeric DNA encoding the hpRNA is provided as SEQ ID NO:98. A second hairpin RNA construct was made encoding a similar hairpin RNA targeting the same 500 nucleotide region except that 102 cytosine nucleotides (C) of the sense sequence were replaced with thymidine nucleotides (T). When the chimeric DNA was transcribed and the resultant G:U substituted hpRNA self-annealed, this provided for 102/500=20.4% of the nucleotides in the dsRNA region being basepaired in a G:U basepair. The nucleotide sequence of the chimeric DNA encoding the G:U-modified hpRNA is provided as SEQ ID NO:99. Further, a chimeric DNA encoding a ledRNA targeting the same region of the FANCM gene of A. thaliana was made. The nucleotide sequence of this chimeric DNA encoding the ledRNA is provided as SEQ ID NO:100.

B. napus has one FANCM gene on each of its A and C subgenomes, designated BnaA05g18180D-1 and BnaC05g27760D-1. The nucleotide sequence of one of the FANCM genes of B. napus is provided by Accession No. XM_022719486.1; SEQ ID NO:101). A chimeric DNA encoding the hairpin RNA was designed and made targeting a 503 nucleotide region of the genes, corresponding to nucleotides 2847-3349 of SEQ ID NO:101. The order of elements in the construct was promoter-sense sequence-loop sequence comprising an intron from Hellsgate vector-antisense sequence-transcription terminator/polyadenylation region. The nucleotide sequence of the chimeric DNA encoding the hpRNA is provided as SEQ ID NO:102. A second hairpin RNA construct was made encoding a similar hairpin RNA targeting the same 503 nucleotide region except that 107 cytosine nucleotides (C) of the sense sequence were replaced with thymidine nucleotides (T). When the chimeric DNA was transcribed and the G:U substituted hpRNA self-annealed, this provided for 107/500=21.4% of the nucleotides in the dsRNA region being basepaired in a G:U basepair. The nucleotide sequence of the chimeric DNA encoding the G:U-modified hpRNA is provided as SEQ ID NO:103. Further, a chimeric DNA encoding a ledRNA targeting the same region of the FANCM gene of B. napus was made. The nucleotide sequence of this chimeric DNA encoding the ledRNA is provided as SEQ ID NO:104.

For production of the RNAs by in vitro transcription, DNA preparations were cleaved with the restriction enzyme HincII which cleaved immediately after the coding region, transcribed in vitro with RNA polymerase T7, the RNA purified and then concentrated in an aqueous buffer solution. LedRNA was used together with Agrobacterium tumefaciens AGL1 to target FANCM transcripts in pre-meiotic buds of a zip4 mutant of A. thaliana. Siliques of the zip4 mutant were shorter, readily observed visually, relative to wild-type siliques due to attenuated crossover formation, thus causing reduced fertility. Repressing FANCM in the zip4 mutant has been shown to restore the fertility and restore silique length.

The A. thaliana zip4 inflorescences containing the pre-meiotic buds were contacted with ledRNA targeting FANCM together with AGL1 or buffer solution with AGL1 as control, in each case in the presence of a surfactant, in this case Silwett-77. Once the seed setting was complete, the siliques developed from pre-meiotic buds were excised to determine the seed numbers. Among the 15 siliques from ledRNA-treated samples, two siliques displayed 10 seeds, one silique had 9 seeds, while the number of seeds in control siliques ranged from 3 to 6. These results indicated that the observed increase in seed number was due to the repression of FANCM transcript levels by the ledRNA, thereby resulting in an increased number of meiotic crossovers and increased fertility.

### Example 17. RNA Constructs for Resistance to Fungal Disease

### LedRNA Targeting Mlo Genes of Barley and Wheat

The fungal disease of cereal plants, powdery mildew, is caused by the ascomycete Blumeria graminis f. sp. hordei in barley and the related Blumeria graminis f. sp. tritici in wheat. B. graminis is an obligate biotrophic fungal pathogen of the order Erysiphales (Glawe, 2008) which requires a plant host for reproduction, involving a close interaction between fungal and host cells in order for the fungus to acquire nutrient from the plant. The fungus initially infects the epidermal layer of leaves, leaf sheaths or ears after fungal ascospores or conidia contact the surface. Leaves remain green and active for some time following infection, then powdery, mycelial masses grow and the leaves gradually become chlorotic and die off. As the disease progresses, the fungal mycelium may become dotted with tiny black points which are the sexual fruiting bodies of the fungus. Powdery mildew disease has a worldwide distribution and is most damaging in cool, wet climates. The disease impacts grain yield mainly by reducing the number of heads as well as reducing kernel size and weight. Currently, disease control is by spraying crops with fungicide which needs to be applied frequently when conditions are cool and damp, and is expensive, or by growing resistant cultivars. Moreover, fungicide resistance has emerged for powdery mildew in wheat in Australia.

The Mlo genes of barley and wheat encode Mlo polypeptides which confer susceptibility to B. graminis by an unknown mechanism. There are multiple, closely related MLO proteins encoded by a Mlo gene family which are unique to plants. Each gene encodes a seven-transmembrane domain protein of unknown biochemical activity localized in the plasma membrane. Significantly, only specific Mlo genes within the family are capable of acting as powdery mildew susceptibility genes and these encode polypeptides with conserved motifs within the cytoplasmic C-terminal domain of the Mlo proteins. The mechanism by which Mlo polypeptides act as powdery mildew susceptibility factors is unknown. Occurrence of natural wheat mlo mutants has not been reported, presumably because of the polyploid nature of wheat. However, artificially generated mlo mutants show some resistance to the disease but often exhibit substantially reduced grain yield or premature leaf senescence (Wang et al., 2014; Acevedo-Garcia et al., 2017).

Hexaploid wheat has three homeologs of Mlo genes, designated as TaMlo-A1, TaMlo-B1 and TaMlo-D1 located on chromosomes 5AL, 4BL and 4DL respectively (Elliott et al., 2002). Nucleotide sequences of cDNAs corresponding to the genes are available as Accession Nos: TaMlo-A1, AF361933 and AX063298; TaMlo-B1, AF361932, AX063294 and AF384145; and TaMlo-D1, AX063296. The nucleotide sequences of the genes on the A, B and D genomes and the amino acid sequences of the encoded polypeptides are approximately 95-97% and 98% identical, respectively. All three genes are expressed in leaves of the plants with the expression levels increasing as the plants grow and mature. The inventors therefore designed and made a ledRNA construct which would be capable of reducing expression of all three genes, taking advantage of the degree of sequence identity between the genes and targeting a gene region with high degree of sequence conservation.

A chimeric DNA encoding a ledRNA construct targeting all three of the TaMlo-A1, TaMlo-B1 and TaMlo-D1 genes was made. The genetic construct was made using the design principles for ledRNAs described above, with the split sequence being the antisense sequence and the contiguous sequence being the sense sequence (FIG. 1A).

A 500 bp nucleotide sequence of a TaMlo target gene was selected, corresponding to nucleotides 916-1248 fused with 1403-1569 of SEQ ID NO: 136. The dsRNA region of each ledRNA was 500 bp in length; the sense sequence in the dsRNA region was an uninterrupted, contiguous sequence, for example corresponded to nucleotides 916-1248 fused with 1403-1569 of SEQ ID NO:136. The nucleotide sequence encoding the ledRNA is provided herein as SEQ ID NO:137.

The ledRNA was prepared by in vitro transcription using T7 RNA polymerase, purified and resuspended in buffer. 10 μg of ledRNA per leaf was applied using a paint brush to a zone of leaves in wheat plants at the Zadoks 23 stage of growth. As controls, some leaves were mock-treated using buffer alone. Treated and control leaf samples were harvested and RNA extracted. QPCR assays on the extracted RNAs showed that TaMlo mRNA levels, being a combination of the three TaMlo mRNAs, were reduced by 95.7%. Plants at the Z73 stage of growth were also treated and assayed. They showed a 91% reduction in TaMlo gene expression by QPCR relative to the control leaf samples. The reduction in TaMlo gene expression observed in the treated leaf areas was specific to the treated zones—there was no reduction in TaMlo mRNA levels in distal, untreated parts of the leaves.

In barley mlo mutants, expression of a variety of disease defence-related genes was observed to be increased. Therefore, the ledRNA-treated wheat leaves were assayed by QPCR for the levels of defence related genes encoding PR4, PR10, β-1,3-glucanase, chitinase, germin and ADP-ribosylation factor. None of these genes were altered significantly in expression level in the treated leaf areas relative to the control leaf areas.

To test for ability of the ledRNA to increase disease resistance by reducing Mlo gene expression, spores of the powdery mildew fungus are applied to the treated and untreated zones of the leaves. Leaves were detached from wheat plants, treated with the ledRNA as before and maintained on medium (50 mg Benzimidazole and 10 g agar per Litre of water) to prevent the leaves from senescencing, under light. Twenty-four hours later, the leaves were inoculated with powdery mildew spores and disease progression followed for 5 to 24 days. Treated leaves showed little to no fungal mycelium growth and no leaf chlorosis relative to control leaves, not having received the ledRNA, which showed extensive mycelial growth surrounded by chlorotic zones.

In further experiments, lower levels of the ledRNA are applied to identify the minimal level of the ledRNA that is effective. Further, leaves are inoculated 2, 4, 7 or more days after the ledRNA treatment to see how long the protective effect remains. Whole plants will also be sprayed with ledRNA preparations and tested for disease resistance after being inoculated with the fungal disease agent.

LedRNA Targeting VvMLO Genes of Vitis vinifera

The MLO genes of Vitis vinifera and Vitis pseudoreticulata encode MLO polypeptides which confer susceptibility to the fungal disease powdery mildew, caused by the ascomycete fungus, Erysiphe necator. E. necator is an obligate biotrophic fungal pathogen which requires a plant host for reproduction, involving a close interaction between fungal and host cells in order for the fungus to acquire nutrient from the plant. There are multiple, closely related MLO proteins encoded by a gene family all of which are unique to plants and encode seven-transmembrane domain proteins of unknown biochemical activity localized in the plasma membrane. Significantly, only specific MLO genes within the family are capable of acting as powdery mildew susceptibility genes and these encode polypeptides with conserved motifs within the cytoplasmic C-terminal domain of the MLO proteins. The mechanism by which MLO polypeptides act as powdery mildew susceptibility factors is unknown.

LedRNA constructs targeting three different but related MLO genes of Vitis species, namely VvMLO6, VvMLO11 and VvMLO15 were designed and made as follows. For the first one, for example, a 600 nucleotide sequence of a VvMLO6 target gene was selected, corresponding to nucleotides 556-1155 of SEQ ID NO:138. Chimeric DNAs encoding three ledRNA constructs targeting VvMLO6, VvMLO7 and VvMLO11 genes were made. The genetic constructs were made using the design principles for ledRNAs described above, with the split sequence being the antisense sequence and the contiguous sequence being the sense sequence (FIG. 1A). The dsRNA region of each ledRNA was 600 bp in length; the sense sequence in the dsRNA region was an uninterrupted, contiguous sequence, for example corresponded to nucleotides 556-1155 of SEQ ID NO:138. The nucleotide sequence encoding one of the ledRNAs is provided herein as SEQ ID NO:139.

The ledRNAs are prepared by in vitro transcription and applied, separately or as a mixture of all three, to Vitis leaves. Subsequently, spores of the powdery mildew fungus are applied to the treated and untreated zones of the leaves. Down-regulation of the target mRNA is observed using QPCR and disease progression is followed over time.

### Example 18. RNA Constructs Targeting Other Genes in Plants

LedRNAs Targeting Tor Genes of A. Thaliana and N. benthamiana

The Target of Rapamycin (TOR) gene encodes a serine-threonine protein kinase polypeptide that controls many cellular functions in eukaryotic cells, for example in response to various hormones, stress and nutrient availability. It is known as a master regulator that regulates the translational machinery to optimise cellular resources for growth (Abraham, 2002). At least in animals and yeast, TOR polypeptide is inactivated by the antifungal agent rapamycin, leading to its designation as Target of Rapamycin. In plants, TOR is essential for embryonic development in the developing seed, as shown by the lethality of homozygous mutants in TOR (Mahfouz et al., 2006), as well as being involved in the coupling of growth cues to cellular metabolism. Down-regulation of TOR gene expression was thought to result in an increase in fatty acid synthesis resulting in increased lipid content in plant tissues.

LedRNA constructs targeting a TOR gene of Nicotiana benthamiana, the nucleotide sequence of the cDNA protein coding region is provided as SEQ ID NO:105, were designed and made using the design principles for ledRNAs with the split sequence being the sense sequence and the contiguous sequence being the antisense sequence (FIG. 1B). The target region was 603 nucleotides in length, corresponding to nucleotides 2595-3197 of SEQ ID NO:105. The dsRNA region of the ledRNA was 603 bp in length; the antisense sequence in the dsRNA region was an uninterrupted, contiguous sequence corresponded to the complement of nucleotides 2595-3197 of SEQ ID NO:105. The nucleotide sequences encoding the ledRNA is provided herein as SEQ ID NO: 106. DNA preparations of the genetic constructs encoding the ledRNA constructs were cleaved with the restriction enzyme MlyI which cleaved the DNA immediately after the coding region, transcribed in vitro with RNA polymerase SP6 and the RNA purified and then concentrated in an aqueous buffer solution. Samples of the ledRNA were applied to the upper surface of N. benthamiana leaves. After 2 days and 4 days, the treated leaf samples were harvested, dried, and the total fatty acid content measured by quantitative gas chromatography (GC). The leaf samples treated with the TOR ledRNAs showed an increase in total fatty acid (TFA) content from 2.5-3.0% (weight of TFA/dry weight) observed in the control (untreated) samples to between 3.5-4.0% for the ledRNA treated samples. That represented an increase of between 17% and 60% in the TFA content relative to the control, indicating that the TOR gene expression had been reduced in the ledRNA treated tissues.

LedRNA Targeting ALS Gene of H. vulgare

Acetolactate synthase (ALS) genes encode an enzyme (EC 2.2.1.6) found in plants and microorganisms which catalyse the first step in the synthesis of the branched chain amino acids leucine, valine and isoleucine. The ALS enzyme catalyses the conversion of pyruvate to acetolactate which is then further converted to the branched chain amino acids by other enzymes. Inhibitors of ALS are used as herbicides such as the sulfonylurea, imidazolinone, triazolopyrimidine, pyrimidinyl oxybenzoate and sulfonylamino carbonyl triazolinones classes of herbicides.

To test whether a ledRNA could reduce ALS gene expression by exogenous delivery of the RNA to plants, a genetic construct encoding a ledRNA was designed and made that targeted an ALS gene in barley, Hordeum vulgare. The H. vulgare ALS gene sequence is provided herein as SEQ ID NO:107 (Accession No. LT601589). The genetic construct was made using the design principles for ledRNAs, with the split sequence being the sense sequence and the contiguous sequence being the antisense sequence (FIG. 1B). The target region was 606 nucleotides in length, corresponding to nucleotides 1333-1938 of SEQ ID NO:107. The dsRNA region of the ledRNA was 606 bp in length; the antisense sequence in the dsRNA region was an uninterrupted, contiguous sequence corresponded to the complement of nucleotides 1333-1938 of SEQ ID NO:107. The nucleotide sequences encoding the ledRNA is provided herein as SEQ ID NO:108. The coding region was under the control of a SP6 RNA polymerase promoter for in vitro transcription.

The genetic construct encoding the ledRNA was digested with the restriction enzyme MlyI, which cleaved downstream of the ledRNA coding region, and transcribed in vitro with RNA polymerase SP6 according to the instructions with the transcription kit. The RNA was applied on the upper surface of leaves of barley plants. RNA was extracted from the treated leaf samples (after 24 hours). Quantitative reverse transcription-PCR (QPCR) assays were carried out on the RNA samples. The assays showed that the level of ALS mRNA was reduced in the ledRNA treated tissues. (Total RNA was extracted for treated and untreated plants, DNase treated, quantified and 2 ug reverse transcribed using primer CTTGCCAATCTCAGCTGGATC. The cDNA was used as template for quantitative PCR using the forward primer TAAGGCTGACCTGTTGCTTGC and reverse primer CTTGCCAATCTCAGCTGGATC. ALS mRNA expression was normalised against the Horendeum chilense isolate H1 lycopene-cyclase gene. ALS expression was reduced by 82% in LED treated plants.

### LedRNAs Targeting NCED1 and NCED2 Genes of Wheat and Barley

In plants, the plant hormone abscisic acid (ABA) is synthesized from carotenoid precursors with the first committed step in the synthesis pathway being catalyzed by the enzyme 9-cis epoxy-carotenoid dioxygenase (NCED) which cleaves 9-cis xanthophylls to xanthoxin (Schwartz et al., 1997). The hormone ABA is known to promote dormancy in seeds (Millar et al., 2006) as well as being involved in other processes such as stress responses. Increased expression of an NCED gene was thought to increase ABA concentration and thereby promote dormancy. There are two NCED isoenzymes in cereals such as wheat and barley, designated NCED1 and NCED2, encoded by separate, homologous genes.

For breakdown of ABA, the enzyme ABA-8-hydroxylase (ABA80H-2, also known as CYP707A2) hydroxylates ABA as a step in its catabolism, resulting in the breaking of dormancy and seed germination.

LedRNA constructs targeting genes encoding HvNCED1 (Accession No. AK361999, SEQ ID NO:109) or HvNCED2 (Accession No. AB239298; SEQ ID NO:110) in barley Hordeum vulgare and the corresponding homologous genes in wheat were designed for transgenic expression in barley and wheat plants. These constructs used a highly conserved region of the wheat and barley NCED1 and NCED2 genes, the wheat and barley nucleotide sequences being about 97% identical in the conserved region. The genetic constructs were made using the design principles for ledRNAs described above, with the split sequence being the antisense sequence and the contiguous sequence being the sense sequence (FIG. 1A). The target region was 602 nucleotides in length, corresponding to nucleotides 435-1035 of SEQ ID NO:109. The dsRNA region of the ledRNA was 602 bp in length; the sense sequence in the dsRNA region was an uninterrupted, contiguous sequence corresponded to nucleotides 435-1035 of SEQ ID NO:110. The nucleotide sequences encoding the NCED1 and NCED2 ledRNAs are provided herein as SEQ ID NO:111 and 112.

In similar fashion, an ledRNA construct was made targeting an ABA-OH-2 gene of wheat T. aestivum and barley H. vulgare (Accession No. DQ145933, SEQ ID NO:113). The target region was 600 nucleotides in length, corresponding to nucleotides 639-1238 of SEQ ID NO:113. The dsRNA region of the ledRNA was 600 bp in length; the sense sequence in the dsRNA region was an uninterrupted, contiguous sequence corresponded to nucleotides 639-1238 of SEQ ID NO:113. The nucleotide sequence of the chimeric DNA encoding the ledRNA is provided as SEQ ID NO: 114.

The chimeric DNAs encoding the ledRNAs were inserted into an expression vector under the control of a Ubi gene promoter that is expressed constitutively in most tissues including in developing seed. The expression cassettes were excised and inserted into a binary vector. These were used to produce transformed wheat plants.

The transgenic wheat plants are grown to maturity, seed obtained from them and analysed for decreased expression of the NCED or ABA-OH-2 genes and for effects on grain dormancy corresponding to decreased gene expression. A range of phenotypes in the extent of altered dormancy is expected. To modulate the extent of the altered phenotypes, modified genetic constructs are produced for expression of ledRNAs having G:U basepairs in the double-stranded RNA regions, particularly for ledRNAs where between 15-25% of the nucleotides in the double-stranded region of the ledRNA are involved in a G:U basepair, as a percentage of the total number of nucleotides in the double-stranded region.

LedRNA Targeting EIN2 Gene of A. thaliana

As described in Example 10, the EIN2 gene of Arabidopsis thaliana encodes a receptor protein involved in ethylene perception. EIN2 mutant seedlings exhibit hypocotyl elongation relative to wild-type seedlings when germinated on ACC. Since the gene is expressed in seedlings soon after germination of seeds, delivery of a ledRNA by transgenic means was considered the most suitable approach for tested the extent of down-regulation of EIN2, relative to exogenous delivery of preformed RNA.

An ledRNA construct targeting the EIN2 gene of Arabidopsis thaliana (SEQ ID NO:115) was designed, targeting a 400 nucleotide region of the target gene mRNA. The construct is made by inserting a sequence (SEQ ID NO: 116) encoding the ledRNA into a vector comprising a 35S promoter to express the ledRNA MA. thaliana plants. Transgenic A. thaliana plants are produced and tested for reduction of expression of the EIN2 gene by QPCR and for the hypocotyl length assay in the presence of ACC. Reduction in EIN2 expression levels and increased hypocotyl lengths are observed in plants of some transgenic lines.

### LedRNA Targeting CHS Gene of A. Thaliana

The chalcone synthase (CHS) gene in plants encodes an enzyme that catalyzes the conversion of 4-coumaroyl-CoA and malonyl-CoA to naringenin chalcone which is the first committed enzyme in flavonoid biosynthesis. Flavanoids are a class of organic compounds found mainly in plants, involved in defense mechanisms and stress tolerance.

An ledRNA construct targeting the CHS gene of Arabidopsis thaliana (SEQ ID NO:117) was designed, targeting a 338 nucleotide region of the target gene mRNA. The construct is made by inserting a DNA sequence (SEQ ID NO:118) encoding the ledRNA into a vector comprising a 35S promoter to express the ledRNA in A. thaliana plants. Transgenic A. thaliana plants are produced by transformation with the genetic construct in a binary vector and tested for reduction of expression of the CHS gene by QPCR and for the reduced flavonoid production. Reduction in CHS expression levels and reduced levels of flavonoids are observed in plants of some transgenic lines, for example in the seed coat of transgenic seeds.

LedRNA targeting LanR gene of Lupinus angustifolius

The LanR gene of narrow-leafed lupin, Lupinus angustifolius L., encodes a polypeptide that is related in sequence to the tobacco N gene, which confers resistance to viral disease caused by tobacco mosaic virus (TMV).

A chimeric DNA for producing ledRNA molecules targeting the LanR gene of L. angustifolius (Accession No. XM_019604347, SEQ ID NO: 119) was designed and made. The genetic construct was made using the design principles for ledRNAs described above, with the split sequence being the antisense sequence and the contiguous sequence being the sense sequence (FIG. 1A). The nucleotide sequence encoding the ledRNA is provided herein as SEQ ID NO: 120. The ledRNA was produced by in vitro transcription, purified and concentrated, and aliquots of the RNA are applied to leaves of L. angustifolius plants which contain the LanR gene. Samples of virus are applied to treated and non-treated plants, and disease symptoms compared after several days.

### Example 19. RNA Constructs Targeting an Insect Gene

### Introduction

Aphids are sap-sucking insects that cause substantial and at times severe damage to plants directly through feeding of plant sap and, in some cases, indirectly through transmitting various viruses that cause disease in the plants. While Bt toxin has in some instances been effective in protecting crop plants from chewing insects, it generally hasn't been effective for sap-sucking insects. Plant cultivars contain resistance genes can be an effective way to control aphids, however, most resistance genes are highly specific to certain aphid species or biotypes and resistance is frequently over-come due to rapid evolution of new biotypes through genetic or epigenetic changes. Moreover, resistance genes are not accessible in many crops or may not exist for certain generalist aphid species such as green peach aphid which infest a broad host species. Aphids are currently controlled primarily through frequent application of pesticides which has led to pesticide resistance in aphids. For example, only one pesticide mode of action group remains effective in Australia against the green peach aphid as it has managed to gain resistance to all the other registered insecticides.

RNAi-mediated gene silencing has been shown in a few studies to be useful as a research tool in a number of aphid species, for reviews see Scott et al., 2013; Yu et al., 2016, but has not been shown to effectively protect plants from aphid attack. In those studies, dsRNAs targeting key genes involved in aphid growth and development, infestation or feeding processes were delivered through direct injection to the aphids or by feeding the aphids on artificial diets containing the dsRNA.

To test the potential of modified RNAi molecules such as the ledRNA molecules described herein for the control of sap-sucking insects, the inventors selected green peach aphid (Myzus persicae) as a model sap-sucking insect, for several reasons. Firstly, green peach aphid is a polyphagous insect which infests a broad range of host plant species including major grain and horticultural crops worldwide. Secondly, green peach aphid is responsible for the transmission of some devastating viruses, such as Beet Western Yellows Virus which has been highly damaging in some canola growing areas. Two aphid genes were selected for this study as target genes for down-regulation, one encoding a key effector protein (C002) and the second encoding a receptor of activated protein kinase C (Rack-1). The C002 protein is an aphid salivary gland protein which is essential for aphid feeding on its host plant (Mutti et al., 2006; Mutti et al., 2008). Rack1 is an intracellular receptor that binds activated protein kinase C, an enzyme primarily involved in signal transduction cascades (McCahill et al., 2002; Seddas et al., 2004). MpC002 is predominantly expressed in the aphid salivary gland and MpRackl is predominantly expressed in the gut. In previous studies, use of RNAi via direct injection or artificial diet feeding led to the death of several aphid species tested (Pitino et al., 2011; Pitino and Hogenhout, 2012; Yu et al., 2016).

### Materials and Methods: Aphid Culture and Plant Materials

Green peach aphids (Myzus persicae) were collected in Western Australia. Before each experiment, aphids were reared on radish plants (Raphanus sativus L.) under ambient light in an insectary room. Aphids were transferred to experimental artificial diet cages with a fine paintbrush.

The components of the artificial diet for the aphid feeding were the same as described in Dadd and Mittler (1966). The apparatus used for the aphid artificial diet used a plastic tube with 1 cm diameter and 1 cm height. The artificial aphid diet, 100 μl with or without ledRNA, was enclosed between two layers of parafilm to create a diet sachet. On top of that sachet, there was a chamber for the aphids to move around and feed from the diet by piercing their stylets through the top layer of the stretched parafilm. Eight first- or second-instar nymphs were gently transferred to the aphid chamber using a fine paint brush. The experiment was carried out in a growth cabinet at 20° C.

The tobacco leaves used in one experiment were collected from plants grown in soil under 16 hr light/8 hr dark cycle at 22° C.

### MpC002 and MpRack-1 Genes and LedRNA Constructs

The green peach aphid MpC002 and MpRack-1 genes tested as target genes were the same as described by Pitino et al. (2011; 2012). The DNA sequences of both genes were obtained from the NCBI website, MpC002 (>MYZPE13164_0_v1.0_000024990.1|894 nt) and MpRack-1 (>MYZPE13164_0 v1.0_000198310.1|960 nt). The cDNA sequences of the two genes are provided herein as SEQ ID NOs: 123 and 124. LedRNA constructs were designed in the same manner as described in earlier Examples. The DNA sequences encoding the ledRNA molecules are provided herein as SEQ ID NOs:125 and 126 were used as transcription templates to synthesize the ledRNA. The vector DNAs encoding the ledRNA molecules targeting the MpC002 and MpRack-1 genes were introduced into E. coli strain DH5a for preparing plasmid DNA for in vitro RNA transcription and into E. coli strain HT115 for in vivo (in bacteria) transcription.

Efficacy of ledRNA Molecules on the Reduction of Aphid Performance

To examine if the ledRNAs targeting the MpC002 or MpRack-1 genes affected aphid performance, each ledRNA was delivered to the aphids through the artificial diet means as described in Example 1. In each experiment, ten biological replicates were set up; each biological replicate had eight one- to two-instar nymphs of green peach aphid. The controls in each experiment used equivalent concentrations of an unrelated ledRNA, namely ledGFP.

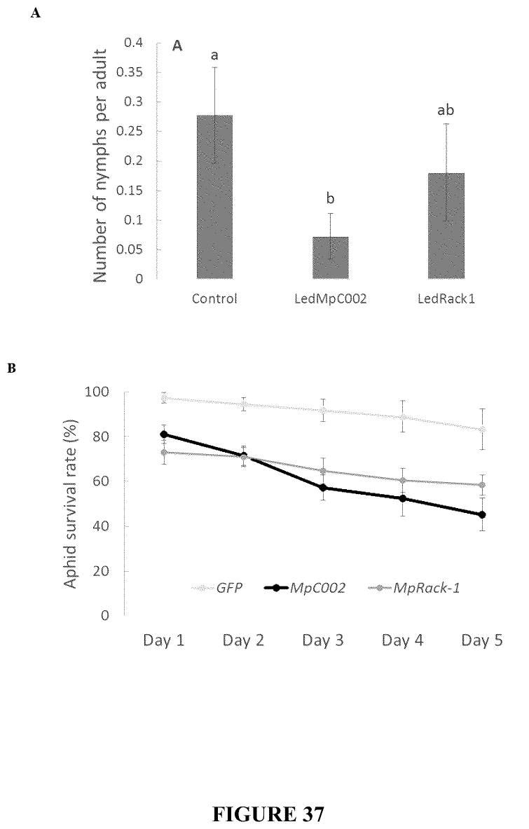

At a lower concentration of 50 ng/μl of each ledRNA molecule, aphid survival after feeding from the artificial diet containing either MpC002 or MpRack-1 ledRNA was not significantly different from the control ledGFP. However, the ledRNA targeting the MpC002 gene significantly (P<0.05) reduced the reproduction rate of green peach aphids (FIG. 37A). The average number of nymphs produced per adult aphid was reduced by about 75% compared to the number of nymphs produced from adults maintained on the control diet having the control ledRNA. At a higher concentration of 200 ng/μl, the ledRNAs targeting either MpC002 or MpRack-1 increased adult aphid mortality (FIG. 37B). The reduction of aphid survival on the diets including the MpC002 or MpRack-1 ledRNAs was also observed after 24 hours and continued over the five-day period of the experiment. The results indicated that use of the ledRNAs targeting the essential aphid genes was able to cause the death of aphids and reduce aphid reproduction. The efficacy of each ledRNA is compared to a hairpin RNA molecule (hpRNAi) targeting the same region of the target gene.

Uptake of ledRNA Molecules by Aphids

To track the uptake and distribution of the ledRNAs inside the aphids, the ledRNAs targeting the MpC002 or MpRack-1 genes were labelled with Cy3 (Cyanine-dye labelled nucleotide triphosphates) during the synthesis process as described in Example 1. The Cy3 labelling has been reported to have no effect on the biological function of conventional dsRNA molecules and so could be used as a label for detection by fluorescence. Aphids which had been fed the labelled ledRNAs were examined using confocal microscopy using a Leica EL 6000 microsystems instrument. The Cy3-labelled ledRNA targeting MpC002 or MpRack-1 was detectable in aphid guts within hours of feeding on the artificial diet and subsequently in the reproduction system and even in newborn nymphs which were the progeny of the adults that had been fed. The results indicated that aphid genes critical for digestive system function or reproduction could be effective targets for the ledRNA molecules through feeding.

### LedRNA Stability

To examine the stability of ledRNA in the diet and as recovered from the fed aphids, RNA was recovered from the artificial diet and from aphid honeydew after feeding on the diets containing the labelled ledRNA molecules. The RNA samples were electrophoresed on gels and examined by fluorescence detection. The ledMpC002 RNA prior to feeding clearly displayed a single product of about 700 bp on the agarose gel. The RNA recovered from the artificial diet showed a smear of RNA from 100-700 bp in size, indicating some degradation after being exposed to the diet at room temperature for 25 days, but still largely intact. RNA recovered from the aphid honeydew showed fluorescence in the RNA range from 350 to 700 bp, so again was largely intact. Despite the degradation of some ledRNA, a large proportion of the ledRNA molecules was able to stay intact in the artificial diet and also in the aphid honeydew for a considerable period of time. This degree of stability of the ledRNA molecules should allow the ledRNA to be active and retain activity when applied exogenously.

Absorbance of Labelled ledRNA by Plant Leaves

The Cy3-labelled ledMpC002 RNA was painted on the upper surface of tobacco leaves in order to see if it was able to penetrate the leaf tissues. Ten microliters of Cy3-labelled ledMpC002 (1 μg/μl concentration) was painted in a circle of 2 cm diameter and the applied region marked with a black marker pen. Images of leaf fluorescence at an excitation of 525 nm were captured over a five hour period using a Leica EL 6000 microsystems instrument, comparing the painted tissues with those not painted. The Cy3 label was clearly detectable in mesophyll tissue within one hour after application, so had clearly penetrated through the waxy cuticle layer on the leaf surface. The level of fluorescence increased at 2 hours and was maintained to the 5 hr time point. It was not clear if the ledRNA molecules got into the cells or into the nuclei of the cells. However, as sap-sucking insects feed specifically from the phloem sieve elements of plant leaves and stems, RNA transmission into the plant cells was not required for the silencing of aphid genes. The experiment indicated that the ledRNA molecules were found in the plant tissues through topical application.

### Conclusions

The aim of this study was to test the application of exogenous RNAi using the ledRNA design for the control of aphids, a major group of sap-sucking insect pests that are a problem throughout the world. Aphids are known to possess the RNAi machinery to process exogenous RNA (Scott et al., 2013; Yu et al., 2016). Here, oral delivery through an artificial diet containing ledRNA molecules targeting the MpC002 or MpRack-1 genes was able to cause aphid mortality and reduce the reproduction of the aphids. The molecules were tested against two different target genes, one encoding effector protein C002 and the other a receptor of activated protein kinase (Rack-1), which are essential for feeding and development of green peach aphid (Myzus persicae). When added to the artificial diet with a concentration as low as 50 ng/μl, the ledRNA molecules targeting these genes significantly reduced aphid reproduction. At a higher concentration of 200 ng/μl, the ledRNAs also increased aphid mortality. When ledRNA uptake was investigated using Cy3 labelling, ledRNA molecules were observed in aphid guts within hours of feeding on the artificial diet and subsequently in the reproduction system and even in newborn nymphs that were progeny of fed adults.

It was also shown that the ledRNA molecules stayed largely intact in the artificial diet for at least three and half weeks. Largely intact ledRNA molecules were also found in the aphid honeydew, an excretion product from the aphids. When labelled ledRNA was applied onto plant leaves, it could get into the phloem where the aphids feed. Together these results indicated the strong potential for ledRNA to be used for the control of aphids and other sap-sucking insects, including by exogenous delivery through the diet, providing a practical approach for management of aphids and other sap-sucking insects. These RNA molecules can also be expressed in transgenic plants, using promoters that favour synthesis of the RNA in phloem tissues, to control aphids and other sap-sucking insects.

### Example 20. RNA Constructs Targeting Other Insect Genes

### LedRNA Targeting Genes of Insect Pests

Helicoverpa armigera is an insect pest in the order Lepidoptera, also known as the cotton bollworm or corn earworm. The larvae of H. armigera feed on a wide range of plants including many important cultivated crops and cause considerable crop damage worth billions of dollars per year. The larvae are polyphagous and cosmopolitan pests which can feed on a wide range of plant species including cotton, maize, tomato, chickpea, pigeon pea, alfalfa, rice, sorghum and cowpea.

The H. armigera ABC transporter white gene (ABCwhite) was selected as a target gene with a readily detected phenotype to test ledRNA and ledRNA(G:U) constructs in an insect larva. ABC transporters belong to the ATP Binding Cassette transporter superfamily—for example, 54 different ABC transporter genes were identified in the Helicoverpa genome. ABC transporters encode membrane-bound proteins that carry any one or more of a wide range of molecules across membranes. The proteins use energy released by ATP hydrolysis to transport the molecules across the membrane. Some ABC transporters were implicated in the degradation of plant secondary metabolites in the cotton bollworm, H. armigera (Khan et al., 2017). The ABCwhite protein transports ommochrome and pteridine pathway precursors into pigment granules in the eye and knockout mutants exhibit white eyes.

The nucleotide sequence of the ABCwhite gene is provided as SEQ ID NO:127 (Accession No. KU754476). To test whether a ledRNA could reduce ABCwhite gene expression by exogenous delivery of the RNA in the larval diet, a genetic construct encoding a ledRNA was designed and made that targeted the gene. The genetic construct was made using the design principles for ledRNAs, with the split sequence being the sense sequence and the contiguous sequence being the antisense sequence (FIG. 1B). The target region was 603 nucleotides in length, corresponding to nucleotides 496-1097 of SEQ ID NO:127. The dsRNA region of the ledRNA was 603 bp in length; the antisense sequence in the dsRNA region was an uninterrupted, contiguous sequence corresponded to the complement of nucleotides 496-1097 of SEQ ID NO:127. The nucleotide sequences encoding the ledRNA is provided herein as SEQ ID NO: 128. The coding region was under the control of a T7 RNA polymerase promoter for in vitro transcription.

The genetic construct encoding the ledRNA was digested with the restriction enzyme SnaBI, which cleaved downstream of the ledRNA coding region, and transcribed in vitro with RNA polymerase T7 according to the instructions with the transcription kit. The RNA is added to an artificial diet and provided to H. armigera larvae.

A corresponding ledRNA construct having G:U basepairs in the double-stranded stem is made and compared to the canonically basepaired ledRNA.

### LedRNAs Targeting a Gene in Ants

Linepithema humile, commonly known as the Argentine ant, is an insect pest that has spread widely in several continents. The L. humile gene encoding pheromone biosynthesis activating neuropeptide (PBAN) neuropeptides-like (LOC105673224) was selected as a target gene, involved in communication between the insects by pheromones.

The nucleotide sequence of the PBAN gene is provided as SEQ ID NO: 129 (Accession No. XM_012368710). To test whether a ledRNA could reduce PBAN gene expression by exogenous delivery of the RNA in the diet in the form of a bait, a genetic construct encoding a ledRNA was designed and made that targeted the gene. The genetic construct was made using the design principles for ledRNAs, with the split sequence being the sense sequence and the contiguous sequence being the antisense sequence (FIG. 1B). The target region was 540 nucleotides in length, corresponding to nucleotides 136-675 of SEQ ID NO: 129. The dsRNA region of the ledRNA was 540 bp in length; the antisense sequence in the dsRNA region was an uninterrupted, contiguous sequence corresponded to the complement of nucleotides 136-675 of SEQ ID NO:129. The nucleotide sequences encoding the ledRNA is provided herein as SEQ ID NO:130. The coding region was under the control of a T7 RNA polymerase promoter for in vitro transcription.

The genetic construct encoding the ledRNA was digested with the restriction enzyme SnaBI, which cleaved downstream of the ledRNA coding region, and transcribed in vitro with RNA polymerase T7 according to the instructions with the transcription kit. The RNA is coated onto corn powder for oral delivery into L. humile ants.

LedRNA Targeting Genes of L. cuprina

Lucilia cuprina is an insect pest more commonly known as the Australian sheep blowfly. It belongs to the blowfly family, Calliphoridae, and is a member of the insect order Diptera. Five target genes were selected for testing with ledRNA constructs, namely genes encoding V-type proton ATPase catalytic subunit A (Accession No. XM_023443547), RNAse 1/2 (Accession No. XM_023448015), chitin synthase (Accession No. XM_023449557), ecdysone receptor (EcR; Accession No. U75355) and gamma-tubulin 1/1-like (Accession No. XM_023449717) of L. cuprina. Each of the genetic constructs was made using the design principles for ledRNAs, with the split sequence being the sense sequence and the contiguous sequence being the antisense sequence (FIG. 1B). In each case, the target region was about 600 nucleotides in length and the antisense sequence in the dsRNA region was an uninterrupted, contiguous sequence. The nucleotide sequence encoding the ledRNA targeting the ATPase-A gene is provided herein as SEQ ID NO: 131. The nucleotide sequence encoding the ledRNA targeting the RNAse 1/2 gene is provided herein as SEQ ID NO:132. The nucleotide sequence encoding the ledRNA targeting the chitin synthase gene is provided herein as SEQ ID NO:133. The nucleotide sequence encoding the ledRNA targeting the EcR gene is provided herein as SEQ ID NO:134. The nucleotide sequence encoding the ledRNA targeting the gamma-tubulin 1/1-like gene is provided herein as SEQ ID NO:135. In each construct, the coding region was under the control of a T7 RNA polymerase promoter for in vitro transcription.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The present application claims priority from AU 2017903773 filed 15 Sep. 2017, AU 2018902840 filed 3 Aug. 2018 and AU 2018902896 filed 8 Aug. 2018 the disclosures of which are incorporated herein by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Abraham (2002). Cell 111:9-12.
Acevedo-Garcia et al., (2017). Plant Biotechnology Journal 15:367-378.
Alvarez et al., (2000). Theor Appl Genet 100:319-327.
Baumlein et al., (1991). Mol. Gen. Genet. 225:459-467.
Baumlein et al., (1992). Plant J. 2:233-239.
Bhattacharyya et al., (1990) Cell 60:155-122.
Brar et al., (1996) Biotech Genet. Eng Rev 13:167-79.
Broothaerts et al., (2005). Nature 433:629-633.
Broun et al., (1998). Plant J. 13:201-210.
Busk et al., (1997). Plant J. 11:1285-1295.
Chen et al., (2005). Functional Plant Biology 32:671-681.
Chikwamba et al., (2003). Proc. Natl. Acad. Sci. U.S.A. 100:11127-11132.
Christou and Klee, Handbook of Plant Biotechnology, John Wiley and Sons (2004).
Chung et al., (2006). BMC Genomics 7:120.
Clough and Bent (1998). Plant J. 16:735-743.
Corrado and Karali (2009). Biotechnol. Adv. 27:733-743.
Courvalin et al., (1995). Life Sci. 318:1209-1212.
Dadd and Mittler (1966). Experientia 22:832-833.
Darji et al., (1997). Cell 91:765-775.
Dong et al., (2011) Plant J. 68:633-45.
Draper and Scott (1988). In: J. Draper et al., (Eds.), Plant Genetic Transformation and Gene Expression: A Laboratory Manual, Alden Press, Oxford, pp. 199-236.
Dunwell (2000). J Exp Botany 51Spec No:487-496.
Ellerstrom et al., (1996). Plant Mol. Biol. 32:1019-1027.
Elliott et al., (2002). Mol. Plant Microbe Interact. 15:1069-1077.
Ellis et al., (1987). EMBO J 6:11-16.
Fehr, In: Breeding Methods for Cultivar Development, Wilcox J. ed., American Society of Agronomy, Madison Wis. (1987).
Gan (1995). Molecular characterization and genetic manipulation of plant senescence. PhD thesis. University of Wisconsin, Madison.
Gan and Amasino (1995). Science 270:1986-1988.
Glawe (2008). Ann. Rev. Phytopathol. 46:27-51.
Gleave (1992). Plant Mol Biol 20: 1203-1207.
Gupta et al., (1988) Plant Mol. Biol. 10:215-224.
Helliwell and Waterhouse (2005). Methods in Enzymology 392:24-35.
Hinchee et al., (1988). Biotechnology 6:915-922.
Horvath et al., (2000). Proc. Natl. Acad. Sci. U.S.A. 97:1914-1919.
Hsieh and Fire (2000) Annu Rev Genet 14:187-204.
Jeddeloh, et al., (1999). Nat. Genet. 22:94-97.
Jefferson et al., (1987). EMBO J 6:3901-3907.
Jepson et al., (1994). Plant Mol. Biol. 26:1855-1866.
Khan (2017). Sci Rep 7, 40025
Kishore and Somerville (1993). Curr Opin Biotechnol. 4:152-158.
Koziel et al., (1996). Plant Mol. Biol. 32:393-405.
Lacroix et al., (2008). Proc. Natl. Acad. Sci. U.S.A. 105: 15429-15434.
Li et al., (1996). FEBS Lett. 379:117-121.
McCahill et al., (2002). Molecular Pharmacology 62:1261-1273.
McCullough and Schuler (1997). Nucl Acids Res. 25:1071-1077.
Mahfouz et al., (2006). Plant Cell 18:477-490.
Matsuoka et al., (1994). Plant J. 6:311-319.
Meier et al., (1997). FEBS Lett. 415:91-95.
Melamed-Bessudo et al., (2012) Proc. Natl. Acad. Sci. USA 109(16):e981-988.
Millar et al., (2006). Plant J. 45:942-954.
Mutti et al., (2006). J Insect Sci 6:38.
Mutti et al., (2008). Proceedings of the National Academy of Sciences 105:9965-9969.
Olive et al., (1989) Plant Mol Biol 12:525-538.
Padidam (2003). Transgenic Res. 12:101-109.
Perrin et al., (2000). Mol Breed 6:345-352.
Pitino et al., (2011). PLoS ONE 6, e25709.
Pitino and Hogenhout (2012). Molecular Plant-Microbe Interactions 26:130-139.
Potenza et al., (2004). In Vitro Cell Dev. Biol. Plant 40:1-22.
Powell et al., (1996). Vaccines 183, Abstract.
Preiss et al., (1987). In: Tailoring Genes for Crop Improvement (Bruening et al., eds.), Plenum Press, S. 133-152.
Sambrook et al., (1989). Molecular Cloning: A Laboratory Manual, Cold Spring Harbour Laboratory Press.
Schaffner (1980). Proc. Natl. Acad. Sci. U.S.A. 77:2163-2167.
Schwartz et al., (1997). Science 276:1872-1874.
Scott et al., (2013). Journal of Insect Physiology 59:1212-1221.
Seddas et al., (2004). Virology 325:399-412.
Shiina et al., (1997). Plant Physiol. 115:477-483.
Shure et al., (1983). Cell 35:225-233.
Sizemore et al., (1995). Science 270:299-302.
Slater et al., Plant Biotechnology—The Genetic Manipulation of Plants, Oxford University Press (2003).
Smith et al., (2000). Nature 407:319-320.
Stalker et al., (1988). Science 242: 419-423.
Stewart et al., (2000). J Mol Biol 298:611-622.
Tan et al., (2011). Plant Physiol. 156:1577-1588.
Thillet et al., (1988). J. Biol. Chem 263:12500-12508.
Timmons et al., (2001). Gen 263:103-112.
Ulmasov et al., (1995). Plant Physiol. 108:919-927.
Wang M B. Isolation of phloem specific gene promoters for use in genetic engineering of insect resistance in rice. PhD thesis, University of Durham, UK.
Wang et al., (1994) Plant Molecular Biology 24:159-170.
Wang et al., (1998) Acta Horticulturae 461:401-407.
Wang et al., (2008) RNA 14: 903-913. Wang et al., (2013). PLoS Genet 9, e1003865.
Wang et al., (2014). Nature Biotechnology 32:9.
Weiss (2003). Int. J. Med. Microbiol. 293:95:106.
Weissbach et al., (1988). In: Methods for Plant Molecular Biology, Academic Press, San Diego, Calif.
Wijnker et al., (2008). Trends in Plant Science 13:640-646.
Yang et al., Particle Bombardment Technology for Gene Transfer, Oxford Press, Oxford,
England (1994).
Yang et al., (2003). Planta 216:597-603.
Yelina et al., (2012). PLoS Genetics 8(8):e1002844. doi:10.1371/journal.pgen.1002844
Yu et al., (2016). Pest Management Science 72:1090-1098.
Zhang et al., (2018). Nat Rev Mol Cell Biol. 19:489-506.

1-96. (canceled)

97. A chimeric ribonucleic acid (RNA) molecule, comprising a double-stranded RNA (dsRNA) region which comprises a sense ribonucleotide sequence of at least 40 contiguous ribonucleotides in length and an antisense ribonucleotide sequence of at least 40 contiguous ribonucleotides in length which hybridise to each other to form the dsRNA region, wherein
i) the sense ribonucleotide sequence consists of, covalently linked in 5′ to 3′ order, a first 5′ ribonucleotide, a first RNA sequence and a first 3′ ribonucleotide,
ii) the antisense ribonucleotide sequence consists of, covalently linked in 5′ to 3′ order, a second 5′ ribonucleotide, a second RNA sequence and a second 3′ ribonucleotide, wherein at least 40 contiguous ribonucleotides of the first antisense ribonucleotide sequence are all capable of basepairing to nucleotides of a first region of a target RNA molecule in a plant, arthropod or fungal cell,
iii) the first 5′ ribonucleotide basepairs with the second 3′ ribonucleotide to form a terminal basepair of the dsRNA region,
iv) the second 5′ ribonucleotide basepairs with the first 3′ ribonucleotide to form a terminal basepair of the dsRNA region,
v) between about 5% and about 40% of the ribonucleotides of the sense ribonucleotide sequence and the antisense ribonucleotide sequence, in total, are either basepaired in a non-canonical basepair or are not basepaired,
vi) the dsRNA region does not comprise 20 contiguous canonical basepairs,
vii) the RNA molecule is capable of being processed in the plant, arthropod or fungal cell or in vitro whereby the antisense ribonucleotide sequence is cleaved to produce short antisense RNA (asRNA) molecules of 20-24 ribonucleotides in length,
viii) the RNA molecule or at least some of the asRNA molecules, or both, are capable of reducing the expression or activity of the target RNA molecule in the plant, arthropod or fungal cell, and
ix) the RNA molecule is capable of being made enzymatically by transcription in vitro or in a cell, or both.

98. A non-human organism comprising an RNA molecule according to claim 97.

99. A method of producing an RNA molecule, comprising expressing a polynucleotide encoding the RNA molecule of claim 97 in a host cell or a cell-free expression system.

100. A method for reducing the level and/or activity of a target RNA molecule in a plant, arthropod or fungus, the method comprising delivering to the plant, arthropod or fungus an RNA molecule according to claim 97.

101. A method of controlling a plant, arthropod or fungus, the method comprising delivering to the plant, arthropod or fungus an RNA molecule according to claim 97, wherein the RNA molecule has a deleterious effect on the plant, arthropod or fungus.

102. A method of preventing or reducing, or treating, a disease in a plant or arthropod, the method comprising administering to the plant or arthropod an RNA molecule according to claim 97, wherein the RNA molecule has an effect of preventing or reducing the disease in the plant or arthropod or has a beneficial effect on at least one symptom of the disease.

103. An RNA molecule comprising a first RNA component, a second RNA component which is covalently linked to the first RNA component and, optionally, one or more or all of (i) a linking ribonucleotide sequence which covalently links the first and second RNA components, (ii) a 5′ leader sequence and (iii) a 3′ trailer sequence,
wherein the first RNA component consists of, in 5′ to 3′ order, a first 5′ ribonucleotide, a first RNA sequence and a first 3′ ribonucleotide,
wherein the first RNA sequence comprises a first sense ribonucleotide sequence of at least 40 contiguous ribonucleotides, a first loop sequence of at least 4 ribonucleotides and a first antisense ribonucleotide sequence of at least 40 contiguous ribonucleotides, wherein the first antisense ribonucleotide sequence hybridises with the first sense ribonucleotide sequence in the RNA molecule to form a first double-stranded RNA (dsRNA) region,
wherein the first 5′ and 3′ ribonucleotides basepair with each other in the first RNA component to form a terminal basepair of the first dsRNA region,
wherein the first dsRNA region comprises one, or two or more, bulges, wherein each bulge is a ribonucleotide, or two or more contiguous ribonucleotides, in the first sense ribonucleotide sequence or the first antisense ribonucleotide sequence which is not basepaired in the first dsRNA region and which does not have a mismatched nucleotide at the corresponding position in the complementary sequence in the first dsRNA region, wherein at least 40 contiguous ribonucleotides of the first antisense ribonucleotide sequence are all capable of basepairing to ribonucleotides of a first region of a target RNA molecule in a plant, arthropod or fungal cell,
wherein the second RNA component is covalently linked, via the linking ribonucleotide sequence if present or directly if the linking ribonucleotide sequence is not present, to the first 5′ ribonucleotide or the first 3′ ribonucleotide,
wherein the second RNA component consists of, in 5′ to 3′ order, a second 5′ ribonucleotide, a second RNA sequence and a second 3′ ribonucleotide,
wherein the second RNA sequence comprises a second antisense ribonucleotide sequence of at least 21 contiguous ribonucleotides, a second loop sequence of at least 4 ribonucleotides and a second sense ribonucleotide sequence of at least 21 contiguous ribonucleotides,
wherein the second sense ribonucleotide sequence hybridises with the second antisense ribonucleotide sequence in the RNA molecule to form a second dsRNA region,
wherein the second 5′ and 3′ ribonucleotides basepair to each other in the RNA molecule to form a terminal basepair of the second dsRNA region,
wherein the 5′ leader sequence, if present, consists of a sequence of ribonucleotides which is covalently linked to the first 5′ ribonucleotide if the second RNA component is linked to the first 3′ ribonucleotide or to the second 5′ ribonucleotide if the second RNA component is linked to the first 5′ ribonucleotide,
wherein the 3′ trailer sequence, if present, consists of a sequence of ribonucleotides which is covalently linked to the second 3′ ribonucleotide if the second RNA component is linked to the first 3′ ribonucleotide or to the first 3′ ribonucleotide if the second RNA component is linked to the first 5′ ribonucleotide, and
wherein the RNA molecule is comprised of a single ribonucleotide strand which comprises a 5′ end and a 3′ end.

104. The RNA molecule of claim 103, which is characterized by one or more of the following:
(i) at least 20 contiguous ribonucleotides of the first sense ribonucleotide sequence are identical in sequence to a first region of the target RNA molecule,
(ii) the first sense ribonucleotide sequence is linked covalently to the first 5′ ribonucleotide without any intervening nucleotides, or the first antisense ribonucleotide sequence is linked covalently to the first 3′ ribonucleotide without any intervening nucleotides, or both,
(iii) the RNA molecule comprises the linking ribonucleotide sequence, wherein the linking ribonucleotide sequence is less than 20 ribonucleotides in length,
(iv) the linking ribonucleotide sequence hybridizes to the target RNA molecule,
(v) the linking ribonucleotide sequence is identical to a portion of the complement of the target RNA molecule,
(vi) the linking ribonucleotide sequence is between 1 and 10 ribonucleotides in length,
(vii) the at least 40 ribonucleotides of the first sense ribonucleotide sequence and the at least 21 ribonucleotides of the second sense ribonucleotide sequence are targeted to or identical in sequence to different regions of the same target RNA molecule,
(viii) the at least 40 ribonucleotides of the first sense ribonucleotide sequence and the at least 21 ribonucleotides of the second sense ribonucleotide sequence are targeted to or identical in sequence to regions of different target RNA molecules,
(ix) the first and second sense ribonucleotide sequences are covalently joined directly without an intervening ribonucleotide sequence,
(x) the first and second antisense ribonucleotide sequences are covalently joined directly without an intervening ribonucleotide sequence,
(xi) the at least 21 ribonucleotides of the second antisense ribonucleotide sequence and the at least 21 ribonucleotides of the second sense ribonucleotide sequence fully basepair within the second dsRNA region,
(xii) the at least 40 ribonucleotides of the first antisense ribonucleotide sequence and the at least 21 ribonucleotides of the second antisense ribonucleotide sequence are targeted to or complementary to different regions of the same target RNA molecule,
(xiii) the second antisense ribonucleotide sequence is targeted to or complementary to a region of a different target RNA molecule than the first antisense ribonucleotide sequence,
(xiv) either the first 5′ ribonucleotide and the second 3′ ribonucleotide are adjacent in the RNA molecule, each basepaired and not directly covalently bonded to each other, or the second 5′ ribonucleotide and the first 3′ ribonucleotide are adjacent in the RNA molecule, each base paired and not directly covalently bonded,
(xv) the first and second antisense ribonucleotide sequences are not contiguous in the RNA molecule but are separated by the linking ribonucleotide sequence,
(xvi) the first sense ribonucleotide sequence is at least 60% identical to a first region of the target RNA molecule, the second sense ribonucleotide sequence is at least 60% identical to a second region of the target RNA molecule, the second region of the target RNA molecule being different to the first region of the target RNA molecule, and wherein the first and second antisense ribonucleotide sequences are either directly covalently linked in the RNA molecule or are joined through the linking ribonucleotide sequence,
(xvii) the RNA molecule has the 5′ leader sequence or the 3′ trailer sequence, or both,
(xviii) at least one of the loop sequences in the RNA molecule, or both the first and second loop sequences, are longer than 20 nucleotides,
(xix) the RNA molecule has three, four or more loops,
(xx) the RNA molecule has only two loops, and
(xxi) at least one of the loops, or all of the loops, are between 4 and 1,000 ribonucleotides, or between 4 and 200 ribonucleotides, or between 4 and 50 ribonucleotides, in length.

105. The RNA molecule of claim 103, wherein the RNA molecule is produced in a cell.

106. The RNA molecule of claim 105 which is produced in a yeast cell.

107. The RNA molecule of claim 105, wherein the cell in which the RNA molecule is produced is different to the plant cell, arthropod cell or fungal cell comprising the target RNA molecule.

108. The RNA molecule of claim 103, wherein
(i) between 5% and 40% of the ribonucleotides of the first sense ribonucleotide sequence and the first antisense ribonucleotide sequence, in total, are basepaired in non-canonical basepairs when the first sense ribonucleotide sequence and the first antisense ribonucleotide sequence hybridize, or
(ii) between 5% and 40% of the ribonucleotides of the second sense ribonucleotide sequence and the second antisense ribonucleotide sequence, in total, are basepaired in non-canonical basepairs when the second sense ribonucleotide sequence and the second antisense ribonucleotide sequence hybridize,
or both (i) and (ii).

109. The RNA molecule of claim 108, which is characterized by one or more of the following:
(i) at least one of the dsRNA regions in the RNA molecule does not comprise 20 contiguous canonical basepairs,
(ii) the RNA molecule is processed in the plant, arthropod or fungal cell, whereby the first or second antisense ribonucleotide sequence, or both, is cleaved to produce short antisense RNA (asRNA) molecules of 20-24 ribonucleotides in length,
(iii) the RNA molecule or at least some of the asRNA molecules, or both, reduce the expression or activity of the target RNA molecule in the plant, arthropod or fungal cell,
(iv) the RNA molecule is made enzymatically by transcription in vitro,
(v) between 15% and 30%, or between 16% and 25%, of the ribonucleotides of (a) the first sense ribonucleotide sequence and the first antisense ribonucleotide sequence, in total, or (b) the ribonucleotides of the second sense ribonucleotide sequence and the second antisense ribonucleotide sequence, in total, or both (a) and (b), are basepaired in non-canonical basepairs when the sense and antisense ribonucleotide sequences hybridize,
(vi) the non-canonical basepairs are G:U basepairs,
(vii) at least 75%, at least 80%, at least 85%, at least 90%, at least 95%, at least 99%, or 100%, of the ribonucleotides, in total, in the dsRNA region formed by the second sense ribonucleotide sequence and the second antisense ribonucleotide sequence are basepaired,
(viii) either (a) every one in four to every one in six ribonucleotides in the first sense ribonucleotide sequence forms a non-canonical basepair with a ribonucleotide in the first antisense ribonucleotide sequence, or (b) every one in four to every one in six ribonucleotides in the second sense ribonucleotide sequence forms a non-canonical basepair with a ribonucleotide in the second antisense ribonucleotide sequence or is not basepaired, or both (a) and (b),
(ix) either (a) the first dsRNA region does not comprise 8 contiguous canonical basepairs, or (b) the second dsRNA region does not comprise 8 contiguous canonical basepairs, or both (a) and (b),
(x) either (a) the first dsRNA region, or (b) the second dsRNA region, or both (a) and (b), comprises at least 8 contiguous canonical basepairs,
(xi) all of the ribonucleotides in the second dsRNA region are base-paired when the sense and antisense ribonucleotide sequences hybridize, wherein each basepair is independently a canonical basepair or a non-canonical basepair,
(xii) one or more ribonucleotides of the second sense ribonucleotide sequence or one or more ribonucleotides of the second antisense ribonucleotide sequence, or both, are not basepaired in the second dsRNA region,
(xiii) the first or second antisense ribonucleotide sequence, or both, are independently less than 100% identical, or between 80% and 99.9% identical, or between 90% and 98% identical, or between 95% and 98% identical, in sequence to the complement of the first or second target region of the target RNA molecule, respectively,
(xiv) the first or second antisense ribonucleotide sequence, or both, are 100% identical in sequence to the complement of the first or second target region of the target RNA molecule, respectively,
(xv) the first sense ribonucleotide sequence and the first antisense ribonucleotide sequence, and/or the second sense ribonucleotide sequence and the second antisense ribonucleotide sequence, are each at least 50, at least 100, or 100 to 1,000, ribonucleotides in length,
(xvi) either (a) the number of ribonucleotides in the first sense ribonucleotide sequence is between 90% and 110% of the number of ribonucleotides in the first antisense ribonucleotide sequence, or (b) the number of ribonucleotides in the second sense ribonucleotide sequence is between 90% and 110% of the number of ribonucleotides in the second antisense ribonucleotide sequence, or both (a) and (b), and
(xvii) either (a) the number of ribonucleotides in the first sense ribonucleotide sequence is the same as the number of ribonucleotides in the first antisense ribonucleotide sequence, or (b) the number of ribonucleotides in the second sense ribonucleotide sequence is the same as the number of ribonucleotides in the second antisense ribonucleotide sequence, or both (a) and (b).

110. An isolated and/or exogenous polynucleotide, or a vector comprising the polynucleotide, encoding an RNA molecule according to claim 103, wherein the polynucleotide is operably linked to a promoter capable of directing expression of the RNA molecule in a host cell.

111. A cell, or a plant or arthropod or a part thereof, comprising (a) the RNA molecule of claim 103, or (b) an exogenous polynucleotide or a vector encoding the RNA molecule, or both (a) and (b).

112. A method of producing the RNA molecule of claim 103, the method comprising expressing a polynucleotide encoding the RNA molecule in a host cell or a cell-free expression system.

113. A method of producing a transgenic cell or plant, the method comprising introducing an exogenous polynucleotide encoding the RNA molecule of claim 103 into a cell so that the exogenous polynucleotide is stably integrated into the genome of the cell, and optionally generating a plant from the cell.

114. A method of producing a cell, or an organism which is a plant or arthropod, or part thereof, comprising (a) the RNA molecule of claim 103, or (b) an exogenous polynucleotide or a vector encoding the RNA molecule, or both (a) and (b), the method comprising contacting the cell, or plant or arthropod or part thereof, with the RNA molecule, exogenous polynucleotide or vector.

115. The method of claim 114, wherein the organism is an insect.

116. An extract of the cell or plant or arthropod or part thereof of claim 111, wherein the extract comprises the RNA molecule and/or an exogenous polynucleotide or vector encoding the RNA molecule.

117. A composition comprising the RNA molecule of claim 103 and one or more suitable carriers, wherein the suitable carrier is a substance which enhances the stability of the RNA molecule and/or which assists in the RNA molecule being taken up by a cell of an organism.

118. A method of reducing the level and/or activity of a target RNA molecule in a plant, arthropod or fungal cell, the method comprising delivering to the plant, arthropod or fungal cell the RNA molecule of claim 103 and/or an exogenous polynucleotide or vector encoding the RNA molecule.

119. An RNA molecule comprising a first RNA component, a second RNA component which is covalently linked to the first RNA component and, optionally, one or more or all of (i) a linking ribonucleotide sequence which covalently links the first and second RNA components, (ii) a 5′ leader sequence and (iii) a 3′ trailer sequence,
wherein the first RNA component consists of, in 5′ to 3′ order, a first 5′ ribonucleotide, a first RNA sequence and a first 3′ ribonucleotide,
wherein the first RNA sequence comprises a first sense ribonucleotide sequence of at least 40 contiguous ribonucleotides, a first loop sequence of at least 4 ribonucleotides and a first antisense ribonucleotide sequence of at least 40 contiguous ribonucleotides, wherein the first antisense ribonucleotide sequence hybridises with the first sense ribonucleotide sequence to form a first double-stranded RNA (dsRNA) region,
wherein the first 5′ and 3′ ribonucleotides basepair with each other in the first RNA component to form a terminal basepair of the first dsRNA region,
wherein at least 40 contiguous ribonucleotides of the first antisense ribonucleotide sequence are all capable of basepairing to ribonucleotides of a first region of a target RNA molecule in a plant, arthropod or fungal cell,
wherein the second RNA component is covalently linked, via the linking ribonucleotide sequence if present or directly if the linking ribonucleotide sequence is not present, to the first 5′ ribonucleotide or the first 3′ ribonucleotide,
wherein the second RNA component consists of, in 5′ to 3′ order, a second 5′ ribonucleotide, a second RNA sequence and a second 3′ ribonucleotide,
wherein the second RNA sequence comprises a second antisense ribonucleotide sequence of at least 21 contiguous ribonucleotides, a second loop sequence of at least 4 ribonucleotides and a second sense ribonucleotide sequence of at least 21 contiguous ribonucleotides,
wherein the second sense ribonucleotide sequence hybridises with the second antisense ribonucleotide sequence in the RNA molecule to form a second dsRNA region,
wherein the second 5′ and 3′ ribonucleotides basepair to each other in the RNA molecule to form a terminal basepair of the second dsRNA region,
wherein either (i) the first and second antisense ribonucleotide sequences flank the first and second sense ribonucleotide sequences such that the first and second sense ribonucleotide sequences are between the first and the second antisense ribonucleotide sequences, or (ii) the first and second sense ribonucleotide sequences flank the first and second antisense ribonucleotide sequences such that the first and second antisense ribonucleotide sequences are between the first and the second sense ribonucleotide sequences,
wherein the 5′ leader sequence, if present, consists of a sequence of ribonucleotides which is covalently linked to the first 5′ ribonucleotide if the second RNA component is linked to the first 3′ ribonucleotide or to the second 5′ ribonucleotide if the second RNA component is linked to the first 5′ ribonucleotide,
wherein the 3′ trailer sequence, if present, consists of a sequence of ribonucleotides which is covalently linked to the second 3′ ribonucleotide if the second RNA component is linked to the first 3′ ribonucleotide or to the first 3′ ribonucleotide if the second RNA component is linked to the first 5′ ribonucleotide, and
wherein the RNA molecule is comprised of a single ribonucleotide strand which comprises a 5′ end and a 3′ end.

120. An isolated and/or exogenous polynucleotide, or a vector comprising the polynucleotide, encoding an RNA molecule according to claim 119, wherein the polynucleotide is operably linked to a promoter capable of directing expression of the RNA molecule in a host cell.

121. A cell, or a plant or arthropod or a part thereof, comprising (a) the RNA molecule of claim 119, or (b) an exogenous polynucleotide or a vector encoding the RNA molecule, or both (a) and (b).

122. A method of producing the RNA molecule of claim 119, the method comprising expressing a polynucleotide encoding the RNA molecule in a host cell or a cell-free expression system.

123. A method of reducing the level and/or activity of a target RNA molecule in a plant, arthropod or fungal cell, the method comprising delivering to the plant, arthropod or fungal cell the RNA molecule of claim 119 and/or an exogenous polynucleotide or vector encoding the RNA molecule.
